SEQUENCE LISTING (17 sequences)

SEQ ID NO: 1 - protein, 7 aa, Simian virus 40
PKKKRKV

SEQ ID NO: 2 - protein, 16 aa, Unknown
  Description of Unknown Nucleoplasmin bipartite NLS sequence
KRPAATKKAGQAKKKK

SEQ ID NO: 3 - protein, 9 aa, Unknown
  Description of Unknown C-myc NLS sequence
PAAKRVKLD

SEQ ID NO: 4 - protein, 11 aa, Unknown
  Description of Unknown C-myc NLS sequence
RQRRNELKRSP

SEQ ID NO: 5 - protein, 38 aa, Homo sapiens
NQSSNFGPMKGGNFGGRSSGPYGGGGQYFAKPRNQGGY

SEQ ID NO: 6 - protein, 42 aa, Unknown
  Description of Unknown IBB domain from importin-alpha sequence
RMRIZFKNKGKDTAELRRRRVEVSVELRKAKKDEQILKRRNV

SEQ ID NO: 7 - protein, 8 aa, Unknown
  Description of Unknown Myoma T protein sequence
VSRKRPRP

SEQ ID NO: 8 - protein, 8 aa, Unknown
  Description of Unknown Myoma T protein sequence
PPKKARED

SEQ ID NO: 9 - protein, 8 aa, Homo sapiens
PQPKKKPL

SEQ ID NO: 10 - protein, 12 aa, Mus musculus
SALIKKKKKMAP

SEQ ID NO: 11 - protein, 5 aa, Influenza virus
DRLRR

SEQ ID NO: 12 - protein, 7 aa, Influenza virus
PKQKKRK

SEQ ID NO: 13 - protein, 10 aa, Hepatitis delta virus
RKLKKKIKKL

SEQ ID NO: 14 - protein, 10 aa, Mus musculus
REKKKFLKRR

SEQ ID NO: 15 - protein, 20 aa, Homo sapiens
KRKGDEVDGVDEVAKKKSKK

SEQ ID NO: 16 - protein, 17 aa, Homo sapiens
RKCLQAGMNLEARKTKK

SEQ ID NO: 17 - protein, 17 aa, Poliovirus
  Type 2
TVDNDAPTKRASKLFAV

(19) United States
(12) Patent Application Publication
(10) Pub. No.: US 2024/0058454 A1
(43) Pub. Date: Feb. 22, 2024

(54) BIOLOGICAL DELIVERY SYSTEMS

(71) Applicant: PARTICELLA, INC., San Francisco, CA (US)

(72) Inventors: Mubhij Ahmad, Walnut Creek, CA (US); Timothy Day, Oakland, CA (US); Ismail Hafez, Concord, CA (US); John Merritt, San Mateo, CA (US)

(21) Appl. No.: 18/267,206

(22) PCT Filed: Dec. 14, 2021
(86) PCT No.: PCT/US2021/063182

§ 371 Date: Jun. 14, 2023

Related U.S. Application Data
(60) Provisional application No. 63/125,075, filed on Dec. 14, 2020.
(60) Provisional application No. 63/194,315, filed on May 28, 2021.
(60) Provisional application No. 63/282,421, filed on Nov. 23, 2021.

(30) Foreign Application Priority Data
Jun. 11, 2021 (WO) PCT/US2021/037011

(51) Int. Cl. A61K 47/54; A61K 47/28; A61K 47/10; A61K 47/64; A61K 47/69

(52) U.S. Cl. CPC A61K 47/544; A61K 47/28; A61K 47/10; A61K 47/543; A61K 47/64; A61K 47/6929

(57) ABSTRACT

Provided are delivery vehicles, and methods of making and using same for reaching epithelial cells, such as cells within mucus-containing environments, and delivery vehicles with improved stability in harsh environments, including the gastrointestinal tract.

### CROSS REFERENCE TO RELATED APPLICATIONS

This application claims priority to U.S. Provisional Application No. 63/125,075 filed on Dec. 14, 2020 entitled COMPOSITIONS AND METHODS FOR BIOLOGICAL DELIVERY VEHICLES, U.S. Provisional Application No. 63/194,315 filed on May 28, 2021 entitled COMPOSITIONS AND METHODS FOR BIOLOGICAL DELIVERY VEHICLES, International Patent Application Number PCT/US2021/037011 filed on Jun. 11, 2021 entitled COMPOSITIONS AND METHODS FOR BIOLOGICAL DELIVERY VEHICLES, and U.S. Provisional Application No. 63/282,421 filed on Nov. 23, 2021 entitled BIOLOGICAL DELIVERY SYSTEMS the contents of each of which are herein incorporated by reference in their entirety.

### STATEMENT AS TO FEDERALLY SPONSORED RESEARCH

This invention was made with the support of the United States government under Contract number 1846078 by the National Science Foundation. The government has certain rights in the invention.

### SEQUENCE LISTING

The present application is being filed along with a Sequence Listing in electronic format. The Sequence Listing file, entitled 2214_1006PCT_SL.txt, was created on Dec. 13, 2021 and is 4,060 bytes in size. The information in electronic format of the Sequence Listing is incorporated herein by reference in its entirety.

### BACKGROUND

Despite advances in gene therapy over the last 50 years, there remain many diseases that are recalcitrant to conventional methods, particularly in cases where a target location for gene therapy may provide challenges for delivery, such as in the gastrointestinal tract. The present disclosure addresses this need and provides a number of advantages as well.

### SUMMARY

The following description and examples illustrate embodiments of the disclosure in detail. It is to be understood that this disclosure is not limited to the particular embodiments described herein and as such can vary. Those of skill in the art will recognize that there are numerous variations and modifications of the disclosure, which are encompassed within its scope.

In some embodiments, the present disclosure provides a delivery vehicle comprising: at least one bile salt, at least one bile acid, or a combination thereof, at least one cationic lipid; at least one structural lipid; and optionally at least one conjugated lipid.

In some embodiments, the at least one bile salt comprises sulfobromophthalein disodium salt hydrate, tauro-3β;5α,6β-trihydroxycholanoic acid, taurochenodeoxycholic acid sodium salt, taurocholic acid sodium salt hydrate, taurocholic acid sodium salt, taurodehydrocholic acid sodium salt, taurodeoxycholic acid sodium salt, taurohyodeoxycholate, taurohyodeoxycholic acid sodium salt, taurolithocholic acid 3-sulfate disodium salt, taurolithocholic acid sodium salt, tauro-β-muricholic acid sodium salt, tauroursodeoxycholic acid sodium salt, tauro-α-muricholic acid sodium salt, tauro-γ-muricholic acid sodium salt, tauro-ω-muricholic acid sodium salt, β-Estradiol 17-(β-D-glucuronide) sodium salt, lithocholic acid 3-sulfate (disodium salt), chenodeoxycholic acid 3-sulfate (disodium salt), chenodeoxycholic acid 7-sulfate (disodium salt), cholic acid 3-sulfate (disodium salt), cholic acid 7-sulfate (disodium salt), cholic acid sodium salt, deoxycholic acid 3-sulfate (disodium salt), deoxycholic acid disulfate (trisodium salt), phenoxymethylpenicillinic acid potassium salt, chenodeoxycholic acid disulfate (trisodium salt), chenodeoxycholic acid sodium salt, cholate, methyl cholate, sodium taurocholate hydrate, 1-naphthyl isothiocyanate, deoxycholate, hyodeoxycholate, glycocholate, sodium glycochenodeoxycholate, sodium cholate hydrate, taurocholate, taurodeoxycholate, taurochenodeoxycholate, chenodeoxycholate, lithocholate, isolithocholate, alloisolithocholate, sodium deoxycholate, sodium deoxycholate monohydrate, dehydrolithochlate, sodium glycodeoxycholate, sodium glycocholate hydrate, sodium taurodeoxycholate hydrate, sodium chenodeoxycholate, glycolithocholate sulfate, glycolithocholate, sodium taurohyodeoxycholate hydrate, sodium taurocholate, sodium tauroursodeoxycholate, sodium taurolithocholate, glycodeoxycholate, and any combination thereof.

In some embodiments, the at least one bile salt comprises cholate deoxycholate, chenodeoxycholate, lithocholate, and any combination thereof.

In some embodiments, the at least one bile acid comprises 3β, 5α, 6β-trihydroxycholanoic acid, 12-ketochenodeoxycholic acid, 12-ketodeoxycholic acid, 12-ketolithocholic acid, 3-oxo chenodeoxycholic acid, 3-oxo deoxycholic acid, 3-oxocholic acid, 3α, 6β, 7α, 12α-tetrahydroxy bile acid, 3α,6α,7α,12α-tetrahydroxy bile acid, 4-bromobenzoic acid, 6,7-diketolithocholic acid, 7-ketodeoxycholic acid, 7-ketolithocholic acid, allocholic acid, alloisolithocholic acid, apocholic acid, apocholic acid (delta 14 isomer), arachidyl amido cholanoic acid, chenodeoxycholic acid, chenodeoxycholic acid-d4, cholic acid, dehydrocholic acid, dehydrolithocholic acid, deoxycholic acid, dioxolithocholic acid, glyco-12-oxolithocholanoic acid, glycochenodeoxycholic acid, glycocholic acid, glycocholic acid hydrate, glycodehydrocholic acid, glycodeoxycholic acid, glycohyodeoxycholic acid, glycolithocholic acid, glycoursodeoxycholic acid, glyco-γ-muricholic acid, hyocholic acid, hyodeoxycholic acid, isodeoxycholic acid, isolithocholic acid, lithocholic acid, murideoxycholic acid, nordeoxycholic acid, obeticholic acid, pentadecanoic acid, ursocholic acid, ursodeoxycholic acid, ursodeoxycholic acid-D4, α-muricholic acid, β-muricholic acid, ω-muricholic acid, and any combination thereof.

In some embodiments, the at least one bile acid comprises ursodiol, 5beta-cholanic acid, 3-oxy-cholenic acid, and any combination thereof.

In some embodiments, the delivery vehicle comprises about 5 to about 40 mole % of the at least one bile salt or the at least one bile acid. In some embodiments, the delivery vehicle comprises about 20 to about 40 mole % of the at least one bile salt or the at least one bile acid. In some embodiments, the delivery vehicle comprises about 30 to about 40 mole % of the at least one bile salt or the at least one bile acid. In some embodiments, the at least one bile salt comprises deoxycholate.

In some embodiments, the at least one bile salt comprises chenodeoxycholate. In some embodiments, the at least one bile salt comprises lithocholate. In some embodiments, the at least one bile alloisolithocholate. In some embodiments, the at least one bile comprises dehydrolithocholate. In some embodiments, the at least one bile acid comprises ursodiol.

In some embodiments, the at least one bile salt comprises isolithocholate. In some embodiments, the at least one bile salt comprises dehydrolithochlate. In some embodiments, the at least one bile acid comprises 5-beta-cholanic acid. In some embodiments, the at least one bile salt comprises taurodeoxycholate. In some embodiments, the at least one bile comprises taurochenodeoxycholate. In some embodiments, the at least one bile salt glycocholate. In some embodiments, the at least one bile acid comprises 3-oxy-cholenic acid. In some embodiments, the delivery vehicle comprises deoxycholate and lithocholate.

In some embodiments, the delivery vehicle comprises about 20 to about 30 mole % deoxycholate and from about 5 to about 10 mole % of lithocholate. In some embodiments, the delivery vehicle comprises at least one bile salt and at least one bile acid.

In some embodiments, the at least one cationic lipid comprises NT-[2-((1 S)-1-[(3-aminopropyl)amino]-4-[di(3-amino-propyl)amino]butylcarboxamido)ethyl]-3,4-di[oleyloxy]-benzamide (MVL5), N4-Cholesteryl-Spermine HCl (GL67), 1,2-dioleyloxy-3-dimethylaminopropane (DODMA), N-[1-(2,3-dioleyloxy)propyl]-N,N,N-trimethylammonium chloride (DOTMA), [1,2-bis(oleoyloxy)-3-(trimethylammonio)propane](DOTAP), dimethyldioctadecylammonium (DDA), 30[N-(N′,N′-dimethylaminoethane)-carbamoyl] cholesterol (DC-Chol), and dioctadecylamidoglycylspermine (DOGS), 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 1,2-Dialkyloxy-N,N-dimethylaminopropane, 4-(2,2-diocta-9,12-dienyl-[1,3]dioxolan-4-ylmethyl)-dimethylamine, O-alkyl ethylphosphocholines, (6Z,9Z,28Z,31Z)-heptatriaconta-6,9,28,31-tetraen-19-yl 3-(dimethylamino)propanoate (MC2), 3β-[N-(N′,N′-dimethylaminoethane)-carbamoyl]cholesterol, N4-Cholesteryl-Spermine, 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 7-(4-(dimethylamino)butyl)-7-hydroxytridecane-1,13-diyl dioleate (CL1H6), 7-(4-(diisopropylamino)butyl)-7-hydroxytride-cane-1,13-diyl dioleate (CL4H6), 1,2-stearoyl-3-trimethylammonium-propane (DSTAP), 1,2-dipalmitoyl-3-trimethylammonium-propane (DPTAP), 1,2-Distearoyl-3-Dimethylammonium-Propane (DSDAP), or any combinations thereof. In some embodiments, the saturated cationic lipid can comprise at least one of 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, and any combination thereof.

In some embodiments, the at least one cationic lipid comprises MVL5; MC2; CL1H6; CL4H6; DODMA, and any combination thereof.

In some embodiments, the delivery vehicle comprises about 5 to about 90 mole % of the at least one cationic lipid. In some embodiments, the delivery vehicle comprises about 5 to about 60 mole % of the at least one cationic lipid. In some embodiments, the delivery vehicle comprises about 10 to about 60 mole % of the at least one cationic lipid. In some embodiments, the delivery vehicle comprises about 10 to about 50 mole % of the at least one cationic lipid. In some embodiments, the delivery vehicle comprises about 10 to about 30 mole % of the at least one cationic lipid.

In some embodiments, the at least one cationic lipid comprises at least one multivalent cationic lipid and at least one ionizable cationic lipid.

In some embodiments, the at least one multivalent cationic lipid comprises MVL5.

In some embodiments, the delivery vehicle comprises about 5 to about 90 mole % of the at least one multivalent cationic lipid. In some embodiments, the delivery vehicle comprises about 5 to about 60 mole % of the at least one multivalent cationic lipid. In some embodiments, the delivery vehicle comprises about 5 to about 30 mole % of the at least one multivalent cationic lipid. In some embodiments, the delivery vehicle comprises about 5 to about 15 mole % of the at least one multivalent cationic lipid. In some embodiments, the at least one multivalent cationic lipid comprises about up to about 100 mole % of the at least one cationic lipid.

In some embodiments, the at least one multivalent cationic lipid comprises about 5-75 mole % of the at least one cationic lipid. In some embodiments, the at least one multivalent cationic lipid comprises about 40-60 mole % of the at least one cationic lipid. In some embodiments, the at least one multivalent cationic lipid comprises about 50 mole % of the at least one cationic lipid.

In some embodiments, the at least one ionizable cationic lipid comprises at least one of MC2, CL1H6, CL4H6, DODMA, and any combination thereof.

In some embodiments, the at least one ionizable cationic lipid comprises MC2. In some embodiments, the at least one ionizable cationic lipid comprises CL1H6. In some embodiments, the at least one ionizable cationic lipid comprises CL4H6. In some embodiments, the at least one ionizable cationic lipid comprises DODMA.

In some embodiments, the delivery vehicle comprises about 5 to about 90 mole % of the at least one ionizable cationic lipid. In some embodiments, the delivery vehicle comprises about 5 to about 60 mole % of the at least one ionizable cationic lipid. In some embodiments, the delivery vehicle comprises about 5 to about 30 mole % of the at least one ionizable cationic lipid. In some embodiments, the delivery vehicle comprises about 5 to about 15 mole % of the at least one ionizable cationic lipid. In some embodiments, the ionizable cationic lipid comprises up to about 100 mole % of the at least one cationic lipid.

In some embodiments, the ionizable cationic lipid comprises about 5-75 mole % of the at least one cationic lipid. In some embodiments, the ionizable cationic lipid comprises about 40-60 mole % of the at least one cationic lipid. In some embodiments, the ionizable cationic lipid comprises about 50 mole % of the at least one cationic lipid. In some embodiments, the delivery vehicle comprises about the same amount of the at least one multivalent cationic lipid and the at least one ionizable cationic lipid.

In some embodiments, the at least one structural lipid comprises at least one neutral lipid, at least one anionic lipid, at least one phospholipid, and any combination thereof.

In some embodiments, the at least one structural lipid is comprises glycerol monooleate (GMO), dioleoylphosphatidylethanolamine (DOPE), 1,2-dimyristoyl-sn-glycero-3-phosphocholine (DMPC), short-chainbis-n-heptadecanoylphosphatidylcholine (DHPC), dihexadecoylphosphoethanolamine (DHPE), 1,2-dilinoleoyl-sn-glycero-3-phosphocholine (DLPC), dimyristoylphosphoethanolamine (DMPE), dimyristoylphosphatidylglycerol (DMPG), dioleoylphosphatidylcholine (DOPC), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylcholine (DPPC), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoylphosphoethanolamineimidazole (DSPEI), 1,2-diundecanoyl-sn-glycero-phosphocholine (DUPC), eggphosphatidylcholine (EPC), hydrogenatedsoybeanphosphatidylcholine (HSPC), mannosializeddipalmitoylphosphatidylethanolamine (ManDOG), 1,2-Dioleoyl-sn-Glycero-3-Phosphoethanolamine-N-[4-(p-maleimidomethyl)cyclohexane-carboxamide] (MCC-PE), 1,2-diphytanoyl-sn-glycero-3-phosphoethanolamine (ME 16.0 PE), 1-myristoyl-2-hydroxy-sn-glycero-phosphocholine (MHPC), 1-oleoyl-2-cholesterylhemisuccinoyl-sn-glycero-3-phosphocholine (OChemsPC), phosphatidicacid (PA), phosphatidylethanolaminelipid (PE), phosphatidylglycerol (PG), partiallyhydrogenatedsoyphosphatidylchloline (PHSPC), phosphatidylinositollipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), 18-1-transPE,1-stearoyl-2-oleoyl-phosphatidyethanolamine (SOPE), soybeanphosphatidylcholine (SPC), 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, and any combination thereof.

In some embodiments, the at least one structural lipid comprises DSPC, DMPC, DOPE, GMO, and any combination thereof.

In some embodiments, the delivery vehicle comprises from about 5 to about 75 mole % of the at least one structural lipid. In some embodiments, the delivery vehicle comprises from about 30 to about 50 mole % of the at least one structural lipid. In some embodiments, the delivery vehicle comprises from about 35 to about 45 mole % of the at least one structural lipid.

In some embodiments, the delivery vehicle does not comprise cholesterol.

In some embodiments, the at least one conjugated lipid comprises at least one conjugated lipid and at least one hydrophilic polymer. In some embodiments, the at least one hydrophilic polymer comprises polyethylene glycol (PEG). In some embodiments, the at least one conjugated lipid comprises at least one phospholipid, at least one neutral lipid, at least one glyceride, at least one diglyceride, at least one anionic lipid, at least one cationic lipid, and any combination thereof.

In some embodiments, the at least one conjugated lipid comprises 1,2-dimyristoyl-rac-glycerol (DMG), 1,2-dimyristoyl-sn-glycero-3-phosphoethanolamine (DMPE), 1,2-distearoyl-rac-glycerol (DSG), 1,2-dipalmitoyl-rac-glycerol (DPG), 1,2-distearoyl-sn-glycero-3-phosphoethanolamine (DSPE), diacylglycerol (DAG), 1,2-dipalmitoryl-sn-glycero-3-phosphoethanolamine (DPPE), and any combination thereof.

In some embodiments, the at least one conjugated lipid comprises at least one of DMG-PEG, DMPE-PEG, DSG-PEG, DPG-PEG, DSPE-PEG, DAG-PEG, DPPE-PEG, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSG-PEGMA, DSG-PEG5000, DPPE-PEG-2K, DPPE-mPEG2000, DPPE-mPEG, DPG-PEGMA, DOPE-PEG2000, DMPE-PEGMA, DMPE-PEG2000, DMPE-mPEG2000, DMG-PEGMA, DMG-PEG2000, C18PEG750, CI8PEG5000, CI8PEG3000, CI8PEG2000, CI6PEG2000, CI4PEG2000, C18-PEG5000, C18PEG, C16PEG, C14-PEG-DSPE200, C14-PEG2000, C14PEG2000, C14-PEG 2000, C14-PEG, C14PEG, (PEG)-C-DOMG, PEG-C-DMA and any combination thereof.

In some embodiments, the at least one conjugated lipid comprises DMG-PEG. In some embodiments, the at least one conjugated lipid comprises DMPE-PEG.

In some embodiments, the delivery vehicle comprises from about 0.5 to about 2.0 mole % of the at least one conjugated lipid. In some embodiments, the delivery vehicle does not comprise at least one conjugated lipid.

In some embodiments, the delivery vehicle comprises: the at least one bile salt or the at least one bile acid; the at least one multivalent cationic lipid; the at least one ionizable cationic lipid; the at least one structural lipid; and the at least one conjugated lipid.

In some embodiments, the delivery vehicle comprises: about 5-40 mole % of the at least one bile salt or the at least one bile acid; about 5-90 mole % of the at least one multivalent cationic lipid; about 5-90 mole % of the at least one ionizable cationic lipid; about 5-75 mole % of the at least one structural lipid component; and about 0.5-2.0 mole % the at least one conjugated lipid component.

In some embodiments, the delivery vehicle comprises: about 5-40 mole % of the at least one bile salt or the at least one bile acid; about 5-60 mole % of the at least one multivalent cationic lipid; about 5-60 mole % of the at least one ionizable cationic lipid; about 5-75 mole % of the at least one structural lipid; and about 0.5-2.0 mole % of the at least one conjugated lipid.

In some embodiments, the delivery vehicle comprises: about 20-40 mole % of the at least one bile salt or the at least one bile acid; about 5-30 mole % of the at least one multivalent cationic lipid; about 5-30 mole % of the at least one ionizable cationic lipid; about 30-50 mole % of the at least one structural lipid; and about 0.5-2.0 mole % of the at least one conjugated lipid.

In some embodiments, the delivery vehicle comprises: about 30-40 mole % of the at least one bile salt or the at least one bile acid; about 5-15 mole % of the at least one multivalent cationic lipid; about 5-15 mole % of the at least one ionizable cationic lipid; about 35-45 mole % of the at least one structural lipid; and about 0.5-2.0 mole % of the at least one conjugated lipid.

In some embodiments, the delivery vehicle comprises: about 33 mole % of the at least one bile salt or the at least one bile acid; about 12.5 mole % of the at least one multivalent cationic lipid; about 12.5 mole % of the at least one ionizable cationic lipid; about 41 mole % of the at least one structural lipid; and about 1 mole % of the at least one conjugated lipid.

In some embodiments, the delivery vehicle comprises any of the compositions disclosed in Table 1B.

In some embodiments, the at least one conjugated lipid is conjugated with at least one polypeptide. In some embodiments, the at least one polypeptide comprises at least one mucus penetrating polypeptide. In some embodiments, the at least one mucus penetrating polypeptide comprises an amino acid sequence according to SEQ ID NO: 17.

In some embodiments, the delivery vehicle comprises a cargo. In some embodiments, the cargo comprises a nucleic acid, a protein, an antibody, a peptide, a small molecule, a biologic, a peptidomimetic, a ribozyme, a chemical agent, a viral particle, a growth factor, a cytokine, an immunomodulating agent, a fluorescent dye, and any combination thereof.

In some embodiments, the cargo comprises a nucleic acid. In some embodiments, the nucleic acid comprises DNA. In some embodiments, the DNA comprises plasmid DNA.

In some embodiments, the molar ratio of total nanoparticle cationic lipids to the total number of nucleotides comprised by the nucleic acid cargo is from about 2 to about 20. In some embodiments, the molar ratio of total nanoparticle cationic lipids to the total number of nucleotides comprised by the nucleic acid cargo is from about 14 to about 18.

In some embodiments, the nucleic acid comprises RNA. In some embodiments, the molar ratio of total nanoparticle cationic lipids to the total number of nucleotides comprised by the nucleic acid cargo is from about 2 to about 20. In some embodiments, the molar ratio of total nanoparticle cationic lipids to the total number of nucleotides comprised by the nucleic acid cargo is from about 2 to about 4. In some embodiments the present disclosure provides a pharmaceutical composition comprises at least one of the delivery vehicles described in herein and an optional pharmaceutically acceptable excipient.

In some embodiments, the pharmaceutically acceptable excipient comprises an excipient, adjuvant, solution, stabilizer, additive, surfactant, lyophilization element, dilutant, and any combination thereof.

In some embodiments, the pharmaceutical composition is formulated for enteric delivery.

In some embodiments the present disclosure provides a method of delivering at least one cargo to a subject, the method comprising introducing at least one of the delivery vehicle described herein or at least one of the pharmaceutical compositions described herein to the gastrointestinal tract of the subject. In some embodiments, the at least one delivery vehicle or the at least one pharmaceutical composition is introduced to the subject gastrointestinal (GI) tract by at least one rout of administration. In some embodiments, the at least one rout of comprises intravenous administration, intraperitoneal administration, intramuscular administration, transdermal administration, ocular administration, oral administration, intrarectal administration, direct injection to the GI tract, and any combination thereof. In some embodiments, the at least one delivery vehicle or at least one pharmaceutical composition targets at least one gastrointestinal cell. In some embodiments, the at least one gastrointestinal cell comprises at least one of an intestinal epithelial cell, a lamina propria cell, an intraepithelial lymphocyte, an intestinal muscle cell, an enteric neuron, or any combination thereof.

In some embodiments, the at least one cargo is delivered to the gastrointestinal cell. In some embodiments, the at least one cargo is delivered to the intracellular space of the gastrointestinal cell. In some embodiments, the at least one cargo, an at least one cargo component, or an at least one expression product of the cargo is secreted from the gastrointestinal cell. In some embodiments, secretion of the at least one cargo, the at least one cargo component, or the at least one expression product of the cargo comprises apical secretion or basal secretion. In some embodiments, the at least one cargo, the at least one cargo component, or the at least one expression product of the cargo remains in an area proximal to the cell after secretion. In some embodiments, the at least one cargo, the at least one cargo component, or the at least one expression product of the cargo is secreted basally from the gastrointestinal cell and enters the circulation. In some embodiments, the at least one cargo, the at least one cargo component, or the at least one expression product of the cargo is distributed systemically after entering the circulation.

In some embodiments, the at least one cargo comprises at least one therapeutic agent. In some embodiments, the at least one therapeutic agent comprises one or more of a nucleic acid, a polypeptide, a protein, a biologic, an antibody, an enzyme, a hormone, a cytokine, an immunogen, and a genetic or epigenetic editing system component. In some embodiments, the at least one therapeutic agent comprises at least one nucleic acid. In some embodiments, the at least one nucleic acid encodes at least one polypeptide. In some embodiments, the at least one nucleic acid comprises DNA. In some embodiments, the at least one nucleic acid comprises plasmid DNA.

In some embodiments, the at least one nucleic acid comprises RNA. In some embodiments, the at least one nucleic acid comprises mRNA, circRNA, saRNA, and any combination thereof.

In some embodiments, the method described herein comprises transfecting the at least one gastrointestinal cell with the at least one nucleic acid. In some embodiments, the at least one gastrointestinal cell expresses at least one polypeptide encoded by the at least one nucleic acid. In some embodiments, the polypeptide comprises Granulocyte Spleeny-Stimulating Factor (G-CSF), Green Florescent Protein (GFP) and any combination thereof.

In some embodiments, the at least one nucleic acid comprises a at least one non-coding RNA. In some embodiments, the at least one non-coding RNA comprises one or more of short interfering RNA (siRNA), microRNA (miRNA), long non-coding RNA, piwi-interacting RNA (piRNA), small nucleolar RNA (snoRNA), small Cajal body-specific RNA (scaRNA), transfer RNA (tRNA), ribosomal RNA (rRNA), and small nuclear RNA (snRNA).

In some embodiments, the present disclosure provides a method for treating at least one therapeutic indication in a subject in need thereof comprising delivering at least one delivery vehicle described herein or at least one pharmaceutical compositions described herein to the subject via at least one of the methods for delivery of a cargo described herein. In some embodiments, the at least one therapeutic indication comprises at least one of a neurodegenerative disease, an ocular disease, a reproductive disease, a gastrointestinal disease, a brain disease, a skin disease, a skeletal disease, a muscoskeletal disease, a pulmonary disease, a thoracic disease, cystic fibrosis, tay-sachs, fragile X, Huntington's, neurofibromatosis, sickle cell, thalassemias, Duchenne's muscular dystrophy, familial adenomatous polyposis (FAP), attenuated FAP, microvillus inclusion disease (MVID), chronic inflammatory bowel disease, chronic inflammatory bowel disease, ileal Crohn's, juvenile polyposis, hereditary diffuse gastric cancer syndrome (HDGC), Peutz-Jeghers syndrome, lynch syndrome, gastric adenocarcinoma and proximal polyposis of the stomach (GAPPS), Li-Fraumeni syndrome, familial gastric cancer, Gilbert's syndrome, telangiectasia, mucopolysaccaride, Osler-Weber-Rendu syndrome, pancreatitis, keratoacanthoma, biliary atresia, Morquio's syndrome, Hurler's syndrome, Hunter's syndrome, Crigler-Najjar, Rotor's, Peutz-Jeghers' syndrome, Dubin-Johnson, Osteochondroses, Osteochondrodysplasias, polyposis, gastrointestinal infections, inflammatory bowel disease (IBD), ulcerative colitis, Crohn's disease, hemophilia, short bowel syndrome (SBS), diabetes, non-alcoholic steatohepatitis (NASH), with Hodgkin's lymphoma, non-Hodgkin's lymphoma, acute lymphoblastic leukemia, or acute myelogenous leukemia (AML), neutropenia, or any combination thereof.

In some embodiments, the at least one therapeutic indication comprises at least one immune-related indication. In some embodiments, the at least one immune-related indication comprises at least one gastrointestinal indication. In some embodiments, the at least one therapeutic indication comprises at least one cancer-related indication.

### BRIEF DESCRIPTION OF THE FIGURES

The figures are not necessarily to scale or comprehensive, with emphasis instead being placed upon illustrating the principles of various embodiments of the present disclosure.

FIG. 1 shows the results of an exemplary assay to measure transfection efficiency of exemplary delivery vehicles of this disclosure, carrying DNA as cargo, in HEK cells.

FIG. 2 shows results of an exemplary assay for measuring stability of exemplary delivery vehicles of this disclosure. “No treatment” indicates the FRET results for a delivery vehicle in a zero bile salt environment. Results for delivery vehicles exposed to the indicated bile salt concentrations are shown as normalized against the “no treatment” condition.

FIG. 3 shows results of an exemplary assay for measuring stability of exemplary delivery vehicles of this disclosure. “No treatment” indicates the FRET results for a delivery vehicle in a zero bile salt environment. Results for delivery vehicles exposed to the indicated bile salt concentrations are shown as normalized against the “no treatment” condition.

FIG. 4 shows results of an exemplary assay for measuring stability of exemplary delivery vehicles of this disclosure. “No treatment” indicates the FRET results for a delivery vehicle in a zero bile salt environment. Results for delivery vehicles exposed to the indicated bile salt concentrations are shown as normalized against the “no treatment” condition.

FIG. 5 shows an agarose gel electrophoresis with an exemplary delivery vehicle of this disclosure (Formulation No. 5 in Table 2). The lanes from left are as follows: lane one shows the ladder; lane 2 shows untreated delivery vehicle; lane three shows delivery vehicle treated with 7% Triton-X 100; lane four shows delivery vehicle treated with 7% Triton-X plus heat (70° C. for 30 mins).

FIG. 6 shows a mouse colon section of a mouse dosed with 30 micrograms of DNA encapsulated in a delivery vehicle that was DiI and DiO labelled. Observed is the distribution of 1% PEG containing vehicle (particle 5 of Table 3) labelled with DiI and DiO as shown by fluorescence imaging from DiI overlaid onto brightfield. See Example 5, Table 3 for descriptions of particle 5 and other referenced particles in the Figures.

FIG. 7 shows a mouse colon section of a mouse dosed with 30 micrograms of DNA encapsulated in a delivery vehicle that was DiI and DiO labelled. Observed is the distribution of 2% PEG containing vehicle (particle 6 of Table 3) labelled with DiI and DiO as shown by fluorescence imaging from DiI overlaid onto brightfield.

FIG. 8 shows a mouse colon section of a mouse dosed with 30 micrograms of DNA encapsulated in a delivery vehicle (particle 7 of Table 3) that was DiI and DiO labelled. Observed is the distribution of 3% PEG containing vehicle labelled with DiI and DiO as shown by fluorescence imaging from DiI overlaid onto brightfield.

FIG. 9 shows a mouse colon section of a mouse dosed with 30 micrograms of DNA encapsulated in a delivery vehicle that was DiI and DiO labelled. Observed is the distribution of 5% PEG containing vehicle (particle 8 of Table 3) labelled with DiI and DiO as shown by fluorescence imaging from DiI overlaid onto brightfield.

FIG. 10 shows a mouse colon section of a mouse dosed with 30 micrograms of DNA encapsulated in a delivery vehicle that was DiI and DiO labelled. Observed is the distribution of 10% PEG containing vehicle (particle 9 of Table 3) labelled with DiI and DiO as shown by fluorescence imaging from DiI overlaid onto brightfield.

FIG. 11A shows distribution of delivery vehicles in representative colon sections from mice administered particles at a ratio of 0%/25% MVL5/DODMA percent mols (particle 1 of Table 3).

FIG. 11B shows distribution of delivery vehicles in representative colon sections from mice administered particles at a ratio of 0%/25% MVL5/DODMA percent mols (particle 1 of Table 3).

FIG. 12A shows distribution of delivery vehicles in representative colon sections from mice administered particles at a ratio of 6.25%/18.75% (MVL5/DODMA) percent mols (particle 2 of Table 3).

FIG. 12B shows distribution of delivery vehicles in representative colon sections from mice administered particles at a ratio of 6.25%/18.75% (MVL5/DODMA) percent mols (particle 2 of Table 3).

FIG. 13A shows distribution of delivery vehicles in representative colon sections from mice administered particles at a ratio of 12.5%/12.5% (MVL5/DODMA) percent mols (particle 3 of Table 3).

FIG. 13B shows distribution of delivery vehicles in representative colon sections from mice administered particles at a ratio of 12.5%/12.5% (MVL5/DODMA) percent mols (particle 3 of Table 3).

FIG. 14A shows distribution of delivery vehicles in representative colon sections from mice administered particles at a ratio of 18.75%/6.25% (MVL5/DODMA) % mols (particle 4 of Table 3).

FIG. 14B shows distribution of delivery vehicles in representative colon sections from mice administered particles at a ratio of 18.75%/6.25% (MVL5/DODMA) % mols (particle 4 of Table 3).

FIG. 15A shows distribution of delivery vehicles in representative colon sections from mice administered particles at a ratio of 25%/0% MVL5/DODMA % mols (particle 10 of Table 3).

FIG. 15B shows distribution of delivery vehicles in representative colon sections from mice administered particles at a ratio of 25%/0% MVL5/DODMA % mols (particle 10 of Table 3).

FIG. 16A shows Swiss roll images of colons of a section of a first mouse administered MVL5/DODMA/DOPC/Deoxycholate/DMG-PEG (particle 11 of Table 3) with DiI and DiO using the BioTek Cytation software. The figure shows the DiI channel.

FIG. 16B show Swiss roll images of colons of a section of a first mouse administered MVL5/DODMA/DOPC/Deoxycholate/DMG-PEG (particle 11 of Table 3) with DiI and DiO using the BioTek Cytation software. The figure shows the DiI channel overlaid onto brightfield.

FIG. 16C shows Swiss roll images of colons of a section of a second mouse administered MVL5/DODMA/DOPC/Deoxycholate/DMG-PEG (particle 11 of Table 3) with DiI and DiO using the BioTek Cytation software. The figure shows the DiI channel.

FIG. 16D show Swiss roll images of colons of a section of a second mouse administered MVL5/DODMA/DOPC/Deoxycholate/DMG-PEG (particle 11 of Table 3) with DiI and DiO using the BioTek Cytation software. The figure shows the DiI channel overlaid onto brightfield.

FIG. 17A shows Swiss roll images of colons of a section of a first mouse administered MVL5/DODMA/GMO/Deoxycholate/DMG-PEG (particle 12 of Table 3) with DiI and DiO using the BioTek Cytation software. The figure shows the DiI channel.

FIG. 17B show Swiss roll images of colons of a section of a first mouse administered MVL5/DODMA/GMO/Deoxycholate/DMG-PEG (particle 12 of Table 3) with DiI and DiO using the BioTek Cytation software. The figure shows the DiI channel overlaid onto brightfield.

FIG. 17C shows Swiss roll images of colons of a section of a second mouse administered MVL5/DODMA/GMO/Deoxycholate/DMG-PEG (particle 12 of Table 3) with DiI and DiO using the BioTek Cytation software. The figure shows the DiI channel.

FIG. 17D show Swiss roll images of colons of a section of a second mouse administered MVL5/DODMA/GMO/Deoxycholate/DMG-PEG (particle 12 of Table 3) with DiI and DiO using the BioTek Cytation software. The figure shows the DiI channel overlaid onto brightfield.

FIG. 18A shows Swiss roll images of colons of a section of a first mouse administered MVL5/DODMA/DSPC/Deoxycholate/DMG-PEG (particle 5 of Table 3) with DiI and DiO using the BioTek Cytation software. The figure shows the DiI channel.

FIG. 18B show Swiss roll images of colons of a section of a first mouse administered MVL5/DODMA/DSPC/Deoxycholate/DMG-PEG (particle 5 of Table 3) with DiI and DiO using the BioTek Cytation software. The figure shows the DiI channel overlaid onto brightfield.

FIG. 18C shows Swiss roll images of colons of a section of a second mouse administered MVL5/DODMA/DSPC/Deoxycholate/DMG-PEG (particle 5 of Table 3) with DiI and DiO using the BioTek Cytation software. The figure shows the DiI channel.

FIG. 18D show Swiss roll images of colons of a section of a second mouse administered MVL5/DODMA/DSPC/Deoxycholate/DMG-PEG (particle 5 of Table 3) with DiI and DiO using the BioTek Cytation software. The figure shows the DiI channel overlaid onto brightfield.

FIG. 19A shows Swiss roll images of colons of a section of a first mouse administered PBS with DiI and DiO using the BioTek Cytation software. The figure shows the DiI channel.

FIG. 19B show Swiss roll images of colons of a section of a first mouse administered PBS with DiI and DiO using the BioTek Cytation software. The figure shows the DiI channel overlaid onto brightfield.

FIG. 19C shows Swiss roll images of colons of a section of a second mouse administered PBS with DiI and DiO using the BioTek Cytation software. The figure shows the DiI channel.

FIG. 19D show Swiss roll images of colons of a section of a second mouse administered PBS with DiI and DiO using the BioTek Cytation software. The figure shows the DiI channel overlaid onto brightfield.

FIG. 20 shows a bar graph comparing the stability of different bile salts incorporating lipid structures in 10 g/L of bile salts (cholate: deoxycholate mixture) by measuring perturbations in the lipid structures using FRET between DiI and DiO. FRET values are normalized to no treatment.

### DETAILED DESCRIPTION

Delivery of agents, such as therapeutic agents to epithelial tissues and cells, such as in the gastrointestinal (GI) tract, vagina and lung, present certain challenges. In these tissues, epithelial cells are covered with a mucosal layer and thus therapeutic agents must penetrate and move through the mucus to reach the epithelial cells. Additionally, therapeutic agents, once within or through the layer of mucus, must come into proximity to the intended target cells and in some embodiments, interact with the cell membrane and/or enter the cells. Accordingly, delivery of an agent (also referred to herein as “cargo”) is improved with a delivery vehicle that not only penetrate and cross through the mucus layer but also come within reach of the intended epithelial cell target. Also, with respect the GI tract and other tissues, harsh environments, such as naturally present bile acids of the GI, can present a challenge for the stability of delivery and for successful delivery of cargo to the intended target cells.

Provided herein are delivery vehicles for delivering a cargo to a desired target in a subject. Also disclosed herein are delivery vehicles that have improved stability in high bile salt environments, such as in the gastrointestinal tract. In some embodiments, the delivery vehicle disclosed herein, may provide stability in harsh environments of the GI tract and can be further be suited for mucus environments. In some embodiments, delivery vehicles herein may provide increased penetration or rate of transmission through mucous layers in or around target tissues or cells. In some embodiments, delivery vehicles herein provide both penetration through mucus thereby reducing or preventing entrapment of the delivery vehicle in the epithelial mucus as well as the epithelial reaching functionality which brings the delivery vehicle in proximity of the epithelial cells, such as within a distance of 20 microns or less. As such, the delivery vehicle can be suitable for delivering a cargo (e.g., a nucleic acid) to mucosal epithelial cells such as intestinal epithelial cells, lung epithelial cells, cervical epithelial cells, rectal epithelial cells, endometrial cells, and the likes. Further, the delivery vehicle can also be suitable for delivery to organs, such as the skin. In some embodiments, the delivery vehicles herein carry a therapeutic cargo (such as a nucleic acid, a protein, or a drug) and may be used to treat a disease affecting the GI tract such as familial polyposis (FAP), attenuated FAP, colorectal cancer, chronic inflammatory bowel disease, ileal Crohn's, Microvillus Inclusion Disease, and congenital diarrheas.

In some embodiments, the delivery vehicles provided herein comprise various lipid components and have the structure of a nanoparticle, e.g., a lipid-nanoparticle (LNP). In some embodiments, the lipid components of the delivery vehicles may be manufactured in such a way as to form a liposome, a micelle, or other lipid structures. In some embodiments, the delivery vehicles described herein are comprised of at least one bile salt or bile acid, at least one cationic lipid, at least one structural lipid, and at least one conjugated lipid. In some embodiments, the delivery vehicles herein comprise at least one bile salt or bile acid, at least two (2) cationic lipids, at least one structural lipid, and at least one conjugated lipid. In some embodiments, the disclosed delivery vehicles comprise at least one bile salt or bile acid, at least one multivalent cationic lipid, at least one ionizable cationic lipid which is not multivalent, at least one structural lipid, and at least one conjugated lipid. In some embodiments, the delivery vehicles lack one, some, or all but one of a bile salt, bile acid, cationic lipid (either multivalent or ionizable), structural lipid, or conjugated lipid. In some embodiments, the delivery vehicles do not contain cholesterol.

In some embodiments, the delivery vehicles provided herein (also referred to herein as “charge-separated” delivery vehicles) include those with a separation of positive and negative charges into separate loci within the vehicle, such that positively charged and negatively charged molecules are separated from one another, rather than interspersed.

In some embodiments, the delivery vehicles provided herein can include additional mucus-penetrating features that may assist in the penetration and movement of the delivery vehicle through the mucus surrounding the epithelial cells. Such additional features include but are not limited to incorporating a polymer such as Polyethylene glycol (PEG), Polyoxazoline polymer with methyl (PMOZ), Polyoxazoline polymer with ethyl (PEOZ) into the delivery vehicle surface and/or by including a mucus penetrating peptide (MPP) linked to the surface of the delivery vehicle. In some embodiments, the delivery vehicles comprise no PEG coating or a low density PEG coating (or a low density coating of another polymer).

Also provided herein are pharmaceutical compositions (also referred to as medicaments) formulated from the delivery vehicles disclosed herein with pharmaceutically acceptable excipients for administration to a subject. The pharmaceutical compositions described herein may be formulated for any rout of administration, including without limitation, oral, injection, parenteral, topical, transdermal, ophthalmic, otic, pulmonary, intranasal, nasal, buccal, rectal, or vaginal.

Also provided herein are methods for delivering a cargo to a subject as well as methods for treating therapeutic indications in subjects in need thereof using the delivery vehicles disclosed herein.

### I. DELIVERY VEHICLES

Provided herein are delivery vehicles which may optionally include at least one cargo. In some embodiments, the delivery vehicles may be or include liposomes, micelles, exosomes, viral particles, polymeric delivery agents, or a nanoparticle, such as lipid nanoparticles (LNPs), and non-lipid nanoparticles. In some embodiments, the delivery vehicles may be or include lipid structures. In some embodiments, the delivery vehicles may be or include at least one lipid nanoparticle (LNP).

### Delivery Vehicle Categories

### Nanoparticles

In some embodiments, the delivery vehicle may be or comprise a nanoparticle. The term “nanoparticle” as used herein refers to any particle ranging in size from 10-1000 nm. In some embodiments, the nanoparticles may be of any size including, but not limited to, about 10-900, about 10-800, about 10-700, about 10-600, about 10-500, about 10-400, about 10-300, about 10-200, about 10-100, about 100-1000, about 100-900, about 100-800, about 100-700, about 100-600, about 100-500, about 100-400, about 100-300, about 100-200, about 200-1000, about 200-900, about 200-800, about 200-700, about 200-600, about 200-500, about 200-400, about 200-300, about 300-1000, about 300-900, about 300-800, about 300-700, about 300-600, about 300-500, about 300-400, about 400-1000, about 400-900, about 400-800, about 400-700, about 400-600, about 400-500, about 500-1000, about 500-900, about 500-800, about 500-700, about 500-600, about 600-1000, about 600-900, about 600-800, about 600-700, about 700-1000, about 700-900, about 700-800, about 800-1000, about 800-900, or about 900-1000 nm. In some embodiments, the nanoparticles may be between about 50 nm and 150 nm in size.

In some embodiments, the nanoparticle may be 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 nm.

In some embodiments, delivery vehicle nanoparticles may have a defined shape. In some embodiments, the shape of the nanoparticles may be, but are not limited to, a sphere, an oval, a disk, a rod, a cone, a geodesic, or any combination thereof.

### Lipid Nanoparticles

In some embodiments, the delivery vehicle may be or comprise a nanoparticle which may be a lipid nanoparticle (LNP). As is known in the art, LNPs can be characterized as small solid or semi-solid particles possessing an exterior lipid layer with a hydrophilic exterior surface that is exposed to the non-LNP environment, an interior space which may aqueous (vesicle like) or non-aqueous (micelle like), and at least one hydrophobic inter-membrane space. The membranes of an LNP may be lamellar or non-lamellar. Also, the membrane of the LNP may be comprised of 1, 2, 3, 4, 5 or more layers. In some embodiments, LNPs may comprise a cargo into their interior space, into the inter membrane space, onto their exterior surface, or any combination thereof.

### Non-Lipid Nanoparticles

In some embodiments, the delivery vehicle may be or comprise a nanoparticle which may be a non-lipid-based nanoparticle. Non-lipid-based nanoparticles include, but are not limited to, carbon-based nanoparticles, polypeptide-based nanoparticles, silicon-based nanoparticles (i.e., porous silicon nanoparticles), nucleotide-based nanoparticles, and gold nanoparticles.

### Liposomes

In some embodiments, the delivery vehicles may be or comprise at least one liposome. As used herein, “liposomes” may refer to small vesicles comprised of at least one lipid bilayer membrane surrounding an aqueous inner-nanoparticle space. Generally, liposomes are not derived from a progenitor or host cell. Liposomes include (large) multilamellar vesicle (MLV) which are potentially hundreds of nanometers in diameter and comprise a series of concentric bilayers separated by narrow aqueous spaces. Liposomes also include small unicellular vesicle (SUV) which are potentially smaller than 50 nm in diameter. Also, liposomes may include large unilamellar vesicle (LUV) which are potentially between 50 and 500 nm in diameter. In some embodiments, liposomes differ from LNPs principally in their method of manufacture and may be comprised of any or all the same components and same component amounts as a lipid nanoparticle.

### Micelles

In some embodiments, the delivery vehicle may be or comprise at least one micelle. As used herein, “micelles” refer to small particles which do not have an aqueous intra-particle space. Without wishing to be bound by theory, the intra-particle space of micelles is occupied by the hydrophobic tails of the lipids comprising the micelle membrane and possible associated cargo, rather than any additional lipid-head groups. In some embodiments, micelles differ principally from LNPs in their method of manufacture and may be comprised of any or all the same components as a lipid-nanoparticle.

### Exosomes

In some embodiments, the delivery vehicle may be or comprises at least one exosome. As used herein, “exosomes” refer to small membrane bound vesicles with an endocytic origin. Without wishing to be bound by theory, exosomes are generally released into an extracellular environment from host/progenitor cells post fusion of multivesicular bodies the cellular plasma membrane. As such, exosomes will tend to include components of the progenitor membrane in addition to designed components and cargos. Exosome membranes are generally lamellar, composed of a bilayer of lipids, with an aqueous inter-nanoparticle space.

### Viral Particles

In some embodiments, the delivery vehicle may be or comprises at least one virus like particle. As used herein, “virus like particles” refer to a vesicle predominantly of a protein capsid, sheath, shell, or coat (all used interchangeably herein) derived from a virus which can be loaded with a cargo moiety. In general, virus like particle are non-infection and may be synthesized using cellular machinery to express viral capsid protein sequences, which then self-assemble and incorporate the associated cargo moiety, though it is possible to form virus like particles by providing the capsid and cargo components without expression related cellular machinery and allowing them to self-assemble.

In some embodiments, the virus like particle may be derived from at least one of species of virus such as, but not limited to, Parvoviridae, Retroviridae, Flaviviridae, Paramyxoviridae, and bacteriophages. In some embodiments, the virus like particle may be derived from an adeno-associated virus, HIV, Hepatitis C virus, HPV, or any combination thereof.

### Polymeric Delivery Technology

In some embodiments, the delivery vehicle may be or comprise at least one polymeric delivery agent. As used herein, “polymeric delivery agents” refer to non-aggregating delivery agents comprised of soluble polymers conjugated to cargo moieties via various linkage groups.

### Lipid Structures

In some embodiments, the delivery vehicle may be or comprise at least one lipid structure. In some embodiments, at least one lipid structure may be or include lipid particles, lipid nanoparticles, vesicles, liposomes, micelles, or any combination thereof. In general vesicles, refer to lipid structures wherein an aqueous volume is encapsulated by amphipathic lipid bilayers (e.g., single; unilamellar or multiple; multilamellar). In some embodiments, lipid structure refers to an arrangement wherein the lipids at least partially coat an interior comprising a therapeutic product. In some embodiments, lipid structure refers to lipid aggregates, wherein the lipid encapsulated therapeutic product is contained within a relatively disordered lipid mixture.

In some embodiments, a lipid structure can be a cationic liposome. In some embodiments, a liposome may be a cationic liposome used to carry negatively charged polynucleic acid, such as DNA. The presence of positively charged amines may facilitate binding with anions such as those found in DNA. A liposome thus formed may be a result of energetic contributions by Van der Waals forces and electrostatic binding to a DNA cargo which may partially contribute to liposome shape.

### Biocompatibility, Biodegradability, and Functional Lifespan

In some embodiments, the delivery vehicle described herein may be biocompatible and biodegradable. In some embodiments, the delivery vehicle may biodegrade after introduction into a subject. In some embodiments, biodegradation may begin immediately after introduction. In some embodiments, biodegradation may occur within the mucosal tract of a subject that has received an administration of the delivery vehicle. In some embodiments, biodegradation may result in release of a cargo. In some embodiments, biodegradation may comprise decomposition of a component of a nanoparticle structure such as a polymer.

In some embodiments, biodegradation may occur under standard bodily conditions such as from about 97.6° F. to about 99° F. In some embodiments, biodegradation may occur under a temperature from about 95° F. to about 106° F. In some embodiments, biodegradation may occur from about 95° F., 96° F., 97° F., 98° F., 99° F., 100° F., 101° F., 102° F., 103° F., 104° F., 105° F., or up to 106° F. In some embodiments, biodegradation may occur from about 50° F. to about 150° F.

In some embodiments, biodegradation may not occur.

In some embodiments, when biodegradation occurs, it may take from about 1 minute to about 100 years after administration of a nanoparticle or a structure to a subject. In some embodiments, biodegradation may take from about 1 minute, 5 minutes, 30 minutes, 1 hour, 3 hours, 7 hours, 10 hours, 15 hours, 20 hours, 25 hours, 2 days, 4 days, 8 days, 12 days, 20 days, 30 days, 1.5 months, 3 months, 4 months, 5 months, 6 months, 7 months, 8 months, 9 months, 10 months, 11 months, 12 months, 1.5 years, 3 years, 5 years, 8 years, 10 years, 15 years, 20 years, 30 years, 40 years, 50 years, 60 years, 70 years, 80 years, 90 years, or at least about 100 years.

In some embodiments, delivery vehicles may be functional for at least or at least about 1, 2, 3, 4, 5, 6, 7, 8, 9, 10, 11, 12, 13, 14, 15, 16, 17, 18, 19, 20, 21, 22, 23, 24, 25, 6, 27, 28, 29, 30, 40, 50, 60, 70, 80, 90, or 100 days after introduction to a subject in need thereof. In some embodiments, delivery vehicles may be functional for at least or at least about 1, 2, 3, 4, 5, 6, 7, 8, 9, 10, 11, or 12 months after introduction into a subject. In some embodiments, a delivery vehicle as provided herein, may be functional for at least or at least about 1, 2, 3, 4, 5, 6, 7, 8, 9, 10, 15, 20, 25, or 30 years after introduction to a subject. In some embodiments, the delivery vehicle may be functional for up to the lifetime of a recipient. In some embodiments, the delivery vehicle may function at 100% of its normal intended operation. In some embodiments, delivery vehicles may also function 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of their normal intended operation. In some embodiments, function of the delivery vehicle may refer to the efficiency of delivery, persistence of a lipid nanoparticles, stability of a lipid nanoparticles, or any combination thereof.

In some embodiments, the delivery vehicles provided herein may deliver a cargo, such as a nucleic acid to a target cell (such as RNA, DNA (for example, minicircle DNA)). In some embodiments, function may include a percent of cells that received a nucleic acid from the delivery vehicle composition. In some embodiments, function may refer to a frequency or efficiency of protein generation from a nucleic acid. In some embodiments, the delivery vehicle composition may deliver a nucleic acid to a cell that encodes for at least a portion of a gene, such as APC, and a frequency of efficiency may describe a functionally complete gene as restored or created by the delivery of the cargo.

### Delivery Vehicle Components

The components of the delivery vehicle may be selected based on the desired target, cargo, size, etc. As non-limiting examples, delivery vehicle components may be chosen to increase the delivery vehicles stability in a high bile salt environment, such as the gastrointestinal tract of a subject; to permit efficient penetration and transit through a mucus layer, to increase the rate of uptake by target cells, or any combination thereof.

It is to be understood that any singular reference to a component used herein can include reference to one and only one, one or more, or at least one of such components. Similarly, any plural reference to a component used herein can include reference to one and only one, one or more, or at least one of such components, unless otherwise noted.

### Lipids

The delivery vehicles herein may include one or more lipids, which may be selected to contribute different advantageous properties. For example, cationic lipids that differ in properties such as amine pKₐ, chemical stability, half-life in circulation, half-life in tissue, net accumulation in tissue, or toxicity may be used in the delivery vehicles disclosed herein.

In some embodiments, the delivery vehicle may comprise at least one lipid. In some embodiments, the delivery vehicle may comprise at least one bile salt or bile acid. In some embodiments, the delivery vehicle may be comprised of at least one bile salt, at least one bile acid, at least on cationic lipid, at least one structural lipid, at least one conjugated lipid, and any combination thereof. In some embodiments, a cationic (and neutral) lipid may be used for gene delivery.

### Bile Salts and Bile Acids

In some embodiments, delivery vehicles may comprise at least one bile salt or bile acid. Without wishing to be bound by theory, it is believed that the inclusion of these lipids increases the stability of the delivery vehicle in high bile salt environments. Without wishing to be bound by theory, the presence of bile salt in the delivery vehicle may prevent inclusion of further bile salt in the delivery vehicle membrane from the intestinal environment, thus preventing disintegration by the absorbed additional bile salts.

### Bile Acid

Delivery vehicles disclosed herein may include at least one bile acid. In general, bile acids are steroid acids, naturally occurring examples of which are synthesized in the liver (i.e., primary bile acids) and colon (i.e., secondary bile acids) of animals. The term “bile acid” as used herein, can include any member of the family of steroid acids, found in the bile of an animal (e.g., a human). Any reference to a bile acid used herein can include reference to an identical compound naturally or synthetically prepared.

Any reference to a bile acid used herein can include reference to a bile acid, one and only one bile acid, one or more bile acids, or to at least one bile acid. Furthermore, pharmaceutically acceptable bile acid esters can be utilized as the “bile acids” described herein, e.g., bile acids conjugated to an amino acid (e.g., glycine or taurine). Other bile acid esters can include, e.g., substituted, or unsubstituted alkyl ester, substituted or unsubstituted heteroalkyl esters, substituted or unsubstituted aryl esters, substituted or unsubstituted heteroaryl esters, or the like.

In some embodiments, the delivery vehicle may comprise a bile acid such as, but not limited to, 3β,5α,6β-trihydroxycholanoic acid, 12-ketochenodeoxycholic acid, 12-ketodeoxycholic acid, 12-ketolithocholic acid, 3-oxo chenodeoxycholic acid, 3-oxo deoxycholic acid, 3-oxocholic acid, 3α,6β,7α,12α-tetrahydroxy bile acid, 3α,6α,7α,12α-tetrahydroxy bile acid, 4-bromobenzoic acid, 6,7-diketolithocholic acid, 7-ketodeoxycholic acid, 7-ketolithocholic acid, allocholic acid, alloisolithocholic acid, apocholic acid, apocholic acid (delta 14 isomer), arachidyl amido cholanoic acid, chenodeoxycholic acid, chenodeoxycholic acid-d4, cholic acid, dehydrocholic acid, dehydrolithocholic acid, deoxycholic acid, dioxolithocholic acid, glyco-12-oxolithocholanoic acid, glycochenodeoxycholic acid, glycocholic acid, glycocholic acid hydrate, glycodehydrocholic acid, glycodeoxycholic acid, glycohyodeoxycholic acid, glycolithocholic acid, glycoursodeoxycholic acid, glyco-γ-muricholic acid, hyocholic acid, hyodeoxycholic acid, isodeoxycholic acid, isolithocholic acid, lithocholic acid, murideoxycholic acid, nordeoxycholic acid, obeticholic acid, pentadecanoic acid, ursocholic acid, ursodeoxycholic acid, ursodeoxycholic acid-D4, α-muricholic acid, β-muricholic acid, ω-muricholic acid, or any combination thereof.

In some embodiments, the delivery vehicle may comprise cholic acid. In some embodiments, the delivery vehicle may comprise chenodeoxycholic acid, lithocholic acid, taurodeoxycholic acid, or combination thereof.

In some embodiments, the delivery vehicle may comprise ursodiol, 5beta-cholanic acid, 3-oxy-cholenic acid, and any combination thereof.

### Bile Salt

Delivery vehicles disclosed herein may include at least one bile salt. In general, bile salts are bile acids which have been conjugated with an amino acid such as glycine or taurine. The term “bile salt,” as used herein, may include any member of the large family of molecules comprised of salts of steroid acids, found in the bile of an animal (e.g., a human). In general, a bile salt may be a conjugated bile acid. In some embodiments, a bile salt may be any bile acid conjugate. In some embodiments, a bile salt may be a bile acid conjugated with taurine or glycine. In some embodiments, the bile salt may be any salt of any bile acid. In some embodiments, the bile salt may be any bile acid conjugate anion.

Any reference to a bile salt used herein can include reference to an identical compound naturally or synthetically prepared.

Any reference to a bile salt used herein can include reference to a bile salt, one and only one bile salt, one or more bile salts, or to at least one bile salt.

In some embodiments, the delivery vehicle may comprise a bile salt, such as, but not limited to, sulfobromophthalein disodium salt hydrate, tauro-3β,5α,6β-trihydroxycholanoic acid, taurochenodeoxycholic acid sodium salt, taurocholic acid sodium salt hydrate, taurocholic acid sodium salt, taurodehydrocholic acid sodium salt, taurodeoxycholic acid sodium salt, taurohyodeoxycholate, taurohyodeoxycholic acid sodium salt, taurolithocholic acid 3-sulfate disodium salt, taurolithocholic acid sodium salt, tauro-β-muricholic acid sodium salt, tauroursodeoxycholic acid sodium salt, tauro-α-muricholic acid sodium salt, tauro-γ-muricholic acid sodium salt, tauro-ω-muricholic acid sodium salt, β-Estradiol 17-(β-D-glucuronide) sodium salt, lithocholic acid 3-sulfate (disodium salt), chenodeoxycholic acid 3-sulfate (disodium salt), chenodeoxycholic acid 7-sulfate (disodium salt), cholic acid 3-sulfate (disodium salt), cholic acid 7-sulfate (disodium salt), cholic acid sodium salt, deoxycholic acid 3-sulfate (disodium salt), deoxycholic acid disulfate (trisodium salt), phenoxymethylpenicillinic acid potassium salt, chenodeoxycholic acid disulfate (trisodium salt), chenodeoxycholic acid sodium salt, cholate, methyl cholate, sodium taurocholate hydrate, 1-naphthyl isothiocyanate, deoxycholate, hyodeoxycholate, glycocholate, sodium glycochenodeoxycholate, sodium cholate hydrate, taurocholate, taurodeoxycholate, taurochenodeoxycholate, chenodeoxycholate, lithocholate, isolithocholate, alloisolithocholate, sodium deoxycholate, sodium deoxycholate monohydrate, dehydrolithochlate, sodium glycodeoxycholate, sodium glycocholate hydrate, sodium taurodeoxycholate hydrate, sodium chenodeoxycholate, glycolithocholate sulfate, glycolithocholate, sodium taurohyodeoxycholate hydrate, sodium taurocholate, sodium tauroursodeoxycholate, sodium taurolithocholate, glycodeoxycholate, and any combination thereof.

In some embodiments, the delivery vehicle may comprise cholate, deoxycholate, their conjugates and derivatives, or combination thereof.

In some embodiments, the delivery vehicle may comprise cholate deoxycholate, chenodeoxycholate, lithocholate, and any combination thereof

In some embodiments, the delivery vehicle may comprise deoxycholate.

### Bile Salt or Bile Acid Amount or Concentration

In some embodiments, the bile salt or bile acid concentration in the delivery vehicle may comprise from about 80 mole % to about 10 mole %, such as from about 80 mole % to about 70 mole %, from about 65 mole % to about 55 mole %, from about 60 mole % to about 50%, from about 55 mole % to about 45 mole %, from about 50 mole % to about 40 mole %, from about 45 mole % to about 35 mole %, from about 40 mole % to about 30 mole %, from about 35 mole % to about 25 mole %, from about 30 mole % to about 20 mole %, from about 25 mole % to about 15 mole %, from about 20 mole % to about 10 mole %, from about 15 mole % to about 10 mole %, from about 60 mole % to about 20 mole %, from about 25.9 mole %, from about 30.4 mole %, about 34.9 mole %, from about 39.4 mole %, from about 37.1 mole %, from about 43.9 mole %, or about 45 mole %. In some embodiments, the bile salt or bile acid concentration in the delivery vehicle may comprise about 5 mole %, 10 mole %, 15 mole %, 20 mole %, 25 mole %, 30 mole %, 35 mole %, 40 mole %, 45 mole %, 50 mole %, 55 mole %, 60 mole %, 65 mole %, 70 mole %, 75 mole %, 80 mole %, or 85 mole %.

In some embodiments, the amount of bile salt or bile acid in the delivery vehicle may comprise between about 5 and about 85 mole % of the total amount of lipids in the delivery vehicle. For example, the amount of bile salt or bile acid in the delivery vehicle may comprise about 5-85 mole %, about 15-85 mole %, about 25-85 mole %, about 35-85 mole %, about 45-85 mole %, about 55-85 mole %, about 65-85 mole %, about 75-85 mole %, about 5-75 mole %, about 15-75 mole %, about 25-75 mole %, about 35-75 mole %, about 45-75 mole %, about 55-75 mole %, about 65-75 mole %, about 5-65 mole %, about 15-65 mole %, about 25-65 mole %, about 35-65 mole %, about 45-65 mole %, about 55-65 mole %, about 5-55 mole %, about 15-55 mole %, about 25-55 mole %, about 35-55 mole %, about 45-55 mole %, about 5-45 mole %, about 15-45 mole %, about 25-45 mole %, about 35-45 mole %, about 5-35 mole %, about 15-35 mole %, about 25-35 mole %, about 5-25 mole %, about 15-25 mole %, about 5-15 mole %, or about 45 mole % of the total amount of lipids in the delivery vehicle. In some embodiments, the amount of bile salt or bile acid in the delivery vehicle may comprise about 5 mole %, 10 mole %, 15 mole %, 20 mole %, 25 mole %, 30 mole %, 35 mole %, 40 mole %, 45 mole %, 50 mole %, 55 mole %, 60 mole %, 65 mole %, 70 mole %, 75 mole %, 80 mole %, or 85 mole % of the total amount of lipids in the delivery vehicle.

In some embodiments, the amount of bile salt or bile acid in the delivery vehicle may be between about 5 and about 40 mole % of the total amount of lipids in the delivery vehicle. In some embodiments, the amount of bile salt or bile acid in the delivery vehicle may be between about 20 and about 40 mole % of the total amount of lipids in the delivery vehicle. In some embodiments, the amount of bile salt or bile acid in the delivery vehicle may be between about 30 and about 40 mole % of the total amount of lipids in the delivery vehicle. In some embodiments the amount of bile salt or bile acid in the delivery vehicle may be about 35 mole % of the total amount of lipids in the delivery vehicle.

Bile salts or bile acids may be included in nanoparticles at levels of from about 5 to about 40 mole % of total nanoparticle lipid (e.g., from about 20 to about 40 or from about 33 to about 37 mole % of total nanoparticle lipid).

### Multiple Bile Acid or Bile Salt Delivery Vehicles

In some embodiments, the delivery vehicle may comprise more than one bile salt or bile acid. In some embodiments, the delivery vehicle may comprise at least two bile salts or bile acids. In some embodiments, a delivery vehicle may comprise at least one bile acid and at least one bile salt.

In some embodiments, the delivery vehicle may comprise cholic acid and deoxycholate.

In some embodiments, when more than one bile salt or bile acid is present in a delivery vehicle, each bile salt or bile acid is present in a different amount.

In some embodiments, the amount of any one bile salt or bile acid in the delivery vehicle may comprise between about 5 and about 85 mole % of the total amount of lipids in the delivery vehicle. For example, the amount of any one bile salt or bile acid in the delivery vehicle may comprise about 5-85 mole %, about 15-85 mole %, about 25-85 mole %, about 35-85 mole %, about 45-85 mole %, about 55-85 mole %, about 65-85 mole %, about 75-85 mole %, about 5-75 mole %, about 15-75 mole %, about 25-75 mole %, about 35-75 mole %, about 45-75 mole %, about 55-75 mole %, about 65-75 mole %, about 5-65 mole %, about 15-65 mole %, about 25-65 mole %, about 35-65 mole %, about 45-65 mole %, about 55-65 mole %, about 5-55 mole %, about 15-55 mole %, about 25-55 mole %, about 35-55 mole %, about 45-55 mole %, about 5-45 mole %, about 15-45 mole %, about 25-45 mole %, about 35-45 mole %, about 5-35 mole %, about 15-35 mole %, about 25-35 mole %, about 5-25 mole %, about 15-25 mole %, about 5-15 mole %, or about 45 mole % of the total amount of lipids in the delivery vehicle. In some embodiments, the amount of any one bile salt or bile acid in the delivery vehicle may comprise about 5 mole %, 10 mole %, 15 mole %, 20 mole %, 25 mole %, 30 mole %, 35 mole %, 40 mole %, 45 mole %, 50 mole %, 55 mole %, 60 mole %, 65 mole %, 70 mole %, 75 mole %, 80 mole %, or 85 mole % of the total amount of lipids in the delivery vehicle.

In some embodiments, when more than one bile salt or bile acid is present in a delivery vehicle, each bile salt or bile acid is present in a about the same amount.

In some embodiments, the total amount of all bile salts or bile acids in the delivery vehicle may comprise between about 5 and about 85 mole % of the total amount of lipids in the delivery vehicle. For example, the total amount of all bile salt or bile acid in the delivery vehicle may comprise about 5-85 mole %, about 15-85 mole %, about 25-85 mole %, about 35-85 mole %, about 45-85 mole %, about 55-85 mole %, about 65-85 mole %, about 75-85 mole %, about 5-75 mole %, about 15-75 mole %, about 25-75 mole %, about 35-75 mole %, about 45-75 mole %, about 55-75 mole %, about 65-75 mole %, about 5-65 mole %, about 15-65 mole %, about 25-65 mole %, about 35-65 mole %, about 45-65 mole %, about 55-65 mole %, about 5-55 mole %, about 15-55 mole %, about 25-55 mole %, about 35-55 mole %, about 45-55 mole %, about 5-45 mole %, about 15-45 mole %, about 25-45 mole %, about 35-45 mole %, about 5-35 mole %, about 15-35 mole %, about 25-35 mole %, about 5-25 mole %, about 15-25 mole %, about 5-15 mole %, or about 45 mole % of the total amount of lipids in the delivery vehicle. In some embodiments, the total amount of all bile salt or bile acid in the delivery vehicle may comprise about 5 mole %, 10 mole %, 15 mole %, 20 mole %, 25 mole %, 30 mole %, 35 mole %, 40 mole %, 45 mole %, 50 mole %, 55 mole %, 60 mole %, 65 mole %, 70 mole %, 75 mole %, 80 mole %, or 85 mole % of the total amount of lipids in the delivery vehicle.

In some embodiments, nanoparticle bile salt may include deoxycholate and/or lithocholate. Nanoparticles may include two bile salts. Nanoparticles may include deoxycholate at a level of from about 20 to about 30 mole % of total nanoparticle lipid and lithocholate at a level of from about 5 to about 10 mole % of total nanoparticle lipid.

### Bile Acid and Bile Salt Derivatives

In some embodiments, the delivery vehicles may comprise a bile acid-like or bile salt-like component. As used herein, the terms “bile acid-like” and “bile salt-like” refer to molecules that, while not known to be found in the bile of an animal, would otherwise be recognized by those skilled in the art as likely members of those respective families. As a non-limiting example, bile acid-like lipids may include stereoisomers of steroid acids with longer or shorter carbon chains than, greater or fewer hydroxy groups as, or variation in the number of carbon rings as naturally occurring bile acids. As a non-limiting example, bile salt-like lipids may include conjugates of bile acid-like molecules or bile salts conjugated with amino acids other than those found in the bile of an animal.

### Cationic Lipid

In some embodiments, a delivery vehicle of this disclosure may comprise a cationic lipid. In some embodiments, a cationic lipid may attain a positive charge through one or more amines present in a polar head group. As a non-limiting example, cationic lipids may include, multivalent cationic lipids, ionizable cationic lipids or any combinations thereof.

In some embodiments, cationic lipids may be chosen so that the properties of the delivery vehicle comprised of multiple different cationic lipids (i.e., a mixed-lipid lipid structure) are more desirable than the properties of a delivery vehicle comprised of a single cationic lipid (i.e., a single-lipid structure of individual lipids). In some embodiments, net tissue accumulation and long-term toxicity (if any) from cationic lipids may be modulated in a favorable way by choosing mixtures of cationic lipids instead of selecting a single cationic lipid in a given formulation. In some embodiments, such mixtures may also provide better encapsulation and/or release of a cargo, such as a nucleic acid. In some embodiments, a combination of cationic lipids also may affect the systemic stability when compared to single cationic lipid in a formulation.

### Multivalent Cationic Lipids

In some embodiments, the delivery vehicle may comprise at least one multivalent cationic lipid. In general, a multivalent cationic lipid is understood to be a cationic lipid whose head group possesses more than a single positive charge. For example, a multivalent cationic lipid may possess 3, 4, 5, or more positive charges. In some embodiments, multivalent cationic lipid may include either bi-, tri-, quad-, penta-valent, hexa-, hepta-, octa-valent, and so on amino headgroups.

Non limiting examples of multivalent cationic lipids which may be included in the delivery vehicle may include N1-[2-((1 S)-1-[(3-aminopropyl)amino]-4-[di(3-amino-propyl)amino]butylcarboxamido)ethyl]-3,4-di[oleyloxy]-benzamide (MVL5), N4-Cholesteryl-Spermine HCl (GL67), salts thereof, and any combinations thereof.

In some embodiments, the delivery vehicle provided herein can be generated using MVL5. In some embodiments, a lipid nanoparticle of the delivery vehicle may comprise a multivalent cationic lipid including, but not limited to, (MVL5), a salt thereof, and any combination thereof.

### Ionizable Cationic Lipids

In some embodiments, the delivery vehicle may include at least one ionizable cationic lipid. In some embodiments, the delivery vehicle may

In some embodiments, an ionizable cationic lipid for use in the delivery vehicles herein (such as in the lipid nanoparticles of a disclosed delivery vehicle) may include but is not limited to, 1,2-dioleyloxy-3-dimethylaminopropane (DODMA), N-[1-(2,3-dioleyloxy)propyl]-N,N,N-trimethylammonium chloride (DOTMA), [1,2-bis(oleoyloxy)-3-(trimethylammonio)propane] (DOTAP), dimethyldioctadecylammonium (DDA), 30[N-(N′,N′-dimethylaminoethane)-carbamoyl] cholesterol (DC-Chol), and dioctadecylamidoglycylspermine (DOGS), 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 1,2-Dialkyloxy-N,N-dimethylaminopropane, 4-(2,2-diocta-9,12-dienyl-[1,3]dioxolan-4-ylmethyl)-dimethylamine, O-alkyl ethylphosphocholines, (6Z,9Z,28Z,31Z)-heptatriacont-6,9,28,31-tetraene-19-yl 4-(dimethylamino)butanoate (MC3), (6Z,9Z,28Z,31Z)-heptatriaconta-6,9,28,31-tetraen-19-yl 3-(dimethylamino)propanoate (MC2), MC4,3β-[N-(N′,N′-dimethylaminoethane)-carbamoyl]cholesterol, N4-Cholesteryl-Spermine, 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 7-(4-(dimethylamino)butyl)-7-hydroxytridecane-1,13-diyl dioleate (CL1H6), 7-(4-(diisopropylamino)butyl)-7-hydroxytridecane-1,13-diyl dioleate (CL4H6), CL1A6, CL1A6, CL3A6, CL4A6, CL5A6, CL6A6, CL7A6, CL8A6, CL9A6, CL10A6, CL11A6, CL12A6, CL13A6, CL14A6, CL15A6, YSK12-C4, (as described in US20200129431A1 and Sato Y et al. Understanding structure-activity relationships of pH-sensitive cationic lipids facilitates the rational identification of promising lipid nanoparticles for delivering siRNAs in vivo. J Control Release. 2019; 295:140-152 both herein incorporated by reference as relates to the disclosure of ionizable cationic lipids), and any combination thereof.

In some embodiments, the ionizable cationic may include, but is not limited to, any one of MC2, CL1H6, CL4H6, DODMA, or any combination thereof.

### Dual Cationic Lipid Delivery Vehicles

In some embodiments, the delivery vehicle includes at least 2 cationic lipids. In some embodiments, the delivery vehicle includes at least two ionizable cationic lipids. In some embodiments, the delivery vehicle comprises at least two multivalent cationic lipids. In some embodiments, the delivery vehicle comprises at least one multivalent cationic lipid and at least one ionizable cationic lipid. In some embodiments, the delivery vehicle includes one multivalent cationic lipid and one ionizable cationic lipid.

In some embodiments, the delivery vehicle includes: (a) MVL5, GL67, or any combination thereof; and (b) MC2, CL1H6, CL4H6, DODMA, or any combination thereof. In some embodiments, the delivery vehicle includes MVL5 and MC2; MVL5 and CL1H6, MVL5 and CL4H6, MVL5 and DODMA, or any combination thereof.

In some embodiments, the cationic lipids are present in the delivery vehicle in different amounts. In some embodiments, the cationic lipids are present in the delivery vehicle in the same amount.

### Amount of Cationic Lipids in the Delivery Vehicles

Total amount of Cationic Lipid

In some embodiments, the total amount of cationic lipid in the delivery vehicle may be about 5-90 mole % of all the delivery vehicle lipids. For example, the delivery vehicle may include about 5-90 mole %, about 5-80 mole %, about 5-70 mole %, about 5-60 mole %, about 5-50 mole %, about 5-40 mole %, about 5-30 mole %, about 5-20 mole %, about 5-10 mole %, about 10-90 mole %, about 10-80 mole %, about 10-70 mole %, about 10-60 mole %, about 10-50 mole %, about 10-40 mole %, about 10-30 mole %, about 10-20 mole %, about 20-90 mole %, about 20-80 mole %, about 20-70 mole %, about 20-60 mole %, about 20-50 mole %, about 20-40 mole %, about 20-30 mole %, about 30-90 mole %, about 30-80 mole %, about 30-70 mole %, about 30-60 mole %, about 30-50 mole %, about 30-40 mole %, about 40-90 mole %, about 40-80 mole %, about 40-70 mole %, about 40-60 mole %, about 40-50 mole %, about 50-90 mole %, about 50-80 mole %, about 50-70 mole %, about 50-60 mole %, about 60-90 mole %, about 60-80 mole %, about 60-70 mole %, about 70-90 mole %, about 70-80 mole %, or about 80-90 mole % of the cationic lipid.

In some embodiments, the delivery vehicle may include about 5-60 mole % of the cationic lipid. In some embodiments, the delivery vehicle may include about 10-60 mole % of the cationic lipid. In some embodiments, the delivery vehicle may include about 10-50 mole % of the cationic lipid. In some embodiments, the delivery vehicle may include about 10-30 mole % of the cationic lipid. In some embodiments, the delivery vehicle may include about 25 mole % of the cationic lipid.

### Amount of Cationic Lipids in Dual Cationic Delivery Vehicles

In some embodiments, wherein more than one cationic lipid is present in the delivery vehicle, any one of the cationic lipids may be present in an amount of about 5-90 mole % of the total amount of delivery vehicle lipids. For example, any one of the cationic lipids may be present in an amount of about 5-90 mole %, about 5-80 mole %, about 5-70 mole %, about 5-60 mole %, about 5-50 mole %, about 5-40 mole %, about 5-30 mole %, about 5-20 mole %, about 5-10 mole %, about 10-90 mole %, about 10-80 mole %, about 10-70 mole %, about 10-60 mole %, about 10-50 mole %, about 10-40 mole %, about 10-30 mole %, about 10-20 mole %, about 20-90 mole %, about 20-80 mole %, about 20-70 mole %, about 20-60 mole %, about 20-50 mole %, about 20-40 mole %, about 20-30 mole %, about 30-90 mole %, about 30-80 mole %, about 30-70 mole %, about 30-60 mole %, about 30-50 mole %, about 30-40 mole %, about 40-90 mole %, about 40-80 mole %, about 40-70 mole %, about 40-60 mole %, about 40-50 mole %, about 50-90 mole %, about 50-80 mole %, about 50-70 mole %, about 50-60 mole %, about 60-90 mole %, about 60-80 mole %, about 60-70 mole %, about 70-90 mole %, about 70-80 mole %, or about 80-90 mole % of total amount of delivery vehicle lipids.

In some embodiments, wherein more than one cationic lipid is present in the delivery vehicle, any one of the cationic lipids may be present in an amount of about 5-60 mole % of total amount of delivery vehicle lipids. In some embodiments, wherein more than one cationic lipid is present in the delivery vehicle, any one of the cationic lipids may be present in an amount of about 5-30 mole % of total amount of delivery vehicle lipids. In some embodiments, wherein more than one cationic lipid is present in the delivery vehicle, any one of the cationic lipids may be present in an amount of about 5-15 mole % of total amount of delivery vehicle lipids. In some embodiments, wherein more than one cationic lipid is present in the delivery vehicle, any one of the cationic lipids may be present in an amount of about 12.5 mole % of total amount of delivery vehicle lipids.

In some embodiments, wherein more than one cationic lipid is present in the delivery vehicle, any one of the cationic lipids may be present in an amount of about 5-75 mole % of the total amount of cationic lipids in the delivery vehicle. For example, any one of the cationic lipids may be present in an amount of about 5-15 mole %, about 5-25 mole %, about 5-35 mole %, about 5-45 mole %, about 5-55 mole %, about 5-65 mole %, about 5-75 mole %, about 10-70 mole %, about 10-60 mole %, about 10-50 mole %, about 10-40 mole %, about 10-30 mole %, about 10-20 mole %, about 20-70 mole %, about 20-60 mole %, about 20-50 mole %, about 20-40 mole %, about 20-30 mole %, about 30-70 mole %, about 30-60 mole %, about 30-50 mole %, about 30-40 mole %, about 30-70 mole %, about 30-60 mole %, about 30-50 mole %, about 40-70 mole %, about 40-60 mole %, about 40-50 mole %, about 50-70 mole %, about 50-60 mole %, or about 60-70 mole % of the total amount of cationic lipids in the delivery vehicle.

In some embodiments, wherein more than one cationic lipid is present in the delivery vehicle, any one of the cationic lipids may be present in an amount of about 40-60 mole % of the total amount of cationic lipids in the delivery vehicle. In some embodiments, wherein more than one cationic lipid is present in the delivery vehicle, any one of the cationic lipids may be present in an amount of about 50 mole % of the total amount of cationic lipids in the delivery vehicle.

In some embodiments, the delivery vehicle may include any of the multivalent cationic lipids provided herein at less than about 50 mole %, 48 mole %, 46 mole %, 44 mole %, 42 mole %, 40 mole %, 38 mole %, 36 mole %, 34 mole %, 32 mole %, 30 mole %, 28 mole %, 26 mole %, 24 mole %, 22 mole %, 20 mole %, 18 mole %, 16 mole %, 14 mole %, 12 mole %, 10 mole %, 8 mole %, 6 mole %, 4 mole %, 2 mole %, or 0 mole % of the total amount of delivery vehicle lipids. In some embodiments, the delivery vehicle may include any of the multivalent cationic lipids provided herein at about 50 mole %, 48 mole %, 46 mole %, 44 mole %, 42 mole %, 40 mole %, 38 mole %, 36 mole %, 34 mole %, 32 mole %, 30 mole %, 28 mole %, 26 mole %, 24 mole %, 22 mole %, 20 mole %, 18 mole %, 16 mole %, 14 mole %, 12 mole %, 10 mole %, 8 mole %, 6 mole %, 4 mole %, 2 mole %, or 0 mole % of the total amount of delivery vehicle lipids. In some embodiments, the delivery vehicle may include any of the multivalent cationic lipids provided herein at a concentration of 5-50 mole %, 5-40 mole %, 5-30 mole %, 5-25 mole %, 5-20 mole %, 5-15 mole %, 10-50 mole %, 10-40 mole %, 10-30 mole %, 10-25 mole %, 15-50 mole %, 15-40 mole %, 15-30 mole % and 15-25 mole % of the total amount of delivery vehicle lipids.

In some embodiments, the delivery vehicle may include any of the ionizable cationic lipids provided herein at less than about 50 mole %, 48 mole %, 46 mole %, 44 mole %, 42 mole %, 40 mole %, 38 mole %, 36 mole %, 34 mole %, 32 mole %, 30 mole %, 28 mole %, 26 mole %, 24 mole %, 22 mole %, 20 mole %, 18 mole %, 16 mole %, 14 mole %, 12 mole %, 10 mole %, 8 mole %, 6 mole %, 4 mole %, 2 mole %, or 0 mole % of the total amount of delivery vehicle lipids. In some embodiments, the delivery vehicle may include any of the ionizable cationic lipids provided herein at about 50 mole %, 48 mole %, 46 mole %, 44 mole %, 42 mole %, 40 mole %, 38 mole %, 36 mole %, 34 mole %, 32 mole %, 30 mole %, 28 mole %, 26 mole %, 24 mole %, 22 mole %, 20 mole %, 18 mole %, 16 mole %, 14 mole %, 12 mole %, 10 mole %, 8 mole %, 6 mole %, 4 mole %, 2 mole %, or 0 mole % of the total amount of delivery vehicle lipids. In some embodiments, the delivery vehicle may include any of the ionizable cationic lipids provided herein at a concentration of 5-50 mole %, 5-40 mole %, 5-30 mole %, 5-25 mole %, 5-20 mole %, 5-15 mole %, 10-50 mole %, 10-40 mole %, 10-30 mole %, 10-25 mole %, 15-50 mole %, 15-40 mole %, 15-30 mole % and 15-25 mole % of the total amount of delivery vehicle lipids.

In some embodiments, nanoparticle cationic lipid may include MVL5. In some embodiments, MVL5 may be present at a level of from about 5 to about 20 mole % of total nanoparticle lipid.

In some embodiments, nanoparticle cationic lipid may include one or more of MC2, CL1H6, and CL4H6, each of which may be present at a level of from about 5 to about 20 mole % of total nanoparticle lipid.

### Structural Lipid

In some embodiments, the delivery vehicle may comprise at least one structural lipid. In some embodiments, the at least one structural lipid component may be any lipid or lipid soluble molecule which, without limitation, increases cellular uptake of the nanoparticle, increases the rate or efficiency of transfection, increases the stability of the nanoparticle during formation, aids in formation of the nanoparticle, is useful for adjusting the overall charge of the nanoparticle, increases stability of the nucleic acid cargo in the gastrointestinal tract, or any combination thereof.

In some embodiments, the structural lipid component may be, but is not limited to, at least one of a neutral lipid, an anionic lipid, a phospholipid, and any combination thereof.

In some embodiments, the at least one structural lipid component may be selected from, but is not limited to, at least one of 1,2-distearoyl-sn-glycero-3-phosphocholine (DSPC), 1,2-dimyristoyl-sn-glycero-3-phosphocholine (DMPC) dioleoylphosphatidylethanolamine (DOPE), glycerol monooleate (GMO), and any combination thereof.

### Phospholipids

In some embodiments, the delivery vehicles can comprise a phosphatidylcholine. Exemplary phosphatidylcholines include but are not limited to dilauroyl phophatidylcholine, dimyristoylphophatidylcholine, dipalmitoylphophatidylcholine, distearoylphophatidyl-choline, diarachidoylphophatidylcholine, dioleoylphophatidylcholine, dilinoleoyl-phophatidylcholine, dierucoylphophatidylcholine, palmitoyl-oleoyl-phophatidylcholine, egg phosphatidylcholine, myristoyl-palmitoylphosphatidylcholine, palmitoyl-myristoyl-phdsphatidylcholine, myristoyl-stearoylphosphatidylcholine, palmitoyl-stearoyl-phosphatidylcholine, stearoyl-palmitoylphosphatidylcholine, stearoyl-oleoyl-phosphatidylcholine, stearoyl-linoleoylphosphatidylcholine and palmitoyl-linoleoyl-phosphatidylcholine.

In some embodiments, the structural lipid may include short-chainbis-n-heptadecanoylphosphatidylcholine (DHPC), dihexadecoylphosphoethanolamine (DHPE), 1,2-dilinoleoyl-sn-glycero-3-phosphocholine (DLPC), dimyristoylphosphoethanolamine (DMPE), dimyristoylphosphatidylglycerol (DMPG), dioleoylphosphatidylcholine (DOPC), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylcholine (DPPC), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoylphosphoethanolamineimidazole (DSPEI), 1,2-diundecanoyl-sn-glycero-phosphocholine (DUPC), eggphosphatidylcholine (EPC), hydrogenatedsoybeanphosphatidylcholine (HSPC), mannosializeddipalmitoylphosphatidylethanolamine (ManDOG), 1,2-Dioleoyl-sn-Glycero-3-Phosphoethanolamine-N-[4-(p-maleimidomethyl)cyclohexane-carboxamide] (MCC-PE), 1,2-diphytanoyl-sn-glycero-3-phosphoethanolamine (ME 16.0 PE), 1-myristoyl-2-hydroxy-sn-glycero-phosphocholine (MHPC), 1-oleoyl-2-cholesterylhemisuccinoyl-sn-glycero-3-phosphocholine (OChemsPC), phosphatidicacid (PA), phosphatidylethanolaminelipid (PE), phosphatidylglycerol (PG), partiallyhydrogenatedsoyphosphatidylchloline (PHSPC), phosphatidylinositollipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), 18-1-transPE,1-stearoyl-2-oleoyl-phosphatidyethanolamine (SOPE), soybeanphosphatidylcholine (SPC), 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, and any combination thereof.

In some embodiments, the delivery vehicles may include an asymmetric phosphatidylcholine. Asymmetric phosphatidylcholines can be referred to as 1-acyl, 2-acyl-sn-glycero-3-phosphocholines, wherein the acyl groups are different from each other.

In some embodiments, the delivery vehicle may include a symmetric phosphatidylcholine. Symmetric phosphatidylcholines can be referred to as 1,2-diacyl-sn-glycero-3-phosphocholines.

As used herein, the abbreviation “PC” refers to phosphatidylcholine. The phosphatidylcholine 1,2-dimyristoyl-sn-glycero-3-phosphocholine can be abbreviated herein as “DMPC.” The phosphatidylcholine 1,2-dioleoyl-sn-glycero-3-phosphocholine can be abbreviated herein as “DOPC.” The phosphatidylcholine 1,2-dipalmitoyl-sn-glycero-3-phosphocholine can be abbreviated herein as “DPPC.” In general, saturated acyl groups found in various lipids include groups having the names propionyl, butanoyl, pentanoyl, caproyl, heptanoyl, capryloyl, nonanoyl, capryl, undecanoyl, lauroyl, tridecanoyl, myristoyl, pentadecanoyl, palmitoyl, phytanoyl, heptadecanoyl, stearoyl, nonadecanoyl, arachidoyl, heneicosanoyl, behenoyl, trucisanoyl and lignoceroyl. The corresponding IUPAC names for saturated acyl groups are trianoic, tetranoic, pentanoic, hexanoic, heptanoic, octanoic, nonanoic, decanoic, undecanoic, dodecanoic, tridecanoic, tetradecanoic, pentadecanoic, hexadecanoic, 3,7,11,15-tetramethylhexadecanoic, heptadecanoic, octadecanoic, nonadecanoic, eicosanoic, heneicosanoic, docosanoic, trocosanoic and tetracosanoic. Unsaturated acyl groups found in both symmetric and asymmetric phosphatidylcholines include myristoleoyl, palmitoleyl, oleoyl, elaidoyl, linoleoyl, linolenoyl, eicosenoyl and arachidonoyl. The corresponding IUPAC names for unsaturated acyl groups are 9-cis-tetradecanoic, 9-cis-hexadecanoic, 9-cis-octadecanoic, 9-trans-octadecanoic, 9-cis-12-cis-octadecadienoic, 9-cis-12-cis-15-cisoctadecatrienoic, 11-cis-eicosenoic and 5˜cis-8-cis-ll-cis-14-cis-eicosatetraenoic.

Exemplary phosphatidylethanolamines include dimyristoyl-phosphatidylethanolamine, dipalmitoyl-phosphatidylethanolamine, distearoyl phosphatidylethanolamine, dioleoyl-phosphatidylethanolamine, and egg phosphatidylethanolamine. Phosphatidylethanolamines may also be referred to under IUPAC naming systems as 1,2-diacyl-sn-glycero-3-phosphoethanolamines or 1-acyl-2-acyl-sn-glycero-3-phosphoethanolamine, depending on whether they are symmetric or asymmetric lipids. Exemplary phosphatidic acids include dimyristoyl phosphatidic acid, dipalmitoyl phosphatidic acid and dioleoyl phosphatidic acid. Phosphatidic acids may also be referred to under IUPAC naming systems as 1,2-diacyl-sn-glycero-3-phosphate or 1-acyl-2-acyl-sn-glycero-3-phosphate, depending on whether they are symmetric or asymmetric lipids.

Exemplary phosphatidylserines include dimyristoyl phosphatidylserine, dipalmitoyl phosphatidylserine, dioleoylphosphatidylserine, distearoyl phosphatidylserine, palmitoyl-oleylphosphatidylserine and brain phosphatidylserine. Phosphatidylserines may also be referred to under IUPAC naming systems as 1,2-diacyl-sn-glycero-3-[phospho-L-serine] or 1-acyl-2-acyl-sn-glycero-3-[phospho-L-serine], depending on whether they are symmetric or asymmetric lipids. As used herein, the abbreviation “PS” refers to phosphatidylserine.

Exemplary phosphatidylglycerols include dilauryloylphosphatidylglycerol, dipalmitoylphosphatidylglycerol, distearoylphosphatidylglycerol, dioleoyl-phosphatidylglycerol, dimyristoylphosphatidylglycerol, palmitoyl-oleoyl-phosphatidylglycerol and egg phosphatidylglycerol. Phosphatidylglycerols may also be referred to under IUPAC naming systems as 1,2-diacyl-sn-glycero-3-[phospho-rac-(1-glycerol)] or 1-acyl-2-acyl-sn-glycero-3-[phospho-rac-(1-glycerol)], depending on whether they are symmetric or asymmetric lipids. The phosphatidylglycerol 1,2-dimyristoyl-sn-glycero-3-[phospho-rac-(1-glycerol)] is abbreviated herein as “DMPG”. The phosphatidylglycerol 1,2-dipalmitoyl-sn-glycero-3-(phospho-rac-1-glycerol) (sodium salt) is abbreviated herein as “DPPG”.

Suitable sphingomyelins might include brain sphingomyelin, egg sphingomyelin, dipalmitoyl sphingomyelin, and distearoyl sphingomyelin.

Other suitable lipids include glycolipids, sphingolipids, ether lipids, glycolipids such as the cerebrosides and gangliosides, and sterols, such as cholesterol or ergosterol.

In some embodiments, dioleoylphosphatidylethanolamine (DOPE), polyethyleneimines (PEI), a neutral lipid, may often be used in conjunction with cationic lipids because of its membrane destabilizing effects at low pH, which can aide in endolysosomal escape.

### Anionic Lipids

In some embodiments, a lipid nanoparticle of a delivery vehicle provided herein can also comprise an anionic lipid. In general, an anionic lipid can contain any of a wide range of fatty acid chains in the hydrophobic region. The specific fatty acids incorporated are responsible for the fluidic characteristics of the lipid structure in terms of phase behavior and elasticity.

In some embodiments, divalent cations can be incorporated into an anionic lipid structure to enable the condensation of nucleic acids prior to envelopment by anionic lipids. Several divalent cations can be used in anionic lipoplexes such as Ca²⁺, Mg²⁺, Mn²⁺, and Ba²⁺.

In some embodiments, Ca²⁺ can be utilized in an anionic lipid structure.

In some embodiments, suitable anionic lipids include but are not limited to: phosphatidylglycerol, a cardiolipin, a diacylphosphatidylserine, a diacylphosphatidic acid, a N-dodecanoyl phosphatidylethanolamine, a N-succinyl phosphatidylethanolamine, a N-glutarylphosphatidylethanolamine, a lysylphosphatidylglycerol, a palmitoyloleyolphosphatidylglyeerol (POPG), or any combinations thereof.

In some embodiments, the anionic lipid in the lipid nanoparticles comprises at least one of phosphatidylglycerol, cardiolipin, dialkylphosphatidylserine, dialkylphosphatidic acid, N-dodecanoyl phosphatidylethanolamine, N-succinyl phosphatidylethanolamine, N-glutarylphosphatidylethanolamine, lysylphosphatidylglycerol, palmitoyloleyolphosphatidylglycerol (POPG), glycerophosphoinositol monophosphate, glycerophosphoinositol bisphosphate, glycerophosphoinositol trisphosphate, glycerophosphate, a glyceropyrophosphate, glycerophosphoglycerophosphoglycerol, cytidine-5′-diphosphate-glycerols, glycosylglycerophospholipid, a glycerophosphoinositolglycan, 1,2-dialkyl-sn-glycero-3-Phosphate, 1,2-dialkyl-sn-glycero-3-phosphomethanol, 1,2-dialkyl-sn-glycero-3-phosphoethanol, 1,2-dialkyl-sn-glycero-3-phosphopropanol, and/or 1,2-dialkyl-sn-glycero-3-phosphobutanol.

In some embodiments, where the anionic lipid is conjugated to an alkyl and the anionic lipid is present in the liquid phase, the alkyl is a conjugated derivative of at least one of oleic acid, elaidic acid, gondoic acid, erucic acid, nervonic acid, mead acid, paullinic acid, vaccenic acid, palmitoleic acid, Docosatetraenoic acid, Arachidonic acid, Dihomo-γ-linolenic acid, γ-Linolenic acid, linolelaidic acid, linoleic acid, Docosahexaenoic acid, Eicosapentaenoic acid, Stearidonic acid, α-Linolenic acid, or salts thereof, or any combinations thereof. In other cases, the alkyl is a conjugated derivative of at least one myristic acid, pentadecylic acid, palmitic acid, heptadecanoic acid, stearic acid, lauric acid, tridecylic acid, nonadecylic acid, arachidic acid, heneicosylic acid, behenic acid, tricosylic acid, lignoceric acid, and/or salts thereof, or any combinations thereof. In the above, if the alkyl has a phase transition temperature of >37C it is considered to be in the gel phase otherwise it is present in the liquid phase.

In some embodiments, an anionic lipid can be a saturated lipid with a phase transition temperature above 37C, such a lipid can be used in a solid phase and a cationic lipid in a liquid phase. In some embodiments, when an anionic lipid is unsaturated or a short chain lipid with a transition temperature below 37C then it may be employed in a liquid phase and a cationic lipid can be used in a gel or solid phase.

In some embodiments, bile salts can be used as an anionic component in the delivery vehicle. In some embodiments, non-bile salts can be used as an anionic component.

In some embodiments, an anionic liposome may be used to deliver other (non-nucleic acid) therapeutic agents.

### Sterols and Sterol Containing Mixtures

In some embodiments, a lipid structure can comprise cholesterol or a derivative thereof, a phospholipid, a mixture of a phospholipid and cholesterol or a derivative thereof, or a combination. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof.

In some embodiments, the structural lipid component is not a sterol. In some embodiments, the at least one structural lipid is not cholesterol. In some embodiments, the delivery vehicle contains less than 10 mole %, less than 5 mole %, less than 1 mole %, less than 0.1 mole %, less than 0.01 mole %, less than 0.001 mole %, less than 0.0001 mole % or cholesterol or a cholesterol derivative. In some embodiments, the delivery vehicle is formed without any cholesterol present in the formulation mixture. In some embodiments, the delivery vehicle contains essentially no cholesterol.

### Saturated Non-Cationic Lipids

In some embodiments, the delivery vehicle (i.e., a lipid delivery vehicle) can comprise (or further comprise) a saturated non-cationic lipid. In some embodiments, the saturated non-cationic lipid may have a phase transition temperature of at least about 20° C., 22° C., 24° C., 26° C., 28° C., 30° C., 32° C., 34° C., 36° C., 38° C., 40° C., 42° C., 44° C., 46° C., 48° C., 50° C., 52° C., 54° C., 56° C., 58° C., and/or up to about 60° C. For example, the saturated non-cationic lipid may have phase transition temperatures of about 30° C.-60° C., 35° C.-60° C., 37° C.-60° C., 37° C.-55° C., 37° C.-50° C., 37° C.-45° C., or 37° C.-40° C.

### Amount of Structural Lipid in the Delivery Vehicle

### Total Amount of Structural Lipid

In some embodiments, the structural lipid may comprise about 5-75 mole % of the total delivery vehicle lipids. For example, the structural lipid may comprise about 5-15 mole %, about 5-25 mole %, about 5-35 mole %, about 5-45 mole %, about 5-55 mole %, about 5-65 mole %, about 5-75 mole %, about 15-25 mole %, about 15-35 mole %, about 15-45 mole %, about 15-55 mole %, about 15-65 mole %, about 15-75 mole %, about 25-35 mole %, about 25-45 mole %, about 25-55 mole %, about 25-65 mole %, about 25-75 mole %, about 35-45 mole %, about 35-55 mole %, about 35-65 mole %, about 35-75 mole %, about 45-55 mole %, about 45-65 mole %, about 45-75 mole %, about 55-65 mole %, about 55-75 mole %, or about 65-75 mole % of the total delivery vehicle lipids.

In some embodiments, the structural lipid may comprise about 30-50 mole % of the delivery vehicle lipids. In some embodiments, the structural lipid may comprise about 35-45 mole % of the delivery vehicle lipids. In some embodiments, the structural lipid component may comprise about 40 mole % of the delivery vehicle lipids.

In some embodiments, nanoparticle structural lipids may include one or more of DSPC and DMPC and may be present at a level of from about 35 to about 45 mole % of total nanoparticle lipid.

### Amounts of Phospholipid Cholesterol Mixtures

In some embodiments, when a lipid structure comprises a mixture of a phospholipid and cholesterol or a cholesterol derivative, the lipid structure may comprise up to about 40, 50, or 60 mole % of the total lipid present in the lipid structure. One or more phospholipids and/or cholesterol may comprise from about 10 mole % to about 60 mole %, from about 15 mole % to about 60 mole %, from about 20 mole % to about 60 mole %, from about 25 mole % to about 60 mole %, from about 30 mole % to about 60 mole %, from about 10 mole % to about 55 mole %, from about 15 mole % to about 55 mole %, from about 20 mole % to about 55 mole %, from about 25 mole % to about 55 mole %, from about 30 mole % to about 55 mole %, from about 13 mole % to about 50 mole %, from about 15 mole % to about 50 mole % or from about 20 mole % to about 50 mole % of the total lipid present in the lipid structure.

### Amounts of Phospholipid Cholesterol Mixtures

In some embodiments, the concentration of at least one unsaturated non-cationic lipid in a lipid nanoparticle can be less than 50 mole %, 45 mole %, 40 mole %, 35 mole %, 30 mole %, 25 mole %, 20 mole %, 15 mole %, 10 mole %, 5 mole %, or 2 mole % of the total lipid concentration of the lipid nanoparticle. In some embodiments, the concentration of at least one unsaturated non-cationic lipid in a lipid nanoparticle can be about 50 mole %, 45 mole %, 40 mole %, 35 mole %, 30 mole %, 25 mole %, 20 mole %, 15 mole %, 10 mole %, 5 mole %, or 2 mole % of the total lipid concentration of the lipid nanoparticle. In some embodiments, the concentration of at least one unsaturated non-cationic lipid in a lipid nanoparticle can be 5-50 mole %, 5-40 mole %, 5-30 mole %, 5-25 mole %, 5-20 mole %, 5-15 mole %, 10-50 mole %, 10-40 mole %, 10-30 mole %, 10-25 mole %, 15-50 mole %, 15-40 mole %, 15-30 mole % and 15-25 mole %.

### Conjugated Lipid

In some embodiments, the delivery vehicles may comprise at least one conjugated lipid. In some embodiments, the at least one conjugated lipid may be comprised of at least one conjugated lipid and at least one hydrophilic polymer.

In some embodiments, the conjugated lipid may comprise at least one lipid selected from but not limited to, a phospholipid, a neutral lipid, a glyceride, a diglyceride, or any combination thereof.

In some embodiments, the conjugated lipid may comprise any of, but not limited to, 1,2-dimyristoyl-rac-glycerol (DMG), 1,2-dimyristoyl-sn-glycero-3-phosphoethanolamine (DMPE), 1,2-distearoyl-rac-glycerol (DSG), 1,2-dipalmitoyl-rac-glycerol (DPG), 1,2-distearoyl-sn-glycero-3-phosphoethanolamine (DSPE), diacylglycerol (DAG), 1,2-dipalmitoryl-sn-glycero-3-phosphoethanolamine (DPPE), or any combination thereof.

In some embodiments, the hydrophilic polymer can comprise polyethylene glycol, a poly (2-alkyl-2-oxazoline), a polyvinyl alcohol, or any combinations thereof. In some embodiments, the hydrophilic polymer can comprise a molecular weight from at least about 500 Da to about 500 kDa.

In some embodiments, the average molecular weight of the hydrophilic polymer (e.g., PEG) may be between 500 and 20,000 daltons. In some embodiments, the molecular weight of the hydrophilic polymer may be about 500 to 20,000, 1,000 to 20,000, 1,500 to 20,000, 2,000 to 20,000, 2,500 to 20,000, 3,000 to 20,000, 3,500 to 20,000, 4,000 to 20,000, 4,500 to 20,000, 5,000 to 20,000, 5,500 to 20,000, 6,000 to 20,000, 6,500 to 20,000, 7,000 to 20,000, 7,500 to 20,000, 8,000 to 20,000, 8,500 to 20,000, 9,000 to 20,000, 9,500 to 20,000, 10,000 to 20,000, 10,500 to 20,000, 11,000 to 20,000, 11,500 to 20,000, 12,000 to 20,000, 12,500 to 20,000, 13,000 to 20,000, 13,500 to 20,000, 14,000 to 20,000, 14,500 to 20,000, 15,000 to 20,000, 15,500 to 20,000, 16,000 to 20,000, 16,500 to 20,000, 17,000 to 20,000, 17,500 to 20,000, 18,000 to 20,000, 18,500 to 20,000, 19,000 to 20,000, 19,500 to 20,000, 500 to 19,500, 1,000 to 19,500, 1,500 to 19,500, 2,000 to 19,500, 2,500 to 19,500, 3,000 to 19,500, 3,500 to 19,500, 4,000 to 19,500, 4,500 to 19,500, 5,000 to 19,500, 5,500 to 19,500, 6,000 to 19,500, 6,500 to 19,500, 7,000 to 19,500, 7,500 to 19,500, 8,000 to 19,500, 8,500 to 19,500, 9,000 to 19,500, 9,500 to 19,500, 10,000 to 19,500, 10,500 to 19,500, 11,000 to 19,500, 11,500 to 19,500, 12,000 to 19,500, 12,500 to 19,500, 13,000 to 19,500, 13,500 to 19,500, 14,000 to 19,500, 14,500 to 19,500, 15,000 to 19,500, 15,500 to 19,500, 16,000 to 19,500, 16,500 to 19,500, 17,000 to 19,500, 17,500 to 19,500, 18,000 to 19,500, 18,500 to 19,500, 19,000 to 19,500, 1,500 to 19,000, 2,000 to 19,000, 2,500 to 19,000, 3,000 to 19,000, 3,500 to 19,000, 4,000 to 19,000, 4,500 to 19,000, 5,000 to 19,000, 5,500 to 19,000, 6,000 to 19,000, 6,500 to 19,000, 7,000 to 19,000, 7,500 to 19,000, 8,000 to 19,000, 8,500 to 19,000, 9,000 to 19,000, 9,500 to 19,000, 10,000 to 19,000, 10,500 to 19,000, 11,000 to 19,000, 11,500 to 19,000, 12,000 to 19,000, 12,500 to 19,000, 13,000 to 19,000, 13,500 to 19,000, 14,000 to 19,000, 14,500 to 19,000, 15,000 to 19,000, 15,500 to 19,000, 16,000 to 19,000, 16,500 to 19,000, 17,000 to 19,000, 17,500 to 19,000, 18,000 to 19,000, 18,500 to 19,000, 1,500 to 18,500, 2,000 to 18,500, 2,500 to 18,500, 3,000 to 18,500, 3,500 to 18,500, 4,000 to 18,500, 4,500 to 18,500, 5,000 to 18,500, 5,500 to 18,500, 6,000 to 18,500, 6,500 to 18,500, 7,000 to 18,500, 7,500 to 18,500, 8,000 to 18,500, 8,500 to 18,500, 9,000 to 18,500, 9,500 to 18,500, 10,000 to 18,500, 10,500 to 18,500, 11,000 to 18,500, 11,500 to 18,500, 12,000 to 18,500, 12,500 to 18,500, 13,000 to 18,500, 13,500 to 18,500, 14,000 to 18,500, 14,500 to 18,500, 15,000 to 18,500, 15,500 to 18,500, 16,000 to 18,500, 16,500 to 18,500, 17,000 to 18,500, 17,500 to 18,500, 18,000 to 18,500, 1,500 to 18,000, 2,000 to 18,000, 2,500 to 18,000, 3,000 to 18,000, 3,500 to 18,000, 4,000 to 18,000, 4,500 to 18,000, 5,000 to 18,000, 5,500 to 18,000, 6,000 to 18,000, 6,500 to 18,000, 7,000 to 18,000, 7,500 to 18,000, 8,000 to 18,000, 8,500 to 18,000, 9,000 to 18,000, 9,500 to 18,000, 10,000 to 18,000, 10,500 to 18,000, 11,000 to 18,000, 11,500 to 18,000, 12,000 to 18,000, 12,500 to 18,000, 13,000 to 18,000, 13,500 to 18,000, 14,000 to 18,000, 14,500 to 18,000, 15,000 to 18,000, 15,500 to 18,000, 16,000 to 18,000, 16,500 to 18,000, 17,000 to 18,000, 17,500 to 18,000, 1,500 to 17,500, 2,000 to 17,500, 2,500 to 17,500, 3,000 to 17,500, 3,500 to 17,500, 4,000 to 17,500, 4,500 to 17,500, 5,000 to 17,500, 5,500 to 17,500, 6,000 to 17,500, 6,500 to 17,500, 7,000 to 17,500, 7,500 to 17,500, 8,000 to 17,500, 8,500 to 17,500, 9,000 to 17,500, 9,500 to 17,500, 10,000 to 17,500, 10,500 to 17,500, 11,000 to 17,500, 11,500 to 17,500, 12,000 to 17,500, 12,500 to 17,500, 13,000 to 17,500, 13,500 to 17,500, 14,000 to 17,500, 14,500 to 17,500, 15,000 to 17,500, 15,500 to 17,500, 16,000 to 17,500, 16,500 to 17,500, 17,000 to 17,500, 1,500 to 17,000, 2,000 to 17,000, 2,500 to 17,000, 3,000 to 17,000, 3,500 to 17,000, 4,000 to 17,000, 4,500 to 17,000, 5,000 to 17,000, 5,500 to 17,000, 6,000 to 17,000, 6,500 to 17,000, 7,000 to 17,000, 7,500 to 17,000, 8,000 to 17,000, 8,500 to 17,000, 9,000 to 17,000, 9,500 to 17,000, 10,000 to 17,000, 10,500 to 17,000, 11,000 to 17,000, 11,500 to 17,000, 12,000 to 17,000, 12,500 to 17,000, 13,000 to 17,000, 13,500 to 17,000, 14,000 to 17,000, 14,500 to 17,000, 15,000 to 17,000, 15,500 to 17,000, 16,000 to 17,000, 16,500 to 17,000, 1,500 to 16,500, 2,000 to 16,500, 2,500 to 16,500, 3,000 to 16,500, 3,500 to 16,500, 4,000 to 16,500, 4,500 to 16,500, 5,000 to 16,500, 5,500 to 16,500, 6,000 to 16,500, 6,500 to 16,500, 7,000 to 16,500, 7,500 to 16,500, 8,000 to 16,500, 8,500 to 16,500, 9,000 to 16,500, 9,500 to 16,500, 10,000 to 16,500, 10,500 to 16,500, 11,000 to 16,500, 11,500 to 16,500, 12,000 to 16,500, 12,500 to 16,500, 13,000 to 16,500, 13,500 to 16,500, 14,000 to 16,500, 14,500 to 16,500, 15,000 to 16,500, 15,500 to 16,500, 16,000 to 16,500, 1,500 to 16,000, 2,000 to 16,000, 2,500 to 16,000, 3,000 to 16,000, 3,500 to 16,000, 4,000 to 16,000, 4,500 to 16,000, 5,000 to 16,000, 5,500 to 16,000, 6,000 to 16,000, 6,500 to 16,000, 7,000 to 16,000, 7,500 to 16,000, 8,000 to 16,000, 8,500 to 16,000, 9,000 to 16,000, 9,500 to 16,000, 10,000 to 16,000, 10,500 to 16,000, 11,000 to 16,000, 11,500 to 16,000, 12,000 to 16,000, 12,500 to 16,000, 13,000 to 16,000, 13,500 to 16,000, 14,000 to 16,000, 14,500 to 16,000, 15,000 to 16,000, 15,500 to 16,000, 1,500 to 15,500, 2,000 to 15,500, 2,500 to 15,500, 3,000 to 15,500, 3,500 to 15,500, 4,000 to 15,500, 4,500 to 15,500, 5,000 to 15,500, 5,500 to 15,500, 6,000 to 15,500, 6,500 to 15,500, 7,000 to 15,500, 7,500 to 15,500, 8,000 to 15,500, 8,500 to 15,500, 9,000 to 15,500, 9,500 to 15,500, 10,000 to 15,500, 10,500 to 15,500, 11,000 to 15,500, 11,500 to 15,500, 12,000 to 15,500, 12,500 to 15,500, 13,000 to 15,500, 13,500 to 15,500, 14,000 to 15,500, 14,500 to 15,500, 15,000 to 15,500, 1,500 to 15,000, 2,000 to 15,000, 2,500 to 15,000, 3,000 to 15,000, 3,500 to 15,000, 4,000 to 15,000, 4,500 to 15,000, 5,000 to 15,000, 5,500 to 15,000, 6,000 to 15,000, 6,500 to 15,000, 7,000 to 15,000, 7,500 to 15,000, 8,000 to 15,000, 8,500 to 15,000, 9,000 to 15,000, 9,500 to 15,000, 10,000 to 15,000, 10,500 to 15,000, 11,000 to 15,000, 11,500 to 15,000, 12,000 to 15,000, 12,500 to 15,000, 13,000 to 15,000, 13,500 to 15,000, 14,000 to 15,000, 14,500 to 15,000, 1,500 to 14,500, 2,000 to 14,500, 2,500 to 14,500, 3,000 to 14,500, 3,500 to 14,500, 4,000 to 14,500, 4,500 to 14,500, 5,000 to 14,500, 5,500 to 14,500, 6,000 to 14,500, 6,500 to 14,500, 7,000 to 14,500, 7,500 to 14,500, 8,000 to 14,500, 8,500 to 14,500, 9,000 to 14,500, 9,500 to 14,500, 10,000 to 14,500, 10,500 to 14,500, 11,000 to 14,500, 11,500 to 14,500, 12,000 to 14,500, 12,500 to 14,500, 13,000 to 14,500, 13,500 to 14,500, 14,000 to 14,500, 1,500 to 14,000, 2,000 to 14,000, 2,500 to 14,000, 3,000 to 14,000, 3,500 to 14,000, 4,000 to 14,000, 4,500 to 14,000, 5,000 to 14,000, 5,500 to 14,000, 6,000 to 14,000, 6,500 to 14,000, 7,000 to 14,000, 7,500 to 14,000, 8,000 to 14,000, 8,500 to 14,000, 9,000 to 14,000, 9,500 to 14,000, 10,000 to 14,000, 10,500 to 14,000, 11,000 to 14,000, 11,500 to 14,000, 12,000 to 14,000, 12,500 to 14,000, 13,000 to 14,000, 13,500 to 14,000, 1,500 to 13,500, 2,000 to 13,500, 2,500 to 13,500, 3,000 to 13,500, 3,500 to 13,500, 4,000 to 13,500, 4,500 to 13,500, 5,000 to 13,500, 5,500 to 13,500, 6,000 to 13,500, 6,500 to 13,500, 7,000 to 13,500, 7,500 to 13,500, 8,000 to 13,500, 8,500 to 13,500, 9,000 to 13,500, 9,500 to 13,500, 10,000 to 13,500, 10,500 to 13,500, 11,000 to 13,500, 11,500 to 13,500, 12,000 to 13,500, 12,500 to 13,500, 13,000 to 13,500, 1,500 to 13,000, 2,000 to 13,000, 2,500 to 13,000, 3,000 to 13,000, 3,500 to 13,000, 4,000 to 13,000, 4,500 to 13,000, 5,000 to 13,000, 5,500 to 13,000, 6,000 to 13,000, 6,500 to 13,000, 7,000 to 13,000, 7,500 to 13,000, 8,000 to 13,000, 8,500 to 13,000, 9,000 to 13,000, 9,500 to 13,000, 10,000 to 13,000, 10,500 to 13,000, 11,000 to 13,000, 11,500 to 13,000, 12,000 to 13,000, 12,500 to 13,000, 1,500 to 12,500, 2,000 to 12,500, 2,500 to 12,500, 3,000 to 12,500, 3,500 to 12,500, 4,000 to 12,500, 4,500 to 12,500, 5,000 to 12,500, 5,500 to 12,500, 6,000 to 12,500, 6,500 to 12,500, 7,000 to 12,500, 7,500 to 12,500, 8,000 to 12,500, 8,500 to 12,500, 9,000 to 12,500, 9,500 to 12,500, 10,000 to 12,500, 10,500 to 12,500, 11,000 to 12,500, 11,500 to 12,500, 12,000 to 12,500, 1,500 to 12,000, 2,000 to 12,000, 2,500 to 12,000, 3,000 to 12,000, 3,500 to 12,000, 4,000 to 12,000, 4,500 to 12,000, 5,000 to 12,000, 5,500 to 12,000, 6,000 to 12,000, 6,500 to 12,000, 7,000 to 12,000, 7,500 to 12,000, 8,000 to 12,000, 8,500 to 12,000, 9,000 to 12,000, 9,500 to 12,000, 10,000 to 12,000, 10,500 to 12,000, 11,000 to 12,000, 11,500 to 12,000, 1,500 to 11,500, 2,000 to 11,500, 2,500 to 11,500, 3,000 to 11,500, 3,500 to 11,500, 4,000 to 11,500, 4,500 to 11,500, 5,000 to 11,500, 5,500 to 11,500, 6,000 to 11,500, 6,500 to 11,500, 7,000 to 11,500, 7,500 to 11,500, 8,000 to 11,500, 8,500 to 11,500, 9,000 to 11,500, 9,500 to 11,500, 10,000 to 11,500, 10,500 to 11,500, 11,000 to 11,500, 1,500 to 11,000, 2,000 to 11,000, 2,500 to 11,000, 3,000 to 11,000, 3,500 to 11,000, 4,000 to 11,000, 4,500 to 11,000, 5,000 to 11,000, 5,500 to 11,000, 6,000 to 11,000, 6,500 to 11,000, 7,000 to 11,000, 7,500 to 11,000, 8,000 to 11,000, 8,500 to 11,000, 9,000 to 11,000, 9,500 to 11,000, 10,000 to 11,000, 10,500 to 11,000, 1,500 to 10,500, 2,000 to 10,500, 2,500 to 10,500, 3,000 to 10,500, 3,500 to 10,500, 4,000 to 10,500, 4,500 to 10,500, 5,000 to 10,500, 5,500 to 10,500, 6,000 to 10,500, 6,500 to 10,500, 7,000 to 10,500, 7,500 to 10,500, 8,000 to 10,500, 8,500 to 10,500, 9,000 to 10,500, 9,500 to 10,500, 10,000 to 10,500, 1,500 to 10,000, 2,000 to 10,000, 2,500 to 10,000, 3,000 to 10,000, 3,500 to 10,000, 4,000 to 10,000, 4,500 to 10,000, 5,000 to 10,000, 5,500 to 10,000, 6,000 to 10,000, 6,500 to 10,000, 7,000 to 10,000, 7,500 to 10,000, 8,000 to 10,000, 8,500 to 10,000, 9,000 to 10,000, 9,500 to 10,000, 1,500 to 9,500, 2,000 to 9,500, 2,500 to 9,500, 3,000 to 9,500, 3,500 to 9,500, 4,000 to 9,500, 4,500 to 9,500, 5,000 to 9,500, 5,500 to 9,500, 6,000 to 9,500, 6,500 to 9,500, 7,000 to 9,500, 7,500 to 9,500, 8,000 to 9,500, 8,500 to 9,500, 9,000 to 9,500, 1,500 to 9,000, 2,000 to 9,000, 2,500 to 9,000, 3,000 to 9,000, 3,500 to 9,000, 4,000 to 9,000, 4,500 to 9,000, 5,000 to 9,000, 5,500 to 9,000, 6,000 to 9,000, 6,500 to 9,000, 7,000 to 9,000, 7,500 to 9,000, 8,000 to 9,000, 8,500 to 9,000, 1,500 to 8,500, 2,000 to 8,500, 2,500 to 8,500, 3,000 to 8,500, 3,500 to 8,500, 4,000 to 8,500, 4,500 to 8,500, 5,000 to 8,500, 5,500 to 8,500, 6,000 to 8,500, 6,500 to 8,500, 7,000 to 8,500, 7,500 to 8,500, 8,000 to 8,500, 1,500 to 8,000, 2,000 to 8,000, 2,500 to 8,000, 3,000 to 8,000, 3,500 to 8,000, 4,000 to 8,000, 4,500 to 8,000, 5,000 to 8,000, 5,500 to 8,000, 6,000 to 8,000, 6,500 to 8,000, 7,000 to 8,000, 7,500 to 8,000, 1,500 to 7,500, 2,000 to 7,500, 2,500 to 7,500, 3,000 to 7,500, 3,500 to 7,500, 4,000 to 7,500, 4,500 to 7,500, 5,000 to 7,500, 5,500 to 7,500, 6,000 to 7,500, 6,500 to 7,500, 7,000 to 7,500, 1,500 to 7,000, 2,000 to 7,000, 2,500 to 7,000, 3,000 to 7,000, 3,500 to 7,000, 4,000 to 7,000, 4,500 to 7,000, 5,000 to 7,000, 5,500 to 7,000, 6,000 to 7,000, 6,500 to 7,000, 1,500 to 6,500, 2,000 to 6,500, 2,500 to 6,500, 3,000 to 6,500, 3,500 to 6,500, 4,000 to 6,500, 4,500 to 6,500, 5,000 to 6,500, 5,500 to 6,500, 6,000 to 6,500, 1,500 to 6,000, 2,000 to 6,000, 2,500 to 6,000, 3,000 to 6,000, 3,500 to 6,000, 4,000 to 6,000, 4,500 to 6,000, 5,000 to 6,000, 5,500 to 6,000, 1,500 to 5,500, 2,000 to 5,500, 2,500 to 5,500, 3,000 to 5,500, 3,500 to 5,500, 4,000 to 5,500, 4,500 to 5,500, 5,000 to 5,500, 1,500 to 5,000, 2,000 to 5,000, 2,500 to 5,000, 3,000 to 5,000, 3,500 to 5,000, 4,000 to 5,000, 4,500 to 5,000, 1,500 to 4,500, 2,000 to 4,500, 2,500 to 4,500, 3,000 to 4,500, 3,500 to 4,500, 4,000 to 4,500, 1,500 to 4,000, 2,000 to 4,000, 2,500 to 4,000, 3,000 to 4,000, 3,500 to 4,000, 1,500 to 3,500, 2,000 to 3,500, 2,500 to 3,500, 3,000 to 3,500, 1,500 to 3,000, 2,000 to 3,000, 2,500 to 3,000, 1,500 to 2,500, 2,000 to 2,500, and 1,500 to 2,000 daltons.

In some embodiments the at least one hydrophilic polymer may comprise polyethylene glycol (PEG). In some embodiments, the hydrophilic polymer can include polyethyleneglycol (PEG), and the conjugated lipid may be a pegylated lipid. In some embodiments, the pegylated lipid can comprise 1,2-distearoyl-sn-glycero-3-phosphoethanolamine (DSPE)-PEG, 1,2-distearoyl-rac-glycerol (DSG)-PEG, 1,2-dipalmitoyl-rac-glycerol (DPG)-PEG, diacylglycerol (DAG)-PEG, 1,2-dimyristoyl-rac-glycerol (DMG)-PEG, 1,2-dipalmitoryl-sn-glycero-3-phosphoethanolamine (DPPE)-PEG, 1,2-dimyristoyl-sn-glycero-3-phosphoethanolamine (DMPE)-PEG, or any combinations thereof.

In some embodiments, the conjugated lipid may comprise Siglec-1L-PEG-DSPE, Siglec-1L-PEG-DSPE, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, C¹8PEG750, CI8PEG5000, CI8PEG3000, CI8PEG2000, CI6PEG2000, CI4PEG2000, C18-PEG5000, C18PEG, C16PEG, C14-PEG-DSPE200, C14-PEG2000, C14PEG2000, C14-PEG 2000, C14-PEG, C14PEG, (PEG)-C-DOMG, PEG-C-DMA, and any combination thereof.

In some embodiments, the conjugated lipid component may comprise DMG-PEG. In some embodiments, the conjugated lipid component may comprise DMPE-PEG.

Without wishing to be bound by theory, it is believed that the conjugated hydrophilic polymer may reduce aggregation of the lipid components, and as such is sometimes referred to as a stability or stabilizing component.

### Amount of Conjugated Lipid in the Delivery Vehicle

In some embodiments, the conjugated lipid may comprise about 0.5-2.0 mole % of the delivery vehicle lipids. For example, the conjugated lipid component may comprise about 0.1-2 mole %, about 0.1-1.8 mole %, about 0.1-1.6 mole %, about 0.1-1.5 mole %, about 0.1-1.4 mole %, about 0.1-1.2 mole %, about 0.1-1 mole %, about 0.1-0.8 mole %, about 0.1-0.6 mole %, about 0.1-0.4 mole %, about 0.1-0.3 mole %, about 0.1-0.2 mole %, about 0.2-2 mole %, about 0.2-1.8 mole %, about 0.2-1.6 mole %, about 0.2-1.5 mole %, about 0.2-1.4 mole %, about 0.2-1.2 mole %, about 0.2-1 mole %, about 0.2-0.8 mole %, about 0.2-0.6 mole %, about 0.2-0.4 mole %, about 0.2-0.3 mole %, about 0.3-2 mole %, about 0.3-1.8 mole %, about 0.3-1.6 mole %, about 0.3-1.5 mole %, about 0.3-1.4 mole %, about 0.3-1.2 mole %, about 0.3-1 mole %, about 0.3-0.8 mole %, about 0.3-0.6 mole %, about 0.3-0.4 mole %, about 0.4-2 mole %, about 0.4-1.8 mole %, about 0.4-1.6 mole %, about 0.4-1.5 mole %, about 0.4-1.4 mole %, about 0.4-1.2 mole %, about 0.4-1 mole %, about 0.4-0.8 mole %, about 0.4-0.6 mole %, about 0.6-2 mole %, about 0.6-1.8 mole %, about 0.6-1.6 mole %, about 0.6-1.5 mole %, about 0.6-1.4 mole %, about 0.6-1.2 mole %, about 0.6-1 mole %, about 0.6-0.8 mole %, about 0.8-2 mole %, about 0.8-1.8 mole %, about 0.8-1.6 mole %, about 0.8-1.5 mole %, about 0.8-1.4 mole %, about 0.8-1.2 mole %, about 0.8-1 mole %, about 1-2 mole %, about 1-1.8 mole %, about 1-1.6 mole %, about 1-1.5 mole %, about 1-1.4 mole %, about 1-1.2 mole %, about 1.2-2 mole %, about 1.2-1.8 mole %, about 1.2-1.6 mole %, about 1.2-1.5 mole %, about 1.2-1.4 mole %, about 1.4-2 mole %, about 1.4-1.8 mole %, about 1.4-1.6 mole %, about 1.4-1.5 mole %, about 1.5-2 mole %, about 1.5-1.8 mole %, about 1.5-1.6 mole %, about 1.6-2 mole %, about 1.6-1.8 mole %, about or 1.8-2 mole % of the delivery vehicle lipids. In some embodiments, the conjugated lipid may comprise about 1 mole % of the delivery vehicle lipids.

In some embodiments, the concentration of the conjugated lipid can more than about 0 mole %, 0.5 mole %, 1 mole %, 1.5 mole %, 2 mole %, 2.5 mole %, 3 mole %, 3.5 mole %, 4 mole %, 4.5 mole %, 5 mole %, 5.5 mole %, 6 mole %, 6.5 mole %, 7 mole %, 7.5 mole %, 8 mole %, 8.5 mole %, 9 mole %, 9.5 mole %, 10 mole %, 10.5 mole %, 11 mole %, 11.5 mole %, 12 mole %, 12.5 mole %, 13 mole %, 13.5 mole %, 14 mole %, 14.5 mole %, 15 mole %, 15.5 mole %, 16 mole %, 16.5 mole %, 17 mole %, 17.5 mole %, 18 mole %, 18.5 mole %, 19 mole %, 19.5 mole %, 20 mole %, 20.5 mole %, 21 mole %, 21.5 mole %, 22 mole %, 22.5 mole %, 23 mole %, 23.5 mole %, 24 mole %, 24.5 mole %, 25 mole %, 25.5 mole %, 26 mole %, 26.5 mole %, 27 mole %, 27.5 mole %, 28 mole %, 28.5 mole %, 29 mole %, 29.5 mole %, or 30 mole %. In some embodiments, a concentration of the conjugated lipid is from about 0.5 mole % to about 20 mole %, 0.5 mole % to about 5 mole %, 0.5 mole % to about 10 mole %, 5 mole % to about 10 mole %, or 10 mole % to about 20 mole %.

In some embodiments, the concentration of the conjugated lipid can be less than about 0.5 mole %, 1 mole %, 1.5 mole %, 2 mole %, 2.5 mole %, 3 mole %, 3.5 mole %, 4 mole %, 4.5 mole %, 5 mole %, 5.5 mole %, 6 mole %, 6.5 mole %, 7 mole %, 7.5 mole %, 8 mole %, 8.5 mole %, 9 mole %, 9.5 mole %, 10 mole %, 10.5 mole %, 11 mole %, 11.5 mole %, 12 mole %, 12.5 mole %, 13 mole %, 13.5 mole %, 14 mole %, 14.5 mole %, 15 mole %, 15.5 mole %, 16 mole %, 16.5 mole %, 17 mole %, 17.5 mole %, 18 mole %, 18.5 mole %, 19 mole %, 19.5 mole %, 20 mole %, 20.5 mole %, 21 mole %, 21.5 mole %, 22 mole %, 22.5 mole %, 23 mole %, 23.5 mole %, 24 mole %, 24.5 mole %, 25 mole %, 25.5 mole %, 26 mole %, 26.5 mole %, 27 mole %, 27.5 mole %, 28 mole %, 28.5 mole %, 29 mole %, 29.5 mole %, or 30 mole %. In some embodiments, a concentration of the conjugated lipid is from about 0.5 mole % to about 20 mole %, 0.5 mole % to about 5 mole %, 0.5 mole % to about 10 mole %, 5 mole % to about 10 mole %, or 10 mole % to about 20 mole %.

### Additional Delivery Vehicle Components

In some embodiments, the delivery vehicles herein, such as the lipid structures described herein for such purposes, further comprise additional components, such as but not limited to, mucus-penetrating peptide (MPPs), cell-penetrating peptides (CPP), a ligand, a mucus penetrating polymer, a targeting agent, or any combinations thereof. In some embodiments, the delivery vehicle may comprise additional components which are conjugated to lipids or modifications of lipids of the delivery vehicle. In some embodiments, the delivery vehicle may comprise additional components which are conjugated to the at least one conjugated lipid of the delivery vehicle.

In some embodiments, nanoparticle conjugated lipids may be conjugated with hydrophilic polymers. Hydrophilic polymers may include PEG. Conjugated lipids may include one or more of DMG-PEG and DMPE-PEG and may be present at a level of from about 0.5 to about 2.0 mole % of total nanoparticle lipid.

### Alternative Lipids

In some embodiments, the delivery vehicles may comprise alternative lipid families or species.

In some embodiments, the delivery vehicles herein can include additional components. For example, a lipid structure for the delivery vehicle can include a lipid bilayer. In certain cases, a lipid bilayer can be generated of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg, and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5 rapeseed sterol. In certain embodiments, the phytosterol comprises a mixture of at least two of the following compositions: sistosterol, camposterol and stigmasterol. In still other embodiments, a lipid layer can be comprised of one or more phosphatidyl groups selected from the group comprising phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol or lyso-phosphatidyl-inositol.

In some embodiments, a lipid bilayer can be comprised of phospholipid selected from a monoacyl or diacylphosphoglyceride. In still other cases, a lipid bilayer can be comprised of one or more phosphoinositides selected from the group comprising phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), phosphatidyl-inositol (PI), or lysophosphatidyl-inositol (LPI).

In some embodiments, lipids of a lipid structure, such as a liposome, may be or may comprise fatty acids, glycerolipids, glycerophospholipids, sphingolipids, saccharolipids, polyketides (derived from condensation of ketoacyl subunits); sterol lipids prenol lipids (derived from condensation of isoprene subunits) or any combination thereof.

### Saturated Cationic Lipids

In some embodiments, the delivery vehicle may comprise at least one saturated cationic lipid. The term “saturated” when used to describe a lipid herein, is used in its broadest sense to refer to a lipid containing the greatest possible number of hydrogen atoms, i.e., containing no carbon-carbon double or triple bonds.

In some embodiments, the saturated cationic lipid may have a phase transition temperature that is at least about 20° C.

In some embodiments, the saturated cationic lipid may comprise a saturated cationic lipid that has a phase transition temperature of at least about 37° C.

In some embodiments, a saturated cationic lipid can be employed in a delivery vehicle provided herein. A saturated cationic lipid can have a positive charge at pH 4, or at a pH greater than pH 4. In examples where the saturated cationic lipid comprises an alkyl, the alkyl can be a conjugated derivative of at least one of: myristoyl, pentadecanoyl, palmitoyl, heptadecanoyl, stearoyl, lauroyl, tridecanoyl, nonadecanoyl, arachidoyl, heneicasnoyl, behenoyl, tricosanoyl, lignoceroyl, or any combinations thereof.

In some embodiments, the delivery vehicle of this disclosure can comprise at least one saturated cationic lipid and at least a bile salt, wherein the at least one saturated cationic lipid can have a phase transition temperature of at least about 37° C. In some embodiments, the saturated cationic lipid has a phase transition temperature of at least about 20° C., 22° C., 24° C., 26° C., 28° C., 30° C., 32° C., 34° C., 36° C., 38° C., 40° C., 42° C., 44° C., 46° C., 48° C., 50° C., 52° C., 54° C., 56° C., 58° C., and/or up to about 60° C. For example, the saturated cationic lipid can have a phase transition temperature of 30° C.-60° C., 35° C.-60° C., 37° C.-60° C., 37° C.-55° C., 37° C.-50° C., 37° C.-45° C., or 37° C.-40° C. In some embodiments, a delivery vehicle of this disclosure can comprise at least one saturated cationic lipid, wherein the at least one saturated cationic lipid can have a phase transition temperature of at least about 37° C. In some embodiments, a cationic lipid may be in a gel phase of a lipid structure and an anionic lipid may be in a liquid phase.

In some embodiments, the saturated cationic lipid may comprise at least one of: 1,2-stearoyl-3-trimethylammonium-propane (DSTAP), 1,2-dipalmitoyl-3-trimethylammonium-propane (DPTAP), 1,2-Distearoyl-3-Dimethylammonium-Propane (DSDAP), or any combinations thereof. In some embodiments, the saturated cationic lipid can comprise at least one of: 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, or any combinations thereof.

### Unsaturated Cationic Lipids

In some embodiments, the delivery vehicle may comprise at least one unsaturated cationic lipid. The term “unsaturated” when used to describe a lipid herein, is used in its broadest sense to refer to a lipid containing the less than the greatest possible number of hydrogen atoms, i.e., containing at least one carbon-carbon double or triple bonds.

In some embodiments, the unsaturated cationic lipid can have a positive charge at about pH 4, or at a pH greater than about pH 4 and less than about pH 8. In examples where the unsaturated cationic lipid comprises an alkyl, the alkyl can be a conjugated derivative of at least one of: oleic acid, elaidic acid, gondoic acid, erucic acid, nervonic acid, mead acid, paullinic acid, vaccenic acid, palmitoleic acid, Docosatetraenoic acid, Arachidonic acid, Dihomo-γ-linolenic acid, γ-Linolenic acid, linolelaidic acid, linoleic acid, Docosahexaenoic acid, Eicosapentaenoic acid, Stearidonic acid, α-Linolenic acid, or any combinations thereof. In some embodiments, a cationic lipid may be in a liquid phase of a lipid structure and an anionic lipid may be in a gel phase or solid phase of a lipid structure.

In some embodiments, the unsaturated cationic lipid can comprise at least one of: 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 1,2-Dialkyloxy-N,N-dimethylaminopropane, 4-(2,2-diocta-9,12-dienyl-[1,3]dioxolan-4-ylmethyl)-dimethylamine, O-alkyl ethylphosphocholines, (6Z,9Z,28Z,31Z)-heptatriacont-6,9,28,31-tetraene-19-yl 4-(dimethylamino)butanoate (MC3), MC2, MC4,3β-[N-(N′,N′-dimethylaminoethane)-carbamoyl]cholesterol, N4-Cholesteryl-Spermine, or salts thereof, or any combinations thereof. In some embodiments, a cationic lipid may comprise or may be 7-(4-(dimethylamino)butyl)-7-hydroxytridecane-1,13-diyl dioleate (CL1H6), CL1A6, CL1A6, CL3A6, CL4A6, CL5A6, CL6A6, CL7A6, CL8A6, CL9A6, CL10A6, CL11A6, CL12A6, CL13A6, CL14A6, CL15A6, YSK12-C4, as described in US20200129431A1 and Sato Y et al. Understanding structure-activity relationships of pH-sensitive cationic lipids facilitates the rational identification of promising lipid nanoparticles for delivering siRNAs in vivo. J Control Release. 2019; 295:140-152 both herein incorporated by reference in relation to unsaturated cationic lipids.

In some embodiments, the concentration of at least one unsaturated cationic lipid in a lipid nanoparticle can be less than 50 mole %, 45 mole %, 40 mole %, 35 mole %, 30 mole %, 25 mole %, 20 mole %, 15 mole %, 10 mole %, 5 mole %, or 2 mole % of the total lipid concentration of the lipid nanoparticle. In some embodiments, the concentration of at least one unsaturated cationic lipid in a lipid nanoparticle can be about 50 mole %, 45 mole %, 40 mole %, 35 mole %, 30 mole %, 25 mole %, 20 mole %, 15 mole %, 10 mole %, 5 mole %, or 2 mole % of the total lipid concentration of the lipid nanoparticle. In some embodiments, the concentration of at least one unsaturated cationic lipid nanoparticle can be 5-50 mole %, 5-40 mole %, 5-30 mole %, 5-25 mole %, 5-20 mole %, 5-15 mole %, 10-50 mole %, 10-40 mole %, 10-30 mole %, 10-25 mole %, 15-50 mole %, 15-40 mole %, 15-30 mole % and 15-25 mole %.

### Lipid Modification

In some embodiments, lipid structures used as delivery vehicles may be modified. In some embodiments, a modification can be a surface modification. In some embodiments, a surface modification can enhance an average rate at which a lipid structure moves in mucus compared to a comparable lipid structure.

In some embodiments, a comparable lipid structure may not be surface modified, or a comparable lipid structure may be modified with a polyethylene glycol (PEG) polymer. In some embodiments, a modification can facilitate protection from degradation in vivo. In some embodiments, a modification may also assist in trafficking of a lipid structure. For example, a modification may allow a lipid structure to traffic within a gastrointestinal (GI) track with an acidic pH due to pH sensitive modifications. In some embodiments, a surface modification can also improve an average rate at which a lipid structure moves in mucous. For example, a modification may enhance a rate by 1×,2×, 3×, 4×, 5×, 6×, 7×, 8×, 9×, 10×, 20×, 30×, 40×, 50×, 60×, 70×, 80×, 90×, 100×, 300×, 500×, 700×, 900×, or up to about 1000× when compared to a comparable lipid structure without a modification or a lipid structure with a modification comprising PEG.

In some embodiments, a modification to a lipid structure occurs via a bond. In some embodiments, a bond can be covalent, noncovalent, polar, ionic, hydrogen, or any combination thereof. In some embodiments, a bond can be considered an association of two groups or portions of groups. For example, a lipid structure can be bonded to a PEG via a linker comprising a covalent bond. In some embodiments, a bond can occur between two adjacent groups. Bonds can be dynamic. A dynamic bond can occur when one group temporarily associates with a second group. For example, a polynucleic acid in suspension within a liposome may bond with portions of a lipid bilayer during its suspension.

In some embodiments, a modification can be a polyethylene glycol (PEG) addition. Methods of modifying lipid structure surfaces with PEG can include its physical adsorption onto a lipid structure surface, its covalent attachment onto a lipid structure, its coating onto a lipid structure, or any combination thereof.

In some embodiments, PEG can be covalently attached to a lipid particle before a lipid structure is formed. A variety of molecular weights of PEG may be used. PEG can range from about 10 to about 100 units of an ethylene PEG component which may be conjugated to phospholipid through an amine group comprising or comprising about 1% to about 20%, preferably about 5% to about 15%, about 10% by weight of the lipids which are included in a lipid structure.

### Targeting Agent

In some embodiments, the delivery vehicle may comprise at least one targeting agent. In some embodiments, the term targeting agent may refer to a moiety, compound, antibody, etc. that specifically binds a particular type or category of cell and/or other particular type of compounds, (e.g., a moiety that targets a specific cell or type of cell).

In some embodiments, a targeting agent may be specific (e.g., have an affinity) for the surface of certain target cells, a target cell surface antigen, a target cell receptor, or a combination thereof.

In some embodiments, a targeting agent may refer to an agent that has a particular action (e.g., cleaves) when exposed to a particular type or category of substances and/or cells, and this action can drive the delivery vehicle to target a particular type or category of cell. In some embodiments, the term targeting agent can refer to an agent that may be part of the delivery vehicle and plays a role in the delivery vehicle's targeting agent, although the agent itself may or may not be specific for the particular type or category of cell itself.

In some embodiments, the efficiency of the cellular uptake of a polynucleic acid delivered by the delivery vehicle can be enhanced and/or made more specific by incorporation of targeting agents into the present delivery vehicles.

In some embodiments, delivery vehicles described herein can comprise one or more small molecule targeting agents (e.g., carbohydrate moieties). In some embodiments, suitable targeting agents may also include, by way of non-limiting example, antibodies, antibody-like molecules, or peptides, such as an integrin-binding peptides such as RGD-containing peptides, or small molecules, such as vitamins, e.g., folate, sugars such as lactose and galactose, or other small molecules.

In some embodiments, cell surface antigens which may be targeted by targeting agents may include a cell surface molecule such as a protein, sugar, lipid, or other antigen on the cell surface. In some embodiments, the cell surface antigen undergoes internalization. Examples of cell surface antigens include, but are not limited to, the transferrin receptor type 1 and 2, the EGF receptor, HER2/Neu, VEGF receptors, integrins, NGF, CD2, CD3, CD4, CDS, CD19, CD20, CD22, CD33, CD43). CD56, CD69, and the leucine-rich repeat-containing G-protein coupled receptor 5 (LGR5).

In some embodiments, a targeting agent can also comprise an artificial affinity molecule, e.g., a peptidomimetic or an aptamer. In some embodiments, peptidomimetics may refer to compounds in which at least a portion of a peptide, such as a therapeutic peptide, is modified, and the three-dimensional structure of the peptidomimetic remains substantially the same as that of the peptide. In some embodiments, peptidomimetics (both peptide and non-peptidyl analogues) may have improved properties (e.g., decreased proteolysis, increased retention, or increased bioavailability). In some embodiments, peptidomimetics generally have improved oral availability, which makes them especially suited to treatment of disorders in a human or animal. In some embodiments, peptidomimetics may or may not have similar two-dimensional chemical structures but share common three-dimensional structural features and geometry.

In some embodiments, the targeting agent can be a proteinaceous targeting agent (e.g., a peptide, and antibody, an antibody fragment). In some specific embodiments, the delivery vehicle can comprise a plurality of different targeting agents. In embodiments, a lipid structure modification can provide biocompatibility and can be modified to possess targeting species including, for example, targeting peptides including antibodies, aptamers, polyethylene, or combinations thereof. In some embodiments, a targeting agent be a receptor. In some embodiments, a T cell receptor (TCR), B cell receptor (BCR), single chain variable fragment (scFv), chimeric antigen receptor (CAR), or combinations thereof may be used as targeting agents.

In some embodiments, one or more targeting agents may be coupled to the polymers that form the delivery vehicle. In some embodiments, the targeting agents may be bound to a polymer that coats the delivery vehicle. In some embodiments, a targeting agent may be covalently coupled to a polymer. In some embodiments, a targeting agent may be bound to a polymer such that a targeting agent can be substantially at or near the surface of the resulting delivery vehicle. In some embodiments, a monomer comprising a targeting agent residue (e.g., a polymerizable derivative of a targeting agent such as an (alkyl) acrylic acid derivative of a peptide) can be co-polymerized to form the copolymer forming the delivery vehicle provided herein.

In some embodiments, one or more targeting agents may be coupled to the polymer of the present delivery vehicles through a linking moiety. In some embodiments, the linking moiety coupling the targeting agent to the membrane-destabilizing polymer may be a cleavable linking moiety (e.g., comprises a cleavable bond). In some embodiments, the linking moiety may be cleavable and/or comprises a bond that may be cleavable in endosomal conditions. In some embodiments, the linking moiety may be cleavable and/or comprise a bond that may be cleaved by a specific enzyme (e.g., a phosphatase, or a protease). In some embodiments, the linking moiety may be cleavable and/or comprise a bond that may be cleavable upon a change in an intracellular parameter (e.g., pH, redox potential), in some embodiments, a linking moiety may be cleavable and/or comprise a bond that may be cleaved upon exposure to a matrix metalloproteinase (MMP) (e.g., MMP—cleavable peptide linking moiety).

In some embodiments, a targeting agent of the delivery vehicle can depend on a cleavage of a cleavable segment in a polymer. For instance, the present polymers can comprise a cleavable segment that, when cleaved, exposes the delivery vehicle and/or the core of the delivery vehicle. The cleavable segment can be located at either or both terminal ends of the present polymers in some embodiments. In some embodiments the cleavable segment is located along a length of a polymer, and optionally can be located between blocks of a polymer. For example, in certain embodiments the cleavable segment can be located between a first block and a second block of a polymer, and when the delivery vehicle can be exposed to a particular cleaving substance the first block can be cleaved from a second block. In some embodiments, a cleavable segment can be an MMP-cleavable peptide that can be cleaved upon exposure to MMP.

In some embodiments, attachment of a targeting agent, such as an antibody or a peptide, to a polymer or a lipid can be achieved in any suitable manner, e.g., by any one of a number of conjugation chemistry approaches including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl tinkers, sulfhydryl-sulfhydryl linkers, carbohydrate-hydroxyl linkers, carbohydrate-carbohydrate linkers, and hydroxyl-hydroxyl linkers. In some embodiments, “click” chemistry can be used to attach the targeting agent to the polymers of the delivery vehicles provided herein. In some embodiments, a large variety of conjugation chemistries are optionally utilized. In some embodiments, targeting agents may be attached to a monomer and the resulting compound may then be used in the polymerization synthesis of at least one polymer (e.g., copolymer) utilized in the delivery vehicle described herein. In some embodiments, a targeting agent can be attached to the sense or antisense strand of siRNA bound to a polymer of the delivery vehicle. In some embodiments, a targeting agent can be attached to a 5′ or a 3′ end of the sense or the antisense strand.

### Mucus and Cell Penetrating Agents

The delivery vehicles herein, such as the lipid structures described herein for such purposes further may also comprise mucus-penetrating peptide (MPPs), cell-penetrating peptides (CPP), or both.

### Mucus Penetration Components

In some embodiments, the delivery vehicle may comprise at least one mucus-penetrating peptides (MPPs) such as those disclosed in PCT/US2019/032484 the contents of which as relates to MPPs and MPP sequences (e.g., any of those listed in Table 3 therein) are herein incorporated by reference in their entirety. In some embodiments, MPPs may have cell-penetrating properties in addition to enhancing penetration through a layer of mucus, such as the naturally-occurring layers of mucus in the colon, lung, eye, and cervix.

In some embodiments, MPP may target delivery vehicles to intracellular components of cells. In some embodiments, MPPs may be designed to specifically target certain cell types.

In some embodiments, MPPs may be conjugated to delivery vehicles to allow enhanced performance of the delivery vehicle when compared to a similar delivery vehicle lacking MPPS. Such enhanced performance may include but is not limited to, increased penetration of the particles through the mucus layer, increased penetration into cells, increased specificity of cells penetrated (i.e., targeting of cells), or any combination thereof.

In some embodiments, a lipid structure that has an MPP can be internalized into a cell with an efficacy of at least about 20%, 25%, 30%, 35%, 40%, 45%, 50%, 55%, 60%, 65%, 70%, 75%, 80%, 85%, 90%, 95%, or up to about 100% as compared to a comparable particle that does not contain an MPP.

In some embodiments, the MPP may be conjugated to the lipid structure. In some embodiments, the MPP may be conjugated to at least one of the delivery vehicles such that the MPP may come into contact, in whole or in part, with a mucus layer, mucus-containing tissue, organ or extracellular surface.

In some embodiments, the MPP may be conjugated to a surface modification of the nanoparticle comprising the delivery vehicle such that the MPP it may come into contact, in whole or in part, with a mucus layer, mucus-containing tissue, organ or extracellular surface.

In some embodiments, the MPP may be conjugated to the cargo comprising the delivery vehicle such that the MPP it may come into contact, in whole or in part, with a mucus layer, mucus-containing tissue, organ or extracellular surface.

In some embodiments, the presence of the MPP can confer improved penetration of the delivery vehicle through the mucus (diffusion and/or movement through). In some embodiments, the penetration may be improved 2-fold, 3-fold, 4-fold, 5-fold, 6-fold, 7-fold, 8-fold, 9-fold, 10-fold, 15-fold, 20-fold, 25-fold, 30-fold, 50-fold, 100-fold, or more as compared to the delivery of the delivery vehicle and/or cargo that does not the MPP.

In some embodiments, an MPP can have an amino acid sequence having from about 3 to 100 amino acids, including without limitation from about 3 to 5, 5 to 10, 10 to 20, 20 to 40, 30 to 60, or 80 to 100 amino acids. In some embodiments, an MPP may have from about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or up to about 100 amino acids.

In some embodiments, an MPP may have the ability to penetrate a mucus-layer that overlays or surrounds a target cell or tissue. In some embodiments, an MPP can be employed to penetrate the mucus layer of a target tissue such as the intestinal epithelium, colon, lung, eye, or cervix of a mammal.

In some embodiments, MPPs can be conjugated to delivery vehicles, including nanoparticles, to allow penetration of the delivery vehicle through the mucus layer and also for interaction with cells so as to result in increased penetration or targeting of cells. In some embodiments, a particle that has an MPP permeates a mucus layer with an efficacy of at least about 20%, 25%, 30%, 35%, 40%, 45%, 50%, 55%, 60%, 65%, 70%, 75%, 80%, 85%, 90%, 95%, or up to about 100% as compared to a comparable particles that does not contain an MPP.

Numerous methods of determining the penetration of a mucus layer are known in the art and may be used to assess the penetration by an MPP or an MPP conjugated directly or indirectly with the delivery vehicle.

### Cell Penetrating Components

In some embodiments, the delivery vehicles herein are designed to be internalized in an epithelial cell, such as an epithelial cell within the gastrointestinal tract. In some embodiments, the delivery vehicles herein include a component for cell internalization. In some embodiments, the component is a peptide, a carbohydrate or ligand. In some embodiments, the delivery vehicles include peptides, in particular, cell penetrating peptides (CPPs) and cell penetrating peptides having mucus-penetrating functionality provide for internalization into the cell of a subject.

In some embodiments, cell penetrating peptides (CPPs) can be short polypeptides that can allow for increased uptake of delivery vehicles and/or cargo into cells. Cell-penetrating peptides (CPPs) can be peptide sequences that facilitate crossing the cytoplasmic membrane efficiently. Exemplary CPPs include those disclosed in PCT/US17/61111 which is herein incorporated by reference as t related to CPPs in its entirety.

### Linkers

In some embodiments, methods for linking compounds may include but are not limited to proteins, labels, and other chemical entities, to nucleotides. In some embodiments, cross-linking reagents such as n-maleimidobutyryloxy-succinimide ester (GMBS) and sulfo-GMBS, have reduced immunogenicity. In some embodiments, substituents have been attached to the 5′ end of preconstructed oligonucleotides using amidite or H-phosphonate chemistry. In some embodiments, substituents may also be attached to the 3′ end of oligomers. This last method utilizes 2,2′-dithioethanol attached to a solid support to displace diisopropylamine from a 3′ phosphonate bearing the acridine moiety and is subsequently deleted after oxidation of the phosphorus.

In some embodiments, an oligonucleotide may include one or more modified nucleotides having a group attached via a linker arm to the base. For example, the attachment of biotin to the C-5 position of dUTP by an allylamine linker arm may be utilized. In some embodiments, the attachment of biotin and other groups to the 5-position of pyrimidines via a linker arm may also be performed.

In some embodiments, chemical cross-linking may include the use of spacer arms, i.e., linkers or tethers. In some embodiments, spacer arms provide intramolecular flexibility or adjust intramolecular distances between conjugated moieties and thereby may help preserve biological activity. In some embodiments, a spacer arm may be in the form of a peptide moiety comprising spacer amino acids. In some embodiments, a spacer arm may be part of the cross-linking reagent, such as in “long-chain SPDP”.

In some embodiments, a variety of coupling or crosslinking agents such as protein A, carbodiimide, dimaleimide, dithio-bis-nitrobenzoic acid (DTNB), N-succinimidyl-5-acetyl-thioacetate (SATA), and N-succinimidyl-3-(2-pyrid-yldithio)propionate (SPDP), 6-hydrazinonicotimide (HYNIC), N3S and N2S2 may be used in well-known procedures to synthesize targeted constructs. For example, biotin may be conjugated to an oligonucleotide via DTPA using a bicyclic anhydride method. In some embodiments, sulfosuccinimidyl 6-(biotinamido)hexanoate (NHS-LC-biotin, which may be purchased from Pierce Chemical Co. Rockford, Ill.), “biocytin,” a lysine conjugate of biotin, may be useful for making biotin compounds due to the availability of a primary amine, corresponding biotin acid chloride or acid precursors may be coupled with an amino derivative of the therapeutic agent by known methods.

In some embodiments, by coupling a biotin moiety to the surface of a particle, another moiety may be coupled to avidin and then coupled to the particle by the strong avidin-biotin affinity, or vice versa. In some embodiments where a polymeric particle comprises PEG moieties on the surface of the particle, the free hydroxyl group of PEG may be used for linkage or attachment (e.g., covalent attachment) of additional molecules or moieties to the particle.

In some embodiments, it may be desirable to release a moiety once a drug such as a polynucleic acid has entered a cell. In some embodiments, a moiety may be utilized to identify a number of cells that have received a polynucleic acid. In some embodiments, a moiety may be an antibody, dye, scFv, peptide, glycoprotein, carbohydrate, ligand, polymer, to name a few. In some embodiments, a moiety may be in contact with a linker.

In some embodiments, a linker may be non-cleavable.

In some embodiments, a linker may be a cleavable linker which may enable a moiety to be released from a lipid structure once contact to a target cell has been made. In some embodiments, a moiety may have a better ability to be absorbed by an intracellular component of a cell, such as an intestinal crypt cell or intestinal crypt stem cell, when separated from a lipid structure. In some embodiments, a linker may comprise a disulfide bond, acyl hydrazone, vinyl ether, orthoester, or a N—PO3.

In some embodiments, it may be necessary or desirable to separate a moiety from a lipid structure so that a moiety may enter an intracellular compartment. In some embodiments, cleavage of a linker releasing a moiety may be as a result of a change in conditions within a cell as compared to outside cells, for example, due to a change in pH within a cell. In some embodiments, cleavage of a linker may occur due to the presence of an enzyme within a cell which cleaves a linker once a drug, such as a polynucleic acid, enters a cell. In some embodiments, cleavage of a linker may occur in response to energy, or a chemical being applied to the cell. In some embodiments, examples of types of energies that may be used to effect cleavage of a linker include, but are not limited to, light, ultrasound, microwave, and radiofrequency energy.

In some embodiments, a linker may be a photolabile linker. In some embodiments, a linker used to link a complex may also be an acid labile linker such as but not limited to, linkers formed by using cis-aconitic acid, cis-carboxylic alkatriene, polymaleic anhydride, and other acid labile linkers.

Delivery Vehicles with Charge Separation

In some embodiments, the delivery vehicles may comprise particles (e.g., nanoparticles) that display charge separation as described herein. In some embodiments, the delivery vehicles as provided herein can be utilized to deliver any type of cargo to a target, for instance a target cell. The delivery vehicles herein with charge separation and epithelial-reaching functionality as provided herein can be utilized to deliver any type of cargo to a target, for instance a target cell.

In some embodiments, delivery vehicles provided herein contain positive and negative charges separated into different loci within the particle, where each locus is comprised of a different polymer (conferring the charge to the locus). In some embodiments, delivery vehicles provided herein contain positively-charged and negatively-charged lipids, where the loci are separated by phase, such as into a liquid phase and a gel phase. In some instances, the delivery vehicle can comprise a positively charged liquid phase and a negatively charged gel phase; or a positively charged gel phase and a negatively charged liquid phase.

In some embodiments, the delivery vehicles herein (such as lipid nanoparticles, liposomes, and micelle-like structures) may have at least two loci and comprise a positive charge and a negative charge that are not interspersed but instead located in separated loci. For example, a negative charge and a positive charge may be present on opposite loci on a lipid structure provided herein at a pH between about 5.5 and 8.0, such as at a pH of about 7.4.

In some embodiments, a positive charge and a negative charge are in two separate loci where each locus is a different phase of a lipid structure, for example a liquid or solid (gel) phase. In some embodiments, a positive charge may be on a liquid phase and a negative charge may be on a solid phase, for example a gel phase or vice versa. Charge separation can allow for both an attraction and repulsion force.

In some embodiments, a positive lipid can be attracted towards a target cell due to its high negative potential. In some embodiments, a repulsive force on a negative face can prevent a positive face from being kinetically trapped in mucus. In some embodiments, a cationic charge, for instance on a lipid on the delivery vehicle, maybe attracted to mucus, en route to a target cell, and may get kinetically trapped in the mucus thereby trapping the delivery vehicle. The mucus will eventually slough off clearing the delivery vehicle.

In some embodiments, an anionic delivery vehicle can be repulsed by mucus and may not make its way through the mucus. A zwitterionic particle can act like a neutral particle absent a net force. Zwitterionic particles may follow the flow of water similar to PEGylated systems and may not become trapped in the mucus but may not reach the epithelial cells.

In some embodiments of the delivery vehicles, the first locus comprises an unsaturated or short-tail lipid. In some embodiments, the unsaturated lipid comprises a cationic or ionizable cationic lipid. In some embodiments, the cationic lipid comprises a multivalent cationic lipid or a monovalent cationic lipid.

In some embodiments, charge separation may result in superior and/or unexpected performance of subject delivery vehicles. For example, utilizing PEG is thought to increase trafficking to target cells, for example intestinal epithelial cells as provided in Maisel K et al., Effect of surface chemistry on nanoparticle interaction with gastrointestinal mucus and distribution in the gastrointestinal tract following oral and rectal administration in the mouse. J Control Release, herein incorporated by reference. In some embodiments, increasing PEGylation results in decreased distribution within or at the intestinal tissue thereby providing support for utilizing delivery vehicles with reduced PEGylation as compared to conventional vehicles. One mechanism by which reducing PEGylation may improve trafficking and/or distribution to and in proximity to a target cell is by increasing the exposure of positive charge at the surface of a subject vehicle by reducing the shielding properties of PEGylation.

In some embodiments, the delivery vehicle that comprises charge separation as provided herein can have improved trafficking, transfection of target cells, epithelial reach, or a combination thereof as compared to a comparable delivery vehicle that lacks the charge separation. In some embodiments, the improvement is from about 1 fold, 50 fold, 99 fold, 148 fold, 197 fold, 246 fold, 295 fold, 344 fold, 393 fold, 442 fold, 491 fold, 540 fold, 589 fold, 638 fold, 687 fold, 736 fold, 785 fold, 834 fold, 883 fold, 932 fold, 981 fold, or up to about 1000 fold as compared to a comparable delivery vehicle that lacks the charge separation.

In some embodiments, the delivery vehicle has a first locus that is positively charged at a pH between about 5.5 and 8.0, and a second locus that is negatively charged at a pH between about 5.5 and 8.0, wherein the first and second loci are separated such that the positive and negative charges are not interspersed, and wherein one or both loci contain a lipid.

In some embodiments, the first locus comprises an unsaturated or short-tail lipid, such as a cationic or ionizable cationic lipid, for example, a multivalent cationic lipid or a monovalent cationic lipid.

In some embodiments, the ratio of a cationic charge in a first locus to an anionic charge in a second locus at pH 7.4 is from about 0.25, 0.45, 0.65, 0.85, 1.05, 1.25, 1.45, 1.65, 1.85, 2.05, 2.25, 2.45, 2.65, or 2.85. In some embodiments, the ratio of a cationic charge in a first locus to an anionic charge in a second locus at pH 7.4 is from about 0.25 to about 1.05, 0.75 to about 1.25, 1.05 to about 1.45, or 0.85 to about 1.85. In some embodiments, a ratio of a multivalent lipid to an ionizable cationic lipid in the delivery vehicle is from about (6%, 6.25%, 6.5%, 6.75%, 7%, 7.25%, 7.5%, 7.75%, or 8%) to (8%, 8.25%, 8.5%, 8.75%, 9%, 9.25%, 9.5%, 9.75%, 10%), (12%, 12.25%, 12.5%, 12.75%, or 13%) to (12%, 12.25%, 12.5%, 12.75%, or 13%), or (18%, 18.25%, 18.5%, 18.75%, 19%, 19.25%, 19.5%, 19.75%, 20%) to (6%, 6.25%, 6.5%, 6.75%, 7%, 7.25%, 7.5%, 7.75%, or 8%). In some aspects, a bile salt is at a concentration from about 10 mole %, 15 mole %, 20 mole %, 25 mole %, 30 mole %, 35 mole %, 40 mole %, 45 mole %, 50 mole %, 55 mole %, 60 mole %, 65 mole %, 70 mole %, 75 mole %, or about 80 mole %. In some embodiments, a bile salt is from about 10 mole % to 30 mole %, 20 mole % to 50 mole %, 30 mole % to 60 mole %, or 40 mole % to 80 mole %. Suitable alternate formulations can comprise multivalent lipid, ionizable cationic lipid, bile salt, structural lipid, and/or lipid-PEG at molar ratios from about 5%, 10%, 15%, 20%, 25%, 30%, 35%, 40%, 45%, 50%, 55%, 60%, 65%, 70%, 75%, 80%, 85%, 90%, or 95% more or less to those provided herein.

In some embodiments, the delivery vehicle may comprise a high temperature phase transition lipid, for example, a high temperature phase transition neutral lipid such as DSPC, and a bile salt such as deoxycholate, cholic acid or a conjugate thereof. Deoxycholate can serve as a solid phase (gel phase) where deoxycholate provides the negative charge. On the same delivery vehicle, a cationic lipid can be present as unsaturated or a short tail lipid and can be present in the liquid phase. Multivalent cationic lipids, like MVL5, can be used to create enough positive to negative charge ratio to provide the system with a balance of attraction and repulsion thereby generating the delivery vehicle containing a charge separation.

In some embodiments delivery vehicles herein, including those with charge separation, are useful to treat diseases and conditions that effect and/or originate in mucosal tissues, such as in the mucosal tissues in the gastrointestinal tract. Non-limiting examples include familial adenomatous polyposis (FAP), attenuated FAP, colorectal cancer, chronic inflammatory bowel disease, chronic inflammatory bowel disease, microvillus inclusion disease and congenital diarrheal diseases. In some embodiments, delivery vehicles with charge separation are useful to provide therapeutic agents and/or nucleic acids to express therapeutic agents in mucosal tissues and such agents may remain in the targeted epithelial cells and/or be transported to other disease-affected cells and tissues within a subject.

### Cryoprotectants, and Preservatives

In some embodiments, the pharmaceutical compositions or delivery vehicles disclosed herein may be frozen, such as for storage or shipment.

In some embodiments, in order to preserve the size and uniformity, as measured by the polydispersity index (PDI), the delivery vehicles may include in some embodiments the pharmaceutical compositions or delivery vehicles may be combined with a cryoprotectant. In some embodiments, the cryoprotectant may be, but is not limited to glycerol, a phosphate buffer, a Tris-sucrose buffer, or any combination thereof.

In some embodiments, a suitable phosphate buffer may comprise 0.001-0.1 mg Potassium Dihydrogen Phosphate, 0.01-0.1 mg Disodium Hydrogen Phosphate Dihydrate, 0.001-0.0.1 mg Potassium Chloride, 0.1-0.5 mg Sodium Chloride, and 1-10 mg Sucrose.

In some embodiments, a suitable phosphate buffer may comprise 0.01 mg Potassium Dihydrogen Phosphate, 0.07 mg Disodium Hydrogen Phosphate Dihydrate, 0.01 mg Potassium Chloride, 0.36 mg Sodium Chloride, and 6 mg Sucrose.

In some embodiments, a suitable Tris-sucrose buffer may comprise 10-30 mM tris(hydroxymethyl)aminomethane (Tris) and 5-15% w/v Sucrose.

In some embodiments, a suitable Tris-sucrose buffer may comprise 20 mM tris(hydroxymethyl)aminomethane (Tris) and 10% w/v Sucrose.

### II. EXEMPLARY DELIVERY VEHICLES

### Exemplary Lipid Combinations

In some embodiments, the delivery vehicles herein may include any of at least one bile salt or bile acid, at least one cationic lipid, at least one structural lipid, at least one conjugated lipid, and any combination thereof. In some embodiments, the delivery vehicles herein may include at least one bile salt or bile acid, at least one cationic lipid, at least one structural lipid, and at least one conjugated lipid. In some embodiments, the delivery vehicles herein may include at least one bile salt or bile acid, at least two cationic lipids, at least one structural lipid, and at least one conjugated lipid. In some embodiments, the delivery vehicles herein may include at least one bile salt or bile acid, at least one multivalent cationic lipid, at least one ionizable cationic lipid, at least one at least one structural lipid, and at least one conjugated lipid.

In some embodiments, the delivery vehicle includes at least one saturated lipid, at least one of an unsaturated cationic lipid or an unsaturated non-cationic lipid, and a bile salt. In some embodiments, the lipid nanoparticle comprises a bile salt and a saturated cationic lipid that has a phase transition temperature of at least about 37° C., and a non-cationic lipid. In some embodiments, the lipid nanoparticle comprises a bile salt and a multivalent cationic lipid and a non-cationic lipid, where the multivalent cationic lipid, the non-cationic lipid, or the multivalent cationic lipid and the non-cationic lipid have a phase transition temperature of at least about 37° C. In some embodiments, the lipid nanoparticle includes at least one saturated lipid, where the saturated lipid comprises a saturated cationic lipid that has a phase transition temperature of at least about 37° C. or a saturated non-cationic lipid that has a phase transition temperature of at least about 37° C. In some aspects, the lipid nanoparticle further includes at least one of: a non-cationic lipid, a multivalent cationic lipid, a permanently charged cationic lipid, or any combinations thereof.

In some embodiments, the lipid nanoparticle comprises a bile salt and a saturated cationic lipid, an unsaturated cationic lipid, and a non-cationic lipid, wherein the unsaturated cationic lipid, the non-cationic lipid, or the unsaturated cationic lipid and the non-cationic lipid have a phase transition temperature of at least about 37° C.

In some embodiments, a lipid structure can include one or more of an anionic lipid or cationic lipid, a neutral lipid, a sterol, and a lipid selected to reduce aggregation of lipid particles during formation. Aggregation may result from steric stabilization of lipid structures which may prevent charge-induced aggregation during formation. Lipid structures can include two or more cationic lipids. In an aspect, a cationic lipid may be on a first phase and an anionic lipid on a second phase such that the lipid structure contains two phases with differentially charged lipids.

In some embodiments, the delivery vehicle can comprise any one of: N1-[2-((1S)-1-[(3-aminopropyl)amino]-4-[di(3-amino-propyl)amino]butylcarboxamido)ethyl]-3,4-di[oleyloxy]-benzamide (MVL5)/(6Z,9Z,28Z,31Z)-heptatriaconta-6,9,28,31-tetraen-19-yl 3-(dimethylamino)propanoate (MC2)/1,2-distearoyl-sn-glycero-3-phosphocholine (DSPC)/Deoxycholate/1,2-dimyristoyl-rac-glycero-3-methoxypolyethylene glycol (DMG-PEG); MVL5/MC2/DSPC/Deoxycholate/1,2-dimyristoyl-sn-glycero-3-phosphoethanolamine (DMPE)-PEG; MVL5/7-(4-(dimethylamino)butyl)-7-hydroxytridecane-1,13-diyl dioleate (CL1H6)/DSPC/Deoxycholate/DMG-PEG; MVL5/7-(4-(diisopropylamino)butyl)-7-hydroxytridecane-1,13-diyl dioleate (CL4H6)/DSPC/Deoxycholate/DMG-PEG; MVL5/MC2/DSPC/Chenodeoxycholate/DMG-PEG; MVL5/MC2/1,2-dimyristoyl-sn-glycero-3-phosphocholine (DMPC)/Deoxycholate/DMG-PEG; MVL5/MC2/DMPC/Deoxycholate/DMPE-PEG; MVL5/CL1H6/DMPC/Deoxycholate/DMG-PEG; MVL5/MC2/DSPC/Deoxycholate/Lithocholate/DMG-PEG; MVL5/CL1H6/DSPC/Deoxycholate/Lithocholate/DMG-PEG; MVL5/MC2/DSPC/Alloisolithocholate/DMG-PEG; or MVL5/MC2/DSPC/Dehydrolithocholate/DMG-PEG.

### Exemplary Compositions of the Delivery Vehicle

In some embodiments, the delivery vehicle provided herein can comprise at least one of a multivalent lipid, cationic lipid, structural lipid, bile salt, bile acid or conjugated lipid (i.e., a lipid-PEG). Any or all of the lipids provided herein can be formulated at any mole % for example, including but not limited to: 0 mole %, 0.5 mole %, 1 mole %, 1.5 mole %, 2 mole %, 2.5 mole %, 3 mole %, 3.5 mole %, 4 mole %, 4.5 mole %, 5 mole %, 5.5 mole %, 6 mole %, 6.5 mole %, 7 mole %, 7.5 mole %, 8 mole %, 8.5 mole %, 9 mole %, 9.5 mole %, 10 mole %, 10.5 mole %, 11 mole %, 11.5 mole %, 12 mole %, 12.5 mole %, 13 mole %, 13.5 mole %, 14 mole %, 14.5 mole %, 15 mole %, 15.5 mole %, 16 mole %, 16.5 mole %, 17 mole %, 17.5 mole %, 18 mole %, 18.5 mole %, 19 mole %, 19.5 mole %, 20 mole %, 20.5 mole %, 21 mole %, 21.5 mole %, 22 mole %, 22.5 mole %, 23 mole %, 23.5 mole %, 24 mole %, 24.5 mole %, 25 mole %, 25.5 mole %, 26 mole %, 26.5 mole %, 27 mole %, 27.5 mole %, 28 mole %, 28.5 mole %, 29 mole %, 29.5 mole %, 30 mole %, 30.5 mole %, 31 mole %, 31.5 mole %, 32 mole %, 32.5 mole %, 33 mole %, 33.5 mole %, 34 mole %, 34.5 mole %, 35 mole %, 35.5 mole %, 36 mole %, 36.5 mole %, 37 mole %, 37.5 mole %, 38 mole %, 38.5 mole %, 39 mole %, 39.5 mole %, 40 mole %, 40.5 mole %, 41 mole %, 41.5 mole %, 42 mole %, 42.5 mole %, 43 mole %, 43.5 mole %, 44 mole %, 44.5 mole %, 45 mole %, 45.5 mole %, 46 mole %, 46.5 mole %, 47 mole %, 47.5 mole %, 48 mole %, 48.5 mole %, 49 mole %, 49.5 mole %, 50 mole %, 50.5 mole %, 51 mole %, 51.5 mole %, 52 mole %, 52.5 mole %, 53 mole %, 53.5 mole %, 54 mole %, 54.5 mole %, 55 mole %, 55.5 mole %, 56 mole %, 56.5 mole %, 57 mole %, 57.5 mole %, 58 mole %, 58.5 mole %, 59 mole %, 59.5 mole %, 60 mole %, 60.5 mole %, 61 mole %, 61.5 mole %, 62 mole %, 62.5 mole %, 63 mole %, 63.5 mole %, 64 mole %, 64.5 mole %, 65 mole %, 65.5 mole %, 66 mole %, 66.5 mole %, 67 mole %, 67.5 mole %, 68 mole %, 68.5 mole %, 69 mole %, 69.5 mole %, 70 mole %, 70.5 mole %, 71 mole %, 71.5 mole %, 72 mole %, 72.5 mole %, 73 mole %, 73.5 mole %, 74 mole %, 74.5 mole %, 75 mole %, 75.5 mole %, 76 mole %, 76.5 mole %, 77 mole %, 77.5 mole %, 78 mole %, 78.5 mole %, 79 mole %, 79.5 mole %, or 80 mole %.

In some embodiments, the delivery vehicles may comprise 5-40 mole % of at least one bile salt or bile acid; 5-90 mole % of at least one cationic lipid; 5-75 mole % of at least one structural lipid; and 0.5-2 mole % of at least one conjugated lipid.

In some embodiments, the delivery vehicles may comprise about 5-40 mole % of the at least one bile salt or bile acid; about 5-60 mole % one cationic lipid; about 5-60 mole % of a second cationic lipid; about 5-75 mole % of at least one structural lipid; and about 0.5-2.0 mole % of at least one conjugated lipid.

In some embodiments, the delivery vehicles may comprise about 20-40 mole % of the at least one bile salt or bile acid; about 5-30 mole % one cationic lipid; about 5-30 mole % of a second cationic lipid; about 30-50 mole % of at least one structural lipid; and about 0.5-2.0 mole % of at least one conjugated lipid.

In some embodiments, the delivery vehicles may comprise about 30-40 mole % of the at least one bile salt or bile acid; about 5-15 mole % one cationic lipid; about 5-15 mole % of a second cationic lipid; about 35-45 mole % of at least one structural lipid; and about 0.5-2.0 mole % of at least one conjugated lipid.

In some embodiments, the delivery vehicle may comprise about 33 mole % of at least one bile salt; about 12.5 mole % of one cationic lipid; about 12.5 mole % of a second cationic lipid; about 41 mole % of at least one structural lipid; and about 1 mole % of at least one conjugated lipid.

In some embodiments, the delivery vehicles may comprise any of the formulations disclosed in Table 1B or any combination thereof.

A delivery vehicle can be generated using a variety of molar ratios. In some embodiments, a delivery vehicle (e.g., one for use in a pharmaceutical formulation) comprises MVL5, MC2, Deoxycholate, DSPC, and DMG-PEG at a molar ratio of about 0.96:0.96:2.592:3.168:0.0768:0.0384:0.0384.

In some embodiments, composition nanoparticles include a molar ratio between components of from about 1 to about 5 of at least one bile salt, from about 0.5 to about 3 of each one of at least one cationic lipid, from about 2 to about 10 of at least one structural lipid, and from about 0.02 to about 0.10 of at least one conjugated lipid.

In some embodiments, the delivery vehicle (e.g., a nanoparticle) bile salts may include one or more of deoxycholate, ursodiol, lithocholate, isolithocholate, alloisolithocholate, dehydrolithocholate, and 5beta-cholanic acid. In some embodiments, the delivery vehicle cationic lipids may include MVL5. In some embodiments, the delivery vehicle cationic lipids may include MC2. In some embodiments, the delivery vehicle structural lipids may include DSPC. In some embodiments, the delivery vehicle conjugated lipids may include DMG-PEG. In some embodiments, molar ratios of bile salt:MVL5:MC2:DSPC:DMG-PEG may be about 2.592:0.96:0.96:3.168:0.768 in the delivery vehicle. In some embodiments, the delivery vehicle bile salts may include deoxycholate.

In some embodiments, composition of the delivery vehicle (e.g., nanoparticles) may include MVL5, MC2, DSPC, deoxycholate, and DMPE-PEG at a molar ratio of 2.4:2.4:7.9:6.48:0.192.

In some embodiments, the delivery vehicle may include MVL5, CL1H6, DSPC, deoxycholate, and DMG-PEG at a molar ratio of about 2.4, 2.4, 7.9, 6.48, and 0.192.

In some embodiments, the delivery vehicle may include MVL5, CL4H6, DSPC, deoxycholate, and DMG-PEG at a molar ratio of about 2.4, 2.4, 7.9, 6.48, and 0.192.

In some embodiments, the delivery vehicle may include MVL5, MC2, DSPC, chenodeoxycholate, and DMG-PEG at a molar ratio of about 2.4, 2.4, 7.9, 6.48, and 0.192.

In some embodiments, the delivery vehicle may include MVL5, MC2, DMPC, deoxycholate, and DMG-PEG at a molar ratio of about 2.4, 2.4, 7.9, 6.48, and 0.192.

In some embodiments, the delivery vehicle may include MVL5, MC2, DMPC, deoxycholate, and DMPE-PEG at a molar ratio of about 2.4, 2.4, 7.9, 6.48, and 0.192.

In some embodiments, the delivery vehicle may include MVL5, CL1H6, DMPC, deoxycholate, and DMG-PEG at a molar ratio of about 2.4, 2.4, 7.9, 6.48, and 0.192.

In some embodiments, the delivery vehicle may include MVL5, MC2, DSPC, deoxycholate, lithocholate, and DMG-PEG at a molar ratio of about 2.4, 2.4, 7.9, 5.2, 1.3, and 0.192.

In some embodiments, the delivery vehicle may include MVL5, CL1H6, DSPC, deoxycholate, lithocholate, and DMG-PEG at a molar ratio of about 2.4, 2.4, 7.9, 5.2, 1.3, and 0.192.

In some embodiments, the delivery vehicle may include MVL5, MC2, DSPC, alloisolithocholate, and DMG-PEG at a molar ratio of about 2.4, 2.4, 7.92, 6.48, and 0.192.

In some embodiments, the delivery vehicle may include MVL5, MC2, DSPC, dehydrolithocholate, and DMG-PEG at a molar ratio of about 2.4, 2.4, 7.92, 6.48, and 0.192.

In some embodiments, compositions of the delivery vehicle may include 12.4 mole 0% of MVL5, 12.4 mole % of MC2, 40.8 mole % of DSPC, 33.4 mole % of at least one bile salt, and 1 mole % of at least one conjugated lipid. In some embodiments, the at least one conjugated lipid may include DMG-PEG or DMPE-PEG. In some embodiments, the at least one bile salt include one or more of taurodeoxycholate, taurochenodeoxycholate, glycocholate, 3-oxy-cholenic acid, and deoxycholate.

Exemplary delivery vehicles are described herein and provided for example at Table 1A, Table 1B, Table 2, Table 3, Table 4, and Table 8. Any one of the delivery vehicles exemplified can be further modified. For example, additional lipids, cargo, modifications to, additions to, subtractions to, can be made. In some embodiments, any one of the delivery vehicles in Table 1 can further comprise lipid-PEG.

TABLE 1A
Exemplary design of delivery vehicles

SN:UC:BS
SN:SMV:BS
SN:UMV:BS
SN:[(UC)x + (MV)x + (UN)x + (SN)x]:BS
SC:BS
SC:UN:BS
SC:UC:BS
SC:UMV:BS
SC:SN:BS
SC:[(UC)x + (MV)x + (SN)x + (SC)x]:BS
SMV:BS
SMV:UN:BS
SMV:SN:BS
SMV:[(UC)x + (MV)x + (SN)x + (SC)x]:BS
UMV:UN:BS
Abbreviations:
BS: bile salt,
SC: saturated cationic,
UC: unsaturated cationic,
SN: saturated non-cationic,
UN: unsaturated non-cationic,
MV: multivalent cationic,
SMV: Multivalent cationic saturated,
UMV: Multivalent cationic unsaturated.
Where “x” appears in the formula, this denotes at least one (i.e., x is equal to or greater than 1).

TABLE 1B
Exemplary Delivery Vehicle Compositions

| | | Component |
|---|---|---|
| Nano- | | Amounts |
| particle | | (Mole % of the |
| ID # | Composition Components | delivery vehicles) |
| MVLP001 | MVL5/CL1H6/Deoxycholate/DSPC/ | 12.4/12.4/33.4/ |
| | DMG-PEG | 40.8/1 |
| MVLP002 | MVL5/CL1H6/Deoxycholate/DSPC/ | 12.4/12.4/33.5/ |
| | DMG-PEG | 40.7/1 |
| MVLP003 | MVL5/CL1H6/Deoxycholate/DSPC/ | 12.4/12.4/33.5/ |
| | DMG-PEG | 40.8/1 |
| MVLP004 | MVL5/CL1H6/Deoxycholate/DSPC/ | 12.4/12.4/37.1/ |
| | DMG-PEG | 37.1/1 |
| MVLP005 | MVL5/CL1H6/Deoxycholate/DSPC/ | 12.4/12.4/18.6/ |
| | DMG-PEG | 55.7/1 |
| MVLP006 | MVL5/CL1H6/Deoxycholate/DSPC/ | 12.4/12.4/55.7/ |
| | DMG-PEG | 118.6/ |
| MVLP007 | MVL5/CL1H6/Deoxycholate/DSPC/ | 12.4/12.4/66.8/7.4/ |
| | DMG-PEG | 1 |
| MVLP008 | MVL5/CL1H6/Deoxycholate/DSPC/ | 11.6/11.6/37.6/ |
| | DMG-PEG | 38.3/0.9 |
| MVLP009 | MVL5/CL1H6/Deoxycholate/DSPC/ | 13.5/13.5/27.3/ |
| | DMG-PEG | 44.6/1.1 |
| MVLP010 | MVL5/CL1H6/Deoxycholate/DSPC/ | 14.9/14.9/20/ |
| | DMG-PEG | 49.1/1.2 |
| MVLP011 | MVL5/CL1H6/Deoxycholate/DSPC/ | 16.5/16.5/11.1/ |
| | DMG-PEG | 54.5/1.3 |
| MVLP012 | MVL5/CL1H6/Deoxycholate/DSPC/ | 17.3/17.3/29/35.4/1 |
| | DMG-PEG | |
| MVLP013 | MVL5/CL1H6/Deoxycholate/DSPC/ | 24.8/24.8/22.3/ |
| | DMG-PEG | 27.2/1 |
| MVLP014 | MVL5/CL1H6/Deoxycholate/DSPC/ | 32.2/32.2/15.6/ |
| | DMG-PEG | 19.1/1 |
| MVLP015 | MVL5/CL1H6/Deoxycholate/DSPC/ | 44.6/44.6/4.5/5.4/1 |
| | DMG-PEG | |
| MVLP016 | MVL5/CL1H6/Deoxycholate/DSPC/ | 5/5/40.1/49/1 |
| | DMG-PEG | |
| MVLP017 | MVL5/CL1H6/Deoxycholate/DMPC/ | 12.4/12.4/33.4/ |
| | DMG-PEG | 40.8/1 |
| MVLP018 | MVL5/CL1H6/Deoxycholate/DOPE/ | 12.4/12.4/33.4/ |
| | DMG-PEG | 140.8/ |
| MVLP019 | MVL5/CL1H6/Deoxycholate/DOPE/ | 12.4/12.4/55.7/ |
| | DMG-PEG | 18.6/1 |
| MVLP020 | MVL5/CL1H6/Deoxycholate/DOPE/ | 12.4/12.4/37.1/ |
| | DMG-PEG | 37.1/1 |
| MVLP021 | MVL5/CL1H6/Deoxycholate/DOPE/ | 12.4/12.4/18.6/ |
| | DMG-PEG | 55.7/1 |
| MVLP022 | MVL5/CL1H6/DMPC/Deoxycholate/ | 12.5/12.5/41.1/ |
| | DMG-PEG | 33.8/1 |
| MVLP023 | MVL5/CL1H6/DOPE/DMG-PEG | 12.4/12.4/74.3/1 |
| MVLP024 | MVL5/CL1H6/DSPC/Deoxycholate/ | 12.5/12.5/41.1/ |
| | DMG-PEG | 33.8/1 |
| MVLP025 | MVL5/CL1H6/DSPC/Deoxycholate/ | 12.5/12.5/41.1/ |
| | Lithocholate/DMG-PEG | 27.1/6.8/1 |
| MVLP026 | MVL5/CL1H6/DSPC/DMG-PEG | 18.6/18.6/61.3/1.5 |
| MVLP027 | MVL5/CL1H6/DSPC/DMG-PEG | 12.4/12.4/74.3/1 |
| MVLP028 | MVL5/CL1H6/Lithocholate/DOPE/ | 12.4/12.4/33.4/ |
| | DMG-PEG | 40.8/1 |
| MVLP029 | MVL5/CL1H6/Lithocholate/DSPC/ | 12.4/12.4/33.4/ |
| | DMG-PEG | 40.8/1 |
| MVLP030 | MVL5/CL4H6/Deoxycholate/DSPC/ | 12.4/12.4/33.4/ |
| | DMG-PEG | 40.8/1 |
| MVLP031 | MVL5/CL4H6/DSPC/Deoxycholate/ | 12.5/12.5/41.1/ |
| | DMG-PEG | 33.8/1 |
| MVLP032 | MVL5/DODMA/Deoxycholate/ | 19.5/19.5/52.8/8 |
| | DMG-PEG | |
| MVLP033 | MVL5/DODMA/DOPC/Deoxycholate/ | 12.4/12.4/40.8/ |
| | DMG-PEG | 33.4/1 |
| MVLP034 | MVL5/DODMA/DSPC/Deoxycholate | 6.25/18.75/41.25/ |
| | | 33.75 |
| MVLP035 | MVL5/DODMA/DSPC/Deoxycholate | 12.5/12.5/41.25/ |
| | | 33.75 |
| MVLP036 | MVL5/DODMA/DSPC/Deoxycholate | 18.75/6.25/41.25/ |
| | | 33.75 |
| MVLP037 | MVL5/DODMA/DSPC/Deoxycholate | 12.5/12.5/41.3/33.7 |
| MVLP038 | MVL5/DODMA/DSPC/Deoxycholate/ | 6.25/18.75/ |
| | DMG-PEG | 337.1/0.4/7.5 |
| MVLP039 | MVL5/DODMA/DSPC/Deoxycholate/ | 12.5/12.5/37.1/ |
| | DMG-PEG | 30.4/7.5 |
| MVLP040 | MVL5/DODMA/DSPC/Deoxycholate/ | 18.75/6.25/37.1/ |
| | DMG-PEG | 30.4/7.5 |
| MVLP041 | MVL5/DODMA/DSPC/Deoxycholate/ | 12.4/12.4/40.8/ |
| | DMG-PEG | 33.4/1 |
| MVLP042 | MVL5/DODMA/DSPC/Deoxycholate/ | 12.25/12.25/40.4/ |
| | DMG-PEG | 33.1/2 |
| MVLP043 | MVL5/DODMA/DSPC/Deoxycholate/ | 12.1/12.1/40.0/ |
| | DMG-PEG | 32.7/3 |
| MVLP044 | MVL5/DODMA/DSPC/Deoxycholate/ | 11.9/11.9/39.2/ |
| | DMG-PEG | 32.1/5 |
| MVLP045 | MVL5/DODMA/DSPC/Deoxycholate/ | 11.25/11.25/37.1/ |
| | DMG-PEG | 30.4/10 |
| MVLP046 | MVL5/DODMA/DSPC/Deoxycholate/ | 12.4/12.4/40.8/ |
| | DSG-PEG | 33.4/1 |
| MVLP047 | MVL5/DODMA/DSPC/Deoxycholate/ | 12.25/12.25/40.4/ |
| | DSG-PEG | 33.1/2 |
| MVLP048 | MVL5/DODMA/DSPC/Deoxycholate/ | 12.1/12.1/40.0/ |
| | DSG-PEG | 32.7/3 |
| MVLP049 | MVL5/DODMA/GMO/Deoxycholate/ | 12.4/12.4/40.8/ |
| | DMG-PEG | 33.4/1 |
| MVLP050 | MVL5/DSPC/Deoxycholate | 25/41.25/33.75 |
| MVLP051 | MVL5/DSPC/Deoxycholate/DMG-PEG | 25/37.1/30.4/7.5 |
| MVLP052 | MVL5/MC2/Deoxycholate/DMG-PEG | 12.4/12.4/74.3/1 |
| MVLP053 | MVL5/MC2/Deoxycholate/DMPC/ | 12.4/12.4/33.4/ |
| | DMG-PEG | 40.8/1 |
| MVLP054 | MVL5/MC2/Deoxycholate/DMPC/ | 12.4/12.4/55.7/ |
| | DMG-PEG | 18.6/1 |
| MVLP055 | MVL5/MC2/Deoxycholate/DMPC/ | 12.4/12.4/37.1/ |
| | DMG-PEG | 37.1/1 |
| MVLP056 | MVL5/MC2/Deoxycholate/DMPC/ | 12.4/12.4/18.6/ |
| | DMG-PEG | 55.7/1 |
| MVLP057 | MVL5/MC2/Deoxycholate/DOPE/ | 12.4/12.4/33.4/ |
| | DMG-PEG | 40.8/1 |
| MVLP058 | MVL5/MC2/Deoxycholate/DOPE/ | 12.4/12.4/66.9/7.4/ |
| | DMG-PEG | 1 |
| MVLP059 | MVL5/MC2/Deoxycholate/DOPE/ | 12.4/12.4/55.7/ |
| | DMG-PEG | 18.6/1 |
| MVLP060 | MVL5/MC2/Deoxycholate/DOPE/ | 12.4/12.4/37.1/ |
| | DMG-PEG | 37.1/1 |
| MVLP061 | MVL5/MC2/Deoxycholate/DOPE/ | 12.4/12.4/18.6/ |
| | DMG-PEG | 55.7/1 |
| MVLP062 | MVL5/MC2/Deoxycholate/DSPC/ | 16.5/16.5/11.1/ |
| | DMG-PEG | 54.5/1.3 |
| MVLP063 | MVL5/MC2/Deoxycholate/DSPC/ | 14.9/14.9/20.1/49/ |
| | DMG-PEG | 1.2 |
| MVLP064 | MVL5/MC2/Deoxycholate/DSPC/ | 13.5/13.5/27.3/ |
| | DMG-PEG | 44.6/1.1 |
| MVLP065 | MVL5/MC2/Deoxycholate/DSPC/ | 12.4/12.4/33.4/ |
| | DMG-PEG | 140.8/ |
| MVLP066 | MVL5/MC2/Deoxycholate/DSPC/ | 11.6/11.6/37.6/ |
| | DMG-PEG | 38.3/0.9 |
| MVLP067 | MVL5/MC2/Deoxycholate/DSPC/ | 12.4/12.4/66.8/7.4/ |
| | DMG-PEG | 1 |
| MVLP068 | MVL5/MC2/Deoxycholate/DSPC/ | 12.4/12.4/55.7/ |
| | DMG-PEG | 18.6/1 |
| MVLP069 | MVL5/MC2/Deoxycholate/DSPC/ | 12.4/12.4/37.1/ |
| | DMG-PEG | 37.1/1 |
| MVLP070 | MVL5/MC2/Deoxycholate/DSPC/ | 12.4/12.4/18.6/ |
| | DMG-PEG | 55.7/1 |
| MVLP071 | MVL5/MC2/Deoxycholate/DSPC/ | 44.6/44.6/4.5/5.4/1 |
| | DMG-PEG | |
| MVLP072 | MVL5/MC2/Deoxycholate/DSPC/ | 32.2/32.2/15.6/ |
| | DMG-PEG | 19.1/1 |
| MVLP073 | MVL5/MC2/Deoxycholate/DSPC/ | 24.8/24.8/22.3/ |
| | DMG-PEG | 27.2/1 |
| MVLP074 | MVL5/MC2/Deoxycholate/DSPC/ | 17.3/17.3/29/35.4/1 |
| | DMG-PEG | |
| MVLP075 | MVL5/MC2/Deoxycholate/DSPC/ | 5/5/40.1/49/1 |
| | DMG-PEG | |
| MVLP076 | MVL5/MC2/Deoxycholate/DSPC/ | 12.4/12.4/74.3/0/1 |
| | DMG-PEG | |
| MVLP077 | MVL5/MC2/Deoxycholate/DSPC/ | 9.3/15.5/33.4/40.8/ |
| | DMG-PEG | 1 |
| MVLP078 | MVL5/MC2/Deoxycholate/DSPC/ | 6.2/18.6/33.4/40.8/ |
| | DMG-PEG | 1 |
| MVLP079 | MVL5/MC2/Deoxycholate/DSPC/ | 3.1/21.7/33.4/ |
| | DMG-PEG | 40.8/1 |
| MVLP080 | MVL5/MC2/Deoxycholate/DSPC/ | 18.6/30.9/22.3/ |
| | DMG-PEG | 27.2/1 |
| MVLP081 | MVL5/MC2/Deoxycholate/DSPC/ | 12.4/37.1/22.3/ |
| | DMG-PEG | 27.2/1 |
| MVLP082 | MVL5/MC2/Deoxycholate/DSPC/ | 6.2/43.3/22.3/27.2/ |
| | DMG-PEG | 1 |
| MVLP083 | MVL5/MC2/DMPC/Deoxycholate/ | 12.5/12.5/41.1/ |
| | DMG-PEG | 33.8/1 |
| MVLP084 | MVL5/MC2/DMPC/Deoxycholate/ | 12.5/12.5/41.1/ |
| | DMPE-PEG | 33.8/1 |
| MVLP085 | MVL5/MC2/DMPC/DMG-PEG | 12.4/12.4/74.3/1 |
| MVLP086 | MVL5/MC2/DOPE/DMG-PEG | 12.4/12.4/74.3/1 |
| MVLP087 | MVL5/MC2/DSPC/3-oxy-cholenic acid/ | 12.4/12.4/40.8/ |
| | DMG-PEG | 33.4/1 |
| MVLP088 | MVL5/MC2/DSPC/Alloisolithocholate/ | 12.5/12.5/41.1/ |
| | DMG-PEG | 33.8/1 |
| MVLP089 | MVL5/MC2/DSPC/Chenodeoxycholate/ | 12.5/12.5/41.1/ |
| | DMG-PEG | 33.8/1 |
| MVLP090 | MVL5/MC2/DSPC/Dehydrolithocholate/ | 12.5/12.5/41.1/ |
| | DMG-PEG | 33.8/1 |
| MVLP091 | MVL5/MC2/DSPC/Deoxycholate/ | 12.4/12.4/40.8/ |
| | DMG-PEG | 33.4/1 |
| MVLP092 | MVL5/MC2/DSPC/Deoxycholate/ | 12.5/12.5/41.1/ |
| | DMPE-PEG | 33.8/1 |
| MVLP093 | MVL5/MC2/DSPC/Deoxycholate/ | 12.4/12.4/40.8/ |
| | DMPE-PEG | 33.4/1 |
| MVLP094 | MVL5/MC2/DSPC/Deoxycholate/ | 12.5/12.5/41.1/ |
| | Lithocholate/DMG-PEG | 27.1/6.8/1 |
| MVLP095 | MVL5/MC2/DSPC/DMG-PEG | 18.6/18.6/61.3/1.5 |
| MVLP096 | MVL5/MC2/DSPC/DMG-PEG | 12.4/12.4/74.3/1 |
| MVLP097 | MVL5/MC2/DSPC/Glycocholate/ | 12.4/12.4/40.8/ |
| | DMPE-PEG | 33.4/1 |
| MVLP098 | MVL5/MC2/DSPC/ | 12.4/12.4/40.8/ |
| | Taurochenodeoxycholate/DMG-PEG | 33.4/1 |
| MVLP099 | MVL5/MC2/DSPC/Taurodeoxycholate/ | 12.4/12.4/40.8/ |
| | DMG-PEG | 33.4/1 |
| MVLP100 | MVL5/MC2/Lithocholate/DSPC | 12.4/12.4/33.4/ |
| | /DMG-PEG | 40.8/1 |
| MVLP101 | MVL5/CL1H6/Deoxycholate/ | 12.4/12.4/74.3/1 |
| | DMG-PEG | |
| DOLP001 | DODMA/DSPC/Deoxycholate | 25/41.25/33.75 |
| DOLP002 | DODMA/DSPC/Deoxycholate/PEG-DMG | 40/31.6/25.9/2.5 |
| DOLP003 | DODMA/DSPC/Deoxycholate/PEG-DMG | 30/37.1/30.4/2.5 |
| DOLP004 | DODMA/DSPC/Deoxycholate/PEG-DMG | 20/42.6/34.9/2.5 |
| DOLP005 | DODMA/DSPC/Deoxycholate/PEG-DMG | 10/48.1/39.4/2.5 |
| DOLP006 | DODMA/DSPC/Deoxycholate/PEG-DMG | 25/37.1/30.4/7.5 |
| DSLP001 | DOPC/Deoxycholate | 55/45 |
| DSLP002 | DSPC/Deoxycholate | 55/45 |
| DSLP003 | DSPC/Deoxycholate/DMG-PEG | 53.6/43.9/2.5 |
| M2LP001 | MC2/Deoxycholate/DSPC/DMG-PEG | 24.8/33.4/40.8/1 |
| M2LP002 | MC2/Deoxycholate/DSPC/DMG-PEG | 49.5/22.3/27.2/1 |

### Exemplary Delivery Vehicle Embodiments

In some embodiments are provided a delivery vehicle comprising a cargo in a lipid structure, for example a lipid nanoparticle, and wherein the lipid nanoparticle comprises a bile salt and at least one of: (a) a saturated cationic lipid that has a phase transition temperature of at least about 37° C., and a non-cationic lipid; (b) a saturated cationic lipid, an unsaturated cationic lipid, a non-cationic lipid, wherein the unsaturated cationic lipid, the non-cationic lipid, or the unsaturated cationic lipid and the non-cationic lipid, have a phase transition temperature of at least about 37° C.; or (c) a multivalent cationic lipid, a non-cationic lipid, wherein the multivalent cationic lipid, the non-cationic lipid, or the multivalent cationic lipid and the non-cationic lipid have a phase transition temperature of at least about 37° C., wherein the delivery vehicle is stable in a high bile salt environment, compared to an otherwise identical delivery vehicle that (i) does not comprise a lipid nanoparticle comprising the bile salt and at least one of (a), (b), or (c); (ii) comprises a lipid nanoparticle comprising at least one of (a), (b), or (c), but does not comprise the bile salt, or (iii) comprises the bile salt but does not comprise at least one of (a), (b), or (c).

In some embodiments are provided a delivery vehicle comprising a cargo and a lipid structure, such as a lipid nanoparticle, wherein the lipid nanoparticle comprises a bile salt and at least one of: (a) a saturated cationic lipid that has a phase transition temperature of at least about 37° C.; (b) a saturated cationic lipid, an unsaturated cationic lipid and a non-cationic lipid, wherein the unsaturated cationic lipid, the non-cationic lipid, or the unsaturated cationic lipid and the non-cationic lipid, have a phase transition temperature of at least about 37° C.; or (c) a multivalent cationic lipid and a non-cationic lipid, wherein the multivalent cationic lipid, the non-cationic lipid, or the multivalent cationic lipid and the non-cationic lipid have a phase transition temperature of at least about 37° C., wherein the delivery vehicle demonstrates an increased stability in a solution containing at least about 5 g/L of cholic acid and deoxycholate, compared to an otherwise identical lipid nanoparticle (i) does not comprise a lipid nanoparticle comprising the bile salt and at least one of (a), (b), or (c); (ii) comprises a lipid nanoparticle comprising at least one of (a), (b), or (c), but does not comprise the bile salt, or (iii) comprises the bile salt but does not comprise at least one of (a), (b), or (c), wherein the stability is measured by relative fluorescence intensity of a fluorescent lipid incorporated into the lipid nanoparticle, in a Forster resonance energy transfer (FRET) assay.

In some embodiments are provided a delivery vehicle comprising (i) a cargo and (ii) a lipid structure, such as a lipid nanoparticle, wherein the lipid nanoparticle comprises at least one saturated cationic lipid and a bile salt, wherein the at least one saturated cationic lipid has a phase transition temperature of at least about 37° C. In some embodiments are provided a delivery vehicle comprising (i) a cargo and a (ii) lipid nanoparticle, wherein the lipid nanoparticle comprises at least one saturated lipid, at least one unsaturated cationic lipid, and a bile salt, wherein the concentration of the at least one unsaturated cationic lipid in the lipid nanoparticle is less than 50 mole %.

In some embodiments, the present disclosure provides compositions that include a cargo; and a nanoparticle, the nanoparticle including: a first cationic lipid and an optional second cationic lipid; at least one bile salt; at least one structural lipid; and at least one conjugated lipid conjugated with a hydrophilic polymer. The at least one bile salt may be selected from one or more of deoxycholate, lithocholate, isolithocholate, alloisolithocholate, dehydrolithocholate, ursodiol, 5beta-cholanic acid, chenodeoxycholate, cholate, taurodeoxycholate, taurochenodeoxycholate, glycocholate, 3-oxy-cholenic acid, and hyodeoxycholate. The at least one bile salt may be included in nanoparticles at levels of from about 5 to about 40 mole % of total nanoparticle lipids. The at least one bile salt may be included in nanoparticles at levels of from about 20 to about 40 mole % of total nanoparticle lipid. The at least one bile salt may include deoxycholate. The first cationic lipid may include CL1H6 or CL4H6. The first cationic lipid may be included at levels of from about 5 to about 40 mole % of the total nanoparticle lipid. The second cationic lipid may include MVL5, MC2, or DODMA. The second cationic lipid may be present at levels of from about 5 to about 20 mole % of total nanoparticle lipid. Each of the first cationic lipid and the second cationic lipid may be present at levels of from about 5 to about 20 mole % of total nanoparticle lipid and may be present in equal amounts. The at least one structural lipid may be selected from one or more of DSPC, DMPC, and dioleoylphosphatidylethanolamine (DOPE). The at least one structural lipid may be present at levels of from about 10 to about 70 mole % of total nanoparticle lipid. The at least one structural lipid may be present at levels of from about 30 to about 50 mole % of total nanoparticle lipid. The at least one structural lipid and the at least one bile salt may be present at combined levels of from about 50 to about 80 mole % of total nanoparticle lipid. The hydrophilic polymer may include PEG. The at least one conjugated lipid may include DMG-PEG. The at least one conjugated lipid may be present at levels of from about 0.5 to about 2.0 mole % of total nanoparticle lipid. The first cationic lipid may be CL1H6. The nanoparticle may include a second cationic lipid that includes MVL5. The at least one bile salt may be deoxycholate. The at least one structural lipid may be DSPC. The at least one conjugated lipid may be DMG-PEG.

In some embodiments, the present disclosure provides compositions that include a cargo and nanoparticles, wherein the nanoparticles include CL1H6, MVL5, and DMG-PEG at molar ratios of about 1:1:0.08; and deoxycholate and DSPC at molar ratios of from about 0.5 to about 5.0. The molar ratios of deoxycholate and DSPC may be from about 2.0 to about 4.0. The nanoparticles may include CL1H6 at levels of from about 10 to about 20 mole % of total nanoparticle lipid; MVL5 at levels of from about 10 to about 20 mole % of total nanoparticle lipid; deoxycholate at levels of from about 10 to about 40 mole % of total nanoparticle lipid; DSPC, DMPC, or DOPE at levels of from about 30 to about 60 mole % of total nanoparticle lipid; and DMG-PEG at levels of from about 0.5 to about 2.0 mole % of total nanoparticle lipid. The nanoparticle may include: CL1H6 and MVL5 at levels of from about 10 to about 15 mole % of total nanoparticle lipid; deoxycholate at levels of from about 20 to about 40 mole % of total nanoparticle lipid; DSPC at levels of from about 35 to about 50 mole % of total nanoparticle lipid; and DMG-PEG at levels of from about 0.75 to about 1.5 mole % of total nanoparticle lipid. The nanoparticle may include: CL1H6 and MVL5 at levels of from about 12 to about 14 mole % of total nanoparticle lipid; deoxycholate at levels of from about 27 to about 38 mole % of total nanoparticle lipid; DSPC at levels of from about 38 to about 45 mole % of total nanoparticle lipid; and DMG-PEG at levels of from about 0.75 to about 1.5 mole % of total nanoparticle lipid. The nanoparticles may include: CL1H6 and MVL5 at levels of about 12 mole % of total nanoparticle lipid; deoxycholate at levels of about 33 mole % of total nanoparticle lipid; DSPC at levels of about 41 mole % of total nanoparticle lipid; and DMG-PEG at levels of about 1 mole % of total nanoparticle lipid. Hydrophilic polymers may be conjugated with polypeptides. Polypeptides may include MPPs, for example, any of those described in Table 3 of International Publication Number WO2019222400, the contents of which are herein incorporated by reference in their entirety. MPPs may include amino acid sequences according to TVDNDAPTKRASKLFAV (SEQ ID NO: 17). Hydrophilic polymers may include PEG. The at least one conjugated lipid may include DMG-PEG. Nanoparticles may include: CL1H6 and MVL5 at levels of from about 12 to about 14 mole % of total nanoparticle lipid; deoxycholate at levels of from about 27 to about 38 mole % of total nanoparticle lipid; DSPC at levels of from about 38 to about 45 mole % of total nanoparticle lipid; and DMG-PEG at levels of from about 0.75 to about 1.5 mole % of total nanoparticle lipid. The cargo may include one or more of a nucleic acid, a protein, an antibody, a peptide, a small molecule, a biologic, a peptidomimetic, a ribozyme, a chemical agent, a viral particle, a growth factor, a cytokine, an immunomodulating agent, and a fluorescent dye. The cargo may include nucleic acids. The nucleic acids may include DNA. The DNA may include plasmid DNA. The molar ratio of nanoparticle cationic lipids to nanoparticle nucleotides may be from about 2 to about 20. The molar ratio of nanoparticle cationic lipids to nanoparticle nucleotides may be from about 14 to about 18. The nucleic acid may include RNA. The molar ratio of nanoparticle cationic lipids to the total number cargo RNA nucleotides may be from about 2 to about 20. The molar ratio of nanoparticle cationic lipids to the cargo RNA nucleotides may be from about 2 to about 4.

In some embodiments, the present disclosure provides a composition that includes a cargo and a nanoparticle, the nanoparticle including: at least one bile salt; at least one cationic lipid; at least one structural lipid; and at least one conjugated lipid, wherein the conjugated lipid is conjugated with a hydrophilic polymer. The at least one bile salt may be selected from one or more of deoxycholate, lithocholate, isolithocholate, alloisolithocholate, dehydrolithocholate, ursodiol, 5beta-cholanic acid, chenodeoxycholate, cholate, taurodeoxycholate, taurochenodeoxycholate, glycocholate, 3-oxy-cholenic acid, and hyodeoxycholate. The at least one bile salt may be included in the nanoparticle at a level of from about 5 to about 40 mole % of total nanoparticle lipid. The at least one bile salt may be included in the nanoparticle at a level of from about 20 to about 40 mole % of total nanoparticle lipid. The at least one bile salt may be included in the nanoparticle at a level of from about 33 to about 37 mole % of total nanoparticle lipid. The at least one bile salt may include deoxycholate. The composition may include two bile salts. At least one of the two bile salts may include lithocholate. The composition may include deoxycholate at a level of from about 20 to about 30 mole % of total nanoparticle lipid; and lithocholate at a level of from about 5 to about 10 mole % of total nanoparticle lipid. The at least one cationic lipid may include N1-[2-((1S)-1-[(3-aminopropyl)amino]-4-[di(3-amino-propyl)amino]butylcarboxamido)ethyl]-3,4-di[oleyloxy]-benzamide (MVLS). The MVL5 may be present at a level of from about 5 to about 20 mole % of total nanoparticle lipid. The at least one cationic lipid may include one or more of (6Z,9Z,28Z,31Z)-heptatriaconta-6,9,28,31-tetraen-19-yl 3-(dimethylamino)propanoate (MC2); 7-(4-(dimethylamino)butyl)-7-hydroxytridecane-1,13-diyl dioleate (CL1H6); and 7-(4-(diisopropylamino)butyl)-7-hydroxytride-cane-1,13-diyl dioleate (CL4H6). Each one of the at least one cationic lipid may be present at a level of from about 5 to about 20 mole % of total nanoparticle lipid. The at least one structural lipid may be selected from one or more of 1,2-distearoyl-sn-glycero-3-phosphocholine (DSPC) and 1,2-dimyristoyl-sn-glycero-3-phosphocholine (DMPC). The at least one structural lipid may be present at a level of from about 35 to about 45 mole % of total nanoparticle lipid. The hydrophilic polymer may include polyethylene glycol (PEG). The at least one conjugated lipid may include one or more of 1,2-dimyristoyl-rac-glycerol (DMG)-PEG and 1,2-dimyristoyl-sn-glycero-3-phosphoethanolamine (DMPE)-PEG. The at least one conjugated lipid may be present at a level of from about 0.5 to about 2.0 mole % of total nanoparticle lipid. The molar ratio between components may be: from about 1 to about 5 of the at least one bile salt; from about 0.5 to about 3 of each one of the at least one cationic lipid; from about 2 to about 10 of the at least one structural lipid; and from about 0.02 to about 0.10 of the at least one conjugated lipid. The at least one bile salt may be selected from one or more of deoxycholate, ursodiol, lithocholate, isolithocholate, alloisolithocholate, dehydrolithocholate, and 5beta-cholanic acid. The at least one cationic lipid may include MVL5. The at least one cationic lipid may include MC2. The at least one structural lipid may include DSPC. The at least one conjugated lipid may include DMG-PEG. The composition may include at least one bile salt, MVL5, MC2, DSPC, and DMG-PEG at a molar ratio of about 2.592:0.96:0.96:3.168:0.768. The at least one bile salt may be deoxycholate. The nanoparticle may include: MVL5, MC2, DSPC, deoxycholate, and DMPE-PEG at a molar ratio of about 2.4:2.4:7.9:6.48:0.192; MVL5, CL1H6, DSPC, deoxycholate, and DMG-PEG at a molar ratio of about 2.4:2.4:7.9:6.48:0.192; MVL5, CL4H6, DSPC, deoxycholate, and DMG-PEG at a molar ratio of about 2.4:2.4:7.9:6.48:0.192; MVL5, MC2, DSPC, chenodeoxycholate, and DMG-PEG at a molar ratio of about 2.4:2.4:7.9:6.48:0.192; MVL5, MC2, DMPC, deoxycholate, and DMG-PEG at a molar ratio of about 2.4:2.4:7.9:6.48:0.192; MVL5, MC2, DMPC, deoxycholate, and DMPE-PEG at a molar ratio of about 2.4:2.4:7.9:6.48:0.192; MVL5, CL1H6, DMPC, deoxycholate, and DMG-PEG at a molar ratio of about 2.4:2.4:7.9:6.48:0.192; MVL5, MC2, DSPC, deoxycholate, lithocholate, and DMG-PEG at a molar ratio of about 2.4:2.4:7.9:5.2:1.3:0.192; MVL5, CL1H6, DSPC, deoxycholate, lithocholate, and DMG-PEG at a molar ratio of about 2.4:2.4:7.9:5.2:1.3:0.192; MVL5, MC2, DSPC, alloisolithocholate, and DMG-PEG at a molar ratio of about 2.4:2.4:7.92:6.48:0.192; or MVL5, MC2, DSPC, dehydrolithocholate, and DMG-PEG at a molar ratio of about 2.4:2.4:7.92:6.48:0.192. The composition may include 12.4 mole % of MVL5, 12.4 mole % of MC2, 40.8 mole % of DSPC, 33.4 mole % of the at least one bile salt, and 1 mole % of the at least one conjugated lipid. The at least one conjugated lipid may include DMG-PEG or DMPE-PEG. The at least one bile salt may be selected from one or more of taurodeoxycholate, taurochenodeoxycholate, glycocholate, 3-oxy-cholenic acid, and deoxycholate. The cargo may include one or more of a nucleic acid, a protein, an antibody, a peptide, a small molecule, a biologic, a peptidomimetic, a ribozyme, a chemical agent, a viral particle, a growth factor, a cytokine, an immunomodulating agent, and a fluorescent dye. The cargo may include a nucleic acid. The nucleic acid may include DNA. The DNA may include plasmid DNA.

In some embodiments, the present disclosure provides a composition that includes a cargo; and a nanoparticle, the nanoparticle including: a first cationic lipid that includes CL1H6 or CL4H6; an optional second cationic lipid; at least one bile salt; at least one structural lipid; and at least one conjugated lipid, wherein the at least one conjugated lipid is conjugated with a hydrophilic polymer. The at least one bile salt may be selected from one or more of deoxycholate, lithocholate, isolithocholate, alloisolithocholate, dehydrolithocholate, ursodiol, 5beta-cholanic acid, chenodeoxycholate, cholate, taurodeoxycholate, taurochenodeoxycholate, glycocholate, 3-oxy-cholenic acid, and hyodeoxycholate. The at least one bile salt may be included in the nanoparticle at a level of from about 5 to about 40 mole % of total nanoparticle lipid. The at least one bile salt may be included in the nanoparticle at a level of from about 20 to about 40 mole % of total nanoparticle lipid. The at least one bile salt may include deoxycholate. The first cationic lipid may include from about 5 to about 40 mole % of the total nanoparticle lipid. The nanoparticle may include a second cationic lipid, the second cationic lipid including MVL5, MC2, or DODMA. The second cationic lipid may be present at a level of from about 5 to about 20 mole % of total nanoparticle lipid. Each of the first cationic lipid and the second cationic lipid may be present at a level of from about 5 to about 20 mole % of total nanoparticle lipid and each may be present in an equal amount. The at least one structural lipid may be selected from one or more of DSPC, DMPC, and dioleoylphosphatidylethanolamine (DOPE). The at least one structural lipid may be present at a level of from about 10 to about 70 mole % of total nanoparticle lipid. The at least one structural lipid may be present at a level of from about 30 to about 50 mole % of total nanoparticle lipid. The at least one structural lipid and the at least one bile salt may be present at a combined level of from about 50 to about 80 mole % of total nanoparticle lipid. The hydrophilic polymer may include PEG. The at least one conjugated lipid may include DMG-PEG. The at least one conjugated lipid may be present at a level of from about 0.5 to about 2.0 mole % of total nanoparticle lipid. The first cationic lipid may include CL1H6. The nanoparticle may include a second cationic lipid that includes MVL5. The at least one bile salt may include deoxycholate. The at least one structural lipid may include DSPC. The at least one conjugated lipid may include DMG-PEG. CL1H6, MVL5, and DMG-PEG may be included at molar ratios of about 1:1:0.08; and deoxycholate and DSPC may be included at molar ratios of from about 0.5 to about 5.0. The molar ratios of deoxycholate and DSPC may be from about 2.0 to about 4.0. CL1H6 may be included at a level of from about 10 to about 20 mole % of total nanoparticle lipid; MVL5 may be included at a level of from about 10 to about 20 mole % of total nanoparticle lipid; deoxycholate may be included at a level of from about 10 to about 40 mole % of total nanoparticle lipid; DSPC, DMPC, or DOPE may be included at a level of from about 30 to about 60 mole % of total nanoparticle lipid; and DMG-PEG may be included at a level of from about 0.5 to about 2.0 mole % of total nanoparticle lipid. The nanoparticle may include: CL1H6 and MVL5 at a level of from about 10 to about 15 mole % of total nanoparticle lipid; deoxycholate at a level of from about 20 to about 40 mole % of total nanoparticle lipid; DSPC at a level of from about 35 to about 50 mole % of total nanoparticle lipid; and DMG-PEG at a level of from about 0.75 to about 1.5 mole % of total nanoparticle lipid. The nanoparticle may include: CL1H6 and MVL5 at a level of from about 12 to about 14 mole % of total nanoparticle lipid; deoxycholate at a level of from about 27 to about 38 mole % of total nanoparticle lipid; DSPC at a level of from about 38 to about 45 mole % of total nanoparticle lipid; and DMG-PEG at a level of from about 0.75 to about 1.5 mole % of total nanoparticle lipid. The nanoparticle may include: CL1H6 and MVL5 at a level of about 12 mole % of total nanoparticle lipid; deoxycholate at a level of about 33 mole % of total nanoparticle lipid; DSPC at a level of about 41 mole % of total nanoparticle lipid; and DMG-PEG at a level of about 1 mole % of total nanoparticle lipid. The hydrophilic polymer may be conjugated with a polypeptide. The polypeptide may be a mucus penetrating polypeptide (MPP). The MPP may include an amino acid sequence according to SEQ ID NO: 17. The hydrophilic polymer may include PEG. The at least one conjugated lipid may include DMG-PEG. The nanoparticle may include: CL1H6 and MVL5 at a level of from about 12 to about 14 mole % of total nanoparticle lipid; deoxycholate at a level of from about 27 to about 38 mole % of total nanoparticle lipid; DSPC at a level of from about 38 to about 45 mole % of total nanoparticle lipid; and DMG-PEG at a level of from about 0.75 to about 1.5 mole % of total nanoparticle lipid. The cargo may include one or more of a nucleic acid, a protein, an antibody, a peptide, a small molecule, a biologic, a peptidomimetic, a ribozyme, a chemical agent, a viral particle, a growth factor, a cytokine, an immunomodulating agent, and a fluorescent dye. The cargo may include a nucleic acid. The nucleic acid may include DNA. The DNA may include plasmid DNA. The molar ratio of total nanoparticle cationic lipids to the total number of nucleotides of the nucleic acid cargo may be from about 2 to about 20. The molar ratio of total nanoparticle cationic lipids to the total number of nucleotides of the nucleic acid cargo may be from about 14 to about 18. The nucleic acid may include RNA. The molar ratio of total nanoparticle cationic lipids to the total number of nucleotides of the nucleic acid cargo may be from about 2 to about 20. The molar ratio of total nanoparticle cationic lipids to the total number of nucleotides of the nucleic acid cargo may be from about 2 to about 4.

### III. IMPROVED FUNCTION OF DESCRIBED DELIVERY VEHICLE

### Improved Stability in Bile Salt Environments

In some embodiments, stability of the delivery vehicle can be measured by a bile salt stability assay, in a high bile salt mimicking environment. For example, bile salt stability can be measured by fluorescence spectroscopy, such as relative fluorescence of delivery vehicles containing varying concentrations of bile salts or bile acids, in a Forster resonance energy transfer (FRET) assay. In some embodiments, the incorporated bile salt (s) and/or bile acid (s) can increase the stability of the delivery vehicle from about 80% to about 10%, such as about 80% to about 70%, about 65% to about 55%, about 60% to about 50%, about 55% to about 45%, about 50% to about 40%, about 45% to about 35%, about 40% to about 30%, about 35% to about 25%, about 30% to about 20%, about 25% to about 15%, about 20% to about 10%, about 15% to about 10, about 60% to about 20%, about 25.9%, about 30.4%, about 34.9%, about 39.4%, about 37.1%, about 43.9%, or about 45%. In some embodiments, the incorporated bile salt (s) and/or bile acid (s) can increase the stability of the delivery vehicle as compared to a delivery vehicle that includes only a single cationic lipid, lacks a bile salt or bile acid, or any combination thereof.

In some embodiments, the delivery vehicle stability can be increased with the incorporation of the bile salt and/or bile acid. For example, bile salt stability can be measured by fluorescence spectroscopy, such as relative fluorescence of delivery vehicles containing varying concentrations of bile salts, in a Forster resonance energy transfer (FRET) assay. In some embodiments, the incorporated bile salt (s) and/or bile acid (s) can increase the stability of the delivery vehicle from about 80% to about 10%, such as about 80% to about 70%, about 65% to about 55%, about 60% to about 50%, about 55% to about 45%, about 50% to about 40%, about 45% to about 35%, about 40% to about 30%, about 35% to about 25%, about 30% to about 20%, about 25% to about 15%, about 20% to about 10%, about 15% to about 10, about 60% to about 20%, about 25.9%, about 30.4%, about 34.9%, about 39.4%, about 37.1%, about 43.9%, or about 45% as compared to the stability of comparable delivery vehicle that includes only a single cationic lipid, lacks a bile salt or bile acid, or any combination thereof.

In some examples, the percent increase in stability can be measured by increased relative fluorescence units or relative luminescence units in an assay, such as FRET. In some embodiments, a FRET assay is performed by (i) incorporating fluorescent dyes that are FRET pairs, such as Dil and DiO into the delivery vehicle; (ii) treating the delivery vehicle with a simulated bile salt environment, such as a mixture of cholic acid and deoxycholate at varying concentrations; (iii) determine the relative fluorescence units (RFU) by exciting the florescent die and reading the emission at wavelengths appropriate for the dyes used; and (iv) normalize the readings relative to the FRET intensity of the system without any treatment. In some embodiments, a lower normalized FRET intensity indicates a lower delivery vehicle stability in the bile salt environment.

In some embodiments, the delivery vehicle demonstrates an increased stability in a solution containing at least about 0.5 g/L, 1 g/L, 5 g/L, 7 g/L, 9 g/L, 11 g/L, 13 g/L, 15 g/L, 17 g/L, 19 g/L, 21 g/L, 23 g/L, or up to about 25 g/L of bile acid, for example, a mixture of about 40%, 45%, 50%, or up to about 55% cholic acid and about 40%, 45%, 50%, 55%, or up to about 60% deoxycholate, as compared to the stability of comparable delivery vehicle that includes only a single cationic lipid, lacks a bile salt or bile acid, or any combination thereof, wherein the stability is measured by relative fluorescence intensity of a fluorescent lipid incorporated into the lipid nanoparticle, in a Forster resonance energy transfer (FRET) assay.

### Improved Mucus Penetration/Trafficking

In some embodiments, the delivery vehicle that comprises a bile salt or bile acid as provided herein can have improved trafficking, transfection of target cells, epithelial reach, or a combination thereof as compared to a comparable delivery vehicle that lacks the bile salt. In some embodiments, the improvement is from about 1 fold, 50 fold, 99 fold, 148 fold, 197 fold, 246 fold, 295 fold, 344 fold, 393 fold, 442 fold, 491 fold, 540 fold, 589 fold, 638 fold, 687 fold, 736 fold, 785 fold, 834 fold, 883 fold, 932 fold, 981 fold, or up to about 1000 fold as comparable delivery vehicle that includes only a single cationic lipid, lacks a bile salt or bile acid, or any combination thereof. In some examples, the percent increase in stability can be measured by increased relative fluorescence units or relative luminescence units in an assay, such as FRET in vivo or ex vivo.

### Improved Cellular Uptake

In some embodiments, the delivery vehicles described herein which include a bile salt or bile acid can permit efficient penetration and transit through the mucus layer to the target cells. In some embodiments, efficient penetration and transit through the mucus layer increases efficient uptake by the target cell(s). For example, the delivery vehicle can be taken up by about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9% of the total number of cells that are contacted. In some embodiments, the compositions can have a higher percent of cellular uptake as compared to a comparable delivery vehicle that does not include a bile salt or bile acid. The improvement can be from about 10%, 15%, 20%, 25%, 30%, 35%, 40%, 45%, 50%, 55%, 60%, 65%, 70%, or up to about 80% better.

In some embodiments, an efficiency of transfection or integration of a polynucleic acid cargo delivered to a cell by the delivery vehicle can be from about 5%, 10%, 15%, 20%, 25%, 30%, 35%, 40%, 45%, 50%, 55%, 60%, or up to 65% better than a comparable delivery vehicle that does not include the bile salt, the bile acid, an MPP (or other additional component), a particular composition of delivery vehicle lipids disclosed herein, or any combination thereof. In some embodiments, an efficiency of transfection or integration of a polynucleic acid cargo delivered to a cell by the delivery vehicle composition as described herein can be from about 5%, 10%, 15%, 20%, 25%, 30%, 35%, 40%, 45%, 50%, 55%, 60%, or up to 65% better than a comparable delivery vehicle that does not include a bile salt, bile acid, or charge separation. In some embodiments, an efficiency of transfection or integration of a polynucleic acid cargo delivered to a cell by the delivery vehicle composition as described herein can be from about 5%, 10%, 15%, 20%, 25%, 30%, 35%, 40%, 45%, 50%, 55%, 60%, or up to 65% better than a comparable delivery vehicle that includes only a single cationic lipid, lacks a bile salt or bile acid, or any combination thereof.

In some embodiments, the delivery vehicle provides a proximity distance to a cell, such as an epithelial cell. In some embodiments, such proximity distance is less than about 50, 40, 30, 25, 20, 19, 18, 17, 16, 15, 14, 13, 12, 11, 10, 9, 8, 7, 6, 5, 4, 3, 2, or 1 microns. In some embodiments, the delivery vehicles herein come in contact with the cell. In some embodiments, the delivery vehicle is internalized into the cell and a cargo carried by the delivery vehicle is released within the cell. In some embodiments, the delivery vehicle contacts the cell (e.g., epithelial cell) and a cargo from the delivery vehicle is released outside of the cell.

In some embodiments, the delivery vehicles provided herein provide a closer proximity distance than comparable delivery vehicle that includes only a single cationic lipid, lacks a bile salt or bile acid, or any combination thereof. In some embodiments, the delivery vehicles provide a proximity distance that is 1×, 5×, 10×, 15×, 20×, 25×, 50×, 75×, 100×, 200×, 300×, 400×, 500×, 1,000×, 5,000×, 10,000× or more times closer to the target cell than comparable delivery vehicle that includes only a single cationic lipid, lacks a bile salt or bile acid, or any combination thereof.

### IV. CARGOS

The delivery vehicles of the present disclosure may comprise, enclose, or be conjugated to a cargo. As used herein, the term “cargo” can refer to one or more molecules or structures encompassed in the delivery vehicle for delivery to or into a cell or tissue. Non-limiting examples of cargo can include a nucleic acid, a polypeptide, peptide, protein, a liposome, a label, a tag, a small chemical molecule, a large biological molecule, an antibody, a biologic, a peptidomimetic, a ribozyme, a chemical agent, a viral particle, a growth factor, a cytokine, an immunomodulating agent, a fluorescent dye and any combinations or fragments thereof. Wherein cargo may include nucleic acids, such nucleic acids may include DNA (e.g., plasmid DNA), RNA, and any combination thereof.

### Therapeutic Agents

In some embodiments, a cargo can comprise a therapeutic agent. Exemplary therapeutic agents may comprise, a nucleic acid, a protein, an antibody, a peptide, a small molecule (including small organic molecules or compounds), a biologic, an antisense oligonucleotide, peptidomimetics, ribozymes, a chemical agent (such as a chemotherapeutic molecule), small molecule drugs, fluorescent dyes, anti-inflammatory compounds, anti-depressants, stimulants, analgesics, antibiotics, birth control medication, antipyretics, vasodilators, anti-angiogenics, cytovascular agents, signal transduction inhibitors, cardiovascular drugs, (e.g., anti-arrhythmic agents, vasoconstrictors, hormones, and steroids), cytokines, growth factors, apoptotic factors, differentiation-inducing factors, cell surface receptors, antibodies, (such as, e.g., polyclonal antibodies, monoclonal antibodies, antibody fragments; humanized antibodies, recombinant antibodies, recombinant human antibodies, and Primatized™ antibodies), viral particles, growth factors cytokines, immunomodulating agents, polysaccharides, saccharides, or any combination thereof.

In some embodiments, the delivery vehicle can include any molecule or compound capable of exerting a desired effect on a cell, tissue, organ, or subject. Such effects may be, for example, biological, physiological, or cosmetic.

In one embodiment, a molecules or compound can be a therapeutic agent, or a salt or derivative thereof. Therapeutic agent derivatives may be therapeutically active themselves or they may be prodrugs, which become active upon further modification. Thus, in one embodiment, a molecules or compound derivative may retain some or all of the therapeutic activity as compared to the unmodified agent, while in another embodiment, a therapeutic derivative lacks therapeutic activity.

In some embodiments, a cargo may be a drug. In some embodiments, a drug may be a substance that when administered may cause a physiological change in a subject. In some embodiments, a drug may be a medication used to treat a disease, such as cancer. In some embodiments, drugs may be entrapped completely in a liposomal lipid bilayer, in an aqueous compartment, or in both a liposomal lipid bilayer and an aqueous compartment. In some embodiments, strongly lipophilic drugs may be entrapped almost completely in a lipid membrane. In some embodiments, strongly hydrophilic drugs may be located exclusively in an inter-nanoparticle space. In some embodiments, drugs with intermediate log P may easily partition between a lipid and aqueous phases, both in a lipid-layer and in an aqueous inter-nanoparticle space. In some embodiments, exemplary drugs may comprise drugs such as, but not limited to, adalimumab, anti-TNF, insulin-like growth factor, interleukin, Mesalamine, GLP-1 analogs, GLP-2 analogs, and combinations thereof.

### Nucleic Acids

In some embodiments, a cargo may be a nucleic acid compound. In some embodiments, a nucleic acid compound may be DNA- or RNA-based. In some embodiments, a nucleic acid may be a vector. In some embodiments, DNA-based vectors may be non-viral, and may include molecules such as plasmids, minicircles, nanoplasmid, closed linear DNA (doggybone), linear DNA, and single-stranded DNA. In some embodiments, the nucleic acid compound may include any form of nucleic acid that is known. In some embodiments, the nucleic acid compound may comprise single-stranded DNA or RNA, or double-stranded DNA or RNA, or DNA-RNA hybrids. In some embodiments, double-stranded DNA may include, but is not limited to structural genes, genes including control and termination regions, and self-replicating systems such as viral or plasmid DNA. In some embodiments, double-stranded RNA may include, but is not limited to, siRNA and other RNA interference reagents. In some embodiments, single-stranded nucleic acids may include, but are not limited to, messenger RNA (mRNA) antisense oligonucleotides, ribozymes, microRNA, and triplex-forming oligonucleotides. In some embodiments, the nucleic acid compounds may include, but is not limited to, one or more of the oligonucleotide modifications described herein.

In some embodiments, nucleic acids may be of various lengths, generally dependent upon the particular form of nucleic acid. In some embodiments, plasmids or genes may be from about 1,000 to 100,000 nucleotide residues in length. In some embodiments, oligonucleotides may range from about 10 to 100 nucleotides in length. In some embodiments, oligonucleotides, single-stranded, double-stranded, and triple-stranded, may range in length from about 10 to about 50 nucleotides, from about 20 to about 50 nucleotides, from about 15 to about 30 nucleotides, from about 20 to about 30 nucleotides in length. In some embodiments, oligonucleotides may range from about 2 nucleotides to 10 nucleotides in length.

In some embodiments, nucleic acid compounds, e.g., polynucleic acids, may be delivered to cells, for example, cells of the intestinal tract. In some embodiments, a nucleic acid compound may be delivered by the delivery vehicles herein to an intestinal crypt stem cell. In some embodiments, a delivered nucleic acid compound may be: (1) not normally found in intestinal epithelial stem cells; (2) normally found in intestinal epithelial stem cells, but not expressed at physiological significant levels; (3) normally found in intestinal epithelial stem cells and normally expressed at physiological desired levels in the stem cells or their progeny; (4) any other DNA which may be modified for expression in intestinal epithelial stem cells; and (5) any combination of the above.

In some embodiments, minicircle (MC) DNA may be delivered as cargo by a delivery vehicle. In some embodiments, MC may be similar to plasmid DNA as both may contain expression cassettes that may permit transgene products to be made at high levels shortly after delivery. In some embodiments, a MC may differ in that MC DNA may be devoid of prokaryotic sequence elements (e.g., bacterial origin of replication and antibiotic-resistance genes). In some embodiments, removal of prokaryotic sequence elements from a backbone plasmid DNA may be achieved via site-specific recombination in Escherichia coli before episomal DNA isolation. In some embodiments, the lack of prokaryotic sequence elements may reduce MC size relative to its parental full-length (FL) plasmid DNA, which may lead to enhanced transfection efficiencies. The result may be that when compared with their FL plasmid DNA counterparts, MCs may transfect more cells and may permit sustained high-level transgene expression upon delivery. In some embodiments, a minicircle DNA may be free of a bacterial origin of replication. In some embodiments, a minicircle DNA or closed linear DNA, may be free of a bacterial origin of replication from about 50% of a bacterial origin of replication sequence or up to 100% of a bacterial origin of replication. In some embodiments, a bacterial origin of replication is truncated or inactive. In some embodiments, a polynucleic acid may be derived from a vector that initially encoded a bacterial origin of replication. In some embodiments, a method may be utilized to remove the entirety of a bacterial origin of replication or a portion thereof, leaving a polynucleic acid free of a bacterial origin of replication. In some embodiments, a bacterial origin of replication may be identified by its high adenine and thymine content. In some embodiments, minicircle DNA vectors may be supercoiled minimal expression cassettes, derived from conventional plasmid DNA by site-specific recombination in vivo in Escherichia coli for the use in non-viral gene therapy and vaccination. In some embodiments, minicircle DNA may lack or have reduced bacterial backbone sequences such as an antibiotic resistance gene, an origin of replication, and/or inflammatory sequences intrinsic to bacterial DNA. In addition to their improved safety profile, minicircles may greatly increase efficiency of transgene expression.

In some embodiments, a portion of a gene may be delivered by a polynucleic acid cargo. In some embodiments, a portion of a gene may be from three nucleotides up to the entire whole genomic sequence. In some embodiments, a portion of a gene may be from about 1% up to about 100% of an endogenous genomic sequence. In some embodiments, a portion of a gene may be from about 1%, 10%, 20%, 30%, 40%, 50%, 60%, 70%, 80%, 90%, or up to about 100% of a whole genomic sequence of a gene.

In some embodiments, nucleic acids encode antibodies. As used herein, the term “antibody” is used in the broadest sense and specifically embraces various antibody formats including, but not limited to, monoclonal antibodies, polyclonal antibodies, multispecific antibodies (e.g., bispecific antibodies formed from at least two intact antibodies), and antibody fragments (e.g., diabodies) so long as they exhibit a desired biological activity (e.g., are “functional” fragments). Encoded antibodies may bind targets that include one or more of IL-18, IL-18 receptor 1 (IL18R1), IL-23, tumor necrosis factor α (TNFα), proprotein convertase subtilisin kexin 9 (PCSK9), and protein 19 (P19). Encoded antibodies may include bispecific antibodies. Bispecific antibodies may bind to cluster of differentiation 3 (CD3) for recruitment of immune cells to targets of a second bispecific antibody epitope.

In some embodiments, a polynucleic acid for use as a cargo with the delivery vehicles herein include nucleic acids encoding for a tumor-suppressor gene. In some embodiments, a tumor-suppressor gene may generally encode for a protein that in one way or another may inhibit cell proliferation.

Without wishing to be bound by theory, loss of one or more of these “brakes” may contribute to the development of a cancer. Five broad classes of proteins may be generally recognized as being encoded by tumor-suppressor genes: Intracellular proteins, such as the p16 cyclin-kinase inhibitor, that may regulate or inhibit progression through a specific stage of the cell cycle, receptors for secreted hormones (e.g., tumor derived growth factor R) that may function to inhibit cell proliferation, checkpoint-control proteins that arrest the cell cycle if DNA may be damaged or chromosomes are abnormal, proteins that may promote apoptosis, enzymes that participate in DNA repair, or a combination thereof. Although DNA-repair enzymes may not directly function to inhibit cell proliferation, cells that have lost the ability to repair errors, gaps, or broken ends in DNA accumulate mutations in many genes, including those that are critical in controlling cell growth and proliferation. Thus loss-of-function mutations in the genes encoding DNA-repair enzymes may promote inactivation of other tumor-suppressor genes as well as activation of oncogenes. Since generally one copy of a tumor-suppressor gene suffices to control cell proliferation, both alleles of a tumor-suppressor gene must be lost or inactivated in order to promote tumor development.

In some embodiments, oncogenic loss-of-function mutations in tumor-suppressor genes act recessively. Tumor-suppressor genes in many cancers have deletions or point mutations that prevent production of any protein or lead to production of a nonfunctional protein.

In some embodiments, introducing a tumor suppressor gene encoding for a protein may ameliorate disease, prevent disease, or treat disease in a subject.

In some embodiments, a tumor suppressor gene may include, but are not limited to, APC, ARHGEF12, ATM, BCL11B, BLM, BMPR1A, BRCA1, BRCA2, CARS, CBFA2T3, CDH1, CDH11, CDK6, CDKN2C, CEBPA, CHEK2, CREB1, CREBBP, CYLD, DDX5, EXT1, EXT2, FBXW7, FH, FLT3, FOXP1, GPC3, IDH1, IL2, JAK2, MAP2K4, MDM4, MEN1, MLH1, MSH2, NF1, NF2, NOTCHI, NPM1, NR4A3, NUP98, PALB2, PML, PTEN, RB1, RUNX1, SDHB, SDHD, SMARCA4, SMARCB1, SOCS1, STK11, SUFU, SUZ12, SYK, TCF3, TNFAIP3, TP53, TSC1, TSC2, VHL, WRN, WT1, and any combination thereof.

In some embodiments, gastrointestinal cells may be transfected with nucleic acid cargo that includes non-coding RNA. As used herein, the term “non-coding RNA” refers to RNA molecules with sequences that do not encode proteins, but typically have significance in some other RNA function. Non-coding RNA may include, but is not limited to, short interfering RNA (siRNA), microRNA (miRNA), long non-coding RNA, piwi-interacting RNA (piRNA), small nucleolar RNA (snoRNA), small Cajal body-specific RNA (scaRNA), transfer RNA (tRNA), ribosomal RNA (rRNA), and small nuclear RNA (snRNA).

### Vectors

In some embodiments, DNA-based vectors may also be viral, and include adeno-associated virus, lentivirus, adenovirus, and others. In some embodiments, vectors may also be RNA. In some embodiments, RNA vectors may be linear or circular forms of unmodified RNA. In some embodiments, the nucleic acid compound may also include various nucleotide modifications designed to increase half-life, decrease immunogenicity, and/or increase level of translation. In some embodiments, a vector may be composed of either DNA or RNA. In some embodiments, a vector may be composed of DNA. In some embodiments, a vector may be composed of RNA. In some embodiments, vectors may be capable of autonomous replication in a prokaryote such as E. coli, which may, for example, be used for growth. In some embodiments, a vector may be stably integrated into a genome of an organism. In some embodiments, a vector may remain separate, either in a cytoplasm or a nucleus. In some embodiments, a vector may contain a targeting sequence. In some embodiments, a vector may contain an antibiotic resistance gene. In some embodiments, a vector may contain regulatory elements for regulating gene expression. In some embodiments, a mini-circle may be enclosed within the delivery vehicle.

### Nuclear Localization Sequence (NLS)

In some embodiments, a polynucleic acid may encode for a heterologous sequence. In some embodiments, a heterologous sequence may provide for subcellular localization (e.g., a nuclear localization signal (NLS) for targeting to a nucleus; a mitochondrial localization signal for targeting to a mitochondria; a chloroplast localization signal for targeting to a chloroplast; an ER retention signal; and the like). In some embodiments, a polynucleic acid, such as minicircle DNA or closed linear DNA, may comprise a nuclear localization sequence (NLS).

In some embodiments, a cargo may comprise one or more nuclear localization sequences (NLSs). In some embodiments, a number of NLS sequences may be from about 1, 2, 3, 4, 5, 6, 7, 8, 9, 10, or more NLSs. In some embodiments, a vector comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., one or more NLS at the amino-terminus and one or more NLS at the carboxy terminus). In some embodiments, when more than one NLS is present, each may be selected independently of the others, such that a single NLS may be present in more than one copy and/or in combination with one or more other NLSs present in one or more copies. In some embodiments, non-limiting examples of NLSs may include an NLS sequence derived from: the NLS of the SV40 virus large T-antigen, having the amino acid sequence PKKKRKV (SEQ ID NO: 1); the NLS from nucleoplasmin (e.g. the nucleoplasmin bipartite NLS with the sequence KRPAATKKAGQAKKKK (SEQ ID NO: 2)); the c-myc NLS having the amino acid sequence PAAKRVKLD (SEQ ID NO: 3) or RQRRNELKRSP (SEQ ID NO: 4); the hRNPA1 M9 NLS having the sequence NQSSNFGPMKGGNFGGRSSGPYGGGGQYFAKPRNQGGY (SEQ ID NO: 5); the sequence RMRIZFKNKGKDTAELRRRRVEVSVELRKAKKDEQILKRRNV (SEQ ID NO: 6) of the IBB domain from importin-alpha; the sequences VSRKRPRP (SEQ ID NO: 7) and PPKKARED (SEQ ID NO: 8) of the myoma T protein; the sequence PQPKKKPL (SEQ ID NO: 9) of human p53; the sequence SALIKKKKKMAP (SEQ ID NO: 10) of mouse c-abl IV; the sequences DRLRR (SEQ ID NO: 11) and PKQKKRK (SEQ ID NO: 12) of the influenza virus NS1; the sequence RKLKKKIKKL (SEQ ID NO: 13) of the Hepatitis virus delta antigen; the sequence REKKKFLKRR (SEQ ID NO: 14) of the mouse M×1 protein; the sequence KRKGDEVDGVDEVAKKKSKK (SEQ ID NO: 15) of the human poly(ADP-ribose) polymerase; and the sequence RKCLQAGMNLEARKTKK (SEQ ID NO: 16) of the steroid hormone receptors (human) glucocorticoid. In some embodiments, the one or more NLSs may be of sufficient strength to drive accumulation of the minicircle DNA vector or short linear DNA vector in a detectable amount in the nucleus of a eukaryotic cell. In some embodiments, a eukaryotic cell may be a human intestinal crypt cell.

In some embodiments, a nanoparticle may contain a DNAse inhibitor. In some embodiments, a DNAse inhibitor may be localized within a nanoparticle or on a nanoparticle. In some embodiments, a polynucleic acid encoding for an inhibitor may be enclosed within a nanoparticle. In some embodiments, an inhibitor may be a DNA methyltransferase inhibitor such as, but not limited to, DNA methyltransferase inhibitors-2 (DMI-2). In some embodiments, DMI-2 may be produced by Streptomyces sp. strain No. 560. In some embodiments, a structure of DMI-2 may be 4″′R,6aR,10S,10aS-8-acetyl-6a,10a-dihydroxy-2-methoxy-12-mefhyl-10-[4′-[3″-hydroxy-3″,5″-dimethyl-4″ (Z-2″′,4″′-dimethyl-2″′-heptenoyloxy) tetrahydropyran-1″-yloxy]-5′-methylcyclohexan-1′-yloxy]-1,4,6,7,9-pentaoxo-1,4,6,6α,7,8,9,10,10α,11-decahydronaphthacene. In some embodiments, other inhibitors, such as chloroquine, may also be enclosed within a nanoparticle or on a nanoparticle, such as on a surface of a nanoparticle.

In some embodiments, detection of accumulation in the nucleus may be performed by any suitable technique. In some embodiments, a detectable marker may be fused to a vector, such that location within a cell may be visualized, such as in combination with a means for detecting the location of the nucleus (e.g., a stain specific for the nucleus such as DAPI). In some embodiments, cell nuclei may also be isolated from cells, the contents of which may then be analyzed by any suitable process for detecting protein, such as, but not limited to, immunohistochemistry, Western blot, or enzyme activity assay. In some embodiments, time dependent pH triggered release of a cargo into a target site may occur. In some embodiments, delivery vehicles may contain and provide cellular delivery of complex multiple cargoes. In some embodiments, an additional cargo may be a small molecule, an antibody, an inhibitor such as a DNAse inhibitor or RNAse inhibitor.

### Nucleic Acid Loading

In some embodiments, a nucleic acid compound cargo concentration in the delivery vehicle may be from 0.5 nanograms to 50 micrograms. In some embodiments, such concentration may be from about 0.5 ng, 1 ng, 2 ng, 5 ng, 10 ng, 50 ng, 100 ng, 150 ng, 200 ng, 300 ng, 400 ng, 500 ng, 600 ng, 700 ng, 800 ng, 900 ng, 1000 ng, 1p g, 2 μg, 5 μg, 10 μg, 20 μg, 30 μg, 40 μg, 50 μg, 60 μg, or up to 50 μg or greater. In some embodiments, the amount of nucleic acid (e.g., ssDNA, dsDNA, RNA) that may be introduced to a cell by the delivery vehicle may be varied to optimize transfection efficiency and/or cell viability. In some embodiments, less than about 100 picograms of nucleic acid may be introduced to a subject. In some embodiments, at least about 100 picograms, at least about 200 picograms, at least about 300 picograms, at least about 400 picograms, at least about 500 picograms, at least about 600 picograms, at least about 700 picograms, at least about 800 picograms, at least about 900 picograms, at least about 1 microgram, at least about 1.5 micrograms, at least about 2 micrograms, at least about 2.5 micrograms, at least about 3 micrograms, at least about 3.5 micrograms, at least about 4 micrograms, at least about 4.5 micrograms, at least about 5 micrograms, at least about 5.5 micrograms, at least about 6 micrograms, at least about 6.5 micrograms, at least about 7 micrograms, at least about 7.5 micrograms, at least about 8 micrograms, at least about 8.5 micrograms, at least about 9 micrograms, at least about 9.5 micrograms, at least about 10 micrograms, at least about 11 micrograms, at least about 12 micrograms, at least about 13 micrograms, at least about 14 micrograms, at least about 15 micrograms, at least about 20 micrograms, at least about 25 micrograms, at least about 30 micrograms, at least about 35 micrograms, at least about 40 micrograms, at least about 45 micrograms, or at least about 50 micrograms, of nucleic acid may be added to each cell sample (e.g., one or more cells being electroporated or otherwise targeted for cargo delivery). In some embodiments, the amount of nucleic acid (e.g., dsDNA, RNA) required for optimal transfection efficiency and/or cell viability may be specific to the cell type.

In some embodiments, the delivery vehicle may contain at least one nucleic acid at a molar ratio of total nanoparticle cationic lipids to the total number of nucleotides comprised by the nucleic acid cargo (abbreviated herein as cationic lipid:nucleotide ratio, cationic lipid:nucleotide molar ratio, or CL:N) of between about 1 and 100. For example, the cationic lipid:nucleotide ratio may be, but is not limited to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, and any combination thereof.

In some embodiments, the molar ratio of total nanoparticle cationic lipids to the total number of nucleotides comprised by the nucleic acid cargo is from about 2 to about 20.

In some embodiments, the molar ratio of total nanoparticle cationic lipids to the total number of nucleotides comprised by the nucleic acid cargo is from about 14 to about 18.

In some embodiments, the molar ratio of total nanoparticle cationic lipids to the total number of nucleotides comprised by the nucleic acid cargo is from about 2 to about 20.

In some embodiments, the molar ratio of total nanoparticle cationic lipids to the total number of nucleotides comprised by the nucleic acid cargo is from about 2 to about 4.

In some embodiments, a polynucleic acid may be condensed to be properly encapsulated by a lipid structure. In some embodiments, condensation of DNA may be performed by divalent metal ions such as Mn²⁺, Ni²⁺, Co²⁺, and Cu²⁺ that may condense DNA through neutralization of phosphate groups of the DNA backbone and distortion of the β-DNA structure through hydrogen bonding with bases, permitting both local bending of the DNA and inter-helical associations. In some embodiments, the concentration of metal ions utilized for condensation may be dependent on the dielectric constant of a medium used in the condensation. In some embodiments, the addition of ethanol or methanol may also reduce the concentration of metal ion required for condensation. In some embodiments, ethanol may be used to condense DNA at concentrations from about 0.5% up to about 60% by volume. In some embodiments, ethanol may be used to condense DNA at concentrations from about 0.5%, 5%, 10%, 15%, 20%, 25%, 30%, 35%, 40%, 45%, 50%, 55% or up to 60% by volume. In some embodiments, calcium may also be used for condensation. In some embodiments, calcium not only binds to DNA phosphates but may also form a complex with the nitrogen and oxygen of guanine, disrupting base pairing.

In some embodiments, a polynucleic acid may be fully encapsulated in a lipid structure. In some embodiments, full encapsulation may indicate that a polynucleic acid in a lipid structures may not be significantly degraded after exposure to serum or a nuclease or protease assay that would significantly degrade free DNA, RNA, or protein. In some embodiments, in a fully encapsulated system, preferably less than about 25% of a polynucleic acid in a lipid structure may be degraded in a treatment that would normally degrade 100% of free polynucleic acid, more preferably less than about 10%, and most preferably less than about 5% of a polynucleic acid in a lipid structure may be degraded. In some embodiments, in the context of polynucleic acids, full encapsulation may be determined by an Oligreen® assay. Oligreen® is an ultra-sensitive fluorescent nucleic acid stain for quantitating oligonucleotides and single-stranded DNA or RNA in solution (available from Invitrogen Corporation; Carlsbad, Calif.). In some embodiments, “fully encapsulated” may also indicate that a lipid structure may be serum-stable, that is, that they do not rapidly decompose into their component parts upon in vivo administration.

### Other Therapeutic Agents

### Cancer Treating

In certain embodiments, a cargo may include a molecule or compound such as an oncology drug, which may also be referred to as an anti-tumor drug, an anti-cancer drug, a tumor drug, an antineoplastic agent, or the like.

Examples of oncology drugs that may be used include, but are not limited to, adriamycin, alkeran, allopurinal, altretamine, amifostine, anastrozole, araC, arsenic trioxide, azathioprine, bexarotene, biCNU, bleomycin, busulfan intravenous, busulfan oral, capecitabine (Xeloda), carboplatin, carmustine, CCNU, celecoxib, chlorambucil, cisplatin, cladribine, cyclosporin A, cytarabine, cytosine arabinoside, daunorubicin, cytoxan, daunorubicin, dexamethasone, dexrazoxane, dodetaxel, doxorubicin, doxorubicin, DTIC, epirubicin, estramustine, etoposide phosphate, etoposide and VP-16, exemestane, FK506, fludarabine, fluorouracil, 5-FU, gemcitabine (Gemzar), gemtuzumab-ozogamicin, goserelin acetate, hydrea, hydroxyurea, idarubicin, ifosfamide, imatinib mesylate, interferon, irinotecan (Camptostar, CPT-111), letrozole, leucovorin, leustatin, leuprolide, levamisole, litretinoin, megastrol, melphalan, L-PAM, mesna, methotrexate, methoxsalen, mithramycin, mitomycin, mitoxantrone, nitrogen mustard, paclitaxel, pamidronate, Pegademase, pentostatin, porfimer sodium, prednisone, rituxan, streptozocin, STI-571, tamoxifen, taxotere, temozolamide, teniposide, VM-26, topotecan (Hycartin), toremifene, tretinoin, ATRA, valrubicin, velban, vinblastine, vincristine, VP16, and vinorelbine. Other examples of oncology drugs that may be used are ellipticin and ellipticin analogs or derivatives, epothilones, intracellular kinase inhibitors and camptothecins.

### Imaging and Diagnostic Agents

In some embodiments, a vehicle may comprise an imaging agent that may be further attached to a detectable label (e.g., the label may be a radioisotope, fluorescent compound, enzyme, or enzyme co-factor). In some embodiments, the active moiety of the imaging agent may be a radioactive agent, such as: radioactive heavy metals such as iron chelates, radioactive chelates of gadolinium or manganese, positron emitters of oxygen, nitrogen, iron, carbon, or gallium, ⁴³K, ⁵²Fe, ⁵⁷Co, ⁶⁷Cu, ⁶⁷Ga, ⁶⁸Ga, ¹²³I, ¹²⁵I, ¹³¹I, ¹³²I, or ⁹⁹Tc. In some embodiments, the delivery vehicle including such a moiety may be used as an imaging agent and be administered in an amount effective for diagnostic use in a mammal such as a human. In some embodiments, the localization and accumulation of the imaging agent may be detected. In some embodiments, the localization and accumulation of the imaging agent may be detected by radioscintiography, nuclear magnetic resonance imaging, computed tomography, or positron emission tomography.

As will be evident to the skilled artisan, the amount of radioisotope to be administered is dependent upon the radioisotope. Those having ordinary skill in the art may readily formulate the amount of the imaging agent to be administered based upon the specific activity and energy of a given radionuclide used as the active moiety. Typically, 0.1-100 millicuries per dose of imaging agent, 1-10 millicuries, and 2-5 millicuries may be administered.

In some embodiments, compositions useful as imaging agents may comprise a targeting moiety conjugated to a radioactive moiety that may comprise 0.1-100 millicuries, 1-10 millicuries, 2-5 millicuries, or 1-5 millicuries.

In some embodiments, the means of detection used to detect the label is dependent of the nature of the label used and the nature of the biological sample used, and may include, but is not limited to, fluorescence polarization, high performance liquid chromatography, antibody capture, gel electrophoresis, differential precipitation, organic extraction, size exclusion chromatography, fluorescence microscopy, or fluorescence activated cell sorting (FACS) assay.

In some embodiments, an imaging agent targeting moiety may also refer to a protein, nucleic acid, nucleic acid analog, carbohydrate, or small molecule. The imaging entity may be, for example, a therapeutic compound such as a small molecule, or a diagnostic entity such as a detectable label. A locale may be a tissue, a particular cell type, or a subcellular compartment. In one embodiment, the targeting moiety may direct the localization of an active entity. The active entity may be a small molecule, protein, polymer, or metal. The active entity, such as a liposome comprising a nucleic acid, may be useful for therapeutic, prophylactic, or diagnostic purposes. In some embodiments, a moiety may allow the delivery vehicle to penetrate a blood brain barrier.

### Loading Capacities

In some embodiments, a lipid structure may carry to a capacity up to over 100% weight: defined as (cargo weight/weight of the lipid structure)×100. In some embodiments, the optimal loading of cargo may be or may be from about 1% to 100% weight of a lipid structure. In some embodiments, a lipid structure may contain a polynucleic acid cargo from, but not limited to, about 1% weight of a structure to about 10%, from about 10% to about 20%, from about 20% to about 30%, from about 30% to about 40%, from about 40% to about 50%, from about 50%, to about 60%, from about 60% to about 70%, from about 70% to about 80%, from about 80% to about 90%, from about 90% to about 100%, from about 100% to about 200%, from about 200% to about 300%, from about 300% to about 400%, from about 400% to about 500% or greater weight of a structure.

### V. PHARMACEUTICAL COMPOSITION AND ROUTE OF ADMINISTRATION

### Pharmaceutical Compositions and Formulations

The delivery vehicles provided herein may be formulated with one or more excipients into a pharmaceutical composition and/or pharmaceutical medicament. In some embodiments, the pharmaceutical composition and/or medicament may be used to treat any human or mammal in need thereof.

A composition to be administered may contain a quantity of the delivery vehicle in a pharmaceutically effective amount for therapeutic use in a biological system, including a patient or subject.

In some embodiments, the delivery vehicles (e.g., liposomes) can be administered as a liquid formulation, such as a solution or suspension, a semi-solid formulation, such as a lotion or ointment, or a solid formulation. In some embodiments, the liposomes can be formulated as liquids, including solutions and suspensions, such as eye drops or as a semi-solid formulation, such as ointment or lotion for topical application to mucosa, such as the eye or vaginally or rectally. The formulation may contain one or more excipients, such as emollients, surfactants, emulsifiers, and penetration enhancers.

As used herein, “pharmaceutically acceptable carrier” includes any and all solvents, dispersion media, coatings, antibacterial and antifungal agents, isotonic and absorption delaying agents, sweeteners, salts, buffers, and the like. The pharmaceutically acceptable carriers may be prepared from a wide range of materials including, but not limited to, flavoring agents, sweetening agents and miscellaneous materials such as buffers and absorbents that may be needed in order to prepare a particular therapeutic composition.

### Pharmaceutical Salts

In some embodiments, a pharmaceutical composition may include a salt. A salt can be relatively non-toxic. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. In some embodiments, if desired, the pharmaceutical composition including the delivery vehicle may also contain minor amounts of non-toxic auxiliary substances such as wetting agents, emulsifying agents, or buffers.

### Coatings

In some embodiments, provided delivery vehicles can comprise a coating. In some embodiments, a coating can be an enteric coating. In general, enteric coatings can be utilized to prevent or minimize dissolution in the stomach but allow dissolution in the small intestine. In some embodiments, a coating can include an enteric coating. In some embodiments, an enteric coating can be a barrier applied to oral medication that prevents release of medication before it reaches the small intestine. Delayed-release formulations, such as enteric coatings, can avoid an irritant effect on the stomach from administration of a medicament by preventing the pharmaceutical composition from dissolving in the stomach. Such coatings are also used to protect acid-unstable drugs from the stomach's acidic exposure, delivering them instead to a basic pH environment (intestine's pH 5.5 and above) where they may not degrade.

In some embodiments, dissolution can occur in an organ. For example, dissolution can occur within a duodenum, jejunum, ilium, and/or colon, or any combination thereof. In some embodiments, dissolution can occur in proximity to a duodenum, jejunum, ilium, and/or colon. Some enteric coatings work by presenting a surface that is stable at a highly acidic pH found in the stomach but break down rapidly at a less acidic (relatively more basic) pH. Therefore, an enteric coated pill may not dissolve in the acidic environment of the stomach but can dissolve in an alkaline environment present in a small intestine. Examples of enteric coating materials include, but are not limited to, methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate and stearic acid.

In some embodiments, an enteric coating can be applied at a functional concentration. In some embodiments, an enteric coating can be cellulose acetate phthalate, Polyvinyl acetate phthalate, Hydroxypropylmethylcellulose acetate succinate, Poly(methacylic acid-co-ethyl acrylate) 1:1, Poly(methacrylic acid-co-ethyl acrylate) 1:1, Poly(methacylic acid-co-methyl methacrylate) 1:1, Poly(methacylic acid-co-methyl methacrylate) 1:1, Poly(methacylic acid-co-methyl methacrylate) 1:2, Poly(methacylic acid-co-methyl methacrylate) 1:2, Poly(methyl acrylate-co-methyl methacrylate-co-methacrylic acid) 7:3:1, or any combination thereof. An enteric coating can be applied from about 6 mg/(cm²) to about 12 mg/(cm²). An enteric coating can also be applied to a structure from about 1 mg/(cm²), 2 mg/(cm²), 3 mg/(cm²), 4 mg/(cm²), 5 mg/(cm²), 6 mg/(cm²), 7 mg/(cm²), 8 mg/(cm²), 9 mg/(cm²), 10 mg/(cm²), 11 mg/(cm²), 12 mg/(cm²), 13 mg/(cm²), 14 mg/(cm²), 15 mg/(cm²), 16 mg/(cm²), 17 mg/(cm²), 18 mg/(cm²), 19 mg/(cm²), to about 20 mg/(cm²).

### Routes of Administration

A composition can be administered orally, by subcutaneous or other injection, intravenously, intracerebrally, intramuscularly, parenterally, transdermally, nasally or rectally. The form in which the compound or composition is administered depends at least in part on the route by which the compound is administered.

### Oral Administration

In some embodiments, a composition can be employed in the form of solid preparations for oral administration; preparations may be tablets, granules, powders, capsules, or the like. In a tablet formulation, a composition is typically formulated with additives, e.g., an excipient such as a saccharide or cellulose preparation, a binder such as starch paste or methyl cellulose, a filler, a disintegrator, and other additives typically used in the manufacture of medical preparations.

For oral administration, the compositions can take the form of, for example, tablets or capsules prepared by a conventional technique with pharmaceutically acceptable excipients such as binding agents (e.g., pregelatinized maize starch, polyvinylpyrrolidone or); fillers (e.g., lactose,); lubricants (e.g., magnesium stearate, talc or); disintegrants (e.g., potato starch or); or wetting agents (e.g., sodium lauryl sulphate).

In some embodiments, solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, microcrystalline cellulose, calcium hydrogen phosphate, or silicic acid), binders (e.g. carboxymethylcellulose, alginates, gelatin, hydroxypropyl methylcellulose, polyvinylpyrrolidone, sucrose, pregelatinized maize starch, and acacia), humectants (e.g. glycerol), disintegrating agents (e.g. agar, calcium carbonate, sodium starch glycollate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate, or silica), and mixtures thereof. In the case of capsules, tablets and pills, the dosage form may comprise buffering agents.

In some embodiments the tablets may be coated.

In some embodiments, for oral administration, an excipient may include pharmaceutical grades of mannitol, lactose, starch, magnesium stearate, sodium saccharine, talcum, cellulose, glucose, gelatin, sucrose, magnesium carbonate, and the like.

Liquid preparations for oral administration can take the form of, for example, solutions, syrups, or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate.

In some embodiments, oral compositions may include adjuvants such as wetting agents, emulsifying and suspending agents, sweetening, flavoring, and/or perfuming agents. In some embodiments, preparations for oral administration can be suitably formulated to give controlled release of the active compound.

### Injectable and Parenteral Administration

In some embodiments, pharmaceutical compositions and/or formulations described herein may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

In some embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropyl cellulose.

In some embodiments, injectable preparations, for example, sterile injectable aqueous or oleaginous suspensions may be formulated according to the known art using suitable dispersing agents, wetting agents, and/or suspending agents. In some embodiments, sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

In some embodiments, injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Suitable formulations can include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. Suitable inert carriers can include sugars such as lactose. In some embodiments, the compositions can take such forms as suspensions, solutions, or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

A carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride. Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof. Suitable surfactants may be anionic, cationic, amphoteric, or nonionic surface-active agents. Suitable anionic surfactants include, but are not limited to, those containing carboxylate, sulfonate, and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimoniuni bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine. The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s). The formulation is typically buffered to a pH of 3-8 for parenteral administration upon reconstitution. Suitable buffers include, but are not limited to, phosphate buffers, acetate buffers, and citrate buffers. Water soluble polymers can be often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions can be prepared by incorporating the various sterilized active ingredients into a sterile vehicle which contains the basic dispersion medium and the required other ingredients from those listed above. In the case of sterile powders for the preparation of sterile injectable solutions, a method of preparation can be vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

### Topical or Transdermal Administration

In some embodiments, the liposomes can be formulated for topical administration to mucosa. Suitable dosage forms for topical administration include creams, ointments, salves, sprays, gels, lotions, emulsions, liquids, and transdermal patches. The formulation may be formulated for transmucosal, transepithelial, transendothelial, or transdermal administration. The compositions may contain one or more chemical penetration enhancers, membrane permeability agents, membrane transport agents, emollients, surfactants, stabilizers, and combination thereof.

In some embodiments, pharmaceutical compositions and/or formulations described herein may be formulated for administration topically. The skin may be an ideal target site for delivery as it is readily accessible. Three routes are commonly considered to deliver pharmaceutical compositions and/or formulations described herein to the skin: (a) topical application (e.g., for local/regional treatment and/or cosmetic applications); (b) intradermal injection (e.g., for local/regional treatment and/or cosmetic applications); and (c) systemic delivery (e.g., for treatment of dermatologic diseases that affect both cutaneous and extracutaneous regions).

In some embodiments, pharmaceutical compositions and/or formulations described herein may be delivered using a variety of dressings (e.g., wound dressings) or bandages (e.g., adhesive bandages) for conveniently and/or effectively carrying out methods described herein. Typically dressing or bandages may comprise sufficient amounts of pharmaceutical compositions and/or formulations described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, contemplated herein is the use of transdermal patches, which often have the added advantage of providing controlled delivery of pharmaceutical compositions and/or formulations described herein to the body. Such dosage forms may be prepared, for example, by dissolving and/or dispensing pharmaceutical compositions and/or formulations described herein in the proper medium. Alternatively, or additionally, rates may be controlled by either providing rate controlling membranes and/or by dispersing pharmaceutical compositions and/or formulations described herein in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

### Ophthalmic or Otic Administration

In some embodiments, pharmaceutical compositions described herein may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmic ally-administrable formulations which are useful include those which comprise active ingredients in microcrystalline form and/or in liposomal preparations. Subretinal inserts may also be used as forms of administration.

Pharmaceutical formulations for ocular administration can be in the form of a sterile aqueous solution or suspension of particles formed from one or more polymer-drug conjugates. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol.

### Depot Administration

In some embodiments, compositions can also be formulated as a preparation for implantation or injection. Thus, for example, a structure can be formulated with suitable polymeric, aqueous, and/or hydrophilic materials, or resins, or as sparingly soluble derivatives (e.g., as a sparingly soluble salt).

In some embodiments, a pharmaceutical compositions and/or formulations described herein are formulated in depots for extended release.

### Intranasal, Nasal, or Buccal Administration

For buccal administration the compositions can take the form of tablets or lozenges formulated in conventional manner.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

### Rectal and Vaginal Administration

The compounds can also be formulated in rectal compositions, creams, or lotions.

In some embodiments, pharmaceutical compositions and/or formulations described herein may be administered rectally and/or vaginally. Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Time Period for Formulation

In some embodiments, a composition comprising the delivery vehicle can be formulated under sterile conditions within a reasonable time prior to administration. For example, a composition comprising the delivery vehicle can be formulated from about 1 month, 2 weeks, 1 week, 5 days, 3 days, 2 days, 1 day, 10 hours, 5 hours, or immediately prior to administration to a subject. In an aspect, the delivery vehicle can be frozen and thawed prior to administration. Provided delivery vehicles can be used in combination with secondary therapies. For example, a secondary therapy such as chemotherapy or radiation therapy may be administered before or subsequent to the administration of the delivery vehicle, for example within 12 hr. to 7 days. A combination of therapies, such as both chemotherapy and radiation therapy may be employed in addition to the administration of the delivery vehicles

### Delayed and Targeted Release

In some embodiments, a pharmaceutical composition comprising a subject delivery vehicle can be orally administered from a variety of drug formulations designed to provide delayed-release. Delayed oral dosage forms include, for example, tablets, capsules, caplets, and may also comprise a plurality of granules, beads, powders, or pellets that may or may not be encapsulated. Tablets and capsules can represent oral dosage forms, in which case solid pharmaceutical carriers can be employed. In a delayed-release formulation, one or more barrier coatings may be applied to pellets, tablets, or capsules to facilitate slow dissolution and concomitant release of drugs into the intestine. Typically, a barrier coating can contain one or more polymers encasing, surrounding, or forming a layer, or membrane around a therapeutic composition or active core. In some embodiments, active agents, such as a polynucleic acid, can be delivered in a formulation to provide delayed-release at a pre-determined time following administration. The delay may be up to about 10 minutes, about 20 minutes, about 30 minutes, about 1 hour, about 2 hours, about 3 hours, about 4 hours, about 5 hours, about 6 hours, or up to 1 week in length. In some embodiments, an enteric coating may not be used to coat a particle.

Polymers or coatings that can be used to achieve enteric release can be anionic polymethacrylates (copoly-merisate of methacrylic acid and either methyl-methacrylate or ethylacrylate (Eudragit®), cellulose based polymers, e.g., cellulose acetatephthalate (Aquateric®) or polyvinyl derivatives, e.g., polyvinyl acetate phthalate (Coateric®) In some embodiments.

### Dose Schedules and Amounts

In some embodiments, a pharmaceutical composition containing the delivery vehicle with its cargo may be administered chronically. In some embodiments, administration may encompass hourly, daily, monthly, or yearly administration of a structure to a subject. In some embodiments, a subject may be administered a pharmaceutical composition daily for the entirety of the subject's life. In some embodiments, a pharmaceutical composition may be administered daily for the duration of the presence of disease in a subject. In some embodiments, a subject may be administered a pharmaceutical composition, such as with the delivery vehicle and a polynucleic acid cargo, to treat a disease or disorder until the disease or disorder is reduced, controlled, or eliminated. In some embodiments, disease control may encompass the stabilization of a disease. In some embodiments, a cancer that is controlled may have stopped growing or spreading as measured by CT scan. In some embodiments, cancer may be a colon cancer.

In some embodiments, an appropriate dosage (“therapeutically effective amount”) of an active agent(s) in a composition may depend, for example, on the severity and course of a condition, a mode of administration, a bioavailability of a particular agent(s), the age and weight of a subject, a subject's clinical history and response to an active agent(s), discretion of a physician, or any combination thereof. In some embodiments, a therapeutically effective amount of an active agent(s) in a composition to be administered to a subject may be in the range of about 100 μg/kg body weight/day to about 1000 mg/kg body weight/day whether by one or more administrations. In some embodiments, the range of each active agent administered daily may be from about 100 μg/kg body weight/day to about 50 mg/kg body weight/day, 100 μg/kg body weight/day to about 10 mg/kg body weight/day, 100 μg/kg body weight/day to about 1 mg/kg body weight/day, 100 μg/kg body weight/day to about 10 mg/kg body weight/day, 500 μg/kg body weight/day to about 100 mg/kg body weight/day, 500 μg/kg body weight/day to about 50 mg/kg body weight/day, 500 μg/kg body weight/day to about 5 mg/kg body weight/day, 1 mg/kg body weight/day to about 100 mg/kg body weight/day, 1 mg/kg body weight/day to about 50 mg/kg body weight/day, 1 mg/kg body weight/day to about 10 mg/kg body weight/day, 5 mg/kg body weight/dose to about 100 mg/kg body weight/day, 5 mg/kg body weight/dose to about 50 mg/kg body weight/day, 10 mg/kg body weight/day to about 100 mg/kg body weight/day, and 10 mg/kg body weight/day to about 50 mg/kg body weight/day.

In some embodiments, a pharmaceutical composition may be administered daily or administered on an as needed basis.

In some embodiments, the delivery vehicles or pharmaceutical compositions disclosed herein may be delivered to the subject more than one, which may be referred to as “redosing” or “re-dosing.” In some embodiments, re-dosing may pe performed 1, 2, 3, 4, or more times without significant decrease in the effectiveness of the delivery vehicle to deliver cargo to the subject.

In some embodiments, medicaments can be co-administered with any additional therapy.

### VI. TARGET AREA, TISSUE OR CELL FOR DELIVERY

The delivery vehicles described herein may deliver cargo both systemically and or to localized targets within a subject. In some embodiments, localized targets may include, but are not limited to, specific cells, tissues, organs, physiological systems, or any combination thereof of a subject. In some embodiments, the localized target may be a tumor.

In some embodiments, delivery vehicles provided herein may be utilized to deliver cargo to a target cell. In some embodiments, a target cell is found in a gastrointestinal tract, reproductive tract, circulatory system, respiratory system, musculoskeletal system, excretory system, nervous system, oculatory system, and combinations thereof. In some embodiments, suitable target cells may be found in any major organ of the body including but not limited to the skin, lungs, heart, liver, stomach, urinary system, reproductive system, intestine, pancreas, kidneys, thymus gland, thyroid, and/or brain. In some embodiments, a target cell is part of the gastrointestinal tract and is in the anus, rectum, large intestine, small intestine, liver, stomach, esophagus, or mouth. In some embodiments, a target cell is an enteroendocrine cell, mast cell, enterocyte, brush cell, Paneth cell, or goblet cell. In some embodiments, a target cell is an enteroendocrine cell and is an EC cell, D cell, CCK cell, L cell, P/D1 cell, or G cell. In some embodiments, a target cell is in the intestinal epithelium and is selected from an intestinal stem cell, Paneth cell, goblet cell, enterocyte, transit amplifying cell, enteroendocrine cell, or any combination thereof. In some embodiments, a target cell is an intestinal stem cell. In some embodiments, a target cell is a crypt cell.

### Cells

In some embodiments, the delivery vehicle may deliver a cargo to a particular cell type. Non-limiting example of cells include adipocytes, adrenergic neural cells, alpha cell, amacrine cells, ameloblast, anterior lens epithelial cell, anterior/intermediate pituitary cells, apocrine sweat gland cell, astrocytes, auditory inner hair cells of organ of corti, auditory outer hair cells of organ of corti, b cell, Bartholin's gland cell, basal cell (stem cell) of cornea, tongue, mouth, nasal cavity, distal anal canal, distal urethra, and distal vagina, basal cells of olfactory epithelium, basket cells, basophil granulocyte and precursors, beta cell, Betz cells, bone marrow reticular tissue fibroblasts, border cells of organ of corti, boundary cells, bowman's gland cell, brown fat cell, Brunner's gland cell, bulbourethral gland cell, bushy cells, c cells, Cajal-Retzius cells, cardiac muscle cell, cardiac muscle cells, cartwheel cells, cells of the zona fasciculata produce glucocorticoids, cells of the zona glomerulosa produce mineralocorticoids, cells of the zona reticularis produce androgens, cells of the adrenal cortex, cementoblast, centroacinar cell, ceruminous gland cell in ear, chandelier cells, chemoreceptor glomus cells of carotid body cell, chief cell, cholinergic neurons, chromaffin cells, club cell, cold-sensitive primary sensory neurons, connective tissue macrophage (all types), comeal fibroblasts (corneal keratocytes), corpus luteum cell of ruptured ovarian follicle secreting progesterone, cortical hair shaft cell, corticotropes, crystallin-containing lens fiber cell, cuticular hair shaft cell, cytotoxic t cell, d cell, delta cell, dendritic cell, double-bouquet cells, duct cell, eccrine sweat gland clear cell, eccrine sweat gland dark cell, efferent ducts cell, elastic cartilage chondrocyte, endothelial cells, enteric glial cells, enterochromaffin cell, enterochromaffin-like cell, enteroendocrine cell, eosinophil granulocyte and precursors, ependymal cells, epidermal basal cell, epidermal Langerhans cell, epididymal basal cell, epididymal principal cell, epithelial reticular cell, epsilon cell, erythrocyte, fibrocartilage chondrocyte, fork neurons, foveolar cell, g cell, gall bladder epithelial cell, germ cells, gland of Littre cell, gland of moll cell in eyelid, glial cells, golgi cells, gonadal stromal cells, gonadotropes, granule cells, granulosa cell, granulosa lutein cells, grid cells, and head direction cells.

### Tissues

In some embodiments, the delivery vehicle may deliver a cargo to a particular tissue. Non-limiting example of tissues are the adrenal medulla, adult fibrous tissue, blood vessels, bone, breast, bronchial lining, carotid body, cartilage, connective tissue, embryonic (myxomatous) fibrous tissue, epithelial, epithelium, fat, glandular epithelium (liver, kidney, bile duct), gonads, hematopoietic cells, lymph vessels, lymphoid tissue, meninges, mesothelium, muscle, nerve sheath, nervous, notochord, ovary, pancreas, parathyroid, pituitary, placenta, renal anlage, smooth muscle, stomach and intestines, stratified squamous, striated muscle, stroma, testis, thyroid, and transitional epithelium. As a non-limiting example, the tissue is stomach and intestine tissue.

### Organs

In some embodiments, the delivery vehicle may deliver a cargo to a particular organ. Non-limiting example of organs include the anal canal, arteries, ascending colon, bladder, bone marrow, brain, bronchi, bronchioles, bulbourethral glands, capillaries, cecum, cerebellum, cerebral hemispheres, cerebrum, cervix, choroid plexus, clitoris, cranial nerves, descending colon, diencephalon, duodenum, ear, enteric nervous system, epididymis, esophagus, external reproductive organs, fallopian tubes, gallbladder, ganglia, gustatory, gut-associated lymphoid tissue, heart, ileum, internal reproductive organs, interstitium, jejunum, joints, kidneys, large intestine, larynx, ligaments, liver, lungs, lymph node, lymphatic vessel, mammary glands, medulla oblongata, mesentery, midbrain, mouth, muscles of breathing, nasal cavity, nerves, olfactory, ovaries, pancreas, parotid glands, penis, pharynx, placenta, pons, prostate, rectum, salivary glands, scrotum, seminal vesicles, sigmoid colon, skeleton, skin, small intestine, spinal nerves, spleen, stomach, subcutaneous tissue, sublingual glands, submandibular glands, teeth, tendons, testes, the brainstem, the spinal cord, the ventricular system, thymus, tongue, tonsils, trachea, transverse colon, ureter, urethra, uterus, vagina, vas deferens, veins, and vulva.

### Physiological Systems

In some embodiments, the delivery vehicle may deliver a cargo to a particular physiological system. Non-limiting example of physiological system include the auditory, cardiovascular, central nervous system, chemo-receptor system, circulatory, digestive, endocrine, enteric nervous system, excretory, exocrine, genital, integumentary, lymphatic, muscular, musculoskeletal, nervous, peripheral nervous system, renal, reproductive, respiratory, urinary, and visual systems.

In some embodiments, the physiological system is the digestive system or the enteric nervous system.

### Tumors

In some embodiments, the delivery vehicle may deliver a cargo to a tumor. The tumor may be a benign tumor or a malignant tumor.

### VI. METHODS OF USE

Provided herein are various methods of using the disclosed delivery vehicles and pharmaceutical compositions. In some embodiments, methods of delivering a cargo to a subject are provided. In some embodiments, methods of treating a subject in need thereof are provided. In some embodiments, methods of preventing the occurrence or the worsening of an indication are provided. In some embodiments, methods of using the disclosed delivery vehicles and pharmaceutical compositions in diagnostics, imaging, or scientific research are provided. In some embodiments, assays either utilizing or evaluating the delivery vehicles and pharmaceutical compositions disclosed are provided.

### Methods of Delivering Cargo

In some embodiments, the method of delivering a cargo to a subject includes administering a delivery vehicle or pharmaceutical composition described herein to a subject. In some embodiments, the method includes delivering a cargo to the cells of a subject.

In some embodiments, the present disclosure provides methods of delivering cargo to target cells by contacting target cells with compositions (e.g., compositions that include cargo and the delivery vehicle nanoparticles) described herein. In some embodiments, target cells may include human cells. In some embodiments, target cells may be part of mucosal tissue. In some embodiments, target cell mucosal tissue may be part of the gastrointestinal tract. In some embodiments, target cells may include gastrointestinal cells. In some embodiments, gastrointestinal cells may include, but are not limited to intestinal epithelial cells, lamina propria cells, intraepithelial lymphocytes, intestinal muscle cells, and enteric neurons. In some embodiments, target cells may include epithelial cells. In some embodiments, epithelial cells may include intestinal epithelial cells.

In some embodiments, the present disclosure provides methods utilizing the delivery vehicles provided herein to introduce cargo to target cells. In some embodiments, introduction comprises contacting the target cell with the cargo. In some embodiments, introduction comprises transfecting or transducing the target cell with the cargo. In some embodiments, the cargo may modify the genome of the cell or exist within the cell extragenomically.

In some embodiments, the methods of delivering may deliver any cargo, such as cargos described throughout the present disclosure including, but not limited to, therapeutic agents, nucleic acids, polypeptides, proteins, biologics, antibodies, enzymes, hormones, cytokines, immunogens, and genetic epigenetic editing system components, or any combination thereof.

### Methods of Delivery Via the Gastrointestinal Tract

In some embodiments, the method of delivering cargo to subjects include introducing compositions to subject gastrointestinal tracts, wherein the compositions include the cargo and the delivery vehicle nanoparticles associated with (e.g., encapsulating) the cargo.

In some embodiments, the method of delivering a cargo to a subject includes administering to the subject at least one delivery vehicle including the cargo, wherein the delivery vehicle is or includes a nanoparticle.

In some embodiments, the methods of delivering cargo to subjects may include introducing compositions (i.e., compositions including the delivery vehicle and at least one cargo) to subject gastrointestinal tract, which may include administering compositions to subjects by, for example oral administration and/or intrarectal administration. In some embodiments, delivery vehicle nanoparticles may target gastrointestinal cells. In some embodiments, targeted gastrointestinal cells may include, but are not limited to, intestinal epithelial cells, lamina propria cells, intraepithelial lymphocytes, intestinal muscle cells, and enteric neurons. In some embodiments, any rout of administration may be used.

In some embodiments of the methods, delivery vehicle cargo may be delivered to the gastrointestinal cells. In some embodiments, cargo may be delivered to the intracellular space of the gastrointestinal cells.

### Methods of Delivering Gene Editing Transgene Cargo

In some embodiments, transfected gastrointestinal cells may express nucleic acid cargo encoding genetic editing system components. As used herein, the term “genetic editing system” refers to any technological approach to modifying nucleic acids together with associated components for carrying out the approach. Genetic editing systems may include, but are not limited to, systems utilizing clustered regularly interspaced short palindromic repeats (CRISPR) and CRISPR-associated (Cas) protein technology.

In some embodiments, genetic editing systems may include epigenetic editing systems. In general, epigenetic editing systems are genetic editing systems that alter non-sequence-related nucleic acid characteristics, for example methylation and organization into chromatin.

In some embodiments, nucleic acid cargo encoding genetic editing system components may be used to correct mutations in epithelial cell genes, including, but not limited to, CFTR gene mutations, GPR35 gene mutations, RNF186 A64T germline mutations associated with increased ulcerative colitis risk (see Beaudoin, M. et al. PLoS Genetics. 2013. 9(9): e1003723, the content of which is herein incorporated by reference in its entirety), mutations associated with very early onset IBD (see Leung, G. and Muise, A. M., Physiology. 2018. 33: 360-9, the content of which is herein incorporated by reference in its entirety, including the genes listed in Table 1 thereof), and/or somatic mutations in genes affecting IL-17 signaling (e.g., NFKBIZ, ZC3H12A, and PIGR; see Nanki, K. et al. Nature. 2020. 577(7789): 254-9, the content of which is herein incorporated by reference in its entirety).

In some embodiments, nucleic acid cargo encoding genetic editing system components may be used to delete or silence genes encoding IL-18 and/or IL-18R1 in gastrointestinal stem cells (in vivo or in vitro) to treat or prevent IBD.

In some embodiments, nucleic acid cargo encoding genetic editing system components may be used to generate RNF186 (179×) mutations in gastrointestinal stem cells to confer protection against IBD. Nucleic acid cargo encoding genetic editing system components may be used to insert transgenes into gastrointestinal cell DNA (e.g., via CRISPR or RNA-mediated retrotransposons) to provide permanent sources for expression of therapeutic proteins or other factors.

In some embodiments, inserted transgenes encode anti-TNFα antibodies, anti-P19 antibodies, or anti-IL-23 antibodies to treat or prevent IBD.

In some embodiments, inserted transgenes express GLP-1 or FGF21 for treatment or prevention of metabolic diseases.

In some embodiments, genes for any of the proteins or peptides which may correct the defects in phenylketonuria, diabetes, organic acidurias, tyrosinemia, urea cycle disorders, familial hypercholesteremia may be introduced into stem cells such that the protein or peptide products are expressed by the intestinal epithelium.

In some embodiments, coagulation factors such as antihemophilic factor (factor 8), Christmas factor (factor 9) and factor 7 may likewise be produced in the intestinal epithelium. In some embodiments, proteins which may be used to treat deficiency of a circulatory protein may also be expressed in the intestinal epithelium. In some embodiments, proteins which may be used to treat deficiency of a circulatory protein may be, for example, albumin for the treatment of an albuminemia, alpha-1-antitrypsin, hormone binding protein.

In some embodiments, the intestinal symptoms of cystic fibrosis may be treated by inserting the gene for the normal cystic fibrosis transmembrane conductance regulator into the stem cells of intestinal epithelium.

In some embodiments, Abetalipoproteinemia may be treated by the insertion of the apolipoprotein B.

In some embodiments, Disaccharidase intolerance may be treated by the insertion of sucrase-isomaltose, lactase-phlorizin hydrolase and maltase-glucoamylase.

In some embodiments, the insertion of the intrinsic factor for the absorption of vitamin B12 or the receptor for the intrinsic factor/cobalamin complex for absorption of vitamin B12, as well as the transporter for bile acids may be inserted into the intestinal epithelium.

In some embodiments, any drug which may be encoded by nucleic acid may be inserted into the stem cell of the intestinal epithelium to be secreted in localized, high concentrations for the treatment of cancer. In this respect, one skilled in the art will readily recognize that antisense RNA may be encoded into the stem cells after production of antisense it may incorporate into the cancerous cells for the treatment of cancer.

Delivery to a Subject Via Expression and or Secretion from Target Cells

In some embodiments, the methods of delivering cargo to subjects may include: (i) introducing compositions to subject gastrointestinal tract, such that (ii), cargo, cargo components, and/or cargo expression products may be secreted from gastrointestinal cells after delivery.

As used herein, the term “expression product” refers to a nucleic acid, amino acid polymer, protein, biomolecule, or other structure synthesized or “expressed” from a coded template (e.g., DNA or RNA).

In some embodiments, cargo expression products may be expressed from nucleic acid cargo components directly or may be expressed by cells in response to some other cargo component or component activity (e.g., enzymatic activity, cell signaling activity, transcriptional/translational activation/repression, etc.).

In some embodiments, secretion of cargo, cargo components, and/or cargo expression products may be secreted by apical secretion or basal secretion from gastrointestinal cells. In some embodiments, cargo, cargo components, and/or cargo expression products may remain in an area proximal to the cell after secretion. In some embodiments, cargo, cargo components, and/or cargo expression products may be secreted basally from gastrointestinal cells and enter the circulation. In some embodiments, cargo, cargo components, and/or cargo expression products may be distributed systemically after entering the circulation.

In some embodiments, therapeutic agent nucleic acids may encode polypeptides or proteins that also act as therapeutic agents. Nucleic acids may include DNA (e.g., plasmid DNA). In some embodiments, nanoparticles may target gastrointestinal cells and transfect them with nucleic acid cargo.

In some embodiments, transfected gastrointestinal cells may express polypeptides encoded by nucleic acid cargo.

### Cell Signaling Factors

In some embodiments, nucleic acids may encode cell signaling factors. As used herein, the term “cell signaling factor” refers to any molecule that elicits a cellular response, including, but not limited to, cytokines, growth factors, and receptor ligands. Cell signaling factors encoded by nucleic acid nanoparticle cargos may include, but are not limited to, interleukin (IL)-2, IL-2 mutein Fc-fusion, IL-10, IL-10 mutein, IL-22, granulocyte-macrophage colony-stimulating factor (GM-CSF), granulocyte colony-stimulating factor (G-CSF), adrenomedullin, glucagon-like peptide 1 (GLP-1), glucagon-like peptide 2 (GLP-2), GLP-2 analog teduglutide, peroxisome proliferator-activated receptor gamma (PPARγ), human growth hormone (HGH), parathyroid hormone (PTH), fibroblast growth factor 21 (FGF21), and relaxin.

### Antibodies

In some embodiments, nucleic acids encode antibodies. As used herein, the term “antibody” is used in the broadest sense and specifically embraces various antibody formats including, but not limited to, monoclonal antibodies, polyclonal antibodies, multispecific antibodies (e.g., bispecific antibodies formed from at least two intact antibodies), and antibody fragments (e.g., diabodies) so long as they exhibit a desired biological activity (e.g., are “functional” fragments). Encoded antibodies may bind targets that include one or more of IL-18, IL-18 receptor 1 (IL18R1), IL-23, tumor necrosis factor α (TNFα), proprotein convertase subtilisin kexin 9 (PCSK9), and protein 19 (P19). Encoded antibodies may include bispecific antibodies. Bispecific antibodies may bind to cluster of differentiation 3 (CD3) for recruitment of immune cells to targets of a second bispecific antibody epitope.

In some embodiments, transfected gastrointestinal cells may express nucleic acid cargo encoding antigens. As used herein, the term “antigen” refers to an entity or structure that can be specifically bound or “recognized” by an antibody binding partner. An antigen which evokes an immune response in organisms is referred to herein as an “immunogen.” Nucleic acid cargo encoding immunogens may be delivered to subjects to promote immune responses to the encoded immunogens. Encoded immunogens may be derived from pathogenic organisms or viruses. Pathogens associated with encoded immunogens may include, but are not limited to, influenza virus, SARS-CoV-2 virus, Ebola virus, and polio virus.

In some embodiments, nucleic acid cargo encodes tumor cell neoantigens. As used herein, the term “neoantigen” refers to an antigen that is expressed by tumor cells (e.g., due to mutation or other mechanism), distinguishing them from non-tumor cells. Expression of neoantigens may be used to promote immune responses in subjects against tumor cells. In some embodiments, nucleic acid cargo encode antigens useful for development of tolerance to the antigens by the subject. Such antigens may include, but are not limited to, antigens associated with peanut allergies, celiac disease, rheumatoid arthritis, and IBD.

In some embodiments, transfected gastrointestinal cells express nucleic acid cargo encoding clotting factors (e.g., Factor VIII). In some embodiments, transfected gastrointestinal cells express nucleic acid cargo encoding enzymes [e.g., β-glucocerebrosidase (GBA)].

In some embodiments, gastrointestinal cells may be transfected with nucleic acid cargo that includes non-coding RNA. As used herein, the term “non-coding RNA” refers to RNA molecules with sequences that do not encode proteins, but typically have significance in some other RNA function. Non-coding RNA may include, but is not limited to, short interfering RNA (siRNA), microRNA (miRNA), long non-coding RNA, piwi-interacting RNA (piRNA), small nucleolar RNA (snoRNA), small Cajal body-specific RNA (scaRNA), transfer RNA (tRNA), ribosomal RNA (rRNA), and small nuclear RNA (snRNA).

In some embodiments, transfected gastrointestinal cells may express nucleic acid cargo encoding antimicrobial agents. As used herein, the term “antimicrobial agent” refers to any substance capable of killing or otherwise slowing or stopping the growth, spread, or reproduction of microbiological organisms or viruses. Antimicrobial agents encoded by nucleic acid cargo may include, but are not limited to, intestinal alkaline phosphatase (IAP) and defensins.

### Additional Methods of Delivering Cargo

In some embodiments, the present disclosure provides a method of delivering a cargo to a target cell, the method including contacting the target cell with a composition (e.g., a cargo and nanoparticle composition) described herein. The target cell may include a human cell. The target cell may include an epithelial cell. The epithelial cell may include an intestinal epithelial cell.

In some embodiments, the present disclosure provides a method of delivering a cargo to a target cell, wherein the target cell is part of a mucosal tissue, the method including contacting the mucosal tissue with a composition described above or herein. The mucosal tissue may be part of a gastrointestinal tract. The target cell may be a gastrointestinal cell. The gastrointestinal cell may include one or more of an intestinal epithelial cell, a lamina propria cell, an intraepithelial lymphocyte, an intestinal muscle cell, and an enteric neuron.

In some embodiments, the present disclosure provides a method of delivering a cargo to a subject, the method including introducing a composition described above or herein to the gastrointestinal tract of the subject. The composition may be introduced to the subject gastrointestinal tract by administering the composition to the subject by an administration route selected from one or more of oral administration and intrarectal administration. The nanoparticle may target a gastrointestinal cell. The gastrointestinal cell may be selected from one or more of an intestinal epithelial cell, a lamina propria cell, an intraepithelial lymphocyte, an intestinal muscle cell, and an enteric neuron. The cargo may be delivered to the gastrointestinal cell. The cargo may be delivered to the intracellular space of the gastrointestinal cell. The cargo, a cargo component, or an expression product of the cargo may be secreted from the gastrointestinal cell. Secretion of the cargo, cargo component, or expression product of the cargo may include apical secretion or basal secretion. The cargo, cargo component, or expression product of the cargo may remain in an area proximal to the cell after secretion. The cargo, cargo component, or expression product of the cargo may be secreted basally from the gastrointestinal cell and enter the circulation. The cargo, cargo component, or expression product of the cargo may be distributed systemically after entering the circulation. The cargo may include a therapeutic agent. The therapeutic agent may include one or more of a nucleic acid, a polypeptide, a protein, a biologic, an antibody, an enzyme, a hormone, a cytokine, an immunogen, and a genetic or epigenetic editing system component. The therapeutic agent may include a nucleic acid. The nucleic acid may encode at least one polypeptide. The nucleic acid may include DNA. The nucleic acid may include plasmid DNA. The nanoparticle may target a gastrointestinal cell and the gastrointestinal cell may be transfected with the nucleic acid. The gastrointestinal cell may express a polypeptide encoded by the nucleic acid. The nucleic acid may encode a cell signaling factor. The cell signaling factor may be selected from one or more of interleukin (IL)-2, IL-2 mutein Fc-fusion, IL-10, IL-10 mutein, IL-22, granulocyte-macrophage colony-stimulating factor (GM-CSF), granulocyte colony-stimulating factor (G-CSF), adrenomedullin, glucagon-like peptide 1 (GLP-1), glucagon-like peptide 2 (GLP-2), GLP-2 analog teduglutide, peroxisome proliferator-activated receptor gamma (PPARγ), human growth hormone (HGH), parathyroid hormone (PTH), fibroblast growth factor 21 (FGF21), and relaxin. The nucleic acid may encode an antibody. The antibody may bind a target selected from one or more of IL-18, IL-18 receptor 1 (IL18R1), IL-23, tumor necrosis factor α (TNFα), proprotein convertase subtilisin kexin 9 (PCSK9), and protein 19 (P19). The antibody may be a bispecific antibody. The bispecific antibody may bind to cluster of differentiation 3 (CD3). The nucleic acid may encode an antimicrobial agent. The antimicrobial agent may be selected from one or more of intestinal alkaline phosphatase (IAP) and a defensin. The nucleic acid may encode a genetic editing system component. The nucleic acid may encode an antigen as an immunogen for promotion of an immune response to the antigen by the subject. The antigen may be derived from one or more of influenza virus, SARS-CoV-2 virus, Ebola virus, and polio virus. The antigen may include a tumor cell neoantigen. The immune response may include development of tolerance to the antigen by the subject. The antigen may be associated with one or more of peanut allergies, celiac disease, rheumatoid arthritis, and IBD. The nucleic acid may encode a clotting factor. The clotting factor may include Factor VIII. The nucleic acid may encode an enzyme. The enzyme may include O-glucocerebrosidase (GBA). The nucleic acid may be a non-coding RNA. The non-coding RNA may include one or more of short interfering RNA (siRNA), microRNA (miRNA), long non-coding RNA, piwi-interacting RNA (piRNA), small nucleolar RNA (snoRNA), small Cajal body-specific RNA (scaRNA), transfer RNA (tRNA), ribosomal RNA (rRNA), and small nuclear RNA (snRNA).

### Methods of Treating an Indication

In some embodiments, the present disclosure provides methods of treating therapeutic indications in subjects by administering delivery vehicles and/or pharmaceutical compositions (e.g., compositions, nanoparticles, and/or cargo) described herein. In some embodiments, the methods of treating a therapeutic indication include at least one of the methods of delivering cargo to a subject described herein. In some embodiments, methods of treating a subject include at least one of the methods for delivery of a cargo to the subject's gastrointestinal tract disclosed herein. For the sake of clarity, “the methods of treating therapeutic indications” may also be referred to herein simply as “the treatment methods.”

In some embodiments, the treatment method includes delivery a cargo (e.g., any of the therapeutic agents described herein) to a target in need thereof. In some embodiments, the treatment method includes delivery of a cargo to a subject in need thereof systemically. In some embodiments, the treatment method includes: (i) delivery of a cargo to at least one cell in a subject; (ii) the cell expressing or producing a therapeutic agent: and optionally (iii) the cell secreting the therapeutic agent either locally or systemically.

As used herein, the term “therapeutic indication” refers to any disease, condition, disorder, or symptom that may be improved, cured, stabilized, alleviated, or otherwise addressed by medical treatment or other intervention. Delivery vehicle cargo used in therapeutic indication treatment may include and/or encode therapeutic agents.

Exemplary therapeutic indications (e.g., diseases) that can be treated with subject delivery vehicles provided herein, particularly such delivery vehicles with a therapeutic cargo, can be cancerous or non-cancerous. Such disease can be cardiovascular disease, a neurodegenerative disease, an ocular disease, a reproductive disease, a gastrointestinal disease, a brain disease, a skin disease, a skeletal disease, a muscoskeletal disease, a pulmonary disease, a thoracic disease, to name a few. A disease can be a genetic disease such as cystic fibrosis, tay-sachs, fragile X, Huntington's, neurofibromatosis, sickle cell, thalassemias, Duchenne's muscular dystrophy, or a combination thereof.

In some embodiments, the treatment method includes screening the subject for the presence of a disease. In some embodiments, screens can be utilized to identify suitable subjects. In some embodiments, a disease can be identified by genetic, phenotypic, molecular, or chromosomal screening. In some embodiments, a suitable subject is positive for a disease provided herein. For example, a genetic screen can identify a mutation in an APC gene that can result in FAP. In some embodiments, a screen can comprise analyzing a gene such as CDH1, STKT1, SMAD4, MLH1, MSH2, EPCAM, MSH6, PMS2, MYO5B, APC, TP53, portions thereof, promoters thereof, and combinations thereof.

### Gastrointestinal Indications

In some embodiments, a disease is a gastrointestinal disease. In some embodiments, a gastrointestinal disease is a monogenic GI disease. In some embodiments, a gastrointestinal disease is inherited. In some embodiments, a gastrointestinal disease is of the epithelium. Exemplary gastrointestinal diseases may include familial adenomatous polyposis (FAP), attenuated FAP, microvillus inclusion disease (MVID), chronic inflammatory bowel disease, chronic inflammatory bowel disease, ileal Crohn's, juvenile polyposis, hereditary diffuse gastric cancer syndrome (HDGC), Peutz-Jeghers syndrome, lynch syndrome, gastric adenocarcinoma, and proximal polyposis of the stomach (GAPPS), Li-Fraumeni syndrome, familial gastric cancer, or a combination thereof. In some embodiments, a GI disease can produce polyps in a gastrointestinal tract. In some embodiments, a disease is FAP. In some embodiments, FAP can progress to cancer. In some embodiments, a gastrointestinal disease can be hereditary. For example, a hereditary gastrointestinal disease may be Gilbert's syndrome, telangiectasia, mucopolysaccaride, Osler-Weber-Rendu syndrome, pancreatitis, keratoacanthoma, biliary atresia, Morquio's syndrome, Hurler's syndrome, Hunter's syndrome, Crigler-Najjar, Rotor's, Peutz-Jeghers' syndrome, Dubin-Johnson, Osteochondroses, Osteochondrodysplasias, polyposis, or a combination thereof.

### Immune-Related Indications

In some embodiments, the therapeutic indication to be treated by the methods disclosed herein may include immune-related indications. As used herein, the term “immune-related indication” refers to any therapeutic indication relating to the immune system.

In some embodiments for treating immune-related indications, methods of the present disclosure may include the delivery of (also referred to herein as the “use of”) at least one nucleic acid cargo encoding one or more of IL-2, IL-2 mutein Fc-fusion, IL-10, IL-10 mutein, IL-22, adrenomedullin, an anti-microbial, and an anti-inflammatory antibody. In some embodiments, nucleic acid cargo may be delivered to gastrointestinal cells. In some embodiments, gastrointestinal cells may express therapeutic agents from nucleic acid cargo. In some embodiments, gastrointestinal cells may secrete therapeutic agents locally or systemically (e.g., via entry into circulation).

In some embodiments, immune-related indications addressed by the treatment methods of the present disclosure include gastrointestinal indications, which may include gastrointestinal diseases and any other disorders involving the gastrointestinal tract and related components. Gastrointestinal indications may include, but are not limited to, gastrointestinal infections, inflammatory bowel disease (IBD), ulcerative colitis, and Crohn's disease. Gastrointestinal cells may express and locally secrete (e.g., into the intestinal lumen) therapeutic agents encoded by cargo nucleic acids for treatment of such gastrointestinal indications.

In some embodiments, immune-related indications addressed by the treatment methods of the present disclosure are systemic or are not specific to the gastrointestinal tract. In some embodiments, the treatment method may include: (i) transfection of gastrointestinal cells; (ii) gastrointestinal cells may express and secrete therapeutic agents into circulation. Non-limiting examples indications to addressed by treatment methods including a secretion step may include graft versus host disease (GVHD), systemic lupus erythematosus (SLE), type I diabetes, rheumatoid arthritis, infections, wounds, and allergies.

### Cancers

In some embodiments, therapeutic indications treated according to methods of the present disclosure include cancer and associated disorders, referred to herein as “cancer-related indications.”

In some embodiments, the method of treatment for cancer-related indications includes secretion of therapeutic agents by subject cells. In some embodiments, therapeutic agents encoded by nucleic acid cargo associated with methods of treating cancer-related indications may include GM-CSF. In some embodiments, gastrointestinal cells may express and secrete nucleic acid cargo encoded GM-CSF locally or into circulation. Cancer-related indications treated according to such methods may include, but are not limited to, Hodgkin's lymphoma, non-Hodgkin's lymphoma, acute lymphoblastic leukemia, and acute myelogenous leukemia.

In some embodiments, subjects receiving the method of treatment have previously received or are receiving concurrent chemotherapy treatment and/or stem cell transplantation treatment.

In some embodiments, GM-CSF is secreted into circulation at a level sufficient to provide circulating GM-CSF concentrations of from about 10 to about 500 μg/m²/day (e.g., from about 50 to about 200, from about 100 to about 250, or from about 150 to about 400 μg/m²/day).

### Specific Indications

In some embodiments, therapeutic indications treated according to methods of the present disclosure may include neutropenia, a disorder characterized by low neutrophil blood levels. Nucleic acid cargo associated with such methods may encode G-CSF, which promotes granulocyte production and neutrophil regulation. In some embodiments, gastrointestinal cells may express and secrete G-CSF locally and/or systemically for neutropenia treatment. In some embodiments, G-CSF is secreted into circulation at a level sufficient to provide subjects with a dose of from about 1 to about 20 μg/kg/day of the G-CSF (e.g., from about 1 to about 10, from about 5 to about 15, or from about 10 to about 20 μg/kg/day). In some embodiments, subjects are treated until neutrophil blood levels reach about 1000/μl.

In some embodiments, therapeutic indications treated according to methods of the present disclosure may include microvillus inclusion disease (MVID). Nucleic acid cargo associated with such methods may encode MYO5B gene product.

In some embodiments, therapeutic indications treated according to methods of the present disclosure may include cystic fibrosis. Nucleic acid cargo associated with such methods may encode cystic fibrosis transmembrane regulator protein (CFTR).

In some embodiments, therapeutic indications treated according to methods of the present disclosure may include hemophilia. Nucleic acid cargo associated with such methods may encode clotting factors. Clotting factors may include Factor VIII. In some embodiments, treated hemophilia may include hemophilia A.

In some embodiments, therapeutic indications treated according to methods of the present disclosure may include Gaucher's disease. Nucleic acid cargo associated with such methods may encode GBA. Nucleic acid cargo encoding GBA may be delivered to gastrointestinal cells. In some embodiments, gastrointestinal cells may secrete GBA into circulation at a level sufficient to provide steady-state subject GBA plasma levels of from about 1 ng/mL to about 10 ng/mL (e.g., about 6 ng/mL).

In some embodiments, therapeutic indications treated according to methods of the present disclosure include short bowel syndrome (SBS). Nucleic acid cargo associated with such methods may encode GLP-2. In some embodiments, nucleic acid cargo encoding GLP-2 may be delivered to and expressed by gastrointestinal cells. GLP-2 may be secreted into circulation at levels sufficient to provide circulating GLP-2 concentrations of from about 10 ng/mL to about 50 ng/mL (e.g., about 36 ng/mL).

In some embodiments, therapeutic indications treated according to methods of the present disclosure may include hormone deficiencies. Nucleic acid cargo delivered according to such methods may encode deficient hormones. Deficient hormones may include, but are not limited to, HGH and PTH. Nucleic acid cargo may be delivered to and expressed by gastrointestinal cells. In some embodiments, expressed hormones may be secreted into circulation. In some embodiments, HGH may be secreted into circulation at a level sufficient to provide circulating HGH concentrations of from about 0.1 to about 100 ng/mL. In some embodiments, levels in adults are from about 1 to about 10 ng/mL. In some embodiments, levels in children are from about 10 to about 50 ng/mL. In some embodiments, PTH may be secreted into circulation at levels sufficient to provide circulating PTH concentrations of from about 50 to about 300 μg/mL (e.g., about 150 μg/mL).

In some embodiments, therapeutic indications treated according to methods of the present disclosure include non-alcoholic steatohepatitis (NASH). Nucleic acid cargo associated with such methods may encode GLP-1 or FGF21.

In some embodiments, therapeutic indications treated according to methods of the present disclosure include elevated circulating low density lipoprotein (LDL) levels. Nucleic acid cargo associated with such methods may encode anti-PCSK9 antibodies. In some embodiments, nucleic acid cargo encoding anti-PCSK9 antibodies may be delivered to and expressed by gastrointestinal cells. In some embodiments, Anti-PCSK9 antibodies may be secreted into circulation at a level sufficient to provide circulating antibody concentrations of from about 1 to about 50 μg/mL (e.g., from about 1 to about 10, from about 6 to about 18, from about 12 to about 19, or from about 15 to about 45 μg/mL).

### Additional Methods of Treating Indications

In some embodiments, the present disclosure provides a method of treating a therapeutic indication in a subject, the method including delivering a cargo to the subject according to any of the methods described above or herein. The therapeutic indication may include an immune-related indication. The cargo may include a nucleic acid encoding a therapeutic agent. The therapeutic agent may be selected from the group consisting of IL-2, IL-2 mutein Fc-fusion, IL-10, IL-10 mutein, IL-22, adrenomedullin, an anti-microbial, and an anti-inflammatory antibody. The cargo may be delivered to a gastrointestinal cell. The gastrointestinal cell may express the therapeutic agent. The gastrointestinal cell may secrete the therapeutic agent locally. The immune-related indication may include a gastrointestinal indication. The gastrointestinal indication may include one or more of gastrointestinal infection, inflammatory bowel disease (IBD), ulcerative colitis, and Crohn's disease. The gastrointestinal cell may secrete the therapeutic agent into circulation. The immune-related indication may include a non-gastrointestinal-specific indication and/or a systemic indication. The immune-related indication may include one or more of graft versus host disease (GVHD), systemic lupus erythematosus (SLE), type I diabetes, rheumatoid arthritis, an infection, a wound, and an allergy. The therapeutic indication may include a cancer-related indication. The cargo may include a nucleic acid encoding a therapeutic agent. The therapeutic agent may include GM-CSF. The cancer-related indication may include one or more of Hodgkin's lymphoma, non-Hodgkin's lymphoma, acute lymphoblastic leukemia, and acute myelogenous leukemia. The subject may have received or may be undergoing chemotherapy and/or stem cell transplantation. The cargo may be delivered to gastrointestinal cells and the gastrointestinal cells may secrete the GM-CSF into circulation at a level sufficient to provide a circulating GM-CSF concentration of about 250 μg/m²/day. The therapeutic indication may include neutropenia. The cargo may include a nucleic acid encoding G-CSF. The cargo may be delivered to gastrointestinal cells and the gastrointestinal cells may secrete the G-CSF into circulation at a level sufficient to provide about 5 μg/kg/day of the G-CSF. The subject may be treated until subject neutrophil blood levels reach 1000/μl. The therapeutic indication may be microvillus inclusion disease (MVID) and the cargo may include a nucleic acid encoding MYO5B gene product. The therapeutic indication may include cystic fibrosis and the cargo may include a nucleic acid encoding cystic fibrosis transmembrane regulator protein (CFTR). The therapeutic indication may include hemophilia and the cargo may include a nucleic acid encoding a clotting factor. The clotting factor may include Factor VIII. The hemophilia may include hemophilia A. The therapeutic indication may include Gaucher's disease, and the cargo may include a nucleic acid encoding GBA. The cargo may be delivered to gastrointestinal cells and the gastrointestinal cells may secrete GBA into circulation at a level sufficient to provide steady-state GBA plasma levels of about 6 ng/mL. The therapeutic indication may include short bowel syndrome (SBS) and the cargo may include a nucleic acid encoding GLP-2. The cargo may be delivered to gastrointestinal cells and the gastrointestinal cells may secrete GLP-2 into circulation at a level sufficient to provide a circulating GLP-2 concentration of about 36 ng/mL. The therapeutic indication may include a hormone deficiency and the cargo may include a nucleic acid encoding the deficient hormone. The deficient hormone may be selected from the group consisting of HGH and PTH. The deficient hormone may be HGH, the cargo may be delivered to gastrointestinal cells, and the gastrointestinal cells may secrete the HGH into circulation at a level sufficient to provide a circulating HGH concentration of from about 1 to about 10 ng/mL in adults or from about 10 to about 50 ng/mL in children. The deficient hormone may be PTH, the cargo may be delivered to gastrointestinal cells, and the gastrointestinal cells may secrete the PTH into circulation at a level sufficient to provide a circulating PTH concentration of about 150 μg/mL. The therapeutic indication may include non-alcoholic steatohepatitis (NASH) and the cargo may include a nucleic acid encoding GLP-1 or FGF21. The therapeutic indication may include elevated circulating low density lipoprotein (LDL) level and the cargo may include a nucleic acid encoding an anti-PCSK9 antibody. The cargo may be delivered to gastrointestinal cells and the gastrointestinal cells may secrete the anti-PCSK9 antibody into circulation at a level sufficient to provide a circulating anti-PCSK9 antibody concentration of from about 18 to about 19 μg/mL.

### Preventative Methods

In some embodiments, a method described herein includes administering the delivery vehicle or pharmaceutical composition as a preventative measure. For example, any of the methods described herein may include administering the delivery vehicle or pharmaceutical composition to a subject that may not have been diagnosed with a disease. In some embodiments of the methods, the subject may appear to be predisposed to a disease. In some embodiments, the disease may be at least one cancer. In some embodiments, a cancer can be a colon cancer.

In some embodiments, prophylactic treatment can prevent a disease, such as cancer. In some embodiments, prevention can be used in relation to: a condition, such as a local recurrence (e.g., pain); a disease such as cancer; a syndrome complex such as heart failure; or any other medical condition.

In some embodiments, prevention can include administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

In some embodiments, prevention of an infection includes, for example, reducing the number of diagnoses of the infection in a treated population versus an untreated control population, and/or delaying the onset of symptoms of the infection in a treated population versus an untreated control population.

In some embodiments, prevention of pain includes, for example, reducing the magnitude of, or alternatively delaying, pain sensations experienced by subjects in a treated population versus an untreated control population.

### Assays, Imaging, and Diagnostics

In some embodiments, the delivery vehicles herein carry a diagnostic cargo and are used to visualize or diagnose the state of cells or tissues or to diagnose or monitor a subject for a condition or a disease. For example, a subject is administered an effective amount of delivery vehicles and a diagnostic method for FAP includes determining a level of APC incorporated into a cell genome whereupon a difference in APC levels before the start of therapy in a patient and during and/or after therapy will evidence the effectiveness of therapy in a patient, including whether a patient has completed therapy or whether the disease state has been inhibited or eliminated.

In some embodiments, the methods described herein may include performing additional procedures on subjects receiving delivery vehicles. In some embodiments, subjects may receive procedures such as blood transfusions, blood draws, computerized tomography scan (CT), magnetic resonance imaging (MRI), X rays, radiation therapy, organ transplants, and any combination thereof. In some embodiments, an evaluation of a lesion, such as a cancerous lesion, may be performed.

In some embodiments of the methods, non-target lesions may be evaluated. In some embodiments, complete response of a non-target lesion may be a disappearance and normalization of tumor marker level. In some embodiments, all lymph nodes must be non-pathological in size (less than 10 mm short axis). In some embodiments, if tumor markers are initially above the upper normal limit, they must normalize for a patient to be considered a complete clinical response. Non-CR/Non-PD is persistence of one or more non-target lesions and or maintenance of tumor marker level above the normal limit. Progressive disease may be defined by appearance of one or more new lesions and or unequivocal progression of existing non-target lesions. Unequivocal progression should not normally trump target lesion status. In some embodiments, a best overall response may be the best response recorded from the start of treatment until disease progression/recurrence.

In some embodiments, a method described herein includes determining the therapeutic effectiveness of the delivery vehicle and cargo. In some embodiments, assays can be utilized to determine therapeutic effectiveness of delivery vehicles provided herein. In some embodiments, an assay can be performed before, during, and/or after administration of subject delivery vehicles. In some embodiments, an assay can be performed for example on days −30, −15, −7, −3, 0, 3, 5, 7, 10, 14, 18, 20, 24, 30, 35, 40, 50, 55, 60, 80, 100, 150, 250, 360, 2 years, 5 years, or 10 years pre or post administration. Suitable assays can be in vivo or ex vivo. In some embodiments, an assay comprises a scan. Suitable scans can comprise CT, PET, MRI, or combinations thereof. In some embodiments, an assay comprises an in vitro assay such as histology, serology, sequencing, ELISA, microscopy, and the like.

In some embodiments, additional procedures may be performed on subjects receiving delivery vehicles. In some embodiments, subjects may receive procedures such as blood transfusions, blood draws, computerized tomography scan (CT), magnetic resonance imaging (MRI), X rays, radiation therapy, organ transplants, and any combination thereof. In some embodiments, an evaluation of a lesion, such as a cancerous lesion, may be performed.

In some embodiments, a protein and/or a protein that is encoded by a nucleic acid compound comprised within a lipid structure may be measured and quantified. In some embodiments, modified cells may be isolated, and a western blot performed on modified cells to determine a presence and a relative amount of protein production as compared to unmodified cells. In some embodiments, intracellular staining of a protein utilizing flow cytometry may be performed to determine a presence and a relative amount of protein production. In some embodiments, additional assays may also be performed to determine if a protein, such as APC, is functional. In some embodiments, modified cells expressing an APC transgene, may be measured for cytosolic β-catenin expression, and compared to unmodified cells. In some embodiments, reduced expression of β-catenin in the cytosol of modified cells as compared to unmodified cells may be indicative of a functional APC transgene. In some embodiments, a murine model of FAP may be utilized to determine functionality of a transgene encoding an APC protein. In some embodiments, mice with FAP may be treated with modified cells, encoding for APC, and a reduction of FAP disease measured versus untreated mice.

### Effective Amounts

In some embodiments, an effective amount of a structure can mean an amount sufficient to increase the expression level of at least one gene which can be decreased in a subject prior to the treatment or an amount sufficient to alleviate one or more symptoms of cancer. For example, an effective amount can be an amount sufficient to increase the expression level of at least one gene selected from the group consisting of gastrointestinal differentiation genes, cell cycle inhibition genes, and tumor suppressor genes by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level without the treatment of any compound.

In some embodiments, an effective amount can mean an amount sufficient to decrease the expression level of at least one gene which may be increased in the subject prior to the treatment or an amount sufficient to alleviate one or more symptoms of cancer. For example, an effective amount can be an amount sufficient to decrease the expression level of a gene by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level without the treatment of any compound.

In some embodiments, treating comprises reduction of the disease in the subject in need thereof by at least about 1 fold, 5 fold, 10 fold, 20 fold, 40 fold, 80 fold, 100 fold, 300 fold, 600 fold, or 1000 fold as measured by an in vitro or in vivo assay as compared to a comparable subject that does not undergo the administering. In an aspect, reduction of the disease can be the result of an increase or decreases in the expression level of at least one gene in the subject. Various gene expression assays can be utilized and include but are not limited to sequencing, PCR, RT-PCR, western blot, northern blot, ELISA, protein quantification, mRNA quantification, FISH, RNA-Seq, SAGE, or a combination thereof. Additional assays that can be utilized include microscopy, histology, in vivo animal experiments, human experiments, or any combination thereof.

In some embodiments, assays may be utilized to determine therapeutic effectiveness of delivery vehicles provided herein. In some embodiments, an assay may be performed before, during, and/or after administration of subject delivery vehicles. In some embodiments, an assay may be performed for example on days −30, −15, −7, −3, 0, 3, 5, 7, 10, 14, 18, 20, 24, 30, 35, 40, 50, 55, 60, 80, 100, 150, 250, 360, 2 years, 5 years, or 10 years pre or post administration. In some embodiments, suitable assays may be in vivo or ex vivo. In some embodiments, an assay comprises a scan. Suitable scans may comprise CT, PET, MRI, or combinations thereof. In some embodiments, an assay comprises an in vitro assay such as histology, serology, sequencing, ELISA, microscopy, and the like.

Exemplary Delivery Vehicles for use with the Methods

In some embodiments, the methods described herein may utilize any of the delivery vehicles or pharmaceutical compositions disclosed herein. For example, suitable delivery vehicles include all those described in section II or Table 1B of the present disclosure.

In some embodiments of the methods, the nanoparticles may include at least one cationic lipid; at least one structural lipid; at least one bile salt; and at least one conjugated lipid, conjugated with a hydrophilic polymer (e.g., PEG). In some embodiments of the methods bile salts may be selected from one or more of deoxycholate, lithocholate, isolithocholate, alloisolithocholate, dehydrolithocholate, ursodiol, 5beta-cholanic acid, chenodeoxycholate, cholate, taurodeoxycholate, taurochenodeoxycholate, glycocholate, 3-oxy-cholenic acid, and hyodeoxycholate. In some embodiments of the methods, bile salts may be included in nanoparticles at levels of from about 5 to about 40 mole % of total nanoparticle lipid (e.g., from about 20 to about 40 or from about 33 to about 37 mole % of total nanoparticle lipid). In some embodiments of the methods nanoparticle bile salt may include deoxycholate and/or lithocholate. In some embodiments of the methods nanoparticles may include two bile salts. In some embodiments of the methods, nanoparticles may include deoxycholate at a level of from about 20 to about 30 mole % of total nanoparticle lipid and lithocholate at a level of from about 5 to about 10 mole % of total nanoparticle lipid. In some embodiments of the methods, nanoparticle cationic lipids may include MVL5. In some embodiments of the methods, MVL5 may be present at a level of from about 5 to about 20 mole % of total nanoparticle lipid. In some embodiments of the methods, nanoparticle cationic lipids may include one or more of MC2, CL1H6, and CL4H6 and each may be present at a level of from about 5 to about 20 mole % of total nanoparticle lipid. In some embodiments of the methods, nanoparticle structural lipids may include one or more of DSPC and DMPC and may be present at a level of from about 35 to about 45 mole % of total nanoparticle lipid. In some embodiments of the methods, nanoparticle conjugated lipids may be conjugated with hydrophilic polymers. In some embodiments of the methods, hydrophilic polymers may include PEG. In some embodiments of the methods, conjugated lipids may include one or more of DMG-PEG and DMPE-PEG and may be present at a level of from about 0.5 to about 2.0 mole % of total nanoparticle lipid.

### VIII. DEFINITIONS

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.” The term “about” or “approximately” can mean within an acceptable error range for the particular value as determined by one of ordinary skill in the art, which will depend in part on how the value is measured or determined, e.g., the limitations of the measurement system. For example, “about” can mean within ±10% the given value. Where particular values are described in the application and claims, unless otherwise stated the term “about” should be assumed to mean an acceptable error range for the particular value.

At various places in the present disclosure, substituents, or properties of compounds of the present disclosure are disclosed in groups or in ranges. It is specifically intended that the present disclosure comprise each and every individual or sub-combination of the members of such groups and ranges.

Unless stated otherwise, the following terms and phrases have the meanings described below. The definitions are not meant to be limiting in nature and serve to provide a clearer understanding of certain aspects of the present disclosure.

About. The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

Active Ingredient: The term “active ingredient”, “therapeutic agent”, and “therapeutic ingredient” refer to the component of a pharmaceutical composition which is biologically active, such as a cannabinoid.

Administering: The term “administering”, and its grammatical equivalents can refer to any method of providing a structure described herein to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a structure disclosed herein can be administered therapeutically. In some instances, a structure can be administered to treat an existing disease or condition. In further various aspects, a structure can be administered prophylactically to prevent a disease or condition.

Adjuvants: The term “adjuvants” as used herein refers to any substance or a combination of substances, that is used to increase the efficacy or potency of another drug.

Approximately: The term “approximately” or “about,” as used herein as applied to one or more values of interest, refers to a value that is similar to a stated reference value. As used herein, the term “about” means+/−10% of the recited value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5% 4% 3% 2%, 1%, or less in either direction (greater than or less than) of the stated reference value unless otherwise stated or otherwise evident from the context (except where such number would exceed 100% of a possible value).

Biodegradable: The term “biodegradable” and its grammatical equivalents can refer to polymers, compositions, and formulations, such as those described herein that are intended to degrade during use. The term “biodegradable” is intended to cover materials and processes also termed “bioerodible.”

Cancer: The term “cancer” and its grammatical equivalents as used herein can refer to a hyperproliferation of cells whose unique trait-loss of normal controls-results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type.

Cargo: The term “cargo” as used herein can refer to one or more molecules or structures encompassed in a delivery vehicle for delivery to or into a cell or tissue. Non-limiting examples of cargo can include a nucleic acid, a dye, a drug, a protein, a liposome, a small chemical molecule, a large biological molecule, and any combinations thereof.

Cell: The term “cell” and its grammatical equivalents as used herein can refer to a structural and functional unit of an organism. A cell can be microscopic in size and can consist of a cytoplasm and a nucleus enclosed in a membrane. A cell can refer to an intestinal crypt cell. A crypt cell can refer to the crypts of Lieberkühn which are pit-like structures that surround the base of the villi in the intestine. A cell can be of human or non-human origin.

Conjugate: The term “Conjugate” as used herein can refer to the association, covalently or non-covalently of two or more molecules or structures, including without limitation, the association of a peptide, such as a mucus-penetrating peptide (MPP) with the delivery vehicle, a polymer, a surface modification, or any combinations thereof.

Function: The term “function” and its grammatical equivalents as used herein can refer to the capability of operating, having, or serving an intended purpose. Functional can comprise any percent from baseline to 100% of an intended purpose. For example, functional can comprise or comprise about 5, 10, 15, 20, 25, 30, 35, 40, 45, 50, 55, 60, 65, 70, 75, 80, 85, 90, 95, or up to about 100% of an intended purpose. In some embodiments, the term functional can mean over or over about 100% of normal function, for example, 125, 150, 175, 200, 250, 300%, 400%, 500%, 600%, 700% or up to about 1000% of an intended purpose.

Gastrointestinal Disease: The term “gastrointestinal disease” as used herein can refer to diseases involving the gastrointestinal tract, including but not limited to esophagus, stomach, small intestine, large intestine and rectum, and the accessory organs of digestion, the liver, gallbladder, and pancreas, and any combinations thereof.

Hydrophilic: The term “hydrophilic” and its grammatical equivalents as used herein refers to substances or structures that have polar groups that readily interact with water.

Hydrophobic: The term “hydrophobic” and its grammatical equivalents as used herein refers to substances or structures that have polar groups that do not readily interact with water.

Mucus: The term “mucus,” and its grammatical equivalents as used herein, can refer to a viscoelastic natural substance containing primarily mucin glycoproteins and other materials, which protects epithelial surface of various organs/tissues, including but not limited to respiratory, nasal, cervicovaginal, gastrointestinal, rectal, visual, and auditory systems.

Lipid Structure: The term “lipid structure” as used herein refers to a lipid composition for delivery to a cell or tissue, such as to deliver a therapeutic product, such as a nucleic acid. The term “lipid structure” and its grammatical equivalents as used herein can refer to a nanoparticle or delivery vehicle. A structure can be a liposomal structure. A structure can be a lipid nanoparticle. A lipid structure can also refer to a particle. A lipid structure or particle can be a nanoparticle or delivery vehicle. A lipid particle or lipid structure can be of any shape having a diameter from about 1 nm up to about 1 micron. A nanoparticle or nanostructure can be or can be about 100 to 200 nm. A nanoparticle or nanostructure can also be up to 500 nm. Nanoparticles or nanostructures having a spherical shape can be referred to as “nanospheres”.

Structure: The term “structure” and its grammatical equivalents as used herein can refer to a nanoparticle or delivery vehicle. A structure can be a liposomal structure. A structure can also refer to a particle. A structure or particle can be a nanoparticle or delivery vehicle. A particle or structure can be of any shape having a diameter from about 1 nm up to about 1 micron. A nanoparticle or nanostructure can be or can be about 100 to 200 nm. A nanoparticle or nanostructure can also be up to 500 nm. Nanoparticles or nanostructures having a spherical shape can be referred to as “nanospheres”.

Nucleic Acid: The term “Nucleic Acid” refers to any compound that is comprised of nucleic acids. The terms “Nucleic Acid,” “nucleic acid,” “polynucleotide,” and “oligonucleotide” and their grammatical equivalents can be used interchangeably and can refer to a deoxyribonucleotide and/or ribonucleotide polymer, in linear or circular conformation, and in either single- or double-stranded form. For the purposes of the present disclosure, these terms should not be construed as limiting with respect to length. The terms can also encompass known analogues of natural nucleotides, as well as nucleotides that are modified in the base, sugar and/or phosphate moieties (e.g., phosphorothioate backbones). In general, an analogue of a particular nucleotide can have the same base-pairing specificity, i.e., an analogue of adenine “A” can base-pair with thymine “T”.

Pharmaceutically Acceptable: The term “pharmaceutically acceptable” as used herein refers to compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically Acceptable Carrier: The term “pharmaceutically acceptable carrier” and their grammatical equivalents can refer to sterile aqueous or non-aqueous solutions, dispersions, suspensions, or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These solutions, dispersions, suspensions, or emulsions can also contain adjuvants such as preservatives, wetting agents, emulsifying agents, and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly (orthoesters) and poly (anhydrides). The term “pharmaceutically acceptable carrier” may refer to any excipient (e.g., vehicles, adjuvants, or dilutants) which are capable of suspending, dissolving, encapsulating, or otherwise carrying an active ingredient in a formulation. Pharmaceutically acceptable carriers can function to improve the selectivity, effectiveness, and/or safety of delivery of an active ingredient.

Pharmaceutical Composition: The term “pharmaceutical composition” as used herein refers to a composition comprising at least one active ingredient (e.g., cannabinoid), and at least one pharmaceutically acceptable carrier or excipient (e.g., formulation mixture).

Predisposed: The term “predisposed” as used herein can be understood to mean an increased probability (e.g., at least 1%, 5%, 10%, 20%, 30%, 40%, 50%, 60%, 70%, 80%, 90%, 100%, 150%, 200%, or more increase in probability) that a subject will suffer from a disease or condition.

Purified, Purify, Purified, & Purification: The terms “purified,” “purify,” “purification,” and their grammatical equivalents as used herein means to make substantially pure or clear from unwanted components, material defilement, admixture, or imperfection. “Purified” refers to the state of being pure. “Purification” refers to the process of making pure.

Subject, Patient & Individual: The terms “subject,” “patient,” or “individual” as used herein refers to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject or patient may seek or need treatment, require treatment, is receiving treatment, will receive treatment, or is under care by a trained professional for a particular disease or condition. The terms “individual,” “patient,” or “subject” are used interchangeably. None of the terms require or are limited to situation characterized by the supervision (e.g., constant or intermittent) of a health care worker (e.g., a doctor, a registered nurse, a nurse practitioner, a physician's assistant, an orderly, or a hospice worker). A subject can be a mammal. A subject can be a human male or a human female. A subject can be of any age. A subject can be an embryo. A subject can be a newborn or up to about 100 years of age. A subject can be in need thereof. A subject can have a disease such as cancer.

Sequence: The term “sequence” and its grammatical equivalents as used herein can refer to a nucleotide sequence, which can be DNA and/or RNA; can be linear, circular, or branched; and can be either single-stranded or double stranded. A sequence can be of any length, for example, between 2 and 1,000,000 or more nucleotides in length (or any integer value there between or there above), e.g., between about 100 and about 10,000 nucleotides or between about 200 and about 500 nucleotides. In some instances, where indicated “sequence” as used herein can refer to an amino acid sequence, such as a sequence of a protein, polypeptide and/or peptide.

Stem Cell: The term “stem cell” as used herein, can refer to an undifferentiated cell of a multicellular organism that is capable of giving rise to indefinitely more cells of the same type. A stem cell can also give rise to other kinds of cells by differentiation. Stem cells can be found in crypts. Stem cells can be progenitors of epithelial cells found on intestinal villi surface. Stem cells can be cancerous. A stem cell can be totipotent, unipotent or pluripotent. A stem cell can be an induced stem cell.

Therapeutically Effective Amount & Effective Amount: The terms “therapeutically effective amount” and “effective amount” as used herein refer to any amount of an active ingredient that can cause the desired effect (e.g., clinical results) when administered to a subject. An effective amount may be determined according to considerations known in the art, and one skilled in the art will recognize that the effective amount can depend on a variety of factors including: the distribution profile within the body, a variety of pharmacological parameters (e.g., half-life in the body), undesired side effects (if any), factors such as age and gender, and other considerations.

Treatment & treating: The terms “treatment,” “treating,” and their grammatical equivalents as used herein refer to partially or completely alleviating, ameliorating, improving, relieving, delaying onset of, inhibiting progression of, reducing severity of, and/or reducing incidence of one or more symptoms or features of a particular infection, disease, disorder, and/or condition. Examples of treatment can include, but are not limited to: to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, to prevent the disease from occurring, or a combination thereof. Treatment may be administered to a subject who does not exhibit signs of a disease, disorder, and/or condition, and/or to a subject who exhibits only early signs of a disease, disorder, and/or condition for the purpose of decreasing the risk of developing pathology associated with the disease, disorder, and/or condition.

Transfection Efficiency: In some embodiments, a delivery vehicle employed may contain a cargo that is delivered to a target cell, for example for expression in the cell and/or to genetically modify a target cell. An efficiency of such delivery, e.g., transfection, with a cargo, such as a polynucleic acid described herein, for example, can be or can be about 20%, 25%, 30%, 35%, 40%4, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9% of the total number of cells that are contacted (in vivo or ex vivo) and/or are present in a tissue or location. An efficiency of such delivery, e.g., transfection, with a cargo, such as a polynucleic acid described herein, for example, can be or can be about 1 fold, 10 fold, 20 fold, 40 fold, 60 fold, 80 fold, 100 fold, 120 fold, 140 fold, 160 fold, 180 fold, 200 fold, 300 fold, 400 fold, 500 fold, or over 1000 fold of the total number of cells that are contacted (in vivo or ex vivo) and/or are present in a tissue or location.

Vehicle: The term “vehicle” as used herein refers to any substance combined with an active ingredient to facilitate administration.

When used in the context of a chemical group, “hydrogen” means —H; “hydroxy” means —OH; “halogen” means independently —F, —Cl, —Br or —I;

For the structures provided herein, the following parenthetical subscripts further define the groups as follows: “(Cn)” defines the exact number (n) of carbon atoms in the group. For example, “(C₂₋₁₀) alkyl designates those alkyl groups having from 2 to 10 carbon atoms (e.g., 2, 3, 4, 5, 6, 7, 8, 9, or 10, or any range derivable therein (e.g., 3 to 10 carbon atoms).

An “alkyl” group can refer to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic. Furthermore, the alkyl moiety, whether saturated or unsaturated, may comprise branched, straight chain, and/or cyclic portions. Depending on the structure, an alkyl group can be a monoradical or a diradical (i.e., an alkylene group). A “heteroalkyl” group is as described for “alkyl” with at least one of the C atoms thereof substituted with an N, S, or O atom. The “heteroalkyl” group may comprise linear, branched, and/or cyclic portions. In certain embodiments, a “lower alkyl” is an alkyl group with 1-6 carbon atoms (i.e., a C₁-C₆ alkyl group). In specific instances, the “lower alkyl” may be straight chained or branched.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. In some embodiments, the term “aryl” can refer to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

“Heteroaryl” refers to a radical derived from a 3- to 12-membered aromatic ring radical that comprises two to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-TH-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

In some embodiments, the term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen, or sulfur, and wherein the monovalent group consists of no atoms other than carbon, hydrogen, aromatic nitrogen, aromatic oxygen, and aromatic sulfur. Non-limiting examples of heteraryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). Substituted heteroaryl refers to a monovalent group with an aromatic carbon atom or nitrogen atom as the point of attachment, said carbon atom or nitrogen atom forming part of an aromatic ring structure wherein at least one of the ring atoms is nitrogen, oxygen, or sulfur, and wherein the monovalent group further has at least one atom independently selected from the group consisting of non-aromatic nitrogen, non-aromatic oxygen, non-aromatic sulfur F, Cl, Br, I, Si, and P.

Substituted: The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched, and unbranched, carbocyclic, and heterocyclic, aromatic, and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—H2), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

### IX. EQUIVALENTS AND SCOPE

Those skilled in the art will recognize or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that comprise “or” between one or more members of a group are considered satisfied if one, more than one, or all of the group members are present in, employed in, or otherwise relevant to a given product or process unless indicated to the contrary or otherwise evident from the context. The present disclosure comprises embodiments in which exactly one member of the group is present in, employed in, or otherwise relevant to a given product or process. The present disclosure comprises embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are comprised. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, comprising definitions, will control. In addition, section headings, the materials, methods, and examples are illustrative only and not intended to be limiting.

### X. INCORPORATION BY REFERENCE

All publications, patents, and patent applications herein are incorporated by reference in their entirety to the same extent as if each individual publication, patent, or patent application was specifically and individually indicated to be incorporated by reference. In the event of a conflict between a term herein and a term in an incorporated reference, the term herein controls.

### XI. ENUMERATED EMBODIMENTS

Embodiment 1. A composition comprising: a cargo; and a nanoparticle, the nanoparticle comprising: at least one bile salt; at least one cationic lipid; at least one structural lipid; and at least one conjugated lipid, wherein the conjugated lipid is conjugated with a hydrophilic polymer.

Embodiment 2. The composition of embodiment 1, wherein the at least one bile salt is selected from one or more of deoxycholate, lithocholate, isolithocholate, alloisolithocholate, dehydrolithocholate, ursodiol, 5beta-cholanic acid, chenodeoxycholate, cholate, taurodeoxycholate, taurochenodeoxycholate, glycocholate, 3-oxy-cholenic acid, and hyodeoxycholate.

Embodiment 3. The composition of embodiment 1 or 2, wherein the at least one bile salt is included in the nanoparticle at a level of from about 5 to about 40 mole % of total nanoparticle lipid.

Embodiment 4. The composition of embodiment 3, wherein the at least one bile salt is included in the nanoparticle at a level of from about 20 to about 40 mole % of total nanoparticle lipid.

Embodiment 5. The composition of embodiment 4, wherein the at least one bile salt is included in the nanoparticle at a level of from about 33 to about 37 mole % of total nanoparticle lipid.

Embodiment 6. The composition of any one of embodiments 1-5, wherein the at least one bile salt comprises deoxycholate.

Embodiment 7. The composition of any one of embodiments 1-6 comprising two bile salts.

Embodiment 8. The composition of embodiment 7, wherein at least one of the two bile salts comprises lithocholate.

Embodiment 9. The composition of embodiment 8 comprising: deoxycholate at a level of from about 20 to about 30 mole % of total nanoparticle lipid; and lithocholate at a level of from about 5 to about 10 mole % of total nanoparticle lipid.

Embodiment 10. The composition of any one of embodiments 1-9, wherein the at least one cationic lipid comprises N1-[2-((1S)-1-[(3-aminopropyl)amino]-4-[di(3-amino-propyl)amino]butylcarboxamido)ethyl]-3,4-di[oleyloxy]-benzamide (MVL5).

Embodiment 11. The composition of embodiment 10, wherein the MVL5 is present at a level of from about 5 to about 20 mole % of total nanoparticle lipid.

Embodiment 12. The composition of any one of embodiments 1-11, wherein the at least one cationic lipid comprises one or more of (6Z,9Z,28Z,31Z)-heptatriaconta-6,9,28,31-tetraen-19-yl 3-(dimethylamino)propanoate (MC2); 7-(4-(dimethylamino)butyl)-7-hydroxytridecane-1,13-diyl dioleate (CL1H6); and 7-(4-(diisopropylamino)butyl)-7-hydroxytride-cane-1,13-diyl dioleate (CL4H6).

Embodiment 13. The composition of embodiment 12, wherein each one of the at least one cationic lipid is present at a level of from about 5 to about 20 mole % of total nanoparticle lipid.

Embodiment 14. The composition of any one of embodiments 1-13, wherein the at least one structural lipid is selected from one or more of 1,2-distearoyl-sn-glycero-3-phosphocholine (DSPC) and 1,2-dimyristoyl-sn-glycero-3-phosphocholine (DMPC).

Embodiment 15. The composition of embodiment 14, wherein the at least one structural lipid is present at a level of from about 35 to about 45 mole % of total nanoparticle lipid.

Embodiment 16. The composition of any one of embodiments 1-15, wherein the hydrophilic polymer comprises polyethylene glycol (PEG).

Embodiment 17. The composition of embodiment 16, wherein the at least one conjugated lipid is selected from one or more of 1,2-dimyristoyl-rac-glycerol (DMG)-PEG and 1,2-dimyristoyl-sn-glycero-3-phosphoethanolamine (DMPE)-PEG.

Embodiment 18. The composition of embodiment 17, wherein the at least one conjugated lipid is present at a level of from about 0.5 to about 2.0 mole % of total nanoparticle lipid.

Embodiment 19. The composition of embodiment 1 comprising a molar ratio between components of from about 1 to about 5 of the at least one bile salt, from about 0.5 to about 3 of each one of the at least one cationic lipid, from about 2 to about 10 of the at least one structural lipid, and from about 0.02 to about 0.10 of the at least one conjugated lipid.

Embodiment 20. The composition of embodiment 19, wherein the at least one bile salt is selected from one or more of deoxycholate, ursodiol, lithocholate, isolithocholate, alloisolithocholate, dehydrolithocholate, and 5beta-cholanic acid.

Embodiment 21. The composition of embodiment 19 or 20, wherein the at least one cationic lipid comprises MVL5.

Embodiment 22. The composition of any one of embodiments 19-21, wherein the at least one cationic lipid comprises MC2.

Embodiment 23. The composition of any one of embodiments 19-22, wherein the at least one structural lipid comprises DSPC.

Embodiment 24. The composition of any one of embodiments 19-23, wherein the at least one conjugated lipid comprises DMG-PEG.

Embodiment 25. The composition of any one of embodiments 19-24 comprising at least one bile salt, MVL5, MC2, DSPC, and DMG-PEG at a molar ratio of about 2.592:0.96:0.96:3.168:0.768.

Embodiment 26. The composition of embodiment 25, wherein the at least one bile salt is deoxycholate.

Embodiment 27. The composition of embodiment 1, wherein the nanoparticle comprises: MVL5, MC2, DSPC, Deoxycholate, and DMPE-PEG at a molar ratio of about 2.4:2.4:7.9:6.48:0.192; MVL5, CL1H6, DSPC, Deoxycholate, and DMG-PEG at a molar ratio of about 2.4:2.4:7.9:6.48:0.192; MVL5, CL4H6, DSPC, Deoxycholate, and DMG-PEG at a molar ratio of about 2.4:2.4:7.9:6.48:0.192; MVL5, MC2, DSPC, Chenodeoxycholate, and DMG-PEG at a molar ratio of about 2.4:2.4:7.9:6.48:0.192; MVL5, MC2, DMPC, Deoxycholate, and DMG-PEG at a molar ratio of about 2.4:2.4:7.9:6.48:0.192; MVL5, MC2, DMPC, Deoxycholate, and DMPE-PEG at a molar ratio of about 2.4:2.4:7.9:6.48:0.192; MVL5, CL1H6, DMPC, Deoxycholate, and DMG-PEG at a molar ratio of about 2.4:2.4:7.9:6.48:0.192; MVL5, MC2, DSPC, Deoxycholate, Lithocholate, and DMG-PEG at a molar ratio of about 2.4:2.4:7.9:5.2:1.3:0.192; MVL5, CL1H6, DSPC, Deoxycholate, Lithocholate, and DMG-PEG at a molar ratio of about 2.4:2.4:7.9:5.2:1.3:0.192; MVL5, MC2, DSPC, Alloisolithocholate, and DMG-PEG at a molar ratio of about 2.4:2.4:7.92:6.48:0.192; or MVL5, MC2, DSPC, Dehydrolithocholate, and DMG-PEG at a molar ratio of about 2.4:2.4:7.92:6.48:0.192.

Embodiment 28. The composition of embodiment 1 comprising 12.4 mole % of MVL5, 12.4 mole % of MC2, 40.8 mole % of DSPC, 33.4 mole % of the at least one bile salt, and 1 mole % of the at least one conjugated lipid.

Embodiment 29. The composition of embodiment 28, wherein the at least one conjugated lipid is DMG-PEG or DMPE-PEG.

Embodiment 30. The composition of embodiment 28 or 29, wherein the at least one bile salt is selected from one or more of taurodeoxycholate, taurochenodeoxycholate, glycocholate, 3-oxy-cholenic acid, and deoxycholate.

Embodiment 31. The composition of any one of embodiments 1-30, wherein the cargo comprises one or more of a nucleic acid, a protein, an antibody, a peptide, a small molecule, a biologic, a peptidomimetic, a ribozyme, a chemical agent, a viral particle, a growth factor, a cytokine, an immunomodulating agent, and a fluorescent dye.

Embodiment 32. The composition of embodiment 31, wherein the cargo comprises a nucleic acid.

Embodiment 33. The composition of embodiment 32, wherein the nucleic acid comprises DNA.

Embodiment 34. The composition of embodiment 33, wherein the DNA comprises plasmid DNA.

Embodiment 35. A composition comprising: a cargo; and a nanoparticle, the nanoparticle comprising: a first cationic lipid comprising CL1H6 or CL4H6; an optional second cationic lipid; at least one bile salt; at least one structural lipid; and at least one conjugated lipid, wherein the at least one conjugated lipid is conjugated with a hydrophilic polymer.

Embodiment 36. The composition of embodiment 35, wherein the at least one bile salt is selected from one or more of deoxycholate, lithocholate, isolithocholate, alloisolithocholate, dehydrolithocholate, ursodiol, 5beta-cholanic acid, chenodeoxycholate, cholate, taurodeoxycholate, taurochenodeoxycholate, glycocholate, 3-oxy-cholenic acid, and hyodeoxycholate.

Embodiment 37. The composition of embodiment 35 or 36, wherein the at least one bile salt is included in the nanoparticle at a level of from about 5 to about 40 mole % of total nanoparticle lipid.

Embodiment 38. The composition of embodiment 37, wherein the at least one bile salt is included in the nanoparticle at a level of from about 20 to about 40 mole % of total nanoparticle lipid.

Embodiment 39. The composition of any one of embodiments 35-38, wherein the at least one bile salt comprises deoxycholate.

Embodiment 40. The composition of any one of embodiments 35-39, wherein the first cationic lipid comprises from about 5 to about 40 mole % of the total nanoparticle lipid.

Embodiment 41. The composition of any one of embodiments 35-40, wherein the nanoparticle comprises a second cationic lipid, the second cationic lipid comprising MVL5, MC2, or DODMA.

Embodiment 42. The composition of embodiment 41, wherein the second cationic lipid is present at a level of from about 5 to about 20 mole % of total nanoparticle lipid.

Embodiment 43. The composition of embodiment 41 or 42, wherein each of the first cationic lipid and the second cationic lipid is present at a level of from about 5 to about 20 mole % of total nanoparticle lipid and wherein each of the first cationic lipid and the second cationic lipid is present in an equal amount.

Embodiment 44. The composition of any one of embodiments 35-43, wherein the at least one structural lipid is selected from one or more of DSPC, DMPC, and dioleoylphosphatidylethanolamine (DOPE).

Embodiment 45. The composition of embodiment 44, wherein the at least one structural lipid is present at a level of from about 10 to about 70 mole % of total nanoparticle lipid.

Embodiment 46. The composition of embodiment 45, wherein the at least one structural lipid is present at a level of from about 30 to about 50 mole % of total nanoparticle lipid.

Embodiment 47. The composition of any one of embodiments 44-46, wherein the at least one structural lipid and the at least one bile salt are present at a combined level of from about 50 to about 80 mole % of total nanoparticle lipid.

Embodiment 48. The composition of any one of embodiments 35-47, wherein the hydrophilic polymer comprises PEG.

Embodiment 49. The composition of any one of embodiments 35-48, wherein the at least one conjugated lipid comprises DMG-PEG.

Embodiment 50. The composition of any one of embodiments 35-49, wherein the at least one conjugated lipid is present at a level of from about 0.5 to about 2.0 mole % of total nanoparticle lipid.

Embodiment 51. The composition of any one of embodiments 35-50, wherein the first cationic lipid comprises CL1H6.

Embodiment 52. The composition of any one of embodiments 35-51, wherein the nanoparticle comprises a second cationic lipid comprising MVL5.

Embodiment 53. The composition of any one of embodiments 35-52, wherein the at least one bile salt comprises deoxycholate.

Embodiment 54. The composition of any one of embodiments 35-53, wherein the at least one structural lipid comprises DSPC.

Embodiment 55. The composition of any one of embodiments 35-54, wherein the at least one conjugated lipid comprises DMG-PEG.

Embodiment 56. The composition of any one of embodiments 35-55 comprising: CL1H6, MVL5, and DMG-PEG at molar ratios of about 1:1:0.08; and deoxycholate and DSPC at molar ratios of from about 0.5 to about 5.0.

Embodiment 57. The composition of embodiment 56, wherein the molar ratios of deoxycholate and DSPC are from about 2.0 to about 4.0.

Embodiment 58. The composition of embodiment 35, wherein the nanoparticle comprises: CL1H6 at a level of from about 10 to about 20 mole % of total nanoparticle lipid; MVL5 at a level of from about 10 to about 20 mole % of total nanoparticle lipid; deoxycholate at a level of from about 10 to about 40 mole % of total nanoparticle lipid; DSPC, DMPC, or DOPE at a level of from about 30 to about 60 mole % of total nanoparticle lipid; and DMG-PEG at a level of from about 0.5 to about 2.0 mole % of total nanoparticle lipid.

Embodiment 59. The composition of embodiment 58, wherein the nanoparticle comprises: CL1H6 and MVL5 at a level of from about 10 to about 15 mole % of total nanoparticle lipid; deoxycholate at a level of from about 20 to about 40 mole % of total nanoparticle lipid; DSPC at a level of from about 35 to about 50 mole % of total nanoparticle lipid; and DMG-PEG at a level of from about 0.75 to about 1.5 mole % of total nanoparticle lipid.

Embodiment 60. The composition of embodiment 59, wherein the nanoparticle comprises: CL1H6 and MVL5 at a level of from about 12 to about 14 mole % of total nanoparticle lipid; deoxycholate at a level of from about 27 to about 38 mole % of total nanoparticle lipid; DSPC at a level of from about 38 to about 45 mole % of total nanoparticle lipid; and DMG-PEG at a level of from about 0.75 to about 1.5 mole % of total nanoparticle lipid.

Embodiment 61. The composition of embodiment 60, wherein the nanoparticle comprises: CL1H6 and MVL5 at a level of about 12 mole % of total nanoparticle lipid; deoxycholate at a level of about 33 mole % of total nanoparticle lipid; DSPC at a level of about 41 mole % of total nanoparticle lipid; and DMG-PEG at a level of about 1 mole % of total nanoparticle lipid.

Embodiment 62. The composition of any one of embodiments 1-61, wherein the hydrophilic polymer is conjugated with a polypeptide.

Embodiment 63. The composition of embodiment 62, wherein the polypeptide is a mucus penetrating polypeptide (MPP).

Embodiment 64. The composition of embodiment 63, wherein the MPP comprises an amino acid sequence according to SEQ ID NO: 17.

Embodiment 65. The composition of any one of embodiments 62-64, wherein the hydrophilic polymer comprises PEG.

Embodiment 66. The composition of embodiment 65, wherein the at least one conjugated lipid comprises DMG-PEG.

Embodiment 67. The composition of embodiment 66, wherein the nanoparticle comprises: CL1H6 and MVL5 at a level of from about 12 to about 14 mole % of total nanoparticle lipid; deoxycholate at a level of from about 27 to about 38 mole % of total nanoparticle lipid; DSPC at a level of from about 38 to about 45 mole % of total nanoparticle lipid; and DMG-PEG at a level of from about 0.75 to about 1.5 mole % of total nanoparticle lipid.

Embodiment 68. The composition of any one of embodiments 1-67, wherein the cargo comprises one or more of a nucleic acid, a protein, an antibody, a peptide, a small molecule, a biologic, a peptidomimetic, a ribozyme, a chemical agent, a viral particle, a growth factor, a cytokine, an immunomodulating agent, and a fluorescent dye.

Embodiment 69. The composition of embodiment 68, wherein the cargo comprises a nucleic acid.

Embodiment 70. The composition of embodiment 69, wherein the nucleic acid comprises DNA.

Embodiment 71. The composition of embodiment 70, wherein the DNA comprises plasmid DNA.

Embodiment 72. The composition of any one of embodiments 69-71, wherein the molar ratio of total nanoparticle cationic lipids to the total number of nucleotides comprised by the nucleic acid cargo is from about 2 to about 20.

Embodiment 73. The composition of embodiment 72, wherein the molar ratio of total nanoparticle cationic lipids to the total number of nucleotides comprised by the nucleic acid cargo is from about 14 to about 18.

Embodiment 74. The composition of embodiment 69, wherein the nucleic acid comprises RNA.

Embodiment 75. The composition of embodiment 74, wherein the molar ratio of total nanoparticle cationic lipids to the total number of nucleotides comprised by the nucleic acid cargo is from about 2 to about 20.

Embodiment 76. The composition of embodiment 75, wherein the molar ratio of total nanoparticle cationic lipids to the total number of nucleotides comprised by the nucleic acid cargo is from about 2 to about 4.

Embodiment 77. A method of delivering a cargo to a target cell, the method comprising contacting the target cell with the composition of any one of embodiments 1-76.

Embodiment 78. The method of embodiment 77, wherein the target cell comprises a human cell.

Embodiment 79. The method of embodiment 77 or 78, wherein the target cell comprises an epithelial cell.

Embodiment 80. The method of embodiment 79, wherein the epithelial cell comprises an intestinal epithelial cell.

Embodiment 81. A method of delivering a cargo to a target cell, wherein the target cell is part of a mucosal tissue, the method comprising contacting the mucosal tissue with the composition of any one of embodiments 1-76.

Embodiment 82. The method of embodiment 81, wherein the mucosal tissue is part of a gastrointestinal tract.

Embodiment 83. The method of embodiment 82, wherein the target cell is a gastrointestinal cell.

Embodiment 84. The method of embodiment 83, wherein the gastrointestinal cell is selected from one or more of an intestinal epithelial cell, a lamina propria cell, an intraepithelial lymphocyte, an intestinal muscle cell, and an enteric neuron.

Embodiment 85. A method of delivering a cargo to a subject, the method comprising introducing the composition of any one of embodiments 1-76 to the gastrointestinal tract of the subject.

Embodiment 86. The method of embodiment 85, wherein the composition is introduced to the subject gastrointestinal tract by administering the composition to the subject by an administration route selected from one or more of oral administration and intrarectal administration.

Embodiment 87. The method of embodiment 85 or 86, wherein the nanoparticle targets a gastrointestinal cell.

Embodiment 88. The method of embodiment 87, wherein the gastrointestinal cell is selected from one or more of an intestinal epithelial cell, a lamina propria cell, an intraepithelial lymphocyte, an intestinal muscle cell, and an enteric neuron.

Embodiment 89. The method of embodiment 87 or 88, wherein the cargo is delivered to the gastrointestinal cell.

Embodiment 90. The method of embodiment 89, wherein the cargo is delivered to the intracellular space of the gastrointestinal cell.

Embodiment 91. The method of embodiment 90, wherein the cargo, a cargo component, or an expression product of the cargo is secreted from the gastrointestinal cell.

Embodiment 92. The method of embodiment 91, wherein secretion of the cargo, cargo component, or expression product of the cargo comprises apical secretion or basal secretion.

Embodiment 93. The method of embodiment 92, wherein the cargo, cargo component, or expression product of the cargo remains in an area proximal to the cell after secretion.

Embodiment 94. The method of embodiment 93, wherein the cargo, cargo component, or expression product of the cargo is secreted basally from the gastrointestinal cell and enters the circulation.

Embodiment 95. The method of embodiment 94, wherein the cargo, cargo component, or expression product of the cargo is distributed systemically after entering the circulation.

Embodiment 96. The method of any one of embodiments 85-95, wherein the cargo comprises a therapeutic agent.

Embodiment 97. The method of embodiment 96, wherein the therapeutic agent comprises one or more of a nucleic acid, a polypeptide, a protein, a biologic, an antibody, an enzyme, a hormone, a cytokine, an immunogen, and a genetic or epigenetic editing system component.

Embodiment 98. The method of embodiment 97, wherein the therapeutic agent comprises a nucleic acid.

Embodiment 99. The method of embodiment 98, wherein the nucleic acid encodes at least one polypeptide.

Embodiment 100. The method of embodiment 98 or 99, wherein the nucleic acid comprises DNA.

Embodiment 101. The method of embodiment 100, wherein the nucleic acid comprises plasmid DNA.

Embodiment 102. The method of any one of embodiments 98-101, wherein the nanoparticle targets a gastrointestinal cell and wherein the gastrointestinal cell is transfected with the nucleic acid.

Embodiment 103. The method of embodiment 102, wherein the gastrointestinal cell expresses a polypeptide encoded by the nucleic acid.

Embodiment 104. The method of embodiment 103, wherein the nucleic acid encodes a cell signaling factor.

Embodiment 105. The method of embodiment 104, wherein the cell signaling factor is selected from one or more of interleukin (IL)-2, IL-2 mutein Fc-fusion, IL-10, IL-10 mutein, IL-22, granulocyte-macrophage colony-stimulating factor (GM-CSF), granulocyte colony-stimulating factor (G-CSF), adrenomedullin, glucagon-like peptide 1 (GLP-1), glucagon-like peptide 2 (GLP-2), GLP-2 analog teduglutide, peroxisome proliferator-activated receptor gamma (PPARγ), human growth hormone (HGH), parathyroid hormone (PTH), fibroblast growth factor 21 (FGF21), and relaxin.

Embodiment 106. The method of embodiment 103, wherein the nucleic acid encodes an antibody.

Embodiment 107. The method of embodiment 106, wherein the antibody binds a target selected from one or more of IL-18, IL-18 receptor 1 (IL18R1), IL-23, tumor necrosis factor α (TNFα), proprotein convertase subtilisin kexin 9 (PCSK9), and protein 19 (P19).

Embodiment 108. The method of embodiment 106, wherein the antibody is a bispecific antibody.

Embodiment 109. The method of embodiment 108, wherein the bispecific antibody binds to cluster of differentiation 3 (CD3).

Embodiment 110. The method of embodiment 103, wherein the nucleic acid encodes an antimicrobial agent.

Embodiment 111. The method of embodiment 110, wherein the antimicrobial agent is selected from one or more of intestinal alkaline phosphatase (IAP) and a defensin.

Embodiment 112. The method of embodiment 103, wherein the nucleic acid encodes a genetic editing system component.

Embodiment 113. The method of embodiment 103, wherein the nucleic acid encodes an antigen as an immunogen for promotion of an immune response to the antigen by the subject.

Embodiment 114. The method of embodiment 113, wherein the antigen is derived from one or more of influenza virus, SARS-CoV-2 virus, Ebola virus, and polio virus.

Embodiment 115. The method of embodiment 113, wherein the antigen comprises a tumor cell neoantigen.

Embodiment 116. The method of embodiment 113, wherein the immune response comprises development of tolerance to the antigen by the subject.

Embodiment 117. The method of embodiment 116, wherein the antigen is associated with one or more of peanut allergies, celiac disease, rheumatoid arthritis, and IBD.

Embodiment 118. The method of embodiment 103, wherein the nucleic acid encodes a clotting factor.

Embodiment 119. The method of embodiment 118, wherein the clotting factor comprises Factor VIII.

Embodiment 120. The method of embodiment 103, wherein the nucleic acid encodes an enzyme.

Embodiment 121. The method of embodiment 120, wherein the enzyme comprises β-glucocerebrosidase (GBA).

Embodiment 122. The method of embodiment 102, wherein the nucleic acid comprises a non-coding RNA.

Embodiment 123. The method of embodiment 122, wherein the non-coding RNA comprises one or more of short interfering RNA (siRNA), microRNA (miRNA), long non-coding RNA, piwi-interacting RNA (piRNA), small nucleolar RNA (snoRNA), small Cajal body-specific RNA (scaRNA), transfer RNA (tRNA), ribosomal RNA (rRNA), and small nuclear RNA (snRNA).

Embodiment 124. A method of treating a therapeutic indication in a subject, the method comprising delivering a cargo to the subject according to the method of any one of embodiments 81-123.

Embodiment 125. The method of embodiment 124, wherein the therapeutic indication comprises an immune-related indication.

Embodiment 126. The method of embodiment 125, wherein the cargo comprises a nucleic acid encoding a therapeutic agent.

Embodiment 127. The method of embodiment 126, wherein the therapeutic agent is selected from the group consisting of IL-2, IL-2 mutein Fc-fusion, IL-10, IL-10 mutein, IL-22, adrenomedullin, an anti-microbial, and an anti-inflammatory antibody.

Embodiment 128. The method of embodiment 127, wherein the cargo is delivered to a gastrointestinal cell.

Embodiment 129. The method of embodiment 128, wherein the gastrointestinal cell expresses the therapeutic agent.

Embodiment 130. The method of embodiment 129, wherein the gastrointestinal cell secretes the therapeutic agent locally.

Embodiment 131. The method of embodiment 130, wherein the immune-related indication comprises a gastrointestinal indication.

Embodiment 132. The method of embodiment 131, wherein the gastrointestinal indication comprises one or more of gastrointestinal infection, inflammatory bowel disease (IBD), ulcerative colitis, and Crohn's disease.

Embodiment 133. The method of embodiment 129, wherein the gastrointestinal cell secretes the therapeutic agent into circulation.

Embodiment 134. The method of embodiment 133, wherein the immune-related indication comprises a non-gastrointestinal-specific indication and/or a systemic indication.

Embodiment 135. The method of embodiment 133 or 134, wherein the immune-related indication comprises one or more of graft versus host disease (GVHD), systemic lupus erythematosus (SLE), type I diabetes, rheumatoid arthritis, an infection, a wound, and an allergy.

Embodiment 136. The method of embodiment 124, wherein the therapeutic indication comprises a cancer-related indication.

Embodiment 137. The method of embodiment 136, wherein the cargo comprises a nucleic acid encoding a therapeutic agent.

Embodiment 138. The method of embodiment 137, wherein the therapeutic agent comprises GM-CSF.

Embodiment 139. The method of embodiment 138, wherein the cancer-related indication comprises one or more of Hodgkin's lymphoma, non-Hodgkin's lymphoma, acute lymphoblastic leukemia, and acute myelogenous leukemia.

Embodiment 140. The method of embodiment 139, wherein the subject has received or is undergoing chemotherapy and/or stem cell transplantation.

Embodiment 141. The method of any one of embodiments 138-140, wherein the cargo is delivered to gastrointestinal cells and wherein the gastrointestinal cells secrete the GM-CSF into circulation at a level sufficient to provide a circulating GM-CSF concentration of about 250 μg/m2/day.

Embodiment 142. The method of embodiment 124, wherein the therapeutic indication comprises neutropenia.

Embodiment 143. The method of embodiment 142, wherein the cargo comprises a nucleic acid encoding G-CSF.

Embodiment 144. The method of embodiment 143, wherein the cargo is delivered to gastrointestinal cells and wherein the gastrointestinal cells secrete the G-CSF into circulation at a level sufficient to provide about 5 μg/kg/day of the G-CSF.

Embodiment 145. The method of embodiment 144, wherein the subject is treated until subject neutrophil blood levels reach 1000/μl.

Embodiment 146. The method of embodiment 124, wherein the therapeutic indication comprises microvillus inclusion disease (MVID) and wherein the cargo comprises a nucleic acid encoding MYO5B gene product.

Embodiment 147. The method of embodiment 124, wherein the therapeutic indication comprises cystic fibrosis and wherein the cargo comprises a nucleic acid encoding cystic fibrosis transmembrane regulator protein (CFTR).

Embodiment 148. The method of embodiment 124, wherein the therapeutic indication comprises hemophilia and wherein the cargo comprises a nucleic acid encoding a clotting factor.

Embodiment 149. The method of embodiment 148, wherein the clotting factor comprises Factor VIII.

Embodiment 150. The method of embodiment 149, wherein the hemophilia comprises hemophilia A.

Embodiment 151. The method of embodiment 124, wherein the therapeutic indication comprises Gaucher's disease and wherein the cargo comprises a nucleic acid encoding GBA.

Embodiment 152. The method of embodiment 151, wherein the cargo is delivered to gastrointestinal cells and wherein the gastrointestinal cells secrete GBA into circulation at a level sufficient to provide steady-state GBA plasma levels of about 6 ng/mL.

Embodiment 153. The method of embodiment 124, wherein the therapeutic indication comprises short bowel syndrome (SBS) and wherein the cargo comprises a nucleic acid encoding GLP-2.

Embodiment 154. The method of embodiment 153, wherein the cargo is delivered to gastrointestinal cells and wherein the gastrointestinal cells secrete GLP-2 into circulation at a level sufficient to provide a circulating GLP-2 concentration of about 36 ng/mL.

Embodiment 155. The method of embodiment 124, wherein the therapeutic indication comprises a hormone deficiency and wherein the cargo comprises a nucleic acid encoding the deficient hormone.

Embodiment 156. The method of embodiment 155, wherein the deficient hormone is selected from the group consisting of HGH and PTH.

Embodiment 157. The method of embodiment 156, wherein the deficient hormone is HGH, wherein the cargo is delivered to gastrointestinal cells and wherein the gastrointestinal cells secrete the HGH into circulation at a level sufficient to provide a circulating HGH concentration of from about 1 to about 10 ng/mL in adults or from about 10 to about 50 ng/mL in children.

Embodiment 158. The method of embodiment 156, wherein the deficient hormone is PTH, wherein the cargo is delivered to gastrointestinal cells and wherein the gastrointestinal cells secrete the PTH into circulation at a level sufficient to provide a circulating PTH concentration of about 150 μg/mL.

Embodiment 159. The method of embodiment 124, wherein the therapeutic indication comprises non-alcoholic steatohepatitis (NASH) and wherein the cargo comprises a nucleic acid encoding GLP-1 or FGF21.

Embodiment 160. The method of embodiment 124, wherein the therapeutic indication comprises elevated circulating low density lipoprotein (LDL) level and wherein the cargo comprises a nucleic acid encoding an anti-PCSK9 antibody.

Embodiment 161. The method of embodiment 160, wherein the cargo is delivered to gastrointestinal cells and wherein the gastrointestinal cells secrete the anti-PCSK9 antibody into circulation at a level sufficient to provide a circulating anti-PCSK9 antibody concentration of from about 18 to about 19 μg/mL.

Embodiment 162. A delivery vehicle comprising: at least one bile salt, at least one bile acid, or a combination thereof; at least one cationic lipid; at least one structural lipid; and optionally at least one conjugated lipid.

Embodiment 163. The delivery vehicle of embodiment 162, wherein the at least one bile salt comprises sulfobromophthalein disodium salt hydrate, tauro-3β,5α,6β-trihydroxycholanoic acid, taurochenodeoxycholic acid sodium salt, taurocholic acid sodium salt hydrate, taurocholic acid sodium salt, taurodehydrocholic acid sodium salt, taurodeoxycholic acid sodium salt, taurohyodeoxycholate, taurohyodeoxycholic acid sodium salt, taurolithocholic acid 3-sulfate disodium salt, taurolithocholic acid sodium salt, tauro-β-muricholic acid sodium salt, tauroursodeoxycholic acid sodium salt, tauro-α-muricholic acid sodium salt, tauro-γ-muricholic acid sodium salt, tauro-ω-muricholic acid sodium salt, β-Estradiol 17-(β-D-glucuronide) sodium salt, lithocholic acid 3-sulfate (disodium salt), chenodeoxycholic acid 3-sulfate (disodium salt), chenodeoxycholic acid 7-sulfate (disodium salt), cholic acid 3-sulfate (disodium salt), cholic acid 7-sulfate (disodium salt), cholic acid sodium salt, deoxycholic acid 3-sulfate (disodium salt), deoxycholic acid disulfate (trisodium salt), phenoxymethylpenicillinic acid potassium salt, chenodeoxycholic acid disulfate (trisodium salt), chenodeoxycholic acid sodium salt, cholate, methyl cholate, sodium taurocholate hydrate, 1-naphthyl isothiocyanate, deoxycholate, hyodeoxycholate, glycocholate, sodium glycochenodeoxycholate, sodium cholate hydrate, taurocholate, taurodeoxycholate, taurochenodeoxycholate, chenodeoxycholate, lithocholate, isolithocholate, alloisolithocholate, sodium deoxycholate, sodium deoxycholate monohydrate, dehydrolithochlate, sodium glycodeoxycholate, sodium glycocholate hydrate, sodium taurodeoxycholate hydrate, sodium chenodeoxycholate, glycolithocholate sulfate, glycolithocholate, sodium taurohyodeoxycholate hydrate, sodium taurocholate, sodium tauroursodeoxycholate, sodium taurolithocholate, glycodeoxycholate, and any combination thereof.

Embodiment 164. The delivery vehicle of embodiment 162, wherein the at least one bile salt comprises cholate deoxycholate, chenodeoxycholate, lithocholate, and any combination thereof.

Embodiment 165 The delivery vehicle of embodiment 162, wherein the at least one bile acid comprises 3β,5α,6β-trihydroxycholanoic acid, 12-ketochenodeoxycholic acid, 12-ketodeoxycholic acid, 12-ketolithocholic acid, 3-oxo chenodeoxycholic acid, 3-oxo deoxycholic acid, 3-oxocholic acid, 3α,6α,7α,12α-tetrahydroxy bile acid, 3α,6α,7α,12α-tetrahydroxy bile acid, 4-bromobenzoic acid, 6,7-diketolithocholic acid, 7-ketodeoxycholic acid, 7-ketolithocholic acid, allocholic acid, alloisolithocholic acid, apocholic acid, apocholic acid (delta 14 isomer), arachidyl amido cholanoic acid, chenodeoxycholic acid, chenodeoxycholic acid-d4, cholic acid, dehydrocholic acid, dehydrolithocholic acid, deoxycholic acid, dioxolithocholic acid, glyco-12-oxolithocholanoic acid, glycochenodeoxycholic acid, glycocholic acid, glycocholic acid hydrate, glycodehydrocholic acid, glycodeoxycholic acid, glycohyodeoxycholic acid, glycolithocholic acid, glycoursodeoxycholic acid, glyco-γ-muricholic acid, hyocholic acid, hyodeoxycholic acid, isodeoxycholic acid, isolithocholic acid, lithocholic acid, murideoxycholic acid, nordeoxycholic acid, obeticholic acid, pentadecanoic acid, ursocholic acid, ursodeoxycholic acid, ursodeoxycholic acid-D4, α-muricholic acid, β-muricholic acid, ω-muricholic acid, and any combination thereof.

Embodiment 166. The delivery vehicle of embodiment 162, wherein the at least one bile acid comprises ursodiol, 5beta-cholanic acid, 3-oxy-cholenic acid, and any combination thereof.

Embodiment 167. The delivery vehicle of any one of embodiments 162-166, wherein the delivery vehicle comprises about 5 to about 40 mole % of the at least one bile salt or the at least one bile acid.

Embodiment 168. The delivery vehicle of any one of embodiments 162-167, wherein the delivery vehicle comprises about 20 to about 40 mole % of the at least one bile salt or the at least one bile acid.

Embodiment 169. The delivery vehicle of any one of embodiments 162-168, wherein the delivery vehicle comprises about 30 to about 40 mole % of the at least one bile salt or the at least one bile acid.

Embodiment 170. The delivery vehicle of any one of embodiments 162-169, wherein the at least one bile salt comprises deoxycholate.

Embodiment 171. The delivery vehicle of any one of embodiments 162-169, wherein the at least one bile salt comprises chenodeoxycholate.

Embodiment 172. The delivery vehicle of any one of embodiments 162-169, wherein the at least one bile salt comprises lithocholate.

Embodiment 173. The delivery vehicle of any one of embodiments 162-169, wherein the at least one bile alloisolithocholate.

Embodiment 174. The delivery vehicle of any one of embodiments 162-169, wherein the at least one bile comprises dehydrolithocholate.

Embodiment 175. The delivery vehicle of any one of embodiments 162-169, wherein the at least one bile acid comprises ursodiol.

Embodiment 176. The delivery vehicle of any one of embodiments 162-169, wherein the at least one bile salt comprises isolithocholate.

Embodiment 177. The delivery vehicle of any one of embodiments 162-169, wherein the at least one bile salt comprises dehydrolithochlate.

Embodiment 178. The delivery vehicle of any one of embodiments 162-169, wherein the at least one bile acid comprises 5-beta-cholanic acid.

Embodiment 179. The delivery vehicle of any one of embodiments 162-169, wherein the at least one bile salt comprises taurodeoxycholate.

Embodiment 180. The delivery vehicle of any one of embodiments 162-169, wherein the at least one bile comprises taurochenodeoxycholate.

Embodiment 181. The delivery vehicle of any one of embodiments 162-169, wherein the at least one bile salt glycocholate.

Embodiment 182. The delivery vehicle of any one of embodiments 162-169, wherein the at least one bile acid comprises 3-oxy-cholenic acid.

Embodiment 183. The delivery vehicle of any one of embodiments 162-169, wherein the delivery vehicle comprises deoxycholate and lithocholate.

Embodiment 184. The delivery vehicle of embodiment 183, wherein the delivery vehicle comprises about 20 to about 30 mole % deoxycholate and from about 5 to about 10 mole % of lithocholate.

Embodiment 185. The delivery vehicle of any one of embodiment 162-183 wherein the delivery vehicle comprises at least one bile salt and at least one bile acid.

Embodiment 186. The delivery vehicle of embodiment 162, wherein the at least one cationic lipid comprises N1-[2-((1 S)-1-[(3-aminopropyl)amino]-4-[di(3-amino-propyl)amino]butylcarboxamido)ethyl]-3,4-di[oleyloxy]-benzamide (MVL5), N4-Cholesteryl-Spermine HCl (GL67), 1,2-dioleyloxy-3-dimethylaminopropane (DODMA), N-[1-(2,3-dioleyloxy)propyl]-N,N,N-trimethylammonium chloride (DOTMA), [1,2-bis(oleoyloxy)-3-(trimethylammonio)propane] (DOTAP), dimethyldioctadecylammonium (DDA), 30[N-(N′,N′-dimethylaminoethane)-carbamoyl] cholesterol (DC-Chol), and dioctadecylamidoglycylspermine (DOGS), 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 1,2-Dialkyloxy-N,N-dimethylaminopropane, 4-(2,2-diocta-9,12-dienyl-[1,3]dioxolan-4-ylmethyl)-dimethylamine, O-alkyl ethylphosphocholines, (6Z,9Z,28Z,31Z)-heptatriacont-6,9,28,31-tetraene-19-yl 4-(dimethylamino)butanoate (MC3), (6Z,9Z,28Z,31Z)-heptatriaconta-6,9,28,31-tetraen-19-yl 3-(dimethylamino)propanoate (MC2), 3β-[N-(N′,N′-dimethylaminoethane)-carbamoyl]cholesterol, N4-Cholesteryl-Spermine, 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 7-(4-(dimethylamino)butyl)-7-hydroxytridecane-1,13-diyl dioleate (CL1H6), 7-(4-(diisopropylamino)butyl)-7-hydroxytride-cane-1,13-diyl dioleate (CL4H6), 1,2-stearoyl-3-trimethylammonium-propane (DSTAP), 1,2-dipalmitoyl-3-trimethylammonium-propane (DPTAP), 1,2-Distearoyl-3-Dimethylammonium-Propane (DSDAP), or any combinations thereof. In some embodiments, the saturated cationic lipid can comprise at least one of 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, and any combination thereof.

Embodiment 187. The delivery vehicle of embodiment 162, wherein the at least one cationic lipid comprises MVL5; MC2; CL1H6; CL4H6; DODMA, and any combination thereof.

Embodiment 188. The delivery vehicle of any one of embodiment 1 or embodiment 25 or embodiment 26, wherein the delivery vehicle comprises about 5 to about 90 mole % of the at least one cationic lipid.

Embodiment 189. The delivery vehicle of any one of embodiment 162 or embodiments 186-188, wherein the delivery vehicle comprises about 5 to about 60 mole % of the at least one cationic lipid.

Embodiment 190. The delivery vehicle of any one of embodiment 162 or embodiments 186-189, wherein the delivery vehicle comprises about 10 to about 60 mole % of the at least one cationic lipid.

Embodiment 191. The delivery vehicle of any one of embodiment 162 or embodiments 186-190, wherein the delivery vehicle comprises about 10 to about 50 mole % of the at least one cationic lipid.

Embodiment 192. The delivery vehicle of any one of embodiment 162 or embodiments 186-191, wherein the delivery vehicle comprises about 10 to about 30 mole % of the at least one cationic lipid.

Embodiment 193. The delivery vehicle of any one of embodiment 162 or embodiments 186-192, wherein the at least one cationic lipid comprises at least one multivalent cationic lipid and at least one ionizable cationic lipid.

Embodiment 194. The delivery vehicle of embodiment 193, wherein the at least one multivalent cationic lipid comprises MVL5.

Embodiment 195. The delivery vehicle of any one of embodiment 193 or embodiment 194, wherein the delivery vehicle comprises about 5 to about 90 mole % of the at least one multivalent cationic lipid.

Embodiment 196. The delivery vehicle of any one of embodiment 162 or embodiments 193-195, wherein the delivery vehicle comprises about 5 to about 60 mole % of the at least one multivalent cationic lipid.

Embodiment 197. The delivery vehicle of any one of embodiment 162 or embodiments 193-196, wherein the delivery vehicle comprises about 5 to about 30 mole % of the at least one multivalent cationic lipid.

Embodiment 198. The delivery vehicle of any one of embodiment 162 or embodiments 193-197, wherein the delivery vehicle comprises about 5 to about 15 mole % of the at least one multivalent cationic lipid.

Embodiment 199. The delivery vehicle of any one of embodiments 193-198, wherein the at least one multivalent cationic lipid comprises about up to about 100 mole % of the at least one cationic lipid.

Embodiment 200. The delivery vehicle of any one of embodiments 193-199, wherein the at least one multivalent cationic lipid comprises about 5-75 mole % of the at least one cationic lipid.

Embodiment 201. The delivery vehicle of any one of embodiments 193-200, wherein the at least one multivalent cationic lipid comprises about 40-60 mole % of the at least one cationic lipid.

Embodiment 202. The delivery vehicle of any one of embodiments 193-201, wherein the at least one multivalent cationic lipid comprises about 50 mole % of the at least one cationic lipid.

Embodiment 203. The delivery vehicle of embodiment 193, wherein the at least one ionizable cationic lipid comprises at least one of MC2, CL1H6, CL4H6, DODMA, and any combination thereof.

Embodiment 204. The delivery vehicle of any one of embodiment 193 or embodiment 203, wherein the at least one ionizable cationic lipid comprises MC2.

Embodiment 205. The delivery vehicle of any one of embodiment 193 or embodiment 203, wherein the at least one ionizable cationic lipid comprises CL1H6.

Embodiment 206. The delivery vehicle of embodiment 193 or embodiment 203, wherein the at least one ionizable cationic lipid comprises CL4H6.

Embodiment 207. The delivery vehicle of embodiment 193 or embodiment 203, wherein the at least one ionizable cationic lipid comprises DODMA.

Embodiment 208. The delivery vehicle of any one of embodiment 162 or embodiments 193-207, wherein the delivery vehicle comprises about 5 to about 90 mole % of the at least one ionizable cationic lipid.

Embodiment 209. The delivery vehicle of any one of embodiment 162 or embodiments 193-208, wherein the delivery vehicle comprises about 5 to about 60 mole % of the at least one ionizable cationic lipid.

Embodiment 210. The delivery vehicle of any one of embodiment 162 or embodiments 193-209, wherein the delivery vehicle comprises about 5 to about 30 mole % of the at least one ionizable cationic lipid.

Embodiment 211. The delivery vehicle of any one of embodiment 162 or embodiments 193-210, wherein the delivery vehicle comprises about 5 to about 15 mole % of the at least one ionizable cationic lipid.

Embodiment 212. The delivery vehicle of any one of embodiment 162 or embodiments 193-211, wherein the ionizable cationic lipid comprises up to about 100 mole % of the at least one cationic lipid.

Embodiment 213. The delivery vehicle of any one of embodiment 162 or embodiments 193-212, wherein the ionizable cationic lipid comprises about 5-75 mole % of the at least one cationic lipid.

Embodiment 214. The delivery vehicle of any one of embodiment 162 or embodiments 193-213, wherein the ionizable cationic lipid comprises about 40-60 mole % of the at least one cationic lipid.

Embodiment 215. The delivery vehicle of any one of embodiment 162 or embodiments 193-214, wherein the ionizable cationic lipid comprises about 50 mole % of the at least one cationic lipid.

Embodiment 216. The delivery vehicle of embodiment 162, wherein the delivery vehicle comprises about the same amount of the at least one multivalent cationic lipid and the at least one ionizable cationic lipid.

Embodiment 217. The delivery vehicle of embodiment 162, wherein the at least one structural lipid comprises at least one neutral lipid, at least one anionic lipid, at least one phospholipid, and any combination thereof.

Embodiment 218. The delivery vehicle of any one of embodiment 162 or embodiment 217, wherein the at least one structural lipid is comprises glycerol monooleate (GMO), dioleoylphosphatidylethanolamine (DOPE), 1,2-dimyristoyl-sn-glycero-3-phosphocholine (DMPC), short-chainbis-n-heptadecanoylphosphatidylcholine (DHPC), dihexadecoylphosphoethanolamine (DHPE), 1,2-dilinoleoyl-sn-glycero-3-phosphocholine (DLPC), dimyristoylphosphoethanolamine (DMPE), dimyristoylphosphatidylglycerol (DMPG), dioleoylphosphatidylcholine (DOPC), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylcholine (DPPC), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoylphosphoethanolamineimidazole (DSPEI), 1,2-diundecanoyl-sn-glycero-phosphocholine (DUPC), eggphosphatidylcholine (EPC), hydrogenatedsoybeanphosphatidylcholine (HSPC), mannosializeddipalmitoylphosphatidylethanolamine (ManDOG), 1,2-Dioleoyl-sn-Glycero-3-Phosphoethanolamine-N-[4-(p-maleimidomethyl)cyclohexane-carboxamide] (MCC-PE), 1,2-diphytanoyl-sn-glycero-3-phosphoethanolamine (ME 16.0 PE), 1-myristoyl-2-hydroxy-sn-glycero-phosphocholine (MHPC), 1-oleoyl-2-cholesterylhemisuccinoyl-sn-glycero-3-phosphocholine (OChemsPC), phosphatidicacid (PA), phosphatidylethanolaminelipid (PE), phosphatidylglycerol (PG), partiallyhydrogenatedsoyphosphatidylchloline (PHSPC), phosphatidylinositollipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), 18-1-transPE,1-stearoyl-2-oleoyl-phosphatidyethanolamine (SOPE), soybeanphosphatidylcholine (SPC), 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, and any combination thereof.

Embodiment 219. The delivery vehicle of any one of embodiment 162 or embodiment 217 or embodiment 218, wherein the at least one structural lipid comprises DSPC, DMPC, DOPE, GMO, and any combination thereof.

Embodiment 220. The delivery vehicle of embodiment 162 or embodiment 217-219, wherein the delivery vehicle comprises from about 5 to about 75 mole % of the at least one structural lipid.

Embodiment 221. The delivery vehicle of embodiment 162 or embodiments 218-220, wherein the delivery vehicle comprises from about 30 to about 50 mole % of the at least one structural lipid.

Embodiment 222. The delivery vehicle of embodiment 162 or embodiments 218-221, wherein the delivery vehicle comprises from about 35 to about 45 mole % of the at least one structural lipid.

Embodiment 223. The delivery vehicle of embodiment 162, wherein the delivery vehicle does not comprise cholesterol.

Embodiment 224. The delivery vehicle of embodiment 162, wherein the at least one conjugated lipid comprises at least one conjugated lipid and at least one hydrophilic polymer.

Embodiment 225. The delivery vehicle of any one of embodiment 162 or embodiment 214, wherein the at least one hydrophilic polymer comprises polyethylene glycol (PEG).

Embodiment 226. The delivery vehicle of any one of embodiment 162 or embodiment 224, wherein the at least one conjugated lipid comprises at least one phospholipid, at least one neutral lipid, at least one glyceride, at least one diglyceride, at least one anionic lipid, at least one cationic lipid, and any combination thereof.

Embodiment 227. The delivery vehicle of any one of embodiments 162 or embodiment 224 or embodiment 226, wherein the at least one conjugated lipid comprises 1,2-dimyristoyl-rac-glycerol (DMG), 1,2-dimyristoyl-sn-glycero-3-phosphoethanolamine (DMPE), 1,2-distearoyl-rac-glycerol (DSG), 1,2-dipalmitoyl-rac-glycerol (DPG), 1,2-distearoyl-sn-glycero-3-phosphoethanolamine (DSPE), diacylglycerol (DAG), 1,2-dipalmitoryl-sn-glycero-3-phosphoethanolamine (DPPE), and any combination thereof.

Embodiment 228. The delivery vehicle of any one of embodiment 162 or embodiments 225-227, wherein the at least one conjugated lipid comprises at least one of DMG-PEG, DMPE-PEG, DSG-PEG, DPG-PEG, DSPE-PEG, DAG-PEG, DPPE-PEG, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSG-PEGMA, DSG-PEG5000, DPPE-PEG-2K, DPPE-mPEG2000, DPPE-mPEG, DPG-PEGMA, DOPE-PEG2000, DMPE-PEGMA, DMPE-PEG2000, DMPE-mPEG2000, DMG-PEGMA, DMG-PEG2000, C18PEG750, C18PEG5000, CI8PEG3000, CI8PEG2000, CI6PEG2000, CI4PEG2000, C18-PEG5000, C18PEG, C16PEG, C14-PEG-DSPE200, C14-PEG2000, C14PEG2000, C14-PEG 2000, C14-PEG, C14PEG, (PEG)-C-DOMG, PEG-C-DMA and any combination thereof.

Embodiment 229. The delivery vehicle of any one of embodiment 162 or embodiments 224-228, wherein the at least one conjugated lipid comprises DMG-PEG.

Embodiment 230. The delivery vehicle of any one of embodiment 162 or embodiments 224-228, wherein the at least one conjugated lipid comprises DMPE-PEG.

Embodiment 231. The delivery vehicle of any one of embodiment 162 or embodiments 224-230, wherein the delivery vehicle comprises from about 0.5 to about 2.0 mole % of the at least one conjugated lipid.

Embodiment 232. The delivery vehicle of embodiment 162, wherein the delivery vehicle does not comprise at least one conjugated lipid.

Embodiment 233. The delivery vehicle of any one of embodiments 162-230, wherein the delivery vehicle comprises: the at least one bile salt or the at least one bile acid; the at least one multivalent cationic lipid; the at least one ionizable cationic lipid; the at least one structural lipid; and the at least one conjugated lipid.

Embodiment 234. The delivery vehicle of embodiment 233, wherein the delivery vehicle comprises: about 5-40 mole % of the at least one bile salt or the at least one bile acid; about 5-90 mole % of the at least one multivalent cationic lipid; about 5-90 mole % of the at least one ionizable cationic lipid; about 5-75 mole % of the at least one structural lipid component; and about 0.5-2.0 mole % the at least one conjugated lipid component.

Embodiment 235. The delivery vehicle of any one of embodiments 233 or 234, wherein the delivery vehicle comprises: about 5-40 mole % of the at least one bile salt or the at least one bile acid; about 5-60 mole % of the at least one multivalent cationic lipid; about 5-60 mole % of the at least one ionizable cationic lipid; about 5-75 mole % of the at least one structural lipid; and about 0.5-2.0 mole % of the at least one conjugated lipid.

Embodiment 236. The delivery vehicle of any one of embodiments 233-235, wherein the delivery vehicle comprises: about 20-40 mole % of the at least one bile salt or the at least one bile acid; about 5-30 mole % of the at least one multivalent cationic lipid; about 5-30 mole % of the at least one ionizable cationic lipid; about 30-50 mole % of the at least one structural lipid; and about 0.5-2.0 mole % of the at least one conjugated lipid.

Embodiment 237. The delivery vehicle of any one of embodiments 233-236, wherein the delivery vehicle comprises: about 30-40 mole % of the at least one bile salt or the at least one bile acid; about 5-15 mole % of the at least one multivalent cationic lipid; about 5-15 mole % of the at least one ionizable cationic lipid; about 35-45 mole % of the at least one structural lipid; and about 0.5-2.0 mole % of the at least one conjugated lipid.

Embodiment 238. The delivery vehicle of any one of embodiments 233-237, wherein the delivery vehicle comprises: about 33 mole % of the at least one bile salt or the at least one bile acid; about 12.5 mole % of the at least one multivalent cationic lipid; about 12.5 mole % of the at least one ionizable cationic lipid; about 41 mole % of the at least one structural lipid; and about 1 mole % of the at least one conjugated lipid.

Embodiment 239. The delivery vehicle of any one of embodiments 162-238 wherein the delivery vehicle comprises any of the compositions disclosed in Table 1B.

Embodiment 240. The delivery vehicle of any one of embodiments 162-239, wherein the at least one conjugated lipid is conjugated with at least one polypeptide.

Embodiment 241. The delivery vehicle of embodiment 240, wherein the at least one polypeptide comprises at least one mucus penetrating polypeptide.

Embodiment 242. The delivery vehicle of any one of embodiment 240 or embodiment 241, wherein the at least one mucus penetrating polypeptide comprises an amino acid sequence according to SEQ ID NO: 17.

Embodiment 243. The delivery vehicle of any one of 162-242, wherein the delivery vehicle comprises a cargo.

Embodiment 244. The delivery vehicle of embodiment 243, wherein the cargo comprises a nucleic acid, a protein, an antibody, a peptide, a small molecule, a biologic, a peptidomimetic, a ribozyme, a chemical agent, a viral particle, a growth factor, a cytokine, an immunomodulating agent, a fluorescent dye, and any combination thereof.

Embodiment 245. The delivery vehicle of embodiment 244, wherein the cargo comprises a nucleic acid.

Embodiment 246. The delivery vehicle of embodiment 245, wherein the nucleic acid comprises DNA.

Embodiment 247. The delivery vehicle of embodiment 246, wherein the DNA comprises plasmid DNA.

Embodiment 248. The delivery vehicle of any one of embodiments 245-247, wherein the molar ratio of total nanoparticle cationic lipids to the total number of nucleotides comprised by the nucleic acid cargo is from about 2 to about 20.

Embodiment 249. The delivery vehicle of embodiment 248, wherein the molar ratio of total nanoparticle cationic lipids to the total number of nucleotides comprised by the nucleic acid cargo is from about 14 to about 18.

Embodiment 250. The delivery vehicle of embodiment 249, wherein the nucleic acid comprises RNA.

Embodiment 251. The delivery vehicle of embodiment 250, wherein the molar ratio of total nanoparticle cationic lipids to the total number of nucleotides comprised by the nucleic acid cargo is from about 2 to about 20.

Embodiment 252. The delivery vehicle of embodiment 251, wherein the molar ratio of total nanoparticle cationic lipids to the total number of nucleotides comprised by the nucleic acid cargo is from about 2 to about 4.

Embodiment 253. A pharmaceutical composition, wherein the pharmaceutical composition comprises at least one of the delivery vehicles described in embodiments 162-252 and an optional pharmaceutically acceptable excipient.

Embodiment 254. The pharmaceutical composition of embodiment 253, wherein the pharmaceutically acceptable excipient comprises an excipient, adjuvant, solution, stabilizer, additive, surfactant, lyophilization element, dilutant, and any combination thereof.

Embodiment 255. The pharmaceutical composition embodiments 253 or 254, wherein the pharmaceutical composition is formulated for enteric delivery.

Embodiment 256. A method of delivering at least one cargo to a subject, the method comprising introducing at least one of the delivery vehicle of any of embodiments 162-252 or at least one of the pharmaceutical compositions of any of embodiments 253-255 to the gastrointestinal tract of the subject.

Embodiment 257. The method of embodiment 256, wherein the at least one delivery vehicle or the at least one pharmaceutical composition is introduced to the subject gastrointestinal (GI) tract by at least one rout of administration.

Embodiment 258. The method of embodiment 257, wherein the at least one rout of comprises intravenous administration, intraperitoneal administration, intramuscular administration, transdermal administration, ocular administration, oral administration, intrarectal administration, direct injection to the GI tract, and any combination thereof.

Embodiment 259. The method of any one of embodiments 256-258, wherein the at least one delivery vehicle or at least one pharmaceutical composition targets at least one gastrointestinal cell.

Embodiment 260. The method of embodiment 259, wherein the at least one gastrointestinal cell comprises at least one of an intestinal epithelial cell, a lamina propria cell, an intraepithelial lymphocyte, an intestinal muscle cell, an enteric neuron, or any combination thereof.

Embodiment 261. The method of embodiment 259 or 260, wherein at least one cargo is delivered to the gastrointestinal cell.

Embodiment 262. The method of embodiment 261, wherein the at least one cargo is delivered to the intracellular space of the gastrointestinal cell.

Embodiment 263. The method of any one of embodiments 261 or 262, wherein the at least one cargo, an at least one cargo component, or an at least one expression product of the cargo is secreted from the gastrointestinal cell.

Embodiment 264. The method of embodiment 263, wherein secretion of the at least one cargo, the at least one cargo component, or the at least one expression product of the cargo comprises apical secretion or basal secretion.

Embodiment 265. The method of embodiment 264, wherein the at least one cargo, the at least one cargo component, or the at least one expression product of the cargo remains in an area proximal to the cell after secretion.

Embodiment 266. The method of embodiment 265, wherein the at least one cargo, the at least one cargo component, or the at least one expression product of the cargo is secreted basally from the gastrointestinal cell and enters the circulation.

Embodiment 267. The method of embodiment 266, wherein the at least one cargo, the at least one cargo component, or the at least one expression product of the cargo is distributed systemically after entering the circulation.

Embodiment 268. The method of any one of embodiments 256-267, wherein the at least one cargo comprises at least one therapeutic agent.

Embodiment 269. The method of embodiment 268, wherein the at least one therapeutic agent comprises one or more of a nucleic acid, a polypeptide, a protein, a biologic, an antibody, an enzyme, a hormone, a cytokine, an immunogen, and a genetic or epigenetic editing system component.

Embodiment 270. The method of embodiment 269, wherein the at least one therapeutic agent comprises at least one nucleic acid.

Embodiment 271. The method of embodiment 270, wherein the at least one nucleic acid encodes at least one polypeptide.

Embodiment 272. The method of any one of embodiments 270 or 271, wherein the at least one nucleic acid comprises DNA.

Embodiment 273. The method of embodiment 272, wherein the at least one nucleic acid comprises plasmid DNA.

Embodiment 274. The method of any one of embodiments 270 or 271, wherein the at least one nucleic acid comprises RNA.

Embodiment 275. The method of embodiment 274, wherein the at least one nucleic acid comprises mRNA, circRNA, saRNA, and any combination thereof.

Embodiment 276. The method of any one of embodiments 269-275, comprising transfecting the at least one gastrointestinal cell with the at least one nucleic acid.

Embodiment 278. The method of embodiment 276, wherein the at least one gastrointestinal cell expresses at least one polypeptide encoded by the at least one nucleic acid.

Embodiment 279. The method of any one of embodiment 276 or embodiment 277, wherein the polypeptide comprises Granulocyte Spleeny-Stimulating Factor (G-CSF), Green Florescent Protein (GFP) and any combination thereof.

Embodiment 280. The method of embodiment 270, wherein the at least one nucleic acid comprises a at least one non-coding RNA.

Embodiment 281. The method of embodiment 271, wherein the at least one non-coding RNA comprises one or more of short interfering RNA (siRNA), microRNA (miRNA), long non-coding RNA, piwi-interacting RNA (piRNA), small nucleolar RNA (snoRNA), small Cajal body-specific RNA (scaRNA), transfer RNA (tRNA), ribosomal RNA (rRNA), and small nuclear RNA (snRNA).

Embodiment 282. A method for treating at least one therapeutic indication in a subject in need thereof comprising delivering at least one delivery vehicle described herein or at least one pharmaceutical compositions described herein to the subject via at least one of the methods for delivery of a cargo described herein.

Embodiment 283. The method of embodiment 282, wherein the at least one therapeutic indication comprises at least one of a neurodegenerative disease, an ocular disease, a reproductive disease, a gastrointestinal disease, a brain disease, a skin disease, a skeletal disease, a muscoskeletal disease, a pulmonary disease, a thoracic disease, cystic fibrosis, tay-sachs, fragile X, Huntington's, neurofibromatosis, sickle cell, thalassemias, Duchenne's muscular dystrophy, familial adenomatous polyposis (FAP), attenuated FAP, microvillus inclusion disease (MVID), chronic inflammatory bowel disease, chronic inflammatory bowel disease, ileal Crohn's, juvenile polyposis, hereditary diffuse gastric cancer syndrome (HDGC), Peutz-Jeghers syndrome, lynch syndrome, gastric adenocarcinoma and proximal polyposis of the stomach (GAPPS), Li-Fraumeni syndrome, familial gastric cancer, Gilbert's syndrome, telangiectasia, mucopolysaccaride, Osler-Weber-Rendu syndrome, pancreatitis, keratoacanthoma, biliary atresia, Morquio's syndrome, Hurler's syndrome, Hunter's syndrome, Crigler-Najjar, Rotor's, Peutz-Jeghers' syndrome, Dubin-Johnson, Osteochondroses, Osteochondrodysplasias, polyposis, gastrointestinal infections, inflammatory bowel disease (IBD), ulcerative colitis, Crohn's disease, hemophilia, short bowel syndrome (SBS), diabetes, non-alcoholic steatohepatitis (NASH), with Hodgkin's lymphoma, non-Hodgkin's lymphoma, acute lymphoblastic leukemia, or acute myelogenous leukemia (AML), neutropenia, or any combination thereof.

Embodiment 284. The method of embodiment 283, wherein the at least one therapeutic indication comprises at least one immune-related indication.

Embodiment 285. The method of embodiment 284, wherein the at least one immune-related indication comprises at least one gastrointestinal indication.

Embodiment 286. The method of embodiment 285, wherein the at least one therapeutic indication comprises at least one cancer-related indication.

### XI. EXAMPLES

### Example 1: Preparation of Exemplary Delivery Vehicles of this Disclosure

This example provides an exemplary method of preparing a delivery vehicle of this disclosure. Lipid components of the delivery vehicle 1,2-dioleyloxy-3-dimethylaminopropane (DODMA) (Sigma Aldrich), Deoxycholate (Sigma Aldrich), MVL5 (Avanti Polar Lipids), DSPC (Avanti Polar Lipids), DMG-PEG 2000 (Avanti Polar Lipids), DOPC (Avanti Polar Lipids), DiI (ThermoFisher Scientific), DiO (ThermoFisher Scientific) were dissolved in ethanol and heated above their phase transition temperature, for instances where the phase transition temperatures were higher than 37° C. For example, when using DSPC, the lipids and aqueous phase were heated to 70° C. When using DOPC, the lipids and aqueous phase were not heated and used at room temperature. Nucleic acids were dissolved in an aqueous buffer heated above the phase transition temperature of the lipids.

The aqueous buffer pH was set at below the pKa of the bile salt and the cationic lipids. In this way, the lipids were strongly cationic when formulated with the nucleic acids. To form the delivery vehicle with cargo, the lipids and nucleic acids were mixed using microfluidic channels followed by removal of ethanol via dialysis. Other suitable methods can also be used for this step. For example, lipid structures such as liposomes can be formed by thin film hydration where the lipids may be dissolved in organic phase and dried using a rotovap under rotation. The thin film that is formed can be hydrated in water. The hydrated lipids can be heated to 70° C. for example, for DSPC or used at room temperature for example, for DOPC and extruded through the appropriate extruder pore size. The nucleic acid cargo can be mixed with the lipids to form lipoplexes.

Another suitable alternate method for the preparation of exemplary delivery vehicles is using the thin film hydration. Lipids are dissolved and mixed in an organic solvent. The solvent is removed, and the formed thin film is hydrated in an aqueous solution. The lipids are sized appropriately using sonication or extrusion. Nucleic acids can be complexed by mixing the lipid mixture and nucleic acids together.

### Formulation of the Exemplary Delivery Vehicles

To prepare exemplary delivery vehicles, containing encapsulated nucleic acid, 300 μg of plasmid DNA encoding for Gaussia luciferase under a cytomegalovirus (CMV) promoter was dissolved in a final volume of 3 mL 50 mM Sodium Acetate buffer (pH 4.8). Appropriate moles of MVL5, DODMA, Deoxycholate, MVL5, DSPC, DMG-PEG2000 and/or DOPC were mixed in ethanol according to their mole and cationic lipid:nucleotide ratio (See Table 2 for mole % of the lipids in the various formulations prepared). The cationic lipid: nucleotide molar ratio was maintained at about 16. Fluorescently labelled lipids, such as DiI and DiO, when used, were added to the mix at 0.5% of the total lipid moles. Ethanol volume was raised to 1 mL.

The nucleic acid is in the aqueous sodium acetate buffer phase in a 3 mL syringe. The lipids are in ethanol in a 1 mL syringe. The two syringes are mounted on to a NanoAssemblr (Precision Nanosystems) and then the two samples are mixed using the microfluidics chip on the NanoAssemblr.

For this study, samples were mounted into syringes (as mentioned above, nucleic acid in the 3 mL syringe and lipids in the 1 mL syringe) on a NanoAssemblr Benchtop and preheated to 65° C. for the DSPC formulations or at room temperature (about 25° C.) for the DOPC formulations. Samples were mixed using the NanoAssemblr Benchtop microfluidic chip system with a flow rate of 6 mL/min. pH was neutralized with 300 mM HEPES buffer at pH 7.5. Ethanol was removed using dialysis overnight. Samples were concentrated using Amicon Ultra-4 with a 100 kDa molecular weight cutoff.

TABLE 2
Example Formulations Prepared

| | Formula- | |
|---|---|---|
| | tion | Ratio of lipids |
| Lipid Components | No. | (in mole %) |
| DODMA/DSPC/Deoxycholate/ | 1 | 40/31.6/25.9/2.5 |
| PEG-DMG | 2 | 30/37.1/30.4/2.5 |
| | 3 | 20/42.6/34.9/2.5 |
| | 4 | 10/48.1/39.4/2.5 |
| | 5 | 25/37.1/30.4/7.5 |
| MVL5/DODMA/DSPC/ | 6 | 6.25/18.75/37.1/30.4/7.5 |
| Deoxycholate/DMG-PEG | 7 | 12.5/12.5/37.1/30.4/7.5 |
| | 8 | 18.75/6.25/37.1/30.4/7.5 |
| MVL5/DSPC/Deoxycholate/ | 9 | 25/37.1/30.4/7.5 |
| DMG-PEG | | |
| DSPC/Deoxycholate | 10 | 55/45 |
| DOPC/Deoxycholate | 11 | 55/45 |
| DSPC/Deoxycholate/DMG-PEG | 12 | 53.6/43.9/2.5 |
| DSPC/Chol | 13 | 55/45 |
| MVL5/DODMA/DSPC/DMG-PEG | 14 | 17.3/17.3/58.1/7.1 |
| MVL5/DODMA/Deoxycholate/ | 15 | 19.5/19.5/52.8/8 |
| DMG-PEG | | |
| MVL5/DODMA/DSPC/Deoxycholate | 16 | 12.5/12.5/41.3/33.7 |

### Example 2: Transfection of Exemplary Delivery Vehicles of this Disclosure

In this study, the transfection efficiency of exemplary delivery vehicles (as prepared using the process described in Example 1 above) were assessed. HEK cells cultured to confluency between 50-80% were used for transfections. 1 μg of Gaussia luciferase expressing plasmid DNA encapsulated in a lipid nanoparticle (as listed above in Table 2) was used per well in a 24 well plate. Transfection efficiency was assessed by taking 30 μl of media after 24 h and performing a flash luciferase assay (Pierce Gaussia Luciferase Assay Kit). Increased value of relative light units (RLU) corresponded to greater transfection efficiency.

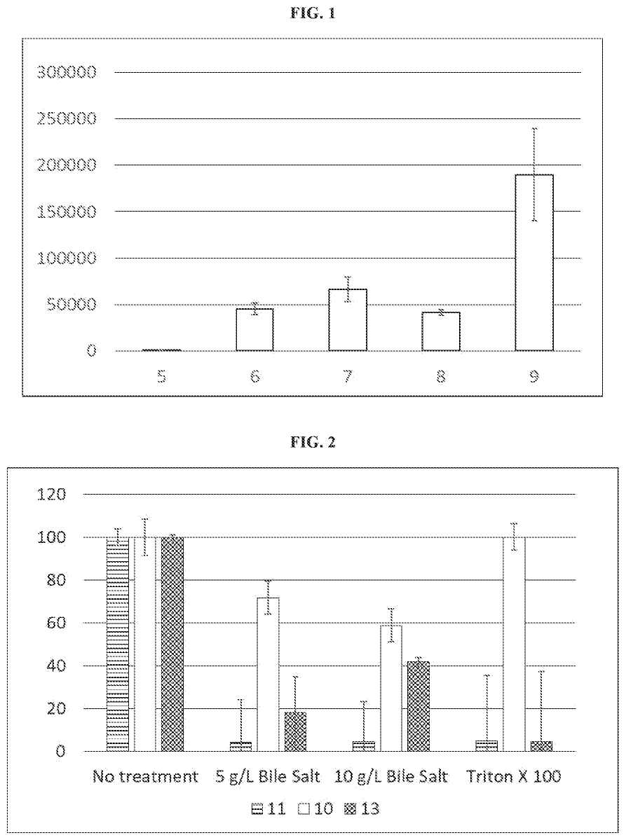

It was observed that the presence of the multivalent cationic lipid MVL5 increased the transfections significantly, possibly by exerting a positive or neutral character on the bile salt stable system. This was likely to due to increased endosomal escape. Due to its multivalency (+3 at physiological pH and +5 at lysosomal pH) and the high molar ratio of the negatively charged bile salts needed for stability, MVL5 and other multivalent lipids can be best suited for this system. Data is shown in FIG. 1.

### Example 3: Stability of Exemplary Delivery Vehicles of this Disclosure

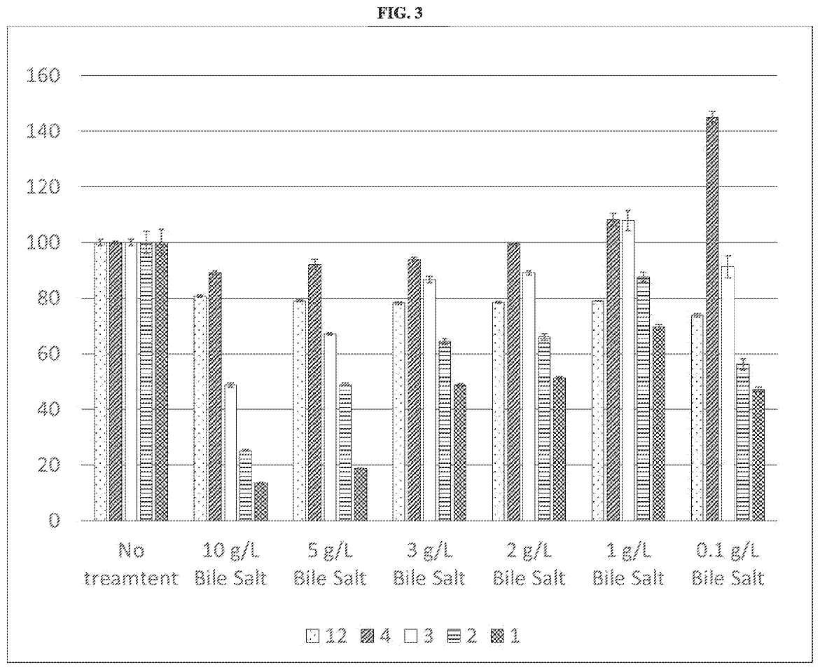

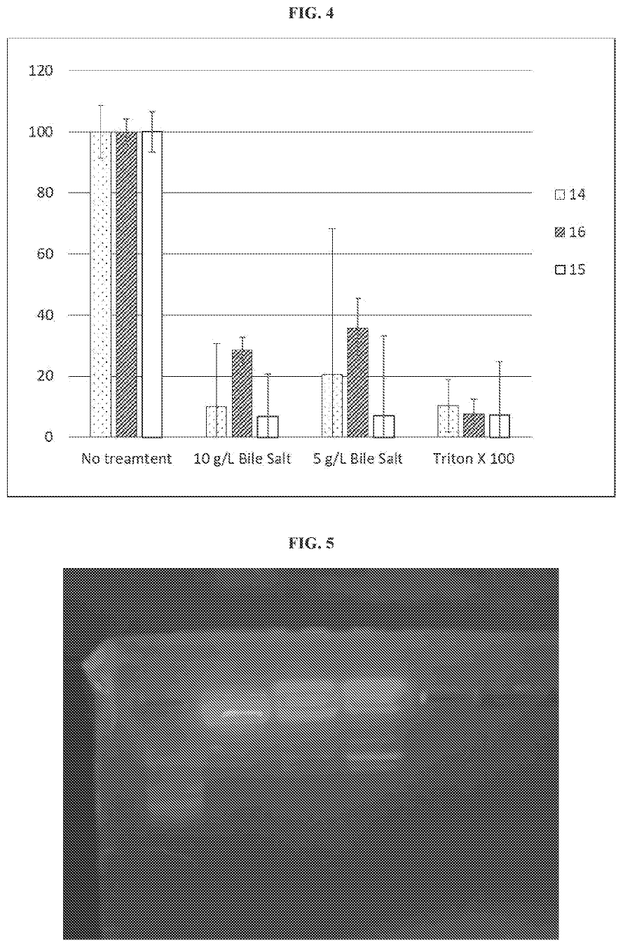

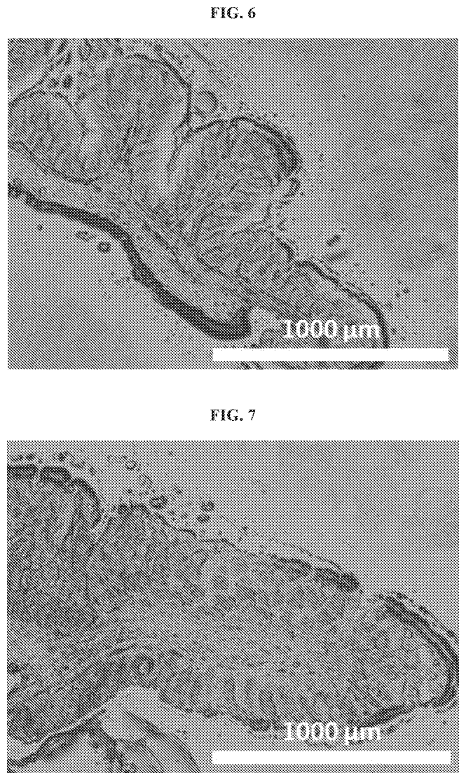

In this study, the stability of exemplary delivery vehicles, in a high bile salt environment, were assessed. To determine delivery vehicle stability, the delivery vehicles used in this assay incorporated 0.5 mol % each of DiI and DiO. DiI and DiO are fluorescent dyes that are FRET pairs. Bile salts were simulated by using an equal mixture of cholic acid and deoxycholate at indicated concentrations (in FIGS. 2-4). It was expected that if the delivery vehicle was susceptible to being disrupted by the bile salts, it would result in decreased FRET intensity. Relative fluorescence units (RFU) was determined by taking exciting at 465 nm and reading emission at 501 nm and 570 nm. The RFU reading at 570 nm was divided by the reading at 501 nm. The readings were normalized to the FRET intensity of the system without any treatment. Data is shown in FIG. 2, FIG. 3, and FIG. 4.

This study demonstrated that DSPC/Deoxycholate (as in Formulation No. 10) but not DOPC/Deoxycholate (as in Formulation No. 11) was stable to bile salts. It should be noted that DOPC/Deoxycholate is analogous to elastic liposomes were found to be highly susceptible to bile salts. In contrast, DSPC/Deoxycholate was found to be highly resistant to bile salt attack. Furthermore, DSPC/Cholesterol (as in Formulation No. 13), was also found to not be resistant to bile salts. This demonstrated that the presence of the saturated lipid tail was not enough to provide stability against bile salts and that the bile salts (e.g., Deoxycholate) must be incorporated within the lipid nanoparticle to provide the stability.

Furthermore, as seen in FIG. 4, it was observed that PEGylation (as in Formulation No. 16), was not necessary for stability but omitting a high phase transition temperature lipid (as in Formulation No. 15), or omission of a bile salt (as in Formulation No. 14), resulted in loss of bile salt stability of the delivery vehicle.

### Example 4: Encapsulation of Nucleic Acid in Exemplary Delivery Vehicles of this Disclosure

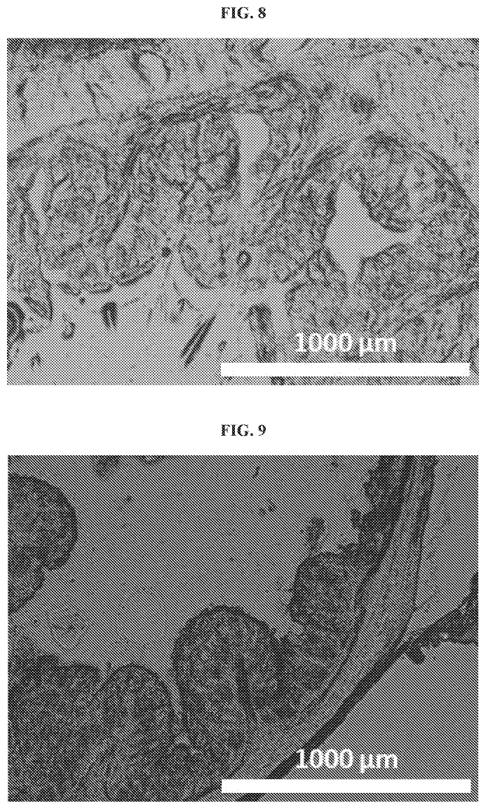

For this study, a delivery vehicle containing 1 μg of DNA encapsulated by the lipid nanoparticle (Formulation No. 5 in Table 2) was loaded to lanes of an agarose gel, either untreated (lane 2 in FIG. 5), (ii) treated with 7% Triton-X 100 (lane 3 in FIG. 5), (iii) treated with 7% Triton-X 100 plus 70° C. for 30 mins (lane 4 in FIG. 5), followed by electrophoresis. SYBR Safe was used to detect the DNA by UV light. No DNA band was found for any of the cationic lipid containing bile salt stable systems (lane 2, untreated), indicating encapsulation and that DNA was not released from the delivery vehicle; however, DNA bands were seen when the system was disrupted using detergent and heat (lanes 3 and 4), indicating that the vehicle was unstable in this environment and DNA was released upon treatment. Data is shown in FIG. 5. This demonstrated the benefit of having a cargo (such as DNA) encapsulated within a delivery vehicle that is stable in a bile salt environment, particularly for efficient protection in a high bile salt environment, such as the gastrointestinal tract.

### Example 5: Preparation of Delivery Vehicle with Cargo

Encapsulation of a nucleic acid cargo was performed as follows: lipids were dissolved in ethanol and heated above their phase transition temperature. The nucleic acid dissolved in an aqueous buffer heated above the phase transition temperature of the lipids. The aqueous buffer pH was set at below the pKa of the bile salt and the cationic lipids. In this way, the lipids were strongly cationic when formulated with the nucleic acids. The lipids and nucleic acids were mixed using microfluidic channels. The pH was raised to neutral, and the sample was concentrated, and ethanol removed using dialysis.

Materials: DODMA (Sigma Aldrich), Deoxycholate (Sigma Aldrich), MVL5 (Avanti Polar Lipids), DSPC (Avanti Polar Lipids), DMG-PEG 2000 (Avanti Polar Lipids), DSG-PEG 2000 (Avanti Polar Lipids), DOPC (Avanti Polar Lipids), DiI (ThermoFisher Scientific), DiO (ThermoFisher Scientific), and glycerol monooleate (GMO) (MP Biomedicals).

### Formulation

375 ug of plasmid DNA encoding for gaussia luciferase under a CMV promoter was dissolved in a final volume of 3 mL 50 mM Sodium Acetate buffer (pH 4.8). Appropriate moles of MVL5, DODMA, Deoxycholate, MVL5, DSPC, GMO, DMG-PEG 2000, DSG-PEG 2000, and/or DOPC were mixed in ethanol according to their mole and cationic lipid:nucleotide ratio. The cationic lipid:nucleotide molar ratio remained constant at 16. When lipids were fluorescently labelled with DiI and DiO, each DiI and DiO was added to the mix at 0.5 mole % of the total lipid moles. Ethanol volume was raised to 1 mL. Samples were mounted into syringes on the Nanoassemblr Benchtop (Precision NanoSystems, CA) and preheated to 65° C. for the DSPC formulations or at room temperature for the DOPC formulations. Samples were mixed using the NanoAssemblr Benchtop microfluidic chip system with a flow rate of 6 mL/min. pH was neutralized and then ethanol was removed using dialysis overnight. Sample was concentrated using Amicon Ultra-4 with a 100 kDa cutoff (Merck Millipore Ltd, Ireland).

The following formulations were made as shown in Table 3.

TABLE 3
Example Formulations Prepared

| | Particle | |
|---|---|---|
| Formulation | # | Molar ratios |
| DODMA/DSPC/Deoxycholate | 1 | 25/41.25/33.75 |
| MVL5/DODMA/DSPC/Deoxycholate | 2 | 6.25/18.75/41.25/33.75 |
| MVL5/DODMA/DSPC/Deoxycholate | 3 | 12.5/12.5/41.25/33.75 |
| MVL5/DODMA/DSPC/Deoxycholate | 4 | 18.75/6.25/41.25/33.75 |
| MVL5/DODMA/DSPC/Deoxycholate/ | 5 | 12.4/12.4/40.8/33.4/1 |
| DMG-PEG | | |
| MVL5/DODMA/DSPC/Deoxycholate/ | 6 | 12.25/12.25/40.4/33.1/2 |
| DMG-PEG | | |
| MVL5/DODMA/DSPC/Deoxycholate/ | 7 | 12.1/12.1/40.0/32.7/3 |
| DMG-PEG | | |
| MVL5/DODMA/DSPC/Deoxycholate/ | 8 | 11.9/11.9/39.2/32.1/5 |
| DMG-PEG | | |
| MVL5/DODMA/DSPC/Deoxycholate/ | 9 | 11.25/11.25/37.1/30.4/10 |
| DMG-PEG | | |
| MVL5/DSPC/Deoxycholate | 10 | 25/41.25/33.75 |
| MVL5/DODMA/DOPC/Deoxycholate/ | 11 | 12.4/12.4/40.8/33.4/1 |
| DMG-PEG | | |
| MVL5/DODMA/GMO/Deoxycholate/ | 12 | 12.4/12.4/40.8/33.4/1 |
| DMG-PEG | | |
| MVL5/DODMA/DSPC/Deoxycholate/ | 13 | 12.4/12.4/40.8/33.4/1 |
| DSG-PEG | | |
| MVL5/DODMA/DSPC/Deoxycholate/ | 14 | 12.25/12.25/40.4/33.1/2 |
| DSG-PEG | | |
| MVL5/DODMA/DSPC/Deoxycholate/ | 15 | 12.1/12.1/40.0/32.7/3 |
| DSG-PEG | | |

In summary, particles with DMG-PEG were stable at even 1% DMG-PEG and did not form aggregates. DSG has a stearic acid lipid tail that is present in the gel phase at 37° C. DMG has a myristolic acid lipid tail that is in the liquid phase at 37° C. DMG-PEG was present in the liquid phase portion(s) of the vehicle and thus stabilized the cationic lipids preventing aggregation whereas DSG-PEG was in the gel phase portion(s) and could not provide the same stabilization effect.

### Example 6: In Vivo Administration of Delivery Vehicles

Mice were dosed intrarectally with approximately 30 micrograms of DNA encapsulated in nanoparticles that were DiI and DiO labelled. 4 hours after dosing, mice were sacrificed, and the intestines were embedded in OCT and frozen in dry ice and stored at −80° C. The tissues were cryosectioned into 30 micrometer slices and imaged using a BioTek Cytation 1. DiI fluorescence was measured in the RFP channel.

### PEGylated Particles Fail to Reach the Intestinal Epithelial Cells

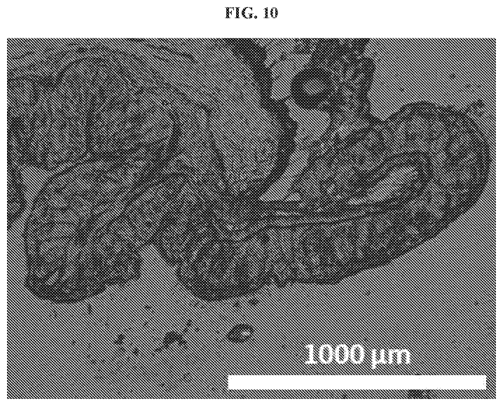

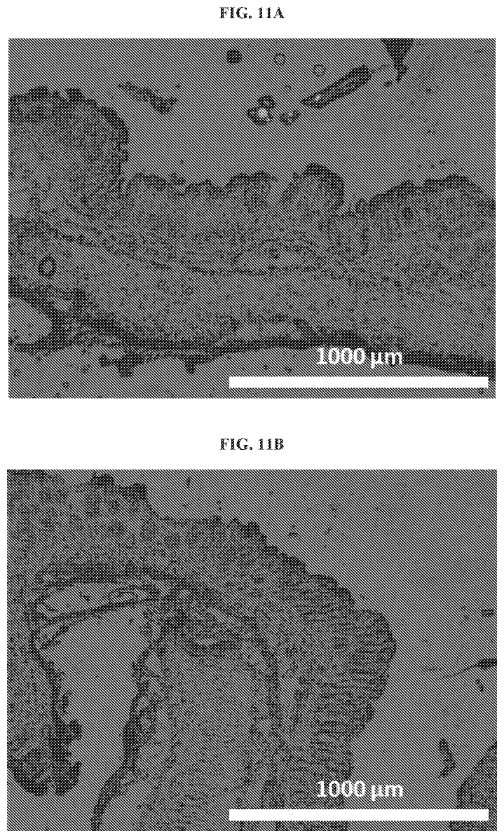

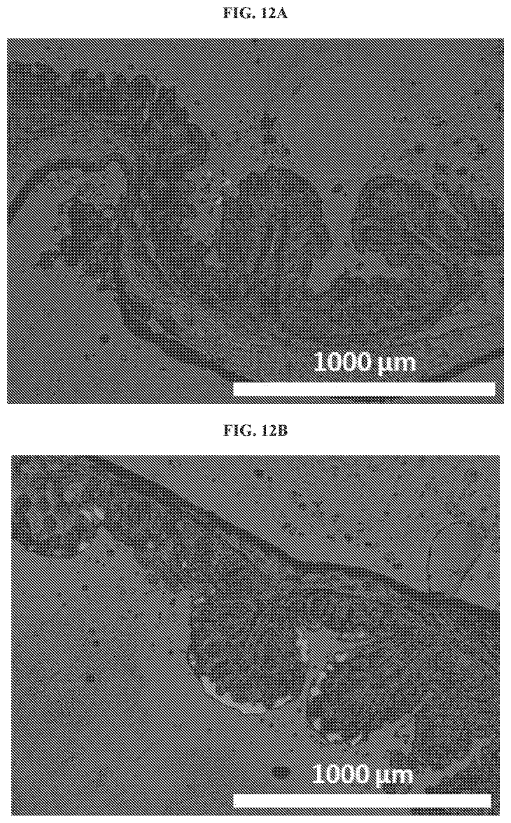

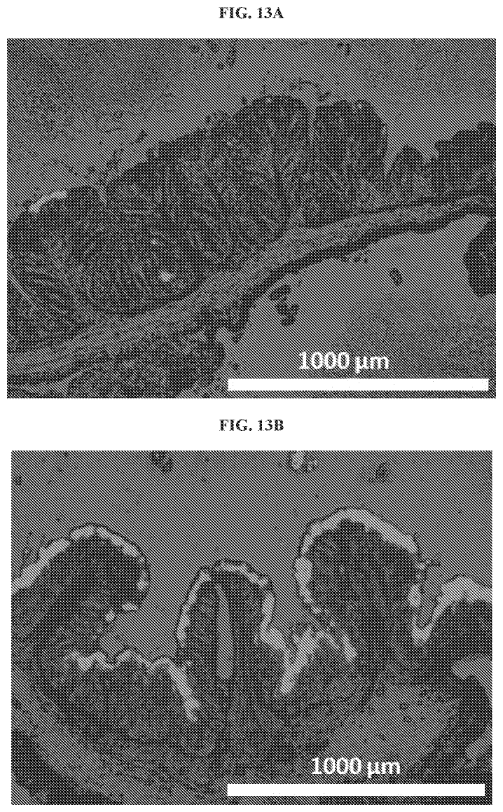

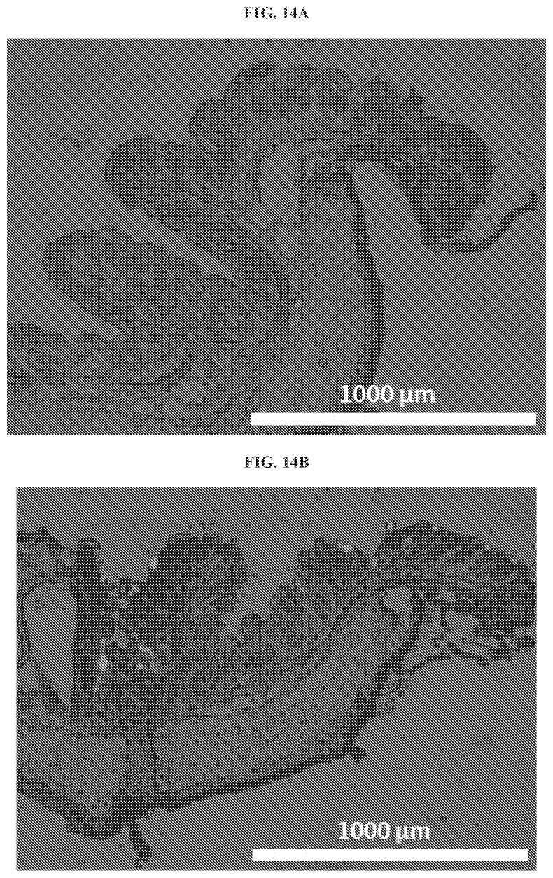

MVL5/DODMA/DSPC/Deoxycholate/DMG-PEG (Particles 5-9) particles were formed with increasing amounts of DMG-PEG and the behavior of the particles was investigated in vivo. An increasing amount of DMG-PEG resulted in decreased distribution at the intestinal tissue. This is in contradiction with the current dogma of increasing PEGylation to increase intestinal epithelial reach. We believe that increased PEGylation reduces the exposure of the positive charge at the surface through its shielding properties. This reduces the dual nature of the particle, as shown in FIG. 6 (Particle 5), FIG. 7 (Particle 6), FIG. 8 (Particle 7), FIG. 9 (Particle 8), and FIG. 10 (Particle 9).

### Example 7: Delivery Vehicle In Vivo Testing

Delivery vehicles were prepared as previously described other than the ratios of MVL5/DODMA were altered in DSPC/Deoxycholate/DMG-PEG/DiI/DiO nanoparticles in order to investigate the effect of increasing positive charge. The following ratios of MVL5/DODMA in the particles were formed (0%/25%), (6.25%/18.75%), (12.5%/12.5%), (18.75%, 6.25%), (25%/0%). As DODMA is mostly neutral at neutral pH and is monovalent, the negative charge of deoxycholate and the multivalent charges of MVL5 dominated the behavior of the particle. Increasing MVL5, thereby increases charge.

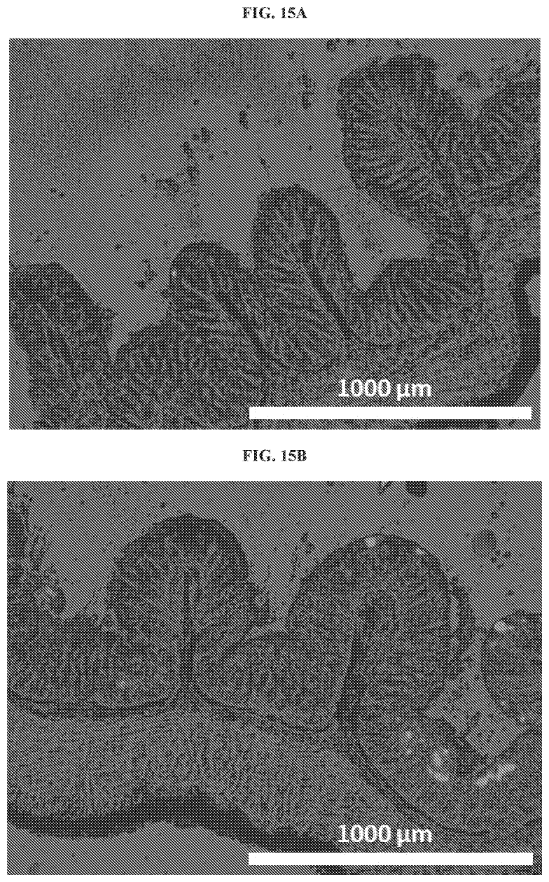

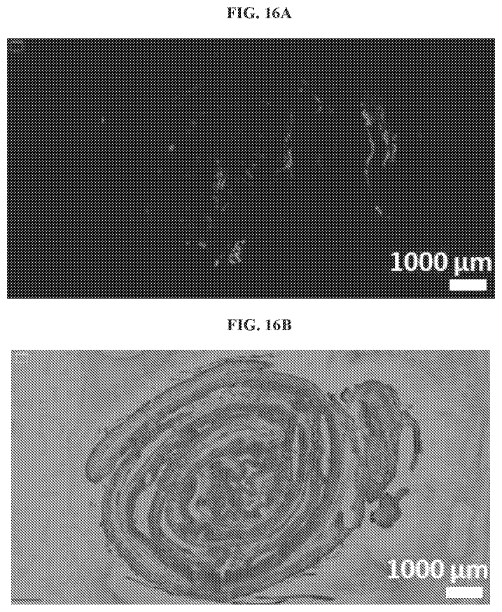

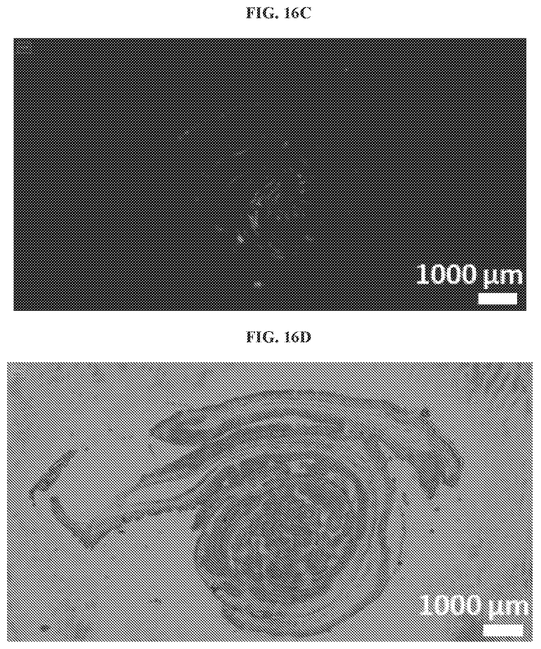

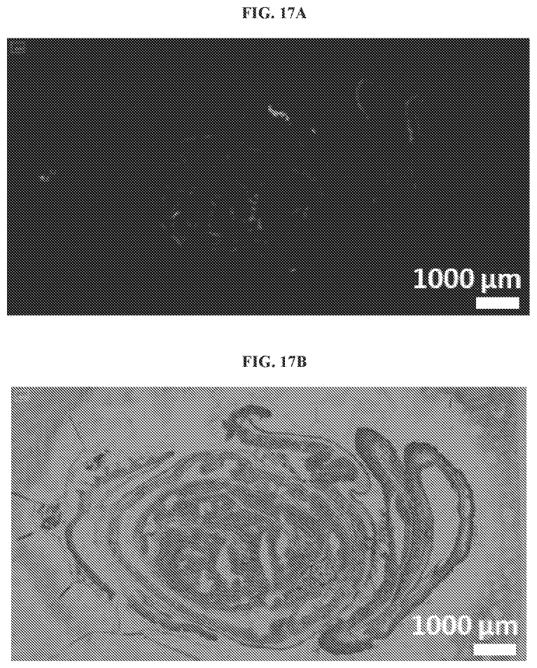

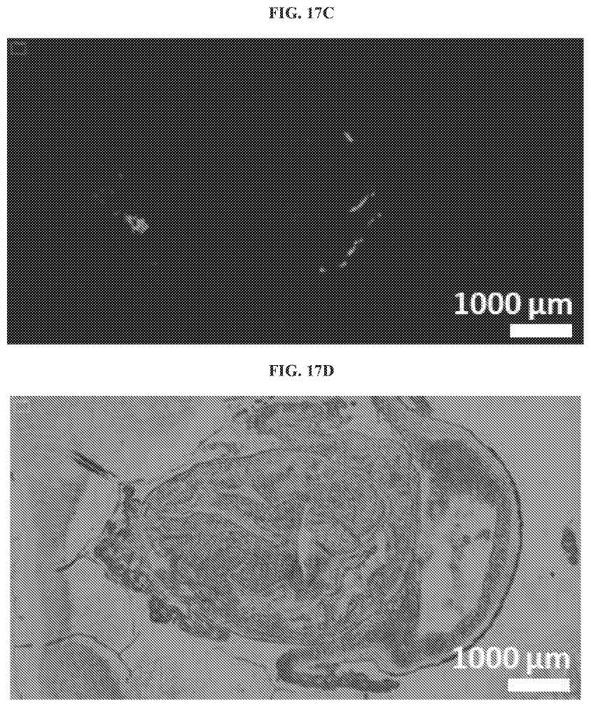

Administration, tissue collection and analysis was conducted as described in example 6. Data, represented in FIG. 11A, FIG. 11B, FIG. 12A, FIG. 12B, FIG. 13A, FIG. 13B, FIG. 14A, FIG. 14B, FIG. 15A, and FIG. 15B, shows the 12.5%/12.5% MVL5/DODMA ratio to be optimal for intestinal epithelia distribution of the particles in vivo. Too much MVL5 provided too strong of a cationic character resulting in adhesion to the negatively charged mucus. Too low of MVL5 resulted in negatively charged particles that may have repelled the mucus or had no interaction. Further, MVL5/DODMA/DSPC/Chol/DMG-PEG particles were made, and they were found to not reach the intestinal epithelial cells. In summary, dual charges are needed to reach the intestinal epithelial cells in a careful balance of charge, as shown in FIG. 11A, FIG. 11B, FIG. 12A, FIG. 12B, FIG. 13A, FIG. 13B, FIG. 14A, FIG. 14B, FIG. 15A, and FIG. 15B.

### Example 8: Zwitterionic Delivery Vehicles Vs. Dual Phase Delivery Vehicles

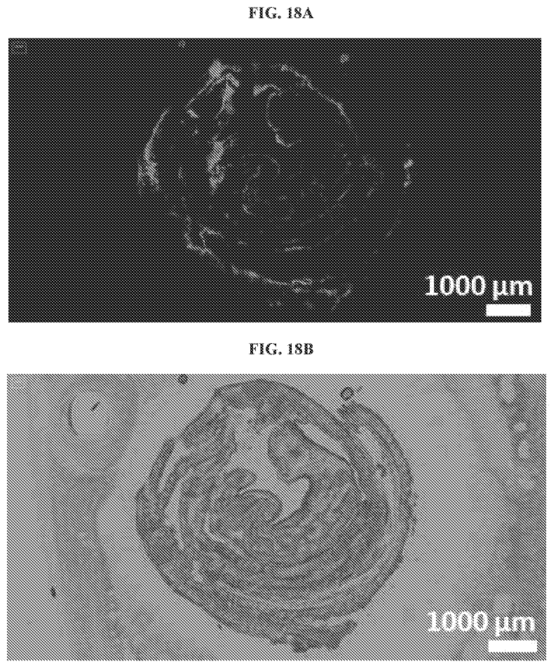

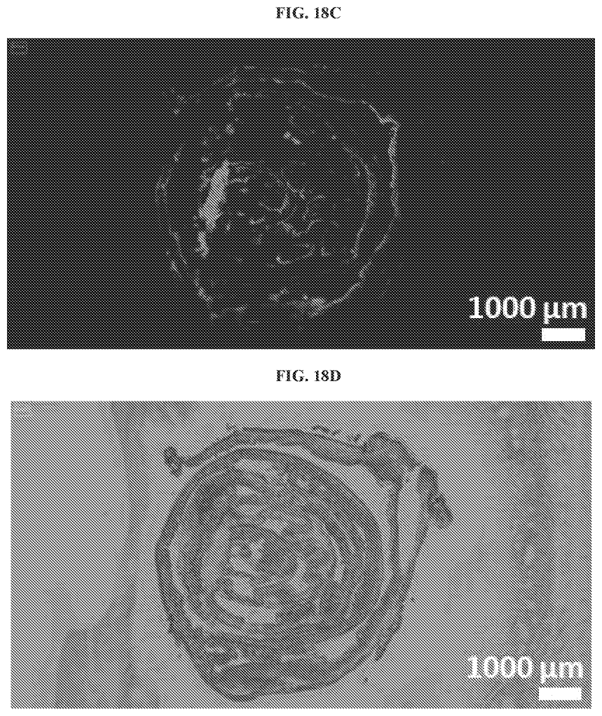

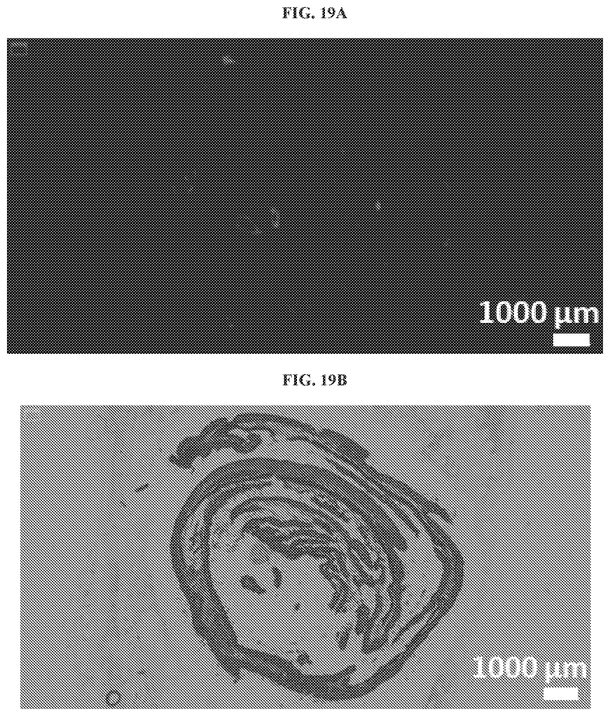

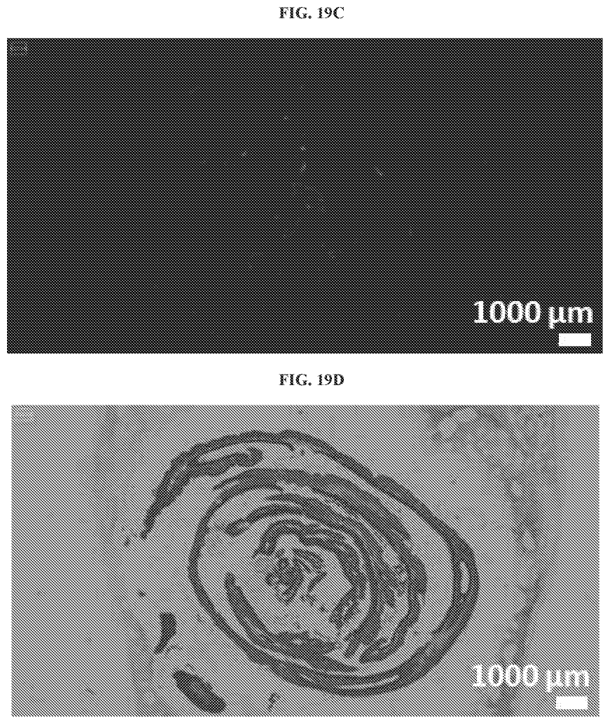

Delivery vehicles were generating as described in Example 6 and were tested in vivo as described in Example 7. Zwitterionicity has been previously shown to increase mucus penetration without the presence of PEG. To investigate if zwitterionicity but not the dual phase nature is sufficient, particles were formulated where the particles were designed to be of a single phase. In order to make single phase particles, low phase transition temperature lipids [i.e., containing DOPC (Table 3, particle 11) or GMO (Table 3, particle 12)] were substituted instead of DSPC (Table 3, particle 5). The charges were held the same across the particles. Particles that were only liquid phase (containing DOPC or GMO instead of DSPC) were found to have significantly reduced or very little intestinal epithelial cell reach, as shown in FIG. 16A, FIG. 16B, FIG. 16C, and FIG. 16D for DOPC particles (particle 11, Table 3); FIG. 17A, FIG. 17B, FIG. 17C, and FIG. 17D for GMO particles (particle 12, Table 3); and FIG. 18A, FIG. 18B, FIG. 18C, and FIG. 18D for DSPC particles (particle 5, Table 3). Results with PBS control particles are shown in FIG. 19A, FIG. 19B, FIG. 19C, and FIG. 19D.

In summary, data shows that the presence of zwitterionicity alone is insufficient to allow for intestinal epithelial cell reach.

### Example 9: Stability of Delivery Vehicles with Bile Salts

The following formulations were made using methods previously described in Example 1: MVL5:MC2 (Biofine International LLC, Vancouver BC Canada): Bile Salt: DSPC:DMG-PEG2000: DiI: DiO in the molar ratio of 0.96:0.96:2.592:3.168:0.0768:0.0384:0.0384 where the bile salt component was either ursodiol, deoxycholate, lithocholate, isolithocholate, alloisolithocholate, dehydrolithocholate or 5beta-cholanic acid. No nucleic acid was incorporated into the lipid nanoparticles. Alternate formulations can also be generated such as those provided in Table 4.

TABLE 4
Suitable alternate bile salt formulations

| Formulation | Molar Ratio |
|---|---|
| MVL5/MC2/DSPC/Deoxycholate/DMPE-PEG | 2.4/2.4/7.9/6.48/0.192 |
| MVL5/CL1H6/DSPC/Deoxycholate/DMG-PEG | 2.4/2.4/7.9/6.48/0.192 |
| MVL5/CL4H6/DSPC/Deoxycholate/DMG-PEG | 2.4/2.4/7.9/6.48/0.192 |
| MVL5/MC2/DSPC/Chenodeoxycholate/DMG-PEG | 2.4/2.4/7.9/6.48/0.192 |
| MVL5/MC2/DMPC/Deoxycholate/DMG-PEG | 2.4/2.4/7.9/6.48/0.192 |
| MVL5/MC2/DMPC/Deoxycholate/DMPE-PEG | 2.4/2.4/7.9/6.48/0.192 |
| MVL5/CL1H6/DMPC/Deoxycholate/DMG-PEG | 2.4/2.4/7.9/6.48/0.192 |
| MVL5/MC2/DSPC/Deoxycholate/Lithocholate/ | 2.4/2.4/7.9/5.2/ |
| DMG-PEG | 1.3/0.192 |
| MVL5/CL1H6/DSPC/Deoxycholate/Lithocholate/ | 2.4/2.4/7.9/5.2/1.3/ |
| DMG-PEG | 0.192 |
| MVL5/MC2/DSPC/Alloisolithocholate/DMG-PEG | 2.4/2.4/7.92/6.48/ |
| | 0.192 |
| MVL5/MC2/DSPC/Dehydrolithocholate/DMG-PEG | 2.4/2.4/7.92/6.48/ |
| | 0.192 |

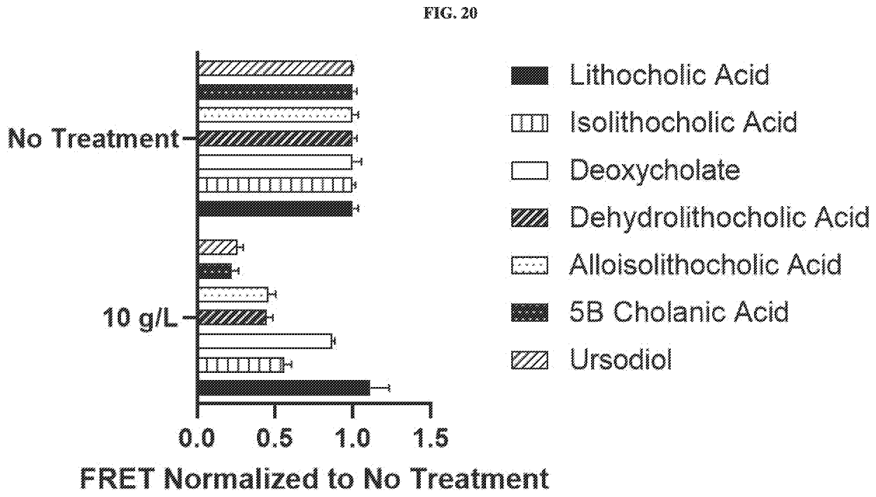

Stability of the lipid nanoparticles in bile salt was measured as discussed previously up to 10 g/L. FRET signals from DiI and DiO were normalized to no treatment. Levels of stability of the vehicles, in salt form, is shown in FIG. 20.

Nanoparticles incorporating various bile salts according to formulations listed in Table 5 were made using methods previously described in Example 1. Mole % values shown are based on percent of total lipid. Bile salts indicated were included at 33.4% and indicated PEG-conjugated lipid at 1%. No nucleic acid was incorporated into lipid nanoparticles N001-N004. Lipid nanoparticles D001 and D002 were prepared with plasmid DNA at a cationic lipid:nucleotide ratio of 16.

TABLE 5
Nanoparticle formulations

| ID# | MVL5 | MC2 | DSPC | Bile Salt | PEG-lipid |
|---|---|---|---|---|---|
| N001 | 12.4% | 12.4% | 40.8% | Taurodeoxycholate | DMG-PEG |
| N002 | 12.4% | 12.4% | 40.8% | Taurochenodeoxycholate | DMG-PEG |
| N003 | 12.4% | 12.4% | 40.8% | Glycocholate | DMPE-PEG |
| N004 | 12.4% | 12.4% | 40.8% | 3-oxy-cholenic acid | DMG-PEG |
| D001 | 12.4% | 12.4% | 40.8% | Deoxycholate | DMG-PEG |
| D002 | 12.4% | 12.4% | 40.8% | Deoxycholate | DMPE-PEG |

Nucleic acid-free lipid nanoparticle stability in different levels of bile salt was measured as described previously. FRET signals from DiI and DiO were normalized to samples assayed in solutions without bile salts. Resulting values showing vehicle stability levels are shown in Table 6. Standard deviation values are shown in parenthesis.

TABLE 6
Nanoparticle stability levels

| | | 10 g/L | 8 g/L | 6 g/L | 5 g/L | 2 g/L | 1 g/L | |
|---|---|---|---|---|---|---|---|---|
| | No | bile | bile | bile | bile | bile | bile | Triton |
| ID# | treatment | salt | salt | salt | salt | salt | salt | X 100 |
| N001 | 1.000 | 0.039 | 0.039 | 0.057 | 1.170 | 1.375 | 1.657 | 0.041 |
| | (0.031) | (0.002) | (0.001) | (0.004) | (0.039) | (0.024) | (0.083) | (0.001) |
| N002 | 1.000 | 0.048 | 0.048 | 0.068 | 1.218 | 1.412 | 1.711 | 0.044 |
| | (0.102) | (0.003) | (0.002) | (0.003) | (0.070) | (0.014) | (0.166) | (0.004) |
| N003 | 1.000 | 0.477 | 0.477 | 0.351 | 1.819 | 2.258 | 2.922 | 0.142 |
| | (0.084) | (0.008) | (0.009) | (0.014) | (0.092) | (0.091) | (0.112) | (0.003) |
| N004 | 1.000 | 0.215 | 0.240 | 0.328 | 3.307 | 3.526 | 4.216 | 0.135 |
| | (0.260) | (0.018) | (0.013) | (0.016) | (0.188) | (0.015) | (0.041) | (0.003) |

Of those tested, N003 and N004 demonstrated the greatest stability levels.

Lipid nanoparticles prepared with plasmid DNA cargo were also assessed for stability in different levels of bile salt as described previously. FRET signals from DiI and DiO were normalized to samples assayed in solutions without bile salts. Resulting values showing vehicle stability levels are shown in Table 7. Standard deviation values are shown in parenthesis.

TABLE 7
DNA nanoparticle stability levels

| | | 10 g/L | 8 g/L | 6 g/L | 5 g/L | 2 g/L | 1 g/L | |
|---|---|---|---|---|---|---|---|---|
| | No | bile | bile | bile | bile | bile | bile | Triton |
| ID# | treatment | salt | salt | salt | salt | salt | salt | X 100 |
| D001 | 1.000 | 0.798 | 0.717 | 1.122 | 1.139 | 1.266 | 1.479 | 0.119 |
| | (0.053) | (0.028) | (0.031) | (0.075) | (0.070) | (0.048) | (0.081) | (0.007) |
| D002 | 1.000 | 0.786 | 0.730 | 1.046 | 1.116 | 1.236 | 1.421 | 0.143 |
| | (0.083) | (0.031) | (0.037) | (0.079) | (0.018) | (0.007) | (0.079) | (0.016) |

Both formulations tested demonstrated similar stability levels.

### Example 10. Nanoparticle Therapy

Nanoparticles according to those described in Example 9 are prepared with therapeutic cargo and administered orally (or by other route that introduces the nanoparticles to the gastrointestinal tract, e.g., intrarectally) for local (gastrointestinal) or systemic delivery of the cargo or cargo expression products. Cargo are selected from nucleic acids, polypeptides, protein biologics (e.g., mAb, enzymes, etc.), short half-life biologics (e.g., hormones), immunogens, and genetic or epigenetic editing system components. Local gastrointestinal delivery includes nanoparticle targeting of intestinal epithelial cells, lamina propria cells, intestinal muscle cells, enteric neurons, and/or other cell types present in intestinal tissue. Systemic delivery includes nanoparticle targeting of gastrointestinal epithelial cells and basal secretion of nanoparticle cargo or cargo expression products into circulation.

### Example 11. Nanoparticle-Mediated Delivery of Cell Signaling Factors

Nanoparticles according to those described in Example 9 are prepared with nucleic acid cargo encoding cell signaling factors (e.g., cytokines). Nanoparticles are introduced to subject gastrointestinal tracts orally or intrarectally to transfect and induce factor expression in gastrointestinal cells. Expressed factors are secreted locally or into the circulation for treatment of systemic disorders. Nanoparticles with nucleic acids encoding interleukin (IL)-2 or IL-2 mutein Fc-fusion (AMG 592, Amgen, Thousand Oaks, CA) are administered to provide low doses of either factor: (1) systemically to treat immune-related disorders, including graft versus host disease (GVHD), systemic lupus erythematosus (SLE), and type I diabetes; or (2) locally to treat gastrointestinal immune-related disorders, including irritable bowel disease (IBD), ulcerative colitis, and Crohn's disease.

### Example 12. Nanoparticle-Mediated Antibody Delivery

Nanoparticles according to those described in Example 9 are prepared with nucleic acid cargo encoding anti-IL18 receptor 1 (IL-18R1) antibody. Nanoparticles are introduced to subject gastrointestinal tracts orally or intrarectally to transfect and induce antibody expression in gastrointestinal cells. Expressed antibodies are secreted locally or into the circulation for treatment of systemic disorders. Antibodies block IL-18 cell signaling activity and resulting inflammation associated with immune-related disorders. Local secretion of the antibody treats or prevents gastrointestinal immune-related disorders, including IBD, ulcerative colitis, and Crohn's disease.

### Example 13. Nanoparticle Treatment of Gastrointestinal Disorders

Nanoparticles according to those described in Example 9 are prepared with nucleic acid cargo encoding IL-10, IL-22, or muteins thereof. Nanoparticles are introduced to subject gastrointestinal tracts orally or intrarectally to transfect and induce factor expression in gastrointestinal cells. Expressed factors are secreted locally to treat gastrointestinal immune-related disorders, including IBD, ulcerative colitis, and Crohn's disease.

### Example 14. Nanoparticle-Mediated Delivery of Granulocyte-Macrophage Colony-Stimulating Factor (GM-CSF)

Nanoparticles according to those described in Example 9 are prepared with nucleic acid cargo encoding GM-CSF. Nanoparticles are introduced to subject gastrointestinal tracts orally or intrarectally to transfect and induce factor expression in gastrointestinal cells. Expressed factors are secreted locally or into the circulation to promote myeloid recovery. Subjects receiving treatment include patients with Hodgkin's lymphoma, non-Hodgkin's lymphoma, acute lymphoblastic leukemia, or acute myelogenous leukemia (AML). Included are subjects that have received or are undergoing other forms of therapy, such as chemotherapy or stem cell transplantation (e.g., autologous, or allogeneic stem cell transplantation from HLA-matched donors). In some subjects, leukapheresis is used to collect hematopoietic progenitor cells mobilized as a result of treatment. In some subjects receiving treatment for neutrophil recovery, nanoparticles are administered at a dose and regimen sufficient to provide GM-CSF at a level of about 250 μg/m²/day and are administered until neutrophil blood levels reach 1000/μL.

### Example 15. Nanoparticle-Mediated Delivery of Granulocyte Colony-Stimulating Factor (G-CSF)

Nanoparticles according to those described in Example 9 are prepared with nucleic acid cargo encoding G-CSF. Nanoparticles are introduced to subject gastrointestinal tracts orally or intrarectally to transfect and induce factor expression in gastrointestinal cells. Expressed factors are secreted locally or into the circulation to promote granulocyte production and neutrophil regulation. Subjects receiving treatment include patients with neutropenia (e.g., chemotherapy-induced febrile neutropenia in non-myeloid malignancies or congenital or acquired severe chronic neutropenia). In subjects receiving treatment for neutrophil recovery, nanoparticles are administered at a dose and regimen sufficient to provide G-CSF at a level of 5 μg/kg/day and are administered until neutrophil blood levels reach 1000/μL. Additional subjects receiving treatment include subjects undergoing bone marrow transplant therapy, subjects undergoing peripheral blood progenitor cell collection and engraftment, and subjects having previously received treatment for AML.

### Example 16. Nanoparticle-Mediated Delivery of Adrenomedullin

Nanoparticles according to those described in Example 9 are prepared with nucleic acid cargo encoding adrenomedullin. Adrenomedullin reduces endothelial cell barrier disfunction associated with inflammation or other conditions. Nanoparticles are introduced to subject gastrointestinal tracts orally or intrarectally to transfect and induce adrenomedullin expression in gastrointestinal cells and local secretion from gastrointestinal cells.

### Example 17. Nanoparticle-Mediated Factor Secretion into the Gastrointestinal Lumen

Nanoparticles according to those described in Example 9 are prepared with nucleic acid cargo encoding antimicrobial agents, In some embodiments intestinal alkaline phosphatase (IAP) or defensins. Nanoparticles are introduced to subject gastrointestinal tracts orally or intrarectally to transfect and induce expression in gastrointestinal cells. Expression products are secreted apically into the gastrointestinal lumen to target infectious agents.

### Example 18. Nanoparticle Use for Transient Gastrointestinal Protein Expression

Nanoparticles according to those described in Example 9 are prepared with nucleic acid cargo encoding proteins implicated in gastrointestinal diseases or systemic diseases with gastrointestinal axis interactions. Nanoparticles are introduced to subject gastrointestinal tracts orally or intrarectally to transfect and induce transient protein expression in gastrointestinal epithelial cells. In some subjects, gastrointestinal epithelial cells are transfected with nucleic acids encoding YO5B gene product for treatment of microvillus inclusion disease (MVID). In some subjects, gastrointestinal epithelial cells are transfected with nucleic acids encoding cystic fibrosis transmembrane regulator protein (CFTR) for treatment of cystic fibrosis.

### Example 19. Nanoparticle-Mediated Delivery of Non-Coding RNA to Gastrointestinal Cells

Nanoparticles according to those described in Example 9 are prepared with non-coding RNA cargo (e.g., siRNA, miRNA, long ncRNA, piRNA, snoRNA, scaRNA, tRNA, rRNA and/or snRNA). Nanoparticles are introduced to subject gastrointestinal tracts orally or intrarectally to alter gene expression in gastrointestinal cells. In some subjects, the nanoparticles are introduced to treat gastrointestinal disorders. In some subjects, the nanoparticles are introduced to treat systemic disorders through gastrointestinal interactions. In some subjects, the non-coding RNA cargo represses SMAD7 gene expression for treatment of IBD.

### Example 20. Nanoparticle-Mediated Delivery of Gene Editing Systems

Nanoparticles according to those described in Example 9 are prepared with genetic or epigenetic editing system components. Nanoparticles are used to contact stem cells in vitro or introduced to subject gastrointestinal tracts orally or intrarectally to edit (e.g., via CRISPR base editing) or modify expression of (e.g., via modified Cas system) cellular genes. In some subjects, the nanoparticles used to correct mutations in epithelial cell genes, including CFTR gene mutations, GPR35 gene mutations, RNF186 A64T germline mutations associated with increased ulcerative colitis risk (see Beaudoin, M. et al. PLoS Genetics. 2013. 9(9): e1003723), mutations associated with very early onset IBD (see Leung, G. and Muise, A. M., Physiology. 2018. 33: 360-9, including the genes listed in Table 1 thereof), and/or somatic mutations in genes affecting IL-17 signaling (e.g., NFKBIZ, ZC3H12A, and PIGR; see Nanki, K. et al. Nature. 2020. 577(7789): 254-9). In some subjects, nanoparticles are used to delete genes encoding IL-18 and/or IL-18R1 in gastrointestinal stem cells to treat or prevent IBD. In some subjects, nanoparticles are used to generate RNF186 (179×) mutations in gastrointestinal stem cells to confer protection against IBD. In some subjects, nanoparticles are used to insert transgenes into gastrointestinal cell DNA (e.g., via CRISPR or RNA-mediated retrotransposons) to provide permanent sources for factor expression, including anti-TNF, anti-P19, or anti-IL-23 to treat or prevent IBD; or GLP-1 or FGF21 to treat or prevent metabolic diseases.

### Example 21. Nanoparticle-Mediated Delivery of Antigens

Nanoparticles according to those described in Example 9 are prepared with nucleic acid cargo encoding antigens as immunogens for generation of local or systemic immune responses. Nanoparticles are introduced to subject gastrointestinal tracts orally or intrarectally to transfect and induce antigen expression in gastrointestinal cells. Expressed antigens are secreted locally or into the circulation to promote an immune response. Some subjects receive nanoparticles with cargo encoding antigens from different infectious agents, including influenza virus, SARS-CoV-2, Ebola, polio, or others (e.g., any of those described in Pasetti, M. F., et al. Immunol Rev. 2011. 239(1): 125-48) to immunize subjects against those agents.

### Example 22. Nanoparticle-Mediated Delivery of Neoantigens

Nanoparticles according to those described in Example 9 are prepared with nucleic acid cargo encoding neoantigens for generation of local or systemic immune responses for oncology applications. Nanoparticles are introduced to subject gastrointestinal tracts orally or intrarectally to transfect and induce neoantigen expression in gastrointestinal cells. Expressed antigens are secreted locally or into the circulation to promote an immune response. Some subjects receive nanoparticles with cargo encoding neoantigens capable of generating immune responses against colorectal cancer and/or non-gastrointestinal cancers.

### Example 23. Nanoparticle-Mediated Delivery of Tolerance-Promoting Antigens

Nanoparticles according to those described in Example 9 are prepared with nucleic acid cargo encoding antigens associated with allergies and/or auto-immune diseases. Nanoparticles are introduced to subject gastrointestinal tracts orally or intrarectally to transfect and induce antigen expression in gastrointestinal cells. Expressed antigens are secreted locally or into the circulation to promote immune system tolerance to the antigens and prevention of associated immune-related indications (e.g., peanut allergies, celiac disease, rheumatoid arthritis, and IBD).

### Example 24. Nanoparticle-Mediated Delivery of Nucleic Acids to Intestinal Immune Cells

Nanoparticles according to those described in Example 9 are prepared with nucleic acid cargo encoding factors for intestinal immune cell expression (e.g., lamina propria mononuclear cells or intraepithelial lymphocytes), either exclusively or non-exclusively. Nanoparticles are introduced to subject gastrointestinal tracts orally or intrarectally to transfect and induce factor expression in the intestinal immune cells. In some subjects, nanoparticles with nucleic acid cargo encoding IL-10 are administered for delivery to gastrointestinal monocytes/macrophages to promote suppressive T regulatory (Treg) cell induction. In some subjects, nanoparticles with nucleic acid cargo encoding IL-22 are administered for delivery to gastrointestinal monocytes/macrophages to promote wound healing. In some subjects, nanoparticles with nucleic acid cargo encoding transcription factors are administered for delivery to gastrointestinal monocytes/macrophages to modulate cellular activities, including nucleic acid cargo encoding peroxisome proliferator-activated receptor gamma (PPARγ) for M2 macrophage polarization.

### Example 25. Nanoparticle-Mediated Delivery of Nucleic Acids to Enteric Neurons

Nanoparticles according to those described in Example 9 are prepared with nucleic acid cargo encoding factors for expression in enteric neurons, either exclusively or non-exclusively. Nanoparticles are introduced to subject gastrointestinal tracts orally or intrarectally to transfect and induce factor expression in enteric neurons. Expressed factors act intracellularly, to influence cellular activities, or are secreted to act locally or systemically.

### Example 26. Nanoparticles for Use with Intestinal Organoids

Nanoparticles according to those described in Example 9 are prepared for cargo delivery to intestinal organoids ex vivo. Some nanoparticle cargos include nucleic acids encoding factors for expression in intestinal organoids. These nanoparticles are introduced to organoid cultures ex vivo to transfect and induce factor expression. Some nanoparticle cargos include genetic or epigenetic editing system components. These nanoparticles are introduced to organoid cultures to edit (e.g., via CRISPR base editing) or modify expression of (e.g., via modified Cas system) genes in organoid cells. In some embodiments, exosomes from nanoparticle treated cells are isolated. The exosomes are used in some instances to deliver therapeutic cargo to other cells or tissues (in vivo or ex vivo).

### Example 27. Nanoparticles for Use in Animal Models

Nanoparticles according to those described in Example 9 are prepared for cargo delivery to research animal subjects (e.g., mice or other species). Some nanoparticle cargos include nucleic acids encoding factors for expression in subject gastrointestinal after oral or intrarectal administration. Some nanoparticle cargos include genetic or epigenetic editing system components. These nanoparticles are introduced to subject gastrointestinal tracts orally or intrarectally to edit (e.g., via CRISPR base editing) or modify expression of (e.g., via modified Cas system) genes in gastrointestinal cells. In some embodiments, the nanoparticle delivery is used to create animal models, e.g., for researching specific diseases or effects associated with nanoparticle treatments. In some embodiments, exosomes from nanoparticle treated research animal subjects are isolated. The exosomes are used, in some instances, to deliver therapeutic cargo to other cells, tissues, and/or subjects, including other animal or human subjects.

### Example 28. Nanoparticle-Mediated Treatment of Hemophilia A

Nanoparticles according to those described in Example 9 are prepared with nucleic acid cargo encoding Factor VIII clotting factor. Nanoparticles are administered to subjects with hemophilia A to provide or replace Factor VIII clotting factor that is absent or defective due to mutation or other mechanism. Nanoparticles are administered orally or intrarectally for transfection and induction of Factor VIII expression in gastrointestinal cells. Factor VIII is secreted locally or into the circulation to restore clotting capabilities. In some subjects, circulating levels of Factor VIII reach from about 10 ng/mL to about 300 ng/mL after nanoparticle administration.

### Example 29. Nanoparticle-Mediated Treatment of Gaucher's Disease

Nanoparticles according to those described in Example 9 are prepared with nucleic acid cargo encoding β-glucocerebrosidase (GBA). Nanoparticles are administered to subjects with Gaucher's disease to provide or replace GBA that is absent or defective due to mutation or other mechanism. Nanoparticles are administered orally or intrarectally to reach subject gastrointestinal tracts for transfection and induction of GBA expression in gastrointestinal cells. GBA is secreted locally or into the circulation to restore GBA enzymatic activity. In some subjects, steady-state GBA levels of about 6 ng/mL are achieved in plasma with nanoparticle treatment.

### Example 30. Nanoparticle-Mediated Treatment of Short Bowel Syndrome

Nanoparticles according to those described in Example 9 are prepared with nucleic acid cargo encoding glucagon-like peptide 2 (GLP-2) or an analog thereof (e.g., teduglutide). Nanoparticles are administered to subjects with short bowel syndrome (SBS) to improve intestinal absorption. Nanoparticles are administered orally or intrarectally to reach subject gastrointestinal tracts for transfection and induction of local GLP-2 expression in gastrointestinal cells. In some subjects, nanoparticle administration provides a maximum circulating GLP-2 concentration of about 36 ng/mL.

### Example 31. Nanoparticle-Mediated Adalimumab Treatment

Nanoparticles according to those described in Example 9 are prepared with nucleic acid cargo encoding adalimumab, an antibody that binds to tumor necrosis factor (TNF)α and prevents TNFα receptor binding. Nanoparticles are administered to subjects with immune-related disorders, including rheumatoid arthritis, IBD, and ankylosing spondylitis. Nanoparticles are administered orally or intrarectally for transfection and induction of adalimumab expression in gastrointestinal cells. Expressed antibodies are secreted locally and/or into the circulation for systemic treatment. In some subjects, maximum circulating antibody concentrations reach about 4-5 μg/mL after nanoparticle administration.

### Example 32. Nanoparticle-Mediated Human Growth Hormone Treatment

Nanoparticles according to those described in Example 9 are prepared with nucleic acid cargo encoding human growth hormone (HGH). Nanoparticles are administered to subjects with HGH deficiency or wasting. Nanoparticles are administered orally or intrarectally for transfection and induction of HGH expression in gastrointestinal cells. Expressed HGH is secreted locally and/or into the circulation for systemic treatment. In some subjects, circulating HGH levels achieved after nanoparticle administration are from about 1 to about 10 ng/mL in normal adults or from about 10 to about 50 ng/mL in children.

### Example 33. Nanoparticle-Mediated GLP-1 Treatment

Nanoparticles according to those described in Example 9 are prepared with nucleic acid cargo encoding glucagon-like peptide 1 (GLP-1), a peptide hormone agonist of the GLP-1 receptor. Nanoparticles are administered to subjects with diabetes, cardiovascular disease, and/or non-alcoholic steatohepatitis (NASH). Nanoparticles are administered orally or intrarectally for transfection and induction of GLP-1 expression in gastrointestinal cells. Expressed GLP-1 is secreted locally and/or into the circulation for systemic treatment.

### Example 34. Nanoparticle-Mediated Treatment of Hypo-Parathyroidism

Nanoparticles according to those described in Example 9 are prepared with nucleic acid cargo encoding parathyroid hormone (PTH). Nanoparticles are administered to subjects with hypo-parathyroidism to raise circulating PTH concentration to normal levels. Nanoparticles are administered orally or intrarectally for transfection and induction of PTH expression in gastrointestinal cells. Expressed PTH is secreted locally and/or into the circulation. In some subjects, a maximum circulating PTH concentration of about 150 μg/mL is achieved after nanoparticle administration.

### Example 35. Nanoparticle-Mediated Anti-PCSK9 Treatment

Nanoparticles according to those described in Example 9 are prepared with nucleic acid cargo encoding antibody inhibitors of proprotein convertase subtilisin kexin 9 (PCSK9). Nanoparticles are administered to subjects to block PCSK9-dependent degradation of low density lipoprotein (LDL) receptors, resulting in lower levels of circulating LDL cholesterol and reduced risk of cardiovascular disease. Nanoparticles are administered orally or intrarectally for transfection and induction of antibody expression in gastrointestinal cells. Expressed antibodies (e.g., evolocumab and alirocumab) are secreted locally and/or into the circulation. In some subjects, a maximum circulating antibody concentration of about 18-19 μg/mL is achieved after nanoparticle administration.

### Example 36. Nanoparticle-Mediated T Cell Redirection

Nanoparticles according to those described in Example 9 are prepared with nucleic acid cargo encoding anti-CD3 bispecific antibodies. Nanoparticles are administered to subjects to direct T cells to tumor cells. Nanoparticles are administered orally or intrarectally for transfection and induction of antibody expression in gastrointestinal cells. Expressed antibodies are secreted locally and/or into the circulation. In some subjects, antibodies are secreted locally to target gastrointestinal tumor cells (e.g., those associated with colon cancer). In some subjects, antibodies are secreted into the circulation to target non-gastrointestinal tumor cells and/or tumor cells that are not gastrointestinal-specific.

### Example 37. Nanoparticle-Mediated Delivery of Fibroblast Growth Factor 21

Nanoparticles according to those described in Example 9 are prepared with nucleic acid cargo encoding fibroblast growth factor (FGF) 21. Nanoparticles are administered to subjects to promote metabolic balance, including subjects with NASH. Nanoparticles are introduced to subject gastrointestinal tracts orally or intrarectally for transfection and factor expression in gastrointestinal cells. Expressed factors are secreted locally and/or into the circulation to promote metabolic balance.

### Example 38. Nanoparticle-Mediated Delivery of Relaxin

Nanoparticles according to those described in Example 9 are prepared with nucleic acid cargo encoding relaxin. Nanoparticles are administered to subjects to promote relaxin-dependent anti-fibrotic activity, including to subjects with cardiovascular disease and/or liver fibrosis. Nanoparticles are introduced to subject gastrointestinal tracts orally or intrarectally for transfection and relaxin expression in gastrointestinal cells. Expressed factors are secreted locally and/or into the circulation to promote systemic activity.

### Example 39. CL1H6 Formulations

LNPs with mRNA cargo were prepared following the methods previously described in Examples 1, 4, and 5. Formulation lipid components are shown in Table 8 with lipid component levels provided in mole percentage units relative to total nanoparticle lipid. Molar ratio of total LNP cationic lipids to total LNP nucleotides is presented as CL:N.

TABLE 8
Additional Nanoparticle Formulations

| ID # | Formulation Components | Mole % | CL:N |
|---|---|---|---|
| D107 | MVL5/CL1H6/DSPC/DMG-PEG | 18.6/18.6/61.3/1.5 | 2.5 |
| D108 | MVL5/CL1H6/Deoxycholate/DSPC/ | 16.5/16.5/11.1/54.5/ | 2.5 |
| | DMG-PEG | 1.3 | |
| D109 | MVL5/CL1H6/Deoxycholate/DSPC/ | 14.9/14.9/20/49.1/1.2 | 2.5 |
| | DMG-PEG | | |
| D110 | MVL5/CL1H6/Deoxycholate/DSPC/ | 13.5/13.5/27.3/44.6/ | 2.5 |
| | DMG-PEG | 1.1 | |
| D111 | MVL5/CL1H6/Deoxycholate/DSPC/ | 12.4/12.4/33.4/40.8/1 | 2.5 |
| | DMG-PEG | | |
| D112 | MVL5/CL1H6/Deoxycholate/DSPC/ | 11.6/11.6/37.6/38.3/ | 2.5 |
| | DMG-PEG | 0.9 | |
| D134 | MVL5/CL1H6/Deoxycholate/DMPC/ | 12.4/12.4/33.4/40.8/1 | 2.5 |
| | DMG-PEG | | |
| D135 | MVL5/CL1H6/Deoxycholate/DOPE/ | 12.4/12.4/33.4/40.8/1 | 2.5 |
| | DMG-PEG | | |
| D140 | MVL5/CL1H6/Deoxycholate/ | 12.4/12.4/74.3/1 | 2.5 |
| | DMG-PEG | | |
| D141 | MVL5/CL1H6/Deoxycholate/DSPC/ | 12.4/12.4/66.8/7.4/1 | 2.5 |
| | DMG-PEG | | |
| D142 | MVL5/CL1H6/Deoxycholate/DSPC/ | 12.4/12.4/55.7/18.6/1 | 2.5 |
| | DMG-PEG | | |
| D143 | MVL5/CL1H6/Deoxycholate/DSPC/ | 12.4/12.4/37.1/37.1/1 | 2.5 |
| | DMG-PEG | | |
| D144 | MVL5/CL1H6/Deoxycholate/DSPC/ | 12.4/12.4/18.6/55.7/1 | 2.5 |
| | DMG-PEG | | |
| D145 | MVL5/CL1H6/DSPC/DMG-PEG | 12.4/12.4/74.3/1 | 2.5 |
| D146 | MVL5/CL1H6/Deoxycholate/DOPE/ | 12.4/12.4/55.7/18.6/1 | 2.5 |
| | DMG-PEG | | |
| D147 | MVL5/CL1H6/Deoxycholate/DOPE/ | 12.4/12.4/37.1/37.1/1 | 2.5 |
| | DMG-PEG | | |
| D148 | MVL5/CL1H6/Deoxycholate/DOPE/ | 12.4/12.4/18.6/55.7/1 | 2.5 |
| | DMG-PEG | | |
| D149 | MVL5/CL1H6/DOPE/DMG-PEG | 12.4/12.4/74.3/1 | 2.5 |
| D180 | MVL5/CL1H6/Deoxycholate/DSPC/ | 12.4/12.4/33.5/40.7/1 | 4 |
| | DMG-PEG | | |
| D181 | MVL5/CL1H6/Deoxycholate/DSPC/ | 12.4/12.4/33.5/40.8/1 | 6 |
| | DMG-PEG | | |
| D101 | MVL5/MC2/DSPC/DMG-PEG | 18.6/18.6/61.3/1.5 | 8 |
| D102 | MVL5/MC2/Deoxycholate/DSPC/ | 16.5/16.5/11.1/54.5/1.3 | 8 |
| | DMG-PEG | | |
| D103 | MVL5/MC2/Deoxycholate/DSPC/ | 14.9/14.9/20.1/49/1.2 | 8 |
| | DMG-PEG | | |
| D104 | MVL5/MC2/Deoxycholate/DSPC/ | 13.5/13.5/27.3/44.6/1.1 | 8 |
| | DMG-PEG | | |
| D105 | MVL5/MC2/Deoxycholate/DSPC/ | 12.4/12.4/33.4/40.8/1 | 8 |
| | DMG-PEG | | |
| D106 | MVL5/MC2/Deoxycholate/DSPC/ | 11.6/11.6/37.6/38.3/0.9 | 8 |
| | DMG-PEG | | |
| D121 | MVL5/CL4H6/Deoxycholate/DSPC/ | 12.4/12.4/33.4/40.8/1 | 2.5 |
| | DMG-PEG | | |
| D136 | MVL5/MC2/Deoxycholate/DMPC/ | 12.4/12.4/33.4/40.8/1 | 2.5 |
| | DMG-PEG | | |
| D137 | MVL5/MC2/Deoxycholate/DOPE/ | 12.4/12.4/33.4/40.8/1 | 2.5 |
| | DMG-PEG | | |
| D150 | MVL5/MC2/Deoxycholate/DMG-PEG | 12.4/12.4/74.3/1 | 2.5 |
| D151 | MVL5/MC2/Deoxycholate/DSPC/ | 12.4/12.4/66.8/7.4/1 | 2.5 |
| | DMG-PEG | | |
| D152 | MVL5/MC2/Deoxycholate/DSPC/ | 12.4/12.4/55.7/18.6/1 | 2.5 |
| | DMG-PEG | | |
| D153 | MVL5/MC2/Deoxycholate/DSPC/ | 12.4/12.4/37.1/37.1/1 | 2.5 |
| | DMG-PEG | | |
| D154 | MVL5/MC2/Deoxycholate/DSPC/ | 12.4/12.4/18.6/55.7/1 | 2.5 |
| | DMG-PEG | | |
| D155 | MVL5/MC2/DSPC/DMG-PEG | 12.4/12.4/74.3/1 | 2.5 |
| D156 | MVL5/MC2/Deoxycholate/DMPC/ | 12.4/12.4/55.7/18.6/1 | 2.5 |
| | DMG-PEG | | |
| D157 | MVL5/MC2/Deoxycholate/DMPC/ | 12.4/12.4/37.1/37.1/1 | 2.5 |
| | DMG-PEG | | |
| D158 | MVL5/MC2/Deoxycholate/DMPC/ | 12.4/12.4/18.6/55.7/1 | 2.5 |
| | DMG-PEG | | |
| D159 | MVL5/MC2/DMPC/DMG-PEG | 12.4/12.4/74.3/1 | 2.5 |
| D160 | MVL5/CL1H6/Deoxycholate/DSPC/ | 44.6/44.6/4.5/5.4/1 | 2.5 |
| | DMG-PEG | | |
| D161 | MVL5/CL1H6/Deoxycholate/DSPC/ | 32.2/32.2/15.6/19.1/1 | 2.5 |
| | DMG-PEG | | |
| D162 | MVL5/CL1H6/Deoxycholate/DSPC/ | 24.8/24.8/22.3/27.2/1 | 2.5 |
| | DMG-PEG | | |
| D163 | MVL5/CL1H6/Deoxycholate/DSPC/ | 17.3/17.3/29/35.4/1 | 2.5 |
| | DMG-PEG | | |
| D164 | MVL5/CL1H6/Deoxycholate/DSPC/ | 5/5/40.1/49/1 | 2.5 |
| | DMG-PEG | | |
| D165 | MVL5/MC2/Deoxycholate/DSPC/ | 44.6/44.6/4.5/5.4/1 | 2.5 |
| | DMG-PEG | | |
| D166 | MVL5/MC2/Deoxycholate/DSPC/ | 32.2/32.2/15.6/19.1/1 | 2.5 |
| | DMG-PEG | | |
| D167 | MVL5/MC2/Deoxycholate/DSPC/ | 24.8/24.8/22.3/27.2/1 | 2.5 |
| | DMG-PEG | | |
| D168 | MVL5/MC2/Deoxycholate/DSPC/ | 17.3/17.3/29/35.4/1 | 2.5 |
| | DMG-PEG | | |
| D169 | MVL5/MC2/Deoxycholate/DSPC/ | 5/5/40.1/49/1 | 2.5 |
| | DMG-PEG | | |
| D170 | MVL5/MC2/Deoxycholate/DSPC/ | 12.4/12.4/74.3/0/1 | 8 |
| | DMG-PEG | | |
| D171 | MVL5/MC2/Deoxycholate/DSPC/ | 12.4/12.4/55.7/18.6/1 | 8 |
| | DMG-PEG | | |
| D172 | MVL5/MC2/Deoxycholate/DSPC/ | 12.4/12.4/37.1/37.1/1 | 8 |
| | DMG-PEG | | |
| D173 | MVL5/MC2/Deoxycholate/DSPC/ | 12.4/12.4/18.6/55.7/1 | 8 |
| | DMG-PEG | | |
| D174 | MVL5/MC2/DSPC/DMG-PEG | 12.4/12.4/74.3/1 | 8 |
| D175 | MVL5/MC2/Deoxycholate/DOPE/ | 12.4/12.4/66.9/7.4/1 | 8 |
| | DMG-PEG | | |
| D176 | MVL5/MC2/Deoxycholate/DOPE/ | 12.4/12.4/55.7/18.6/1 | 8 |
| | DMG-PEG | | |
| D177 | MVL5/MC2/Deoxycholate/DOPE/ | 12.4/12.4/37.1/37.1/1 | 8 |
| | DMG-PEG | | |
| D178 | MVL5/MC2/Deoxycholate/DOPE/ | 12.4/12.4/18.6/55.7/1 | 8 |
| | DMG-PEG | | |
| D179 | MVL5/MC2/DOPE/DMG-PEG | 12.4/12.4/74.3/1 | 8 |
| D188 | MVL5/CL1H6/Lithocholate/DOPE/ | 12.4/12.4/33.4/40.8/1 | 2.5 |
| | DMG-PEG | | |
| D189 | MVL5/CL1H6/Lithocholate/DSPC/ | 12.4/12.4/33.4/40.8/1 | 2.5 |
| | DMG-PEG | | |
| D191 | MVL5/MC2/Deoxycholate/DSPC/ | 9.3/15.5/33.4/40.8/1 | 8 |
| | DMG-PEG | | |
| D192 | MVL5/MC2/Deoxycholate/DSPC/ | 6.2/18.6/33.4/40.8/1 | 8 |
| | DMG-PEG | | |
| D193 | MVL5/MC2/Deoxycholate/DSPC/ | 3.1/21.7/33.4/40.8/1 | 8 |
| | DMG-PEG | | |
| D194 | MC2/Deoxycholate/DSPC/DMG-PEG | 24.8/33.4/40.8/1 | 8 |
| D195 | MVL5/MC2/Deoxycholate/DSPC/ | 24.8/24.8/22.3/27.2/1 | 8 |
| | DMG-PEG | | |
| D196 | MVL5/MC2/Deoxycholate/DSPC/ | 18.6/30.9/22.3/27.2/1 | 8 |
| | DMG-PEG | | |
| D197 | MVL5/MC2/Deoxycholate/DSPC/ | 12.4/37.1/22.3/27.2/1 | 8 |
| | DMG-PEG | | |
| D198 | MVL5/MC2/Deoxycholate/DSPC/ | 6.2/43.3/22.3/27.2/1 | 8 |
| | DMG-PEG | | |
| D199 | MC2/Deoxycholate/DSPC/ | 49.5/22.3/27.2/1 | 8 |
| | DMG-PEG | | |
| D200 | MVL5/MC2/Lithocholate/DSPC/ | 12.4/12.4/33.4/40.8/1 | 8 |
| | DMG-PEG | | |

In addition to the above components, mucus penetrating peptides (MPPs) with the amino acid sequence of TVDNDAPTKRASKLFAV (SEQ ID NO: 17) and in an amount of about 0.2 to about 0.3 mole % of total nanoparticle lipid were incorporated by conjugation to nanoparticle PEGs. Nanoparticles were further fluorescently labelled with Dil and DiO. LNPs were prepared with mRNA via microfluidic mixing using a Precision Nanosystems NANOASSEMBLR®. Lipids were dissolved in ethanol and the mRNA in sodium acetate. The assembled particles were then dialyzed in HEPES buffer to reduce the % ethanol and spin concentrated using a 100 kDa Amicon filter.

Nanoparticle characteristics, including size (diameter in nanometers), polydispersity index (PDI), and stability in various concentrations of bile salts was determined (see Table 9). Size and PDI were determined by dynamic light scattering (DLS) via a Malvern Zetasizer Nano ZS-90. For PDI, a value of 1 indicates maximum heterogeneity. Stability in different concentrations of bile salts (or TritonX 100 buffered control solution) was assessed as described previously in Example 3 by measuring the FRET intensity signals from Dil and DiO. Mean values for size, PDI, and FRET signal (relative light units) are reported in the Table, with standard deviation values shown in parenthesis.

TABLE 9
Nanoparticle characteristics

| | | | Stability (mean RLUs) | | |
|---|---|---|---|---|---|
| | | | 0.625 g/L | 10 g/L bile | Tx100 buffer |
| ID# | Size - nm | PDI | bile salts | salts | (control) |
| D107 | 102.97 | 0.135 | 6.6425 | 2.1445 | -0.1986 |
| | (1.3) | (0.023) | (0.0726) | (0.0117) | (0.0243) |
| D108 | 94.2 | 0.172 | 7.5738 | 2.2503 | -0.1015 |
| | (0.35) | (0.012) | (0.21) | (0.027) | (0.0243) |
| D109 | 61.99 | 0.193 | 6.8322 | 1.9995 | -0.0043 |
| | (0.54) | (0.006) | (0.4783) | (0.0614) | (0.0243) |
| D110 | 50.41 | 0.221 | 7.3698 | 2.7193 | 0.0928 |
| | (0.12) | (0.004) | (0.552) | (0.1053) | (0.0243) |
| D111 | 62.62 | 0.153 | 6.4357 | 2.8416 | 0.19 |
| | (0.36) | (0.02) | (1.151) | (0.128) | (0.0243) |
| D112 | 55.16 | 0.212 | 7.2242 | 3.2078 | 0.2871 |
| | (0.85) | (0.021) | (0.2482) | (0.0427) | (0.0243) |
| D134 | 30.64 | 0.267 | 2.8325 | 0.3905 | 0.5483 |
| | (0.08) | (0.017) | (0.0231) | (0.0127) | (0.0012) |
| D135 | 39.56 | 0.235 | 4.2307 | 0.5497 | 0.7819 |
| | (0.39) | (0.006) | (0.2012) | (0.0024) | (0.0079) |
| D140 | 78.12 | 0.108 | 4.1353 | 0.4301 | 0.8133 |
| | (0.47) | (0.009) | (0.0908) | (0.0039) | (0.0266) |
| D141 | 66.16 | 0.237 | 3.3636 | 0.3373 | 0.4491 |
| | (0.64) | (0.004) | (0.0158) | (0.0098) | (0.0027) |
| D142 | 75.52 | 0.261 | 4.9687 | 1.0739 | 0.4307 |
| | (0.28) | (0.017) | (0.0682) | (0.0332) | (0.0026) |
| D143 | 52.95 | 0.252 | 5.8293 | 0.8129 | 0.4767 |
| | (1.1) | (0.034) | (0.2276) | (0.013) | (0.0006) |
| D144 | 71.25 | 0.247 | 2.9595 | 0.739 | 0.3388 |
| | (0.31) | (0.006) | (0.0277) | (0.0387) | (0.0051) |
| D145 | 125.77 | 0.194 | 3.1074 | 1.2239 | 0.396 |
| | (0.49) | (0.015) | (0.0454) | (0.0796) | (0.0009) |
| D146 | 34.29 | 0.185 | 3.1808 | 0.4256 | 0.6964 |
| | (0.53) | (0.002) | (0.0889) | (0.0136) | (0.0085) |
| D147 | 45.99 | 0.276 | 3.0126 | 0.476 | 0.6847 |
| | (0.82) | (0.024) | (0.1307) | (0.0099) | (0.0051) |
| D148 | 56.52 | 0.324 | 1.8377 | 0.324 | 0.4644 |
| | (0.65) | (0.021) | (0.0064) | (0.0042) | (0.004) |
| D149 | 86.94 | 0.415 | 1.5543 | 0.3647 | 0.5148 |
| | (1.28) | (0.013) | (0.0403) | (0.0224) | (0.0046) |
| D180 | 51.18 | 0.123 | 2.7722 | 0.6751 | 0.2775 |
| | (0.1) | (0.011) | (0.0522) | (0.0407) | (0.0014) |
| D181 | 68.74 | 0.141 | 3.7747 | 1.8638 | 0.3179 |
| | (0.49) | (0.012) | (0.1819) | (0.1147) | (0.0004) |

LNPs tested displayed relative uniformity within each population (PDI around 2 or less for most formulations) and mean diameters of from about 30 to about 100 nm (smaller than typical gastrointestinal mucus pore size of about 200 nm).

In general, all formulations tested were stable in a low bile salt environment (0.625 g/L). LNPs D110-D112 with from about 27 to about 38 mole % deoxycholate and from about 38 to about 45 mole % DSPC demonstrated the greatest stability in high bile salt (10 g/L). LNPs lacking DSPC or with substitution of DSPC for DMPC or DOPE were less stable in high bile salt conditions.

For LNPs D107-D112, transfection efficiency was assessed with mRNA cargo encoding G-CSF. Human embryonic kidney (HEK) and Caco 2 intestinal epithelial cell lines were seeded at 10,000 cells/well in a 96 well plate in G-CSF free media. 24 hours later, LNPs were used to transfect cells in triplicate with incubation at 37° C. for 24 hours. G-CSF protein levels in the media were then determined via standard immunological assay. Mean concentrations of G-CSF protein (pg/mL) detected are presented in Table 10 with standard deviations listed in parenthesis.

TABLE 10
G-CSF concentrations in transfected cell media

| | Cell Lines | |
|---|---|---|
| ID # | HEK | Caco 2 |
| D107 | 0 (0) | 7.3 (13.78) |
| D108 | 8.71 (4.37) | 25.2 (3.25) |
| D109 | 13.2 (3.5) | 12.6 (2.34) |
| D110 | 16.5 (4.34) | 21.5 (3.25) |
| D111 | 7.77 (1.37) | 17.3 (2.79) |
| D112 | 15.4 (2.91) | 22.2 (1.36) |

With the exception of D107 (which did not include deoxycholate), all LNPs demonstrated effective transfection with both cell lines.

Further transfection efficiency assays were carried out generally according to the procedure described in Example 2 and utilizing mRNA cargo encoding luciferase. HEK and Caco 2 cell lines were transfected with LNPs and cultured for 24 hours before being lysed with RIPA buffer and assayed for luciferase activity. Mean luciferase activity (relative light units) associated with transfected cells is shown in Table 11. Standard deviation values are shown in parenthesis.

TABLE 11
Luciferase activity in transfected cells

| | Cell Lines | |
|---|---|---|
| ID # | HEK | Caco 2 |
| D134 | 69855 (17240) | 41264 (5723) |
| D135 | 508277 (52412) | 221189 (58943) |
| D140 | 88133 (3288) | 24000 (18130) |
| D141 | 127784 (25252) | 6834 (2670) |
| D142 | 96170 (78018) | 13884 (5803) |
| D143 | 124843 (75346) | 9578 (4146) |
| D144 | 21973 (6085) | 9361 (3421) |
| D145 | 3541 (5531) | 2946 (1046) |
| D146 | 299489 (137517) | 155686 (4334) |
| D147 | 528111 (451571) | 390733 (29270) |
| D148 | 797413 (56053) | 617169 (7776) |
| D149 | 497071 (124209) | 19725 (708.7) |
| D180 | 3572 (1264) | 3531 (247.4) |
| D181 | 11367 (3610) | 11946 (814.3) |

All LNPs successfully transfected treated cells, with formulations including DOPE yielding the highest transfection efficiency values. LNPs with higher cationic lipid:nucleotide ratios (D180 and D181) yielded lower transfection efficiency compared to counterpart formulations with standard ratios of 2.5.

### Example 40. LNP Storage

### Glycerol Preparation

LNPs were prepared as previously described in either a; 12.4 mole % MVL5, 12.4 mole CL1H6, 33.4 mole % deoxycholic acid, 40.8 mole % DSPC and 1 mole % DMG-PEG (GI-LNP); or 50 mole % MC3, 38.5 mole % cholesterol, 10 mole % DSPC, and 1.5 mole % DMG-PEG (Liver0LNP) formulation, with nano luciferase mRNA. Nano luciferase mRNA expression construct was synthesized from a template DNA plasmid Accession #JQ437372 (Genescript Biotech).

LNPs were formulated in 10 mM HEPES buffer with 0 or 20% glycerol and then each particle was aliquoted. The 0% glycerol LNP was stored at 4° C., and the 20% glycerol particle was stored at −20° C. Over the course of a week the sized, PDI, and in vitro transfection efficiency was determined. The 0% Glycerol, 4° C. showed a 21% increase in size and 95.6% decrease in expression after 1 week. The 20% Glycerol, −20° C. showed a 62% increase in size and 52% decrease in expression after 1 week. In vitro transfection was accomplished by transfecting mRNA in 96 well plate of HEK293 cells and evaluating luminescence 24 hours post-transfection normalized to a lipofectamine mRNA transfected control.

### Phosphate and Tris-Sucrose Preparation

Lipid nanoparticles were prepared as described above and formulated in either phosphate or Tris-Sucrose buffer. Phosphate buffer was composed of 0.0 mg Potassium Dihydrogen Phosphate, 0.07 mg Disodium Hydrogen Phosphate Dihydrate, 0.01 mg Potassium Chloride, 0.36 mg Sodium Chloride, and 6 mg Sucrose. Tris-Sucrose Buffer was composed of 20 mM tris(hydroxymethyl)aminomethane (Tris) from Sigma Aldrich and 10% w/v Sucrose.

Nanoparticle size and PDI was tested before and after storage as previously described. Following storage, LNPs were then used to transfect HEK cell lines as described above.

In order to determine the optimal storage conditions, the storage procedures, both with and without cryo-protectants, listed in Table 12 were tested. The size in nm and PDI results are displayed in Table 13A, with standard deviations in parentheses. Table 13B lists concentrations of secreted mRNA (pg/ml) for both pre and post freezing transfection.

TABLE 12
LNP Storage Conditions

| ID | Cryo-Protectant | Temp (° C.) | Freezing Process |
|---|---|---|---|
| ID | Cryo-Protectant | Temp (° C.) | Freezing Process |
| G1 | 0% Glycerol | 4 | NA |
| G2 | 20% | -20 | NA |
| NA1 | None (HEPES Buffer) | -20 | Slow Freeze |
| NA2 | None (HEPES Buffer) | -20 | Flash Freeze |
| NA3 | None (HEPES Buffer) | -20 | Direct In-Freezer |
| NB1 | None (HEPES Buffer) | Room | NA |
| NB2 | None (HEPES Buffer) | 4 | NA |
| PA1 | Phosphate Buffer | -80 | Slow Freeze |
| PA2 | Phosphate Buffer | -80 | Flash Freeze |
| PA3 | Phosphate Buffer | -80 | Direct In-Freezer |
| PB1 | Phosphate Buffer | Room | NA |
| PB2 | Phosphate Buffer | 4 | NA |
| TA1 | Tris-Sucrose Buffer | -80 | Slow Freeze |
| TA2 | Tris-Sucrose Buffer | -80 | Flash Freeze |
| TA3 | Tris-Sucrose Buffer | -80 | Direct In-Freezer |
| TB1 | Tris-Sucrose Buffer | Room | NA |

TABLE 13A
LNP Storage Results

| | Storage | Pre-Storage | | Post-Storage | |
|---|---|---|---|---|---|
| LNP ID | ID | Size (nm) | PDI | Size (nm) | PDI |
| GI-LNP | G1 | 54.71 | 0.196 | 66.12 | 0.173 |
| GI-LNP | G2 | 61.48 | 0.243 | 99.79 | 0.192 |
| GI-LNP | NA1 | 81.09 | 0.25 (0.03) | 246.45 (2.05) | 0.21 (0.01) |
| GI-LNP | NA2 | (21.06) | | 235.95 (0.21) | 0.18 (0.01) |
| GI-LNP | NA3 | | | 249.60 (7.35) | 0.19 (0.01) |
| GI-LNP | NB1 | | | 82.57 (15.90) | 0.32 (0.06) |
| GI-LNP | NB2 | | | 91.09 (14.30) | 0.28 (0.01) |
| GI-LNP | PA1 | 244.27 | 0.38 (0.05) | 377.85 (3.61) | 0.62 (0.04) |
| GI-LNP | PA2 | (17.41) | | 507.05 (38.11) | 0.75 (0.01) |
| GI-LNP | PA3 | | | 576.6 | 0.82 |
| GI-LNP | PB1 | | | 381.7 | 0.63 |
| GI-LNP | PB2 | | | 438.6 (130.96) | 0.67 (0.13) |
| GI-LNP | TA1 | 107.95 | 0.35 (0.04) | 111 (9.48) | 0.37 (0.03) |
| GI-LNP | TA2 | (12.45) | | 123.05 (8.84) | 0.39 (0.03) |
| GI-LNP | TA3 | | | 118.00 (13.86) | 0.36 (0.4) |
| GI-LNP | TB1 | | | 105.86 (8.68) | 0.37 (0.04) |
| GI-LNP | TB2 | | | 110 | 0.34 |
| Liver- | G1 | NT | NT | NT | NT |
| LNP | | | | | |
| Liver- | G2 | NT | NT | NT | NT |
| LNP | | | | | |
| Liver- | NA1 | 84.18 | 0.27 (0.03) | 222.95 (5.59) | 0.22 (0.01) |
| LNP | | (3.15) | | | |
| Liver- | NA2 | | | 291.35 (2.05) | 0.36 (0.06) |
| LNP | | | | | |
| Liver- | NA3 | | | 241.5 (3.11) | 0.23 (0.02) |
| LNP | | | | | |
| Liver- | NB1 | | | 80.76 (0.77) | 0.25 (0.00) |
| LNP | | | | | |
| Liver- | NB2 | | | 84.52 (0.03) | 0.30 (0.03) |
| LNP | | | | | |
| Liver- | PA1 | 94.38 | 0.29 (0.02) | 118.65 (2.05) | 0.23 (0.00) |
| LNP | | (2.77) | | | |
| Liver- | PA2 | | | 128.2 (2.26) | 0.32 (0.01) |
| LNP | | | | | |
| Liver- | PA3 | | | 112.00 (2.26) | 0.33 (0.02) |
| LNP | | | | | |
| Liver- | PB1 | | | 96.08 (3.75) | 0.31 (0.02) |
| LNP | | | | | |
| Liver- | PB2 | | | 95.06 (3.63) | 0.28 (0.05) |
| LNP | | | | | |
| Liver- | TA1 | 105.36 | 0.31 (0.03) | 147.85 (13.36) | 0.40 (0.01) |
| LNP | | (3.02) | | | |
| Liver- | TA2 | | | 192.1 (13.29) | 0.48 (0.06) |
| LNP | | | | | |
| Liver- | TA3 | | | 149.8 (4.24) | 0.37 (0.02) |
| LNP | | | | | |
| Liver- | TB1 | | | 149.7 (0.85) | 0.35 (0.00) |
| LNP | | | | | |
| Liver- | TB2 | | | 114.5 (2.40) | 0.34 (0.030) |
| LNP | | | | | |

TABLE 13B
Transfection Results Pre and Post Storage

| | | Pre-Storage mRNA | Post-Storage mRNA |
|---|---|---|---|
| LNP ID | Storage ID | transfection (RFU) | transfection (RFU) |
| GI-LNP | G1 | 370334 (193283) | 101903 (64039) |
| GI-LNP | G2 | 181018 (1932) | 59486 (14493) |
| GI-LNP | TA2 | 1171171.67 (112435.95) | 1433495.67 (165577.51) |

These results show that for CL1H6 particle, HEPES buffer (without cryoprotectant) does not protect from freezing and Phosphate Buffer leads to high PDI. Further, 20 mM Tris 10% Sucrose buffer was the most effective, with little changes in size, PDI, and in vitro transfection.

1. A delivery vehicle comprising:
at least one bile salt, at least one bile acid, or a combination thereof;
at least one cationic lipid;
at least one structural lipid; and
optionally at least one conjugated lipid.

2. The delivery vehicle of claim 1, wherein the at least one bile salt comprises sulfobromophthalein disodium salt hydrate, tauro-3β,5α,6β-trihydroxycholanoic acid, taurochenodeoxycholic acid sodium salt, taurocholic acid sodium salt hydrate, taurocholic acid sodium salt, taurodehydrocholic acid sodium salt, taurodeoxycholic acid sodium salt, taurohyodeoxycholate, taurohyodeoxycholic acid sodium salt, taurolithocholic acid 3-sulfate disodium salt, taurolithocholic acid sodium salt, tauro-β-muricholic acid sodium salt, tauroursodeoxycholic acid sodium salt, tauro-α-muricholic acid sodium salt, tauro-γ-muricholic acid sodium salt, tauro-ω-muricholic acid sodium salt, β-Estradiol 17-(β-D-glucuronide) sodium salt, lithocholic acid 3-sulfate (disodium salt), chenodeoxycholic acid 3-sulfate (disodium salt), chenodeoxycholic acid 7-sulfate (disodium salt), cholic acid 3-sulfate (disodium salt), cholic acid 7-sulfate (disodium salt), cholic acid sodium salt, deoxycholic acid 3-sulfate (disodium salt), deoxycholic acid disulfate (trisodium salt), phenoxymethylpenicillinic acid potassium salt, chenodeoxycholic acid disulfate (trisodium salt), chenodeoxycholic acid sodium salt, cholate, methyl cholate, sodium taurocholate hydrate, 1-naphthyl isothiocyanate, deoxycholate, hyodeoxycholate, glycocholate, sodium glycochenodeoxycholate, sodium cholate hydrate, taurocholate, taurodeoxycholate, taurochenodeoxycholate, chenodeoxycholate, lithocholate, isolithocholate, alloisolithocholate, sodium deoxycholate, sodium deoxycholate monohydrate, dehydrolithochlate, sodium glycodeoxycholate, sodium glycocholate hydrate, sodium taurodeoxycholate hydrate, sodium chenodeoxycholate, glycolithocholate sulfate, glycolithocholate, sodium taurohyodeoxycholate hydrate, sodium taurocholate, sodium tauroursodeoxycholate, sodium taurolithocholate, glycodeoxycholate, and any combination thereof.

3. The delivery vehicle of claim 1, wherein the at least one bile salt comprises cholate deoxycholate, chenodeoxycholate, lithocholate, and any combination thereof.

4. The delivery vehicle of claim 1, wherein the at least one bile acid comprises 3β, 5α, 6β-trihydroxycholanoic acid, 12-ketochenodeoxycholic acid, 12-ketodeoxycholic acid, 12-ketolithocholic acid, 3-oxo chenodeoxycholic acid, 3-oxo deoxycholic acid, 3-oxocholic acid, 3α,6β,7α,12α-tetrahydroxy bile acid, 3α,6α,7α,12α-tetrahydroxy bile acid, 4-bromobenzoic acid, 6,7-diketolithocholic acid, 7-ketodeoxycholic acid, 7-ketolithocholic acid, allocholic acid, alloisolithocholic acid, apocholic acid, apocholic acid (delta 14 isomer), arachidyl amido cholanoic acid, chenodeoxycholic acid, chenodeoxycholic acid-d4, cholic acid, dehydrocholic acid, dehydrolithocholic acid, deoxycholic acid, dioxolithocholic acid, glyco-12-oxolithocholanoic acid, glycochenodeoxycholic acid, glycocholic acid, glycocholic acid hydrate, glycodehydrocholic acid, glycodeoxycholic acid, glycohyodeoxycholic acid, glycolithocholic acid, glycoursodeoxycholic acid, glyco-γ-muricholic acid, hyocholic acid, hyodeoxycholic acid, isodeoxycholic acid, isolithocholic acid, lithocholic acid, murideoxycholic acid, nordeoxycholic acid, obeticholic acid, pentadecanoic acid, ursocholic acid, ursodeoxycholic acid, ursodeoxycholic acid-D4, α-muricholic acid, β-muricholic acid, ω-muricholic acid, and any combination thereof.

5. The delivery vehicle of claim 1, wherein the at least one bile acid comprises ursodiol, 5beta-cholanic acid, 3-oxy-cholenic acid, and any combination thereof.

6. The delivery vehicle of any one of claims 1-5, wherein the delivery vehicle comprises about 5 to about 40 mole % of the at least one bile salt or the at least one bile acid.

7. The delivery vehicle of any one of claims 1-6, wherein the delivery vehicle comprises about 20 to about 40 mole % of the at least one bile salt or the at least one bile acid.

8. The delivery vehicle of any one of claims 1-7, wherein the delivery vehicle comprises about 30 to about 40 mole % of the at least one bile salt or the at least one bile acid.

9. The delivery vehicle of any one of claims 1-8, wherein the at least one bile salt comprises deoxycholate.

10. The delivery vehicle of any one of claims 1-8, wherein the at least one bile salt comprises chenodeoxycholate.

11. The delivery vehicle of any one of claims 1-8, wherein the at least one bile salt comprises lithocholate.

12. The delivery vehicle of any one of claims 1-8, wherein the at least one bile alloisolithocholate.

13. The delivery vehicle of any one of claims 1-8, wherein the at least one bile comprises dehydrolithocholate.

14. The delivery vehicle of any one of claims 1-8, wherein the at least one bile acid comprises ursodiol.

15. The delivery vehicle of any one of claims 1-8, wherein the at least one bile salt comprises isolithocholate.

16. The delivery vehicle of any one of claims 1-8, wherein the at least one bile salt comprises dehydrolithochlate.

17. The delivery vehicle of any one of claims 1-8, wherein the at least one bile acid comprises 5-beta-cholanic acid.

18. The delivery vehicle of any one of claims 1-8, wherein the at least one bile salt comprises taurodeoxycholate.

19. The delivery vehicle of any one of claims 1-8, wherein the at least one bile comprises taurochenodeoxycholate.

20. The delivery vehicle of any one of claims 1-8, wherein the at least one bile salt glycocholate.

21. The delivery vehicle of any one of claims 1-8, wherein the at least one bile acid comprises 3-oxy-cholenic acid.

22. The delivery vehicle of any one of claims 1-8, wherein the delivery vehicle comprises deoxycholate and lithocholate.

23. The delivery vehicle of claim 22, wherein the delivery vehicle comprises about 20 to about 30 mole % deoxycholate and from about 5 to about 10 mole % of lithocholate.

24. The delivery vehicle of any one of claim 1-21, wherein the delivery vehicle comprises at least one bile salt and at least one bile acid.

25. The delivery vehicle of claim 1, wherein the at least one cationic lipid comprises N1-[2-((1 S)-1-[(3-aminopropyl)amino]-4-[di(3-amino-propyl)amino]butylcarboxamido)ethyl]-3,4-di[oleyloxy]-benzamide (MVLS), N4-Cholesteryl-Spermine HCl (GL67), 1,2-dioleyloxy-3-dimethylaminopropane (DODMA), N-[1-(2,3-dioleyloxy)propyl]-N,N,N-trimethylammonium chloride (DOTMA), [1,2-bis(oleoyloxy)-3-(trimethylammonio)propane] (DOTAP), dimethyldioctadecylammonium (DDA), 30[N-(N′,N′-dimethylaminoethane)-carbamoyl] cholesterol (DC-Chol), and dioctadecylamidoglycylspermine (DOGS), 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 1,2-Dialkyloxy-N,N-dimethylaminopropane, 4-(2,2-diocta-9,12-dienyl-[1,3]dioxolan-4-ylmethyl)-dimethylamine, O-alkyl ethylphosphocholines, (6Z,9Z,28Z,31Z)-heptatriaconta-6,9,28,31-tetraen-19-yl 3-(dimethylamino)propanoate (MC2), 3β-[N-(N′,N′-dimethylaminoethane)-carbamoyl]cholesterol, N4-Cholesteryl-Spermine, 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, 7-(4-(dimethylamino)butyl)-7-hydroxytridecane-1,13-diyl dioleate (CL1H6), 7-(4-(diisopropylamino)butyl)-7-hydroxytride-cane-1,13-diyl dioleate (CL4H6), 1,2-stearoyl-3-trimethylammonium-propane (DSTAP), 1,2-dipalmitoyl-3-trimethylammonium-propane (DPTAP), 1,2-Distearoyl-3-Dimethylammonium-Propane (DSDAP), or any combinations thereof. In some embodiments, the saturated cationic lipid can comprise at least one of: 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-((1S)-1-[(3-aminopropyl)amino]-4-[di(3-amino-propyl)amino]butylcarboxamido)ethyl]-3,4-di[alkyl]-benzamide, and any combination thereof.

26. The delivery vehicle of claim 1, wherein the at least one cationic lipid comprises MVL5; MC2; CL1H6; CL4H6; DODMA, and any combination thereof.

27. The delivery vehicle of any one of claim 1 or claim 25 or claim 26, wherein the delivery vehicle comprises about 5 to about 90 mole % of the at least one cationic lipid.

28. The delivery vehicle of any one of claim 1 or claims 25-27, wherein the delivery vehicle comprises about 5 to about 60 mole % of the at least one cationic lipid.

29. The delivery vehicle of any one of claim 1 or claims 25-28, wherein the delivery vehicle comprises about 10 to about 60 mole % of the at least one cationic lipid.

30. The delivery vehicle of any one of claim 1 or claims 25-29, wherein the delivery vehicle comprises about 10 to about 50 mole % of the at least one cationic lipid.

31. The delivery vehicle of any one of claim 1 or claims 25-30, wherein the delivery vehicle comprises about 10 to about 30 mole % of the at least one cationic lipid.

32. The delivery vehicle of any one of claim 1 or claims 25-31, wherein the at least one cationic lipid comprises at least one multivalent cationic lipid and at least one ionizable cationic lipid.

33. The delivery vehicle of claim 32, wherein the at least one multivalent cationic lipid comprises MVL5.

34. The delivery vehicle of any one of claim 32 or claim 33, wherein the delivery vehicle comprises about 5 to about 90 mole % of the at least one multivalent cationic lipid.

35. The delivery vehicle of any one of claim 1 or claims 32-34, wherein the delivery vehicle comprises about 5 to about 60 mole % of the at least one multivalent cationic lipid.

36. The delivery vehicle of any one of claim 1 or claims 32-35, wherein the delivery vehicle comprises about 5 to about 30 mole % of the at least one multivalent cationic lipid.

37. The delivery vehicle of any one of claim 1 or claims 32-36, wherein the delivery vehicle comprises about 5 to about 15 mole % of the at least one multivalent cationic lipid.

38. The delivery vehicle of any one of claim 1 or claims 32-37, wherein the at least one multivalent cationic lipid comprises about up to about 100 mole % of the at least one cationic lipid.

39. The delivery vehicle of any one of claims 32-38, wherein the at least one multivalent cationic lipid comprises about 5-75 mole % of the at least one cationic lipid.

40. The delivery vehicle of any one of claims 32-39, wherein the at least one multivalent cationic lipid comprises about 40-60 mole % of the at least one cationic lipid.

41. The delivery vehicle of any one of claims 32-40, wherein the at least one multivalent cationic lipid comprises about 50 mole % of the at least one cationic lipid.

42. The delivery vehicle of any one of claim 32, wherein the at least one ionizable cationic lipid comprises at least one of MC2, CL1H6, CL4H6, DODMA, and any combination thereof.

43. The delivery vehicle of any one of claim 32 or claim 42, wherein the at least one ionizable cationic lipid comprises MC2.

44. The delivery vehicle of any one of claim 32 or claim 42, wherein the at least one ionizable cationic lipid comprises CL1H6.

45. The delivery vehicle of any one of claim 32 or claim 42, wherein the at least one ionizable cationic lipid comprises CL4H6.

46. The delivery vehicle of any one of claim 32 or claim 42, wherein the at least one ionizable cationic lipid comprises DODMA.

47. The delivery vehicle of any one of claim 1 or claims 32-46, wherein the delivery vehicle comprises about 5 to about 90 mole % of the at least one ionizable cationic lipid.

48. The delivery vehicle of any one of claim 1 or claims 32-47, wherein the delivery vehicle comprises about 5 to about 60 mole % of the at least one ionizable cationic lipid.

49. The delivery vehicle of any one of claim 1 or claims 32-48, wherein the delivery vehicle comprises about 5 to about 30 mole % of the at least one ionizable cationic lipid.

50. The delivery vehicle of any one of claim 1 or claims 32-49, wherein the delivery vehicle comprises about 5 to about 15 mole % of the at least one ionizable cationic lipid.

51. The delivery vehicle of any one of claim 1 or claims 32-50, wherein the ionizable cationic lipid comprises up to about 100 mole % of the at least one cationic lipid.

52. The delivery vehicle of any one of claim 1 or claims 32-51, wherein the ionizable cationic lipid comprises about 5-75 mole % of the at least one cationic lipid.

53. The delivery vehicle of any one of claim 1 or claims 32-52, wherein the ionizable cationic lipid comprises about 40-60 mole % of the at least one cationic lipid.

54. The delivery vehicle of any one of claim 1 or claims 32-53, wherein the ionizable cationic lipid comprises about 50 mole % of the at least one cationic lipid.

55. The delivery vehicle of claim 32, wherein the delivery vehicle comprises about the same amount of the at least one multivalent cationic lipid and the at least one ionizable cationic lipid.

56. The delivery vehicle of claim 1, wherein the at least one structural lipid comprises at least one neutral lipid, at least one anionic lipid, at least one phospholipid, and any combination thereof.

57. The delivery vehicle of any one of claim 1 or claim 56, wherein the at least one structural lipid is comprises glycerol monooleate (GMO), dioleoylphosphatidylethanolamine (DOPE), 1,2-dimyristoyl-sn-glycero-3-phosphocholine (DMPC), short-chainbis-n-heptadecanoylphosphatidylcholine (DHPC), dihexadecoylphosphoethanolamine (DHPE), 1,2-dilinoleoyl-sn-glycero-3-phosphocholine (DLPC), dimyristoylphosphoethanolamine (DMPE), dimyristoylphosphatidylglycerol (DMPG), dioleoylphosphatidylcholine (DOPC), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylcholine (DPPC), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoylphosphoethanolamineimidazole (DSPEI), 1,2-diundecanoyl-sn-glycero-phosphocholine (DUPC), eggphosphatidylcholine (EPC), hydrogenatedsoybeanphosphatidylcholine (HSPC), mannosializeddipalmitoylphosphatidylethanolamine (ManDOG), 1,2-Dioleoyl-sn-Glycero-3-Phosphoethanolamine-N-[4-(p-maleimidomethyl)cyclohexane-carboxamide] (MCC-PE), 1,2-diphytanoyl-sn-glycero-3-phosphoethanolamine (ME 16.0 PE), 1-myristoyl-2-hydroxy-sn-glycero-phosphocholine (MHPC), 1-oleoyl-2-cholesterylhemisuccinoyl-sn-glycero-3-phosphocholine (OChemsPC), phosphatidicacid (PA), phosphatidylethanolaminelipid (PE), phosphatidylglycerol (PG), partiallyhydrogenatedsoyphosphatidylchloline (PHSPC), phosphatidylinositollipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), 18-1-transPE,1-stearoyl-2-oleoyl-phosphatidyethanolamine (SOPE), soybeanphosphatidylcholine (SPC), 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, and any combination thereof.

58. The delivery vehicle any one of claim 1 or claim 56 or claim 57, wherein the at least one structural lipid comprises DSPC, DMPC, DOPE, GMO, and any combination thereof.

59. The delivery vehicle of claim 1 or claims 56-58, wherein the delivery vehicle comprises from about 5 to about 75 mole % of the at least one structural lipid.

60. The delivery vehicle of claim 1 or claims 57-59, wherein the delivery vehicle comprises from about 30 to about 50 mole % of the at least one structural lipid.

61. The delivery vehicle of claim 1 or claims 57-60, wherein the delivery vehicle comprises from about 35 to about 45 mole % of the at least one structural lipid.

62. The delivery vehicle of claim 1, wherein the delivery vehicle does not comprise cholesterol.

63. The delivery vehicle of claim 1, wherein the at least one conjugated lipid comprises at least one conjugated lipid and at least one hydrophilic polymer.

64. The delivery vehicle of any one of claim 1 or claim 63, wherein the at least one hydrophilic polymer comprises polyethylene glycol (PEG).

65. The delivery vehicle of any one of claim 1 or claim 63, wherein the at least one conjugated lipid comprises at least one phospholipid, at least one neutral lipid, at least one glyceride, at least one diglyceride, at least one anionic lipid, at least one cationic lipid, and any combination thereof.

66. The delivery vehicle of any one of claim 1 or claim 63 or claim 65, wherein the at least one conjugated lipid comprises 1,2-dimyristoyl-rac-glycerol (DMG), 1,2-dimyristoyl-sn-glycero-3-phosphoethanolamine (DMPE), 1,2-distearoyl-rac-glycerol (DSG), 1,2-dipalmitoyl-rac-glycerol (DPG), 1,2-distearoyl-sn-glycero-3-phosphoethanolamine (DSPE), diacylglycerol (DAG), 1,2-dipalmitoryl-sn-glycero-3-phosphoethanolamine (DPPE), and any combination thereof.

67. The delivery vehicle of any one of claim 1 or claims 64-66, wherein the at least one conjugated lipid comprises at least one of DMG-PEG, DMPE-PEG, DSG-PEG, DPG-PEG, DSPE-PEG, DAG-PEG, DPPE-PEG, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSG-PEGMA, DSG-PEG5000, DPPE-PEG-2K, DPPE-mPEG2000, DPPE-mPEG, DPG-PEGMA, DOPE-PEG2000, DMPE-PEGMA, DMPE-PEG2000, DMPE-mPEG2000, DMG-PEGMA, DMG-PEG2000, C18PEG750, C18PEG5000, C18PEG3000, CI8PEG2000, C16PEG2000, C14PEG2000, C18-PEG5000, C18PEG, C16PEG, C14-PEG-DSPE200, C14-PEG2000, C14PEG2000, C14-PEG 2000, C14-PEG, C14PEG, (PEG)-C-DOMG, PEG-C-DMA and any combination thereof.

68. The delivery vehicle of any one of claim 1 or claims 63-67, wherein the at least one conjugated lipid comprises DMG-PEG.

69. The delivery vehicle of any one of claim 1 or claims 63-67, wherein the at least one conjugated lipid comprises DMPE-PEG.

70. The delivery vehicle of any one of claim 1 or claims 63-69, wherein the delivery vehicle comprises from about 0.5 to about 2.0 mole % of the at least one conjugated lipid.

71. The delivery vehicle of claim 1, wherein the delivery vehicle does not comprise at least one conjugated lipid.

72. The delivery vehicle of any one of claims 1-70, wherein the delivery vehicle comprises:
the at least one bile salt or the at least one bile acid;
the at least one multivalent cationic lipid;
the at least one ionizable cationic lipid;
the at least one structural lipid; and
the at least one conjugated lipid.

73. The delivery vehicle of claim 72, wherein the delivery vehicle comprises:
about 5-40 mole % of the at least one bile salt or the at least one bile acid;
about 5-90 mole % of the at least one multivalent cationic lipid;
about 5-90 mole % of the at least one ionizable cationic lipid;
about 5-75 mole % of the at least one structural lipid component; and
about 0.5-2.0 mole % the at least one conjugated lipid component.

74. The delivery vehicle of any one of claims 72 or 73, wherein the delivery vehicle comprises:
about 5-40 mole % of the at least one bile salt or the at least one bile acid;
about 5-60 mole % of the at least one multivalent cationic lipid;
about 5-60 mole % of the at least one ionizable cationic lipid;
about 5-75 mole % of the at least one structural lipid; and
about 0.5-2.0 mole % of the at least one conjugated lipid.

75. The delivery vehicle of any one of claims 72-74, wherein the delivery vehicle comprises:
about 20-40 mole % of the at least one bile salt or the at least one bile acid;
about 5-30 mole % of the at least one multivalent cationic lipid;
about 5-30 mole % of the at least one ionizable cationic lipid;
about 30-50 mole % of the at least one structural lipid; and
about 0.5-2.0 mole % of the at least one conjugated lipid.

76. The delivery vehicle of any one of claims 72-75, wherein the delivery vehicle comprises:
about 30-40 mole % of the at least one bile salt or the at least one bile acid;
about 5-15 mole % of the at least one multivalent cationic lipid;
about 5-15 mole % of the at least one ionizable cationic lipid;
about 35-45 mole % of the at least one structural lipid; and
about 0.5-2.0 mole % of the at least one conjugated lipid.

77. The delivery vehicle of any one of claims 72-76, wherein the delivery vehicle comprises:
about 33 mole % of the at least one bile salt or the at least one bile acid;
about 12.5 mole % of the at least one multivalent cationic lipid;
about 12.5 mole % of the at least one ionizable cationic lipid;
about 41 mole % of the at least one structural lipid; and
about 1 mole % of the at least one conjugated lipid.

78. The delivery vehicle of any one of claims 1-77 wherein the delivery vehicle comprises any of the compositions disclosed in Table 1B.

79. The delivery vehicle of any one of claims 1-78, wherein the at least one conjugated lipid is conjugated with at least one polypeptide.

80. The delivery vehicle of claim 79, wherein the at least one polypeptide comprises at least one mucus penetrating polypeptide.

81. The delivery vehicle of any one of claim 79 or claim 80, wherein the at least one mucus penetrating polypeptide comprises an amino acid sequence according to SEQ ID NO: 17.

82. The delivery vehicle of any one of 1-81, wherein the delivery vehicle comprises a cargo.

83. The delivery vehicle of claim 82, wherein the cargo comprises a nucleic acid, a protein, an antibody, a peptide, a small molecule, a biologic, a peptidomimetic, a ribozyme, a chemical agent, a viral particle, a growth factor, a cytokine, an immunomodulating agent, a fluorescent dye, and any combination thereof.

84. The delivery vehicle of claim 83, wherein the cargo comprises a nucleic acid.

85. The delivery vehicle of claim 84, wherein the nucleic acid comprises DNA.

86. The delivery vehicle of claim 85, wherein the DNA comprises plasmid DNA.

87. The delivery vehicle of any one of claims 84-86, wherein the molar ratio of total nanoparticle cationic lipids to the total number of nucleotides comprised by the nucleic acid cargo is from about 2 to about 20.

88. The delivery vehicle of claim 87, wherein the molar ratio of total nanoparticle cationic lipids to the total number of nucleotides comprised by the nucleic acid cargo is from about 14 to about 18.

89. The delivery vehicle of claim 84, wherein the nucleic acid comprises RNA.

90. The delivery vehicle of claim 89, wherein the molar ratio of total nanoparticle cationic lipids to the total number of nucleotides comprised by the nucleic acid cargo is from about 2 to about 20.

91. The delivery vehicle of claim 90, wherein the molar ratio of total nanoparticle cationic lipids to the total number of nucleotides comprised by the nucleic acid cargo is from about 2 to about 4.

92. A pharmaceutical composition, wherein the pharmaceutical composition comprises at least one of the delivery vehicles described in claims 1-91 and an optional pharmaceutically acceptable excipient.

93. The pharmaceutical composition of claim 92, wherein the pharmaceutically acceptable excipient comprises an excipient, adjuvant, solution, stabilizer, additive, surfactant, lyophilization element, dilutant, and any combination thereof.

94. The pharmaceutical composition claims 92 or 93, wherein the pharmaceutical composition is formulated for enteric delivery.

95. A method of delivering at least one cargo to a subject, the method comprising introducing at least one of the delivery vehicle of any of claims 1-91 or at least one of the pharmaceutical compositions of any of claims 92-94 to the gastrointestinal tract of the subject.

96. The method of claim 95, wherein the at least one delivery vehicle or the at least one pharmaceutical composition is introduced to the subject gastrointestinal (GI) tract by at least one rout of administration.

97. The method of claim 96, wherein the at least one rout of comprises intravenous administration, intraperitoneal administration, intramuscular administration, transdermal administration, ocular administration, oral administration, intrarectal administration, direct injection to the GI tract, and any combination thereof.

98. The method of any one of claims 95-97, wherein the at least one delivery vehicle or at least one pharmaceutical composition targets at least one gastrointestinal cell.

99. The method of claim 98, wherein the at least one gastrointestinal cell comprises at least one of an intestinal epithelial cell, a lamina propria cell, an intraepithelial lymphocyte, an intestinal muscle cell, an enteric neuron, or any combination thereof.

100. The method of claim 98 or 99, wherein the at least one cargo is delivered to the gastrointestinal cell.

101. The method of claim 100, wherein the at least one cargo is delivered to the intracellular space of the gastrointestinal cell.

102. The method of any one of claims 100 or 101, wherein the at least one cargo, an at least one cargo component, or an at least one expression product of the cargo is secreted from the gastrointestinal cell.

103. The method of claim 102, wherein secretion of the at least one cargo, the at least one cargo component, or the at least one expression product of the cargo comprises apical secretion or basal secretion.

104. The method of claim 103, wherein the at least one cargo, the at least one cargo component, or the at least one expression product of the cargo remains in an area proximal to the cell after secretion.

105. The method of claim 104, wherein the at least one cargo, the at least one cargo component, or the at least one expression product of the cargo is secreted basally from the gastrointestinal cell and enters the circulation.

106. The method of claim 105, wherein the at least one cargo, the at least one cargo component, or the at least one expression product of the cargo is distributed systemically after entering the circulation.

107. The method of any one of claims 95-106, wherein the at least one cargo comprises at least one therapeutic agent.

108. The method of claim 107, wherein the at least one therapeutic agent comprises one or more of a nucleic acid, a polypeptide, a protein, a biologic, an antibody, an enzyme, a hormone, a cytokine, an immunogen, and a genetic or epigenetic editing system component.

109. The method of claim 108, wherein the at least one therapeutic agent comprises at least one nucleic acid.

110. The method of claim 109, wherein the at least one nucleic acid encodes at least one polypeptide.

111. The method of any one of claims 109 or 110, wherein the at least one nucleic acid comprises DNA.

112. The method of claim 111, wherein the at least one nucleic acid comprises plasmid DNA.

113. The method of any one of claims 109 or 110, wherein the at least one nucleic acid comprises RNA.

114. The method of claim 113, wherein the at least one nucleic acid comprises mRNA, circRNA, saRNA, and any combination thereof.

115. The method of any one of claims 108-114, comprising transfecting the at least one gastrointestinal cell with the at least one nucleic acid.

116. The method of claim 115, wherein the at least one gastrointestinal cell expresses at least one polypeptide encoded by the at least one nucleic acid.

117. The method of any one of claim 115 or claim 116, wherein the polypeptide comprises Granulocyte Spleeny-Stimulating Factor (G-CSF), Green Florescent Protein (GFP) and any combination thereof.

118. The method of claim 109, wherein the at least one nucleic acid comprises a at least one non-coding RNA.

119. The method of claim 118, wherein the at least one non-coding RNA comprises one or more of short interfering RNA (siRNA), microRNA (miRNA), long non-coding RNA, piwi-interacting RNA (piRNA), small nucleolar RNA (snoRNA), small Cajal body-specific RNA (scaRNA), transfer RNA (tRNA), ribosomal RNA (rRNA), and small nuclear RNA (snRNA).

120. A method for treating at least one therapeutic indication in a subject in need thereof comprising delivering at least one delivery vehicle described herein or at least one pharmaceutical compositions described herein to the subject via at least one of the methods for delivery of a cargo described herein.

121. The method of claim 120, wherein the at least one therapeutic indication comprises at least one of a neurodegenerative disease, an ocular disease, a reproductive disease, a gastrointestinal disease, a brain disease, a skin disease, a skeletal disease, a muscoskeletal disease, a pulmonary disease, a thoracic disease, cystic fibrosis, tay-sachs, fragile X, Huntington's, neurofibromatosis, sickle cell, thalassemias, Duchenne's muscular dystrophy, familial adenomatous polyposis (FAP), attenuated FAP, microvillus inclusion disease (MVID), chronic inflammatory bowel disease, chronic inflammatory bowel disease, ileal Crohn's, juvenile polyposis, hereditary diffuse gastric cancer syndrome (HDGC), Peutz-Jeghers syndrome, lynch syndrome, gastric adenocarcinoma and proximal polyposis of the stomach (GAPPS), Li-Fraumeni syndrome, familial gastric cancer, Gilbert's syndrome, telangiectasia, mucopolysaccaride, Osler-Weber-Rendu syndrome, pancreatitis, keratoacanthoma, biliary atresia, Morquio's syndrome, Hurler's syndrome, Hunter's syndrome, Crigler-Najjar, Rotor's, Peutz-Jeghers' syndrome, Dubin-Johnson, Osteochondroses, Osteochondrodysplasias, polyposis, gastrointestinal infections, inflammatory bowel disease (IBD), ulcerative colitis, Crohn's disease, hemophilia, short bowel syndrome (SBS), diabetes, non-alcoholic steatohepatitis (NASH), with Hodgkin's lymphoma, non-Hodgkin's lymphoma, acute lymphoblastic leukemia, or acute myelogenous leukemia (AML), neutropenia, or any combination thereof.

122. The method of claim 121, wherein the at least one therapeutic indication comprises at least one immune-related indication.

123. The method of claim 122, wherein the at least one immune-related indication comprises at least one gastrointestinal indication.

124. The method of claim 123, wherein the at least one therapeutic indication comprises at least one cancer-related indication.
